(19) United States
(12) Patent Application Publication
(10) Pub. No.: US 2026/0308625 A1
(43) Pub. Date: Oct. 8, 2026

(54) COMPOSITIONS AND METHODS FOR TREATING GJB2-RELATED HEARING LOSS

(71) Applicants: Decibel Therapeutics, Inc., Boston, MA (US); Regeneron Pharmaceuticals, Inc., Tarrytown, NY (US)

(72) Inventors: Joseph BURNS, Newton, MA (US); Kathryn ELLIS, Arlington, MA (US); Tyler GIBSON, Boston, MA (US); Kevin LEBO, Weymouth, MA (US); Aayushi MANCHANDA, Aubumdale, MA (US); Gabriela PREGERNIG, Cambridge, MA (US); Leah SABIN, Goldens Bridge, NY (US); Meghan DRUMMOND SAMUELSON, Katonah, NY (US)

(21) Appl. No.: 19/484,939

(22) PCT Filed: May 17, 2024
(86) PCT No.: PCT/US2024/029870

§ 371 Date: Nov. 17, 2025

Related U.S. Application Data
(60) Provisional application No. 63/467,371, filed on May 18, 2023.

(51) Int. Cl. C12N 15/86; A61P 27/16; C07K 14/47

(52) U.S. Cl. CPC C12N 15/86; A61P 27/16; C07K 14/47; C12N 2750/14143; C12N 2830/50

(57) ABSTRACT

The disclosure provides polynucleotides and nucleic acid vectors containing a gap junction protein beta 2 (GJB2) regulatory construct operably linked to a polynucleotide encoding a Gjb2 protein (e.g., a wild-type human Gjb2 protein). These polynucleotides and vectors can be used to express Gjb2 in GJB2-expressing cells, including cochlear supporting cells, and can, thus, be used for the treatment of subjects having or at risk of developing GJB2-related hearing loss.

### SEQUENCE LISTING

The instant application contains a Sequence Listing which has been submitted electronically in XML file format and is hereby incorporated by reference in its entirety. Said XML copy, created on May 8, 2024, is named 51471-015WO2_Sequence_Listing_5_8_24.xml and is 128,752 bytes in size.

### BACKGROUND

Hearing loss is a major public health issue that is estimated to affect nearly 15% of school-age children and one out of three people by age sixty-five. The most common type of hearing loss is sensorineural hearing loss, a type of hearing loss caused by defects in the cells of the inner ear, such as cochlear hair cells and cochlear supporting cells, or the neural pathways that project from the inner ear to the brain. Although sensorineural hearing loss is often acquired, it can also be caused by genetic mutations. Mutations in the GJB2 gene, which encodes gap junction protein beta 2 (Gjb2, also known as Connexin 26), are the most common cause of sensorineural hearing loss due to a genetic mutation.

In recent years, efforts to treat hearing loss have increasingly focused on gene therapy as a possible solution; however, GJB2 is endogenously expressed in an assortment of inner ear cell types that have proven difficult to specifically target. This is a concern because gene therapy approaches for hearing loss that induce expression of an exogenous gene in all cells of the inner ear may have off-target effects or result in toxicity. Accordingly, there is a need for new therapeutics for the treatment of GJB2-related hearing loss.

### SUMMARY OF THE INVENTION

The invention provides compositions and methods for promoting the expression of a gene encoding a wild-type gap junction protein beta 2 (Gjb2) protein, such as a CpG-depleted GJB2 gene, a codon-optimized GJB2 gene, or a CpG depleted and codon-optimized GJB2 gene, in GJB2-expressing cells (e.g., GJB2-expressing inner ear cells). The compositions described herein include nucleic acid vectors containing regulatory elements that can induce expression of a transgene encoding a wild-type Gjb2 protein in GJB2-expressing cells with minimal off-target expression in non-GJB2 expressing cells. Accordingly, the compositions described herein can be administered to a subject, such as a human subject, to induce expression of a wild-type Gjb2 protein in a GJB2-expressing cell (e.g., in a GJB2-expressing inner ear cell, for example, in a subject having a mutation in GJB2) and/or to treat or prevent hearing loss (e.g., sensorineural hearing loss, such as GJB2-related hearing loss).

In a first aspect, the invention provides a nucleic acid vector including, in 5′-to-3′ order: (a) a GJB2 regulatory construct having at least 90% sequence identity (e.g., 90%, 91%, 92%, 93%, 94%, 95%, 96%, 97%, 98%, 99%, or more sequence identity) to the sequence of SEQ ID NO: 1 or SEQ ID NO: 2 operably linked to: (b) a human GJB2 coding sequence selected from the group including SEQ ID NO: 3, SEQ ID NO: 4, SEQ ID NO: 28, a sequence having at least 90% sequence identity (e.g., 90%, 91%, 92%, 93%, 94%, 95%, 96%, 97%, 98%, 99%, or more sequence identity) to SEQ ID NO: 3 and containing at least 50% fewer CG dinucleotides (e.g., 50%, 60%, 70%, 75%, 80%, 85%, 90%, 95%, or fewer CG dinucleotides) than wild-type GJB2, and a sequence having at least 90% sequence identity (e.g., 90%, 91%, 92%, 93%, 94%, 95%, 96%, 97%, 98%, 99%, or more sequence identity) to SEQ ID NO: 4 or SEQ ID NO: 28 operably linked to: (c) a polyadenylation (polyA) signal sequence.

In another aspect, the invention provides a polynucleotide including a human GJB2 coding sequence selected from the group including SEQ ID NO: 3, SEQ ID NO: 4, SEQ ID NO: 28, a sequence having at least 90% sequence identity (e.g., 90%, 91%, 92%, 93%, 94%, 95%, 96%, 97%, 98%, 99%, or more sequence identity) to SEQ ID NO: 3 and containing at least 50% fewer CG dinucleotides (e.g., 50%, 60%, 70%, 75%, 80%, 85%, 90%, 95%, or fewer CG dinucleotides) than wild-type GJB2, and a sequence having at least 90% sequence identity (e.g., 90%, 91%, 92%, 93%, 94%, 95%, 96%, 97%, 98%, 99%, or more sequence identity) to SEQ ID NO: 4 or SEQ ID NO: 28. In some embodiments, a GJB2 promoter is operably linked to the GJB2 coding sequence. In some embodiments, the GJB2 promoter is positioned 5′ of the GJB2 coding sequence. In some embodiments, the GJB2 promoter is contained in a GJB2 regulatory construct. In some embodiments, the GJB2 coding sequence is operably linked to a polyA signal sequence. In some embodiments, the polyA signal sequence is positioned 3′ of the GJB2 coding sequence.

In another aspect, the invention provides a nucleic acid vector containing the polynucleotide of the foregoing aspect.

In some embodiments of any of the foregoing aspects, the GJB2 regulatory construct has at least 90% sequence identity (e.g., 90%, 91%, 92%, 93%, 94%, 95%, 96%, 97%, 98%, 99%, or more sequence identity) to the sequence of SEQ ID NO: 1. In some embodiments of any of the foregoing aspects, the GJB2 regulatory construct has the sequence of SEQ ID NO: 1.

In some embodiments of any of the foregoing aspects, the GJB2 regulatory construct has at least 90% sequence identity (e.g., 90%, 91%, 92%, 93%, 94%, 95%, 96%, 97%, 98%, 99%, or more sequence identity) to the sequence of SEQ ID NO: 2. In some embodiments of any of the foregoing aspects, the GJB2 regulatory construct has the sequence of SEQ ID NO: 2.

In some embodiments of any of the foregoing aspects, the human GJB2 coding sequence has at least 90% sequence identity (e.g., 90%, 91%, 92%, 93%, 94%, 95%, 96%, 97%, 98%, 99%, or more sequence identity) to the sequence of SEQ ID NO: 3 and contains at least 50% fewer CG dinucleotides (e.g., 50%, 60%, 70%, 75%, 80%, 85%, 90%, 95%, or fewer CG dinucleotides) than wild-type GJB2. In some embodiments of any of the foregoing aspects, the human GJB2 coding sequence has at least 90% sequence identity (e.g., 90%, 91%, 92%, 93%, 94%, 95%, 96%, 97%, 98%, 99%, or more sequence identity) to the sequence of SEQ ID NO: 3 and contains no CG dinucleotides. In some embodiments of any of the foregoing aspects, the human GJB2 coding sequence has the sequence of SEQ ID NO: 3.

In some embodiments of any of the foregoing aspects, the human GJB2 coding sequence has at least 90% sequence identity (e.g., 90%, 91%, 92%, 93%, 94%, 95%, 96%, 97%, 98%, 99%, or more sequence identity) to the sequence of SEQ ID NO: 4. In some embodiments of any of the foregoing aspects, the human GJB2 coding sequence has the sequence of SEQ ID NO: 4.

In some embodiments of any of the foregoing aspects, the human GJB2 coding sequence has at least 90% sequence identity (e.g., 90%, 91%, 92%, 93%, 94%, 95%, 96%, 97%, 98%, 99%, or more sequence identity) to the sequence of SEQ ID NO: 28. In some embodiments of any of the foregoing aspects, the human GJB2 coding sequence has the sequence of SEQ ID NO: 28.

In some embodiments of any of the foregoing aspects, a stop codon is positioned 3′ of the GJB2 coding sequence (e.g., the stop codon is directly linked to the 3′ end of the GJB2 coding sequence).

In some embodiments of any of the foregoing aspects, the GJB2 regulatory construct has the sequence of SEQ ID NO: 1 and the human GJB2 coding sequence has the sequence of SEQ ID NO: 3. In some embodiments of any of the foregoing aspects, the GJB2 regulatory construct has the sequence of SEQ ID NO: 1 and the human GJB2 coding sequence has the sequence of SEQ ID NO: 4. In some embodiments of any of the foregoing aspects, the GJB2 regulatory construct has the sequence of SEQ ID NO: 1 and the human GJB2 coding sequence has the sequence of SEQ ID NO: 28. In some embodiments of any of the foregoing aspects, the GJB2 regulatory construct has the sequence of SEQ ID NO: 2 and the human GJB2 coding sequence has the sequence of SEQ ID NO: 3. In some embodiments of any of the foregoing aspects, the GJB2 regulatory construct has the sequence of SEQ ID NO: 2 and the human GJB2 coding sequence has the sequence of SEQ ID NO: 4. In some embodiments of any of the foregoing aspects, the GJB2 regulatory construct has the sequence of SEQ ID NO: 2 and the human GJB2 coding sequence has the sequence of SEQ ID NO: 28.

In some embodiments of any of the foregoing aspects, the polyA signal sequence has at least 90% sequence identity (e.g., 90%, 91%, 92%, 93%, 94%, 95%, 96%, 97%, 98%, 99%, or more sequence identity) to the sequence of SEQ ID NO: 6 or SEQ ID NO: 7. In some embodiments, the polyA signal sequence has at least 90% sequence identity (e.g., 90%, 91%, 92%, 93%, 94%, 95%, 96%, 97%, 98%, 99%, or more sequence identity) to the sequence of SEQ ID NO: 6. In some embodiments, the polyA signal sequence has the sequence of SEQ ID NO: 6. In some embodiments, the polyA signal sequence has at least 90% sequence identity (e.g., 90%, 91%, 92%, 93%, 94%, 95%, 96%, 97%, 98%, 99%, or more sequence identity) to the sequence of SEQ ID NO: 7. In some embodiments, the polyA signal sequence has the sequence of SEQ ID NO: 7.

In some embodiments of any of the foregoing aspects, the nucleic acid vector or polynucleotide further includes a first polynucleotide having at least 90% sequence identity (e.g., 90%, 91%, 92%, 93%, 94%, 95%, 96%, 97%, 98%, 99%, or more sequence identity) to SEQ ID NO: 8 that contains a first member of an inverted terminal repeat (ITR) pair and is positioned 5′ of the GJB2 regulatory construct sequence or the GJB2 promoter sequence and a second polynucleotide having at least 90% sequence identity (e.g., 90%, 91%, 92%, 93%, 94%, 95%, 96%, 97%, 98%, 99% or more sequence identity) to SEQ ID NO: 9 that contains a second member of an ITR pair and is positioned 3′ of the polyA signal sequence. In some embodiments, the first polynucleotide has the sequence of SEQ ID NO: 8 and the second polynucleotide has the sequence of SEQ ID NO: 9.

In some embodiments of any of the foregoing aspects, the nucleic acid vector or polynucleotide further includes a first polynucleotide having at least 90% sequence identity (e.g., 90%, 91%, 92%, 93%, 94%, 95%, 96%, 97%, 98%, 99%, or more sequence identity) to SEQ ID NO: 10 that contains a first member of an ITR pair and is positioned 5′ of the GJB2 regulatory construct sequence or the GJB2 promoter sequence and a second polynucleotide having at least 90% sequence identity (e.g., 90%, 91%, 92%, 93%, 94%, 95%, 96%, 97%, 98%, 99%, or more sequence identity) to SEQ ID NO: 11 that contains a second member of an ITR pair and is positioned 3′ of the polyA signal sequence. In some embodiments, the first polynucleotide has the sequence of SEQ ID NO: 8 and the second polynucleotide has the sequence of SEQ ID NO: 9.

In some embodiments of any of the foregoing aspects, the nucleic acid vector or polynucleotide contains a polynucleotide sequence including nucleotides 157 to 2383 of SEQ ID NO: 12. In some embodiments, the nucleic acid vector or polynucleotide contains a polynucleotide sequence including nucleotides 1 to 2531 of SEQ ID NO: 12.

In some embodiments of any of the foregoing aspects, the nucleic acid vector or polynucleotide contains polynucleotide sequence including nucleotides 157 to 2383 of SEQ ID NO: 13. In some embodiments, the nucleic acid vector or polynucleotide contains a polynucleotide sequence including nucleotides 1 to 2531 of SEQ ID NO: 13.

In some embodiments of any of the foregoing aspects, the nucleic acid vector or polynucleotide contains polynucleotide sequence including nucleotides 157 to 3459 of SEQ ID NO: 14. In some embodiments, the nucleic acid vector or polynucleotide contains a polynucleotide sequence including nucleotides 1 to 3607 of SEQ ID NO: 14.

In some embodiments of any of the foregoing aspects, the nucleic acid vector or polynucleotide contains polynucleotide sequence including nucleotides 157 to 3459 of SEQ ID NO: 15. In some embodiments, the nucleic acid vector or polynucleotide contains a polynucleotide sequence including nucleotides 1 to 3607 of SEQ ID NO: 15.

In some embodiments of any of the foregoing aspects, the nucleic acid vector or polynucleotide contains polynucleotide sequence including nucleotides 157 to 2462 of SEQ ID NO: 16. In some embodiments, the nucleic acid vector or polynucleotide contains a polynucleotide sequence including nucleotides 1 to 2610 of SEQ ID NO: 16.

In some embodiments of any of the foregoing aspects, the nucleic acid vector or polynucleotide contains polynucleotide sequence including nucleotides 157 to 2462 of SEQ ID NO: 17. In some embodiments, the nucleic acid vector or polynucleotide contains a polynucleotide sequence including nucleotides 1 to 2610 of SEQ ID NO: 17.

In some embodiments of any of the foregoing aspects, the nucleic acid vector or polynucleotide contains polynucleotide sequence including nucleotides 157 to 3538 of SEQ ID NO: 18. In some embodiments, the nucleic acid vector or polynucleotide contains a polynucleotide sequence including nucleotides 1 to 3686 of SEQ ID NO: 18.

In some embodiments of any of the foregoing aspects, the nucleic acid vector or polynucleotide contains polynucleotide sequence including nucleotides 157 to 3538 of SEQ ID NO: 19. In some embodiments, the nucleic acid vector or polynucleotide contains a polynucleotide sequence including nucleotides 1 to 3686 of SEQ ID NO: 19.

In some embodiments of any of the foregoing aspects, the nucleic acid vector or polynucleotide contains polynucleotide sequence including nucleotides 212 to 2438 of SEQ ID NO: 20. In some embodiments, the nucleic acid vector or polynucleotide contains a polynucleotide sequence including nucleotides 1 to 2650 of SEQ ID NO: 20.

In some embodiments of any of the foregoing aspects, the nucleic acid vector or polynucleotide contains polynucleotide sequence including nucleotides 212 to 2438 of SEQ ID NO: 21. In some embodiments, the nucleic acid vector or polynucleotide contains a polynucleotide sequence including nucleotides 1 to 2650 of SEQ ID NO: 21.

In some embodiments of any of the foregoing aspects, the nucleic acid vector or polynucleotide contains polynucleotide sequence including nucleotides 212 to 3514 of SEQ ID NO: 22. In some embodiments, the nucleic acid vector or polynucleotide contains a polynucleotide sequence including nucleotides 1 to 3726 of SEQ ID NO: 22.

In some embodiments of any of the foregoing aspects, the nucleic acid vector or polynucleotide contains polynucleotide sequence including nucleotides 212 to 3514 of SEQ ID NO: 23. In some embodiments, the nucleic acid vector or polynucleotide contains a polynucleotide sequence including nucleotides 1 to 3726 of SEQ ID NO: 23.

In some embodiments of any of the foregoing aspects, the nucleic acid vector or polynucleotide contains polynucleotide sequence including nucleotides 212 to 2517 of SEQ ID NO: 24. In some embodiments, the nucleic acid vector or polynucleotide contains a polynucleotide sequence including nucleotides 1 to 2729 of SEQ ID NO: 24.

In some embodiments of any of the foregoing aspects, the nucleic acid vector or polynucleotide contains polynucleotide sequence including nucleotides 212 to 2517 of SEQ ID NO: 25. In some embodiments, the nucleic acid vector or polynucleotide contains a polynucleotide sequence including nucleotides 1 to 2729 of SEQ ID NO: 25.

In some embodiments of any of the foregoing aspects, the nucleic acid vector or polynucleotide contains polynucleotide sequence including nucleotides 212 to 3593 of SEQ ID NO: 26. In some embodiments, the nucleic acid vector or polynucleotide contains a polynucleotide sequence including nucleotides 1 to 3805 of SEQ ID NO: 26.

In some embodiments of any of the foregoing aspects, the nucleic acid vector or polynucleotide contains polynucleotide sequence including nucleotides 212 to 3593 of SEQ ID NO: 27. In some embodiments, the nucleic acid vector or polynucleotide contains a polynucleotide sequence including nucleotides 1 to 3805 of SEQ ID NO: 27.

In some embodiments of any of the foregoing aspects, the nucleic acid vector is a viral vector, plasmid, cosmid, or artificial chromosome. In some embodiments, the nucleic acid vector is a viral vector. In some embodiments, the viral vector is an adeno-associated virus (AAV) vector, an adenovirus vector, or a lentivirus vector. In some embodiments, the viral vector is an AAV vector. In some embodiments, the AAV vector has an AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ, DJ/8, DJ/9, 7m8, PHP.B, PHP.eB, or PHP.S capsid. In some embodiments, the AAV vector has an AAV1 capsid. In some embodiments, the AAV vector has an AAV9 capsid. In some embodiments, the AAV vector has a 7m8 capsid. In some embodiments, the AAV vector has a PHP.S capsid. In some embodiments, the AAV vector has an AAV-DJ capsid. In some embodiments, the AAV vector has an Anc80 capsid. In some embodiments, the AAV vector has an Anc80L65 capsid. In some embodiments, the AAV vector has an AAV2 capsid. In some embodiments, the AAV vector has an AAV2quad(Y-F) capsid. In some embodiments, the AAV vector has a PHP.eB capsid. In some embodiments, the AAV vector has an AAV3 capsid. In some embodiments, the AAV vector has an AAV4 capsid. In some embodiments, the AAV vector has an AAV5 capsid. In some embodiments, the AAV vector has an AAV6 capsid. In some embodiments, the AAV vector has an AAV7 capsid. In some embodiments, the AAV vector has an AAV8 capsid. In some embodiments, the AAV vector has a PHP.B capsid.

In another aspect, the invention provides a composition containing the nucleic acid vector of any of the foregoing aspects and embodiments. In some embodiments, the composition further includes a pharmaceutically acceptable carrier, diluent, or excipient.

In another aspect, the invention provides a cell containing the polynucleotide or vector of any of the foregoing aspects and embodiments. In some embodiments, the cell is a GJB2-expressing cell. In some embodiments, the cell is a GJB2-expressing inner ear cell. In some embodiments, the cell is a mammalian cell. In some embodiments, the mammalian cell is a human cell. In some embodiments, the cell is a cochlear supporting cell.

In another aspect, the invention provides a method of expressing human GJB2 in a GJB2-expressing cell by contacting the GJB2-expressing cell with the nucleic acid vector of or composition of any of the foregoing aspects and embodiments. In some embodiments, the GJB2-expressing cell is a GJB2-expressing inner ear cell (e.g., a cochlear supporting cell). In some embodiments, the contacting is in a subject (e.g., in vivo).

In another aspect, the invention provides a method of treating a subject having or at risk of developing GJB2-related hearing loss by administering to an inner ear of the subject a therapeutically effective amount of the nucleic acid vector composition of any of the foregoing aspects and embodiments.

In some embodiments, the GJB2-related hearing loss is DFNB1, DFNA3, or hearing loss associated with Bart-Pumphrey syndrome, hystrix-like ichthyosis with deafness, keratitis-ichthyosis-deafness syndrome, palmoplantar keratoderma with deafness, or Vohwinkel syndrome. In some embodiments, the GJB2-related hearing loss is DFNB1 or DFNA3. In some embodiments, the subject has a mutation in GJB2, a mutation in GJB6, or a mutation in both GJB2 and GJB6.

In another aspect, the invention provides a method of improving cochlear supporting cell function or cochlear supporting cell survival by contacting the cochlear supporting cell with the nucleic acid vector or composition of any of the foregoing aspects and embodiments. In some embodiments, the contacting is in a subject.

In another aspect, the invention provides a method of improving cochlear supporting cell function or cochlear supporting cell survival in a subject in need thereof by administering to an inner ear of the subject a therapeutically effective amount of the nucleic acid vector or composition of any of the foregoing aspects and embodiments.

In some embodiments of any of the foregoing aspects, the subject has or is at risk of developing GJB2-related hearing loss. In some embodiments, the GJB2-related hearing loss is DFNB1, DFNA3, or hearing loss associated with Bart-Pumphrey syndrome, hystrix-like ichthyosis with deafness, keratitis-ichthyosis-deafness syndrome, palmoplantar keratoderma with deafness, or Vohwinkel syndrome. In some embodiments, the hearing loss is DFNB1 or DFNA3.

In some embodiments of any of the foregoing aspects, the cochlear supporting cell is a mammalian cochlear supporting cell. In some embodiments, the mammalian cochlear supporting cell is a human cochlear supporting cell.

In some embodiments of any of the foregoing aspects, the method further includes evaluating the hearing of the subject prior to administering the nucleic acid vector or composition.

In some embodiments of any of the foregoing aspects, the method further includes evaluating the hearing of the subject after administering the nucleic acid vector or composition.

In some embodiments of any of the foregoing aspects, the nucleic acid vector or composition is locally administered. In some embodiments, the nucleic acid vector or composition is administered to the inner ear. In some embodiments, the nucleic acid vector or composition is administered to the middle ear. In some embodiments, the nucleic acid vector or composition is administered transtympanically or intratympanically. In some embodiments, the nucleic acid vector or composition is administered into the perilymph. In some embodiments, the nucleic acid vector or composition is administered into the endolymph. In some embodiments, the nucleic acid vector or composition is administered to or through the oval window. In some embodiments, the nucleic acid vector or composition is administered to or through the round window.

In some embodiments of any of the foregoing aspects, the nucleic acid vector or composition is administered in an amount sufficient to prevent or reduce hearing loss, delay the development of hearing loss, slow the progression of hearing loss, improve hearing, increase or induce expression of human GJB2 in GJB2-expressing cells, promote or increase cochlear supporting cell survival, or improve cochlear supporting cell function.

In some embodiments of any of the foregoing aspects, the subject is a human subject.

In another aspect, the invention provides a kit including the polynucleotide, nucleic acid vector, or composition of any of the foregoing aspects and embodiments.

### Definitions

As used herein, the term “about” refers to a value that is within 10% above or below the value being described.

As used herein, “administration” refers to providing or giving a subject a therapeutic agent (e.g., a nucleic acid vector containing a GJB2 regulatory construct operably linked to a polynucleotide encoding a wild-type Gjb2 protein), by any effective route. Exemplary routes of administration are described herein below.

As used herein, the phrase “administering to the inner ear” refers to providing or giving a therapeutic agent described herein to a subject by any route that allows for transduction of inner ear cells. Exemplary routes of administration to the inner ear include administration into the perilymph or endolymph, such as to or through the oval window, round window, or semicircular canal (e.g., horizontal canal), or by transtympanic or intratympanic injection, e.g., administration to a GJB2-expressing inner ear cell.

As used herein, the term “cell type” refers to a group of cells sharing a phenotype that is statistically separable based on gene expression data. For instance, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, the terms “conservative mutation,” “conservative substitution,” and “conservative amino acid substitution” refer to a substitution of one or more amino acids for one or more different amino acids that exhibit similar physicochemical properties, such as polarity, electrostatic charge, and steric volume. These properties are summarized for each of the twenty naturally occurring amino acids in Table 1.

TABLE 1
Representative physicochemical properties of naturally occurring amino acids

| | | | | Electrostatic | |
|---|---|---|---|---|---|
| | | | | character at | |
| | 3 Letter | 1 Letter | Side-chain | physiological pH | Steric |
| Amino Acid | Code | Code | Polarity | (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral and | large |
| | | | | cationic forms in | |
| | | | | equilibrium at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | non-polar | neutral | intermediate |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include (i) G, A, V, L, and I; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein, the terms “effective amount,” “therapeutically effective amount,” and a “sufficient amount” of a composition, vector construct, or viral vector described herein refer to a quantity sufficient to, when administered to the subject, including a mammal, for example a human, effect beneficial or desired results, including clinical results, and, as such, an “effective amount” or synonym thereto depends upon the context in which it is being applied. For example, in the context of treating sensorineural hearing loss, it is an amount of the composition, vector construct, or viral vector sufficient to achieve a treatment response as compared to the response obtained without administration of the composition, vector construct, or viral vector. The amount of a given composition described herein that will correspond to such an amount will vary depending upon various factors, such as the given agent, the pharmaceutical formulation, the route of administration, the type of disease or disorder, the identity of the subject (e.g., age, sex, weight) or host being treated, and the like, but can nevertheless be routinely determined by one skilled in the art. Also, as used herein, a “therapeutically effective amount” of a composition, vector construct, or viral vector of the present disclosure is an amount which results in a beneficial or desired result in a subject as compared to a control. As defined herein, a therapeutically effective amount of a composition, vector construct, or viral vector of the present disclosure may be readily determined by one of ordinary skill by routine methods known in the art. Dosage regimen may be adjusted to provide the optimum therapeutic response.

As used herein, the term “endogenous” refers to a molecule (e.g., a polypeptide, nucleic acid, or cofactor) that is found naturally in a particular organism (e.g., a human) or in a particular location within an organism (e.g., an organ, a tissue, or a cell, such as a human cell, e.g., a human cochlear supporting cell).

As used herein, the term “express” refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein. The term “expression product” refers to a protein or RNA molecule produced by any of these events.

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell, e.g., a human cochlear supporting cell). Exogenous materials include those that are provided from an external source to an organism or to cultured matter extracted therefrom.

As used herein, the terms “Gjb2” and “GJB2” (also known as connexin 26 and CX26) refer to a protein encoded by the GJB2 gene and to the gene encoding this protein, respectively. GJB2 is a member of the connexin gene family. Nearly half of all hearing loss is attributed to mutations in one of four members of the connexin gene family, and GJB2 mutations are the most common. More than 100 different mutations in GJB2 have been identified that cause non-syndromic hearing loss, which is loss of hearing that is not associated with other signs and symptoms. The term “Gjb2” refers to a wild-type Gjb2 protein, such as a wild-type human Gjb2 protein (e.g., a protein having the amino acid sequence of SEQ ID NO: 29), while the term “GJB2” also refers to polynucleotides that are codon-optimized and/or CpG-depleted relative to the sequence of a wild-type GJB2 gene (e.g., SEQ ID NO: 5), such as polynucleotides having at least 90% sequence identity (e.g., 90%, 91%, 92%, 93%, 94%, 95%, 96%, 97%, 98%, 99%, or 99.9% identity, or more sequence identity) to any one of SEQ ID NOs: 3, 4, and 28, provided that they encode a wild-type Gjb2 protein.

As used herein, the term “GJB2-expressing cell” refers to a cell type in the body that is known to endogenously express GJB2 (e.g., in a subject expressing a wild-type copy of GJB2). GJB2-expressing cells include epithelial cells of the esophagus, cervical cells (ectocervix), cells of the minor salivary gland, epithelial cells of the skin, epithelial cells of the vagina, respiratory epithelial cells, liver hepatocytes, epithelial cells of the kidney, cells of the testes, luminal epithelial cells of the mammary gland, pancreatic acinar cells, bladder urothelial cells, epithelial cells of the intestine, and GJB2-expressing inner ear cells. The term “GJB2-expressing cell” encompasses the same cell types in a subject having a mutation in GJB2.

As used herein, the term “GJB2-expressing inner ear cell” refers to a cell within the inner ear that endogenously expresses GJB2 (e.g., in a subject expressing a wild-type copy of GJB2). GJB2-expressing cells within the ear are found in both the cochlea and the vestibule. Cochlear GJB2-expressing cells include inner phalangeal cells, inner border cells, inner pillar cells, outer pillar cells, Deiter cells, Hensen's cells, Claudius cells, interdental cells, inner sulcus cells, outer sulcus cells, cells of the spiral limbus, spiral prominence cells, root cells, basal cells of the stria vascularis, intermediate cells of the stria vascularis, fibrocytes of the spiral limbus and spiral ligament, and mesenchymal cells lining the scala vestibuli. Vestibular GJB2-expressing cells include supporting cells, dark cells, fibrocytes, and mesenchymal cells. The term “GJB2-expressing inner ear cell” encompasses the same cell types in a subject having a mutation in GJB2.

As used herein, the term “GJB2 regulatory construct” refers to a polynucleotide that is capable of expressing a transgene specifically in GJB2-expressing cells, or a variant thereof, such as a polynucleotide having at least 85% sequence identity (e.g., 85%, 86%, 87%, 88%, 89%, 90%, 91%, 92%, 93%, 94%, 95%, 96%, 97%, 98%, 99%, or more sequence identity) to a GJB2 regulatory construct described herein. The GJB2 regulatory constructs of the disclosure contain one or more regulatory elements, such as a GJB2 promoter and a GJB2 enhancer, and have at least 85% sequence identity (e.g., 85%, 86%, 87%, 88%, 89%, 90%, 91%, 92%, 93%, 94%, 95%, 96%, 97%, 98%, 99%, or more sequence identity) to SEQ ID NO: 1 or SEQ ID NO: 2.

As used herein, the term “GJB2-related hearing loss” refers to diseases and conditions that feature hearing loss associated with a mutation in GJB2, such as DFNB1, which is characterized by moderate to profound prelingual hearing loss and is inherited in an autosomal recessive pattern, and DFNA3, which is characterized by moderate to severe prelingual or postlingual hearing loss that becomes more severe over time and is inherited in an autosomal dominant pattern. GJB2-related hearing loss also occurs in Bart-Pumphrey syndrome, hystrix-like ichthyosis with deafness, keratitis-ichthyosis-deafness syndrome, palmoplantar keratoderma with deafness, and Vohwinkel syndrome, all of which are characterized by hearing loss and skin abnormalities and associated with mutations in GJB2. Two types of GJB2-related hearing loss, DFNB1 and DFNA3, can also be associated with mutations in GJB6, either alone or in combination with mutations in GJB2. For example, subjects with DFNB1 may have a mutation in GJB2, a mutation in GJB6, or a mutation in both genes.

As used herein, the term “heterologous” refers to a combination of elements that is not naturally occurring. For example, a heterologous transgene refers to a transgene that is not naturally expressed by the promoter to which it is operably linked.

As used herein, the terms “increasing” and “decreasing” refer to modulating resulting in, respectively, greater or lesser amounts, of function, expression, or activity of a metric relative to a reference. For example, subsequent to administration of a composition in a method described herein, the amount of a marker of a metric (e.g., transgene expression, ABR, or DPOAE) as described herein may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the marker prior to administration. Generally, the metric is measured subsequent to administration at a time that the administration has had the recited effect, e.g., at least one week, one month, 3 months, or 6 months, after a treatment regimen has begun.

As used herein, “locally” or “local administration” means administration at a particular site of the body intended for a local effect and not a systemic effect. Examples of local administration are epicutaneous, inhalational, intra-articular, intrathecal, intravaginal, intravitreal, intrauterine, intra-lesional administration, lymph node administration, intratumoral administration, administration to the inner ear, and administration to a mucous membrane of the subject, wherein the administration is intended to have a local and not a systemic effect.

As used herein, the term “operably linked” refers to a first molecule joined to a second molecule, wherein the molecules are so arranged that the first molecule affects the function of the second molecule. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. Additionally, two portions of a transcription regulatory element are operably linked to one another if they are joined such that the transcription-activating functionality of one portion is not adversely affected by the presence of the other portion. Two transcription regulatory elements may be operably linked to one another by way of a linker polynucleotide (e.g., an intervening non-coding polynucleotide) or may be operably linked to one another with no intervening nucleotides present.

As used herein, the term “plasmid” refers to a to an extrachromosomal circular double stranded DNA molecule into which additional DNA segments may be ligated. A plasmid is a type of vector, a nucleic acid molecule capable of transporting another nucleic acid to which it has been linked. Certain plasmids are capable of autonomous replication in a host cell into which they are introduced (e.g., bacterial plasmids having a bacterial origin of replication and episomal mammalian plasmids). Other vectors (e.g., non-episomal mammalian vectors) can be integrated into the genome of a host cell upon introduction into the host cell, and thereby are replicated along with the host genome. Certain plasmids are capable of directing the expression of genes to which they are operably linked.

As used herein, the terms “nucleic acid” and “polynucleotide,” used interchangeably herein, refer to a polymeric form of nucleosides in any length. Typically, a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However, the term encompasses molecules containing nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e., the succession of letters used as abbreviations for bases) that biochemically characterizes a specific nucleic acid. A polynucleotide sequence presented herein is presented in a 5′ to 3′ direction unless otherwise indicated.

As used herein, the term “promoter” refers to a recognition site on DNA that is bound by an RNA polymerase. The polymerase drives transcription of the transgene.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “pharmaceutical composition” refers to a mixture containing a therapeutic agent, optionally in combination with one or more pharmaceutically acceptable excipients, diluents, and/or carriers, to be administered to a subject, such as a mammal, e.g., a human, in order to prevent, treat, or control a particular disease or condition affecting or that may affect the subject.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio. Preferably, the term “pharmaceutically acceptable” means approved by a regulatory agency of the Federal or a state government or listed in the U.S. Pharmacopeia or other generally recognized pharmacopeia for use in mammals, and more particularly in humans.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) isolated from a subject.

As used herein, the terms “subject” and “patient” refer to an animal (e.g., a mammal, such as a human). A subject to be treated according to the methods described herein may be one who has been diagnosed with sensorineural hearing loss (e.g., GJB2-related hearing loss) or one at risk of developing this condition (e.g., due to a genetic mutation). Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

As used herein, the terms “transcription regulatory element” and “regulatory sequence” refer to a polynucleotide that controls, at least in part, the transcription of a gene of interest. Transcription regulatory elements may include promoters, enhancers, and other polynucleotides (e.g., polyadenylation signals) that control or help to control gene transcription. Examples of transcription regulatory elements are described, for example, in Lorence, Recombinant Gene Expression: Reviews and Protocols (Humana Press, New York, NY, 2012).

As used herein, the term “transfection” refers to any of a wide variety of techniques commonly used for the introduction of exogenous DNA into a prokaryotic or eukaryotic host cell, e.g., electroporation, lipofection, calcium phosphate precipitation, DEAE-dextran transfection, Nucleofection, squeeze-poration, sonoporation, optical transfection, magnetofection, impalefection and the like.

As used herein, the terms “transduction” and “transduce” refer to a method of introducing a vector construct or a part thereof into a cell. Wherein the vector construct is contained in a viral vector such as for example an AAV vector, transduction refers to viral infection of the cell and subsequent transfer and integration of the vector construct or part thereof into the cell genome.

As used herein, “treatment” and “treating” in reference to a disease or condition, refer to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the term “vector” refers to a nucleic acid vector, e.g., a DNA vector, such as a plasmid, cosmid, or artificial chromosome, an RNA vector, a virus, or any other suitable replicon (e.g., viral vector). A variety of vectors have been developed for the delivery of polynucleotides encoding exogenous proteins into a prokaryotic or eukaryotic cell. Examples of such expression vectors are described in, e.g., Gellissen, Production of Recombinant Proteins: Novel Microbial and Eukaryotic Expression Systems (John Wiley & Sons, Marblehead, MA, 2006). Expression vectors suitable for use with the compositions and methods described herein contain a polynucleotide sequence as well as, e.g., additional sequence elements used for the expression of proteins and/or the integration of these polynucleotide sequences into the genome of a mammalian cell. Certain vectors that can be used for the expression of transgene as described herein include vectors that contain regulatory sequences, such as promoter and enhancer regions, which direct gene transcription. Other useful vectors for expression of a transgene contain polynucleotide sequences that enhance the rate of translation of the transgene or improve the stability or nuclear export of the mRNA that results from gene transcription. These sequence elements include, e.g., 5′ and 3′ untranslated regions and a polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

As used herein, the term “wild-type” refers to a genotype with the highest frequency for a particular gene in a given organism.

### BRIEF DESCRIPTION OF THE DRAWINGS

FIG. 1 is a plasmid map of plasmid P1588.

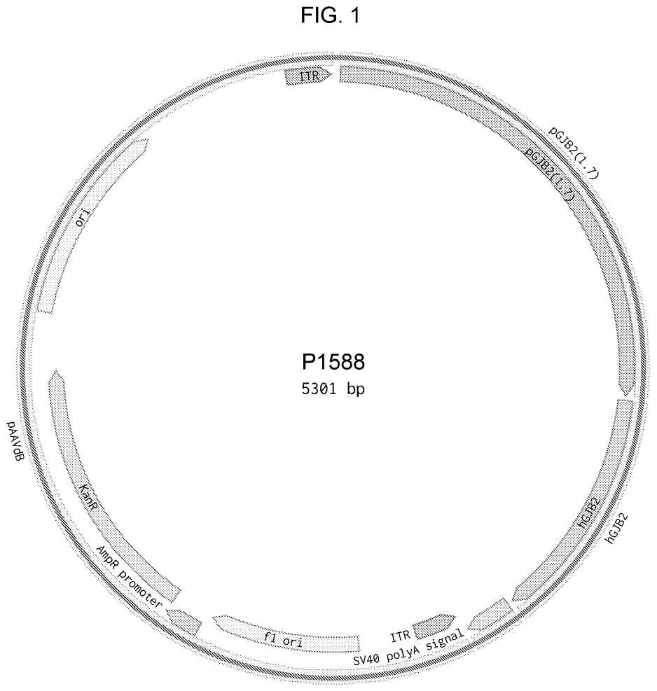

FIG. 2 is a plasmid map of plasmid P1589.

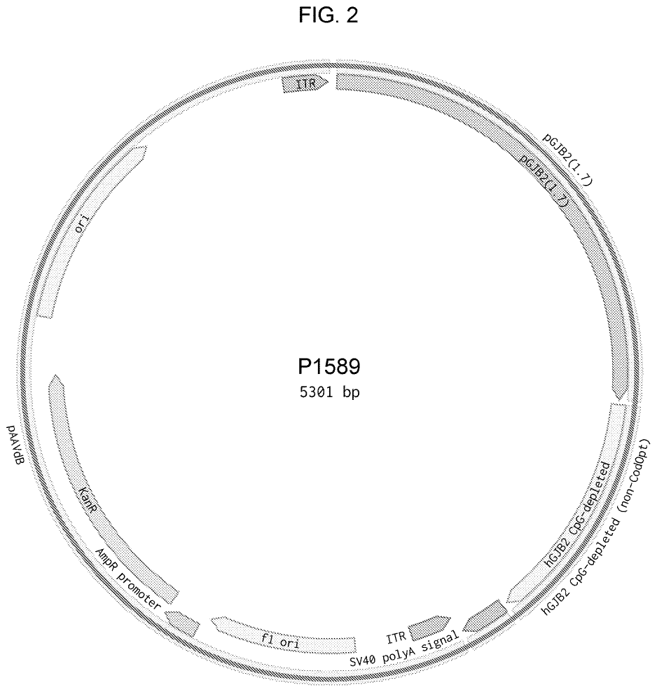

FIG. 3 is a plasmid map of plasmid P1590.

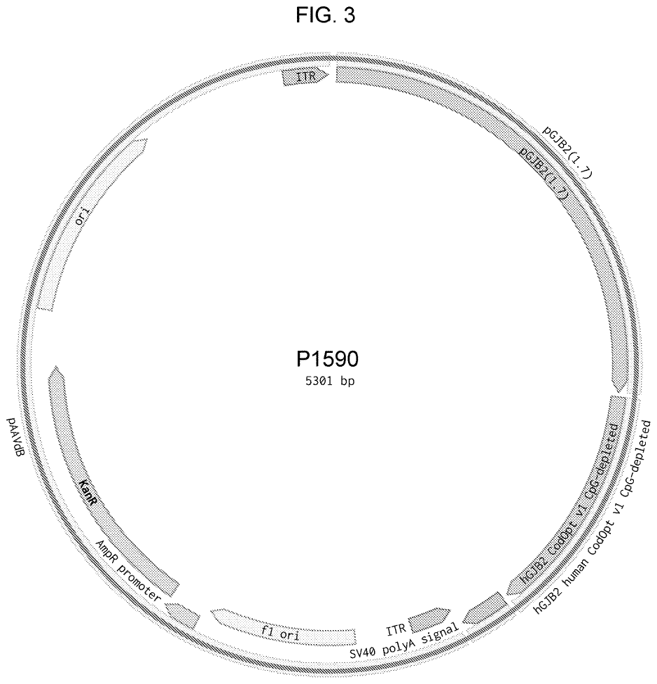

FIG. 4 is a plasmid map of plasmid P1592.

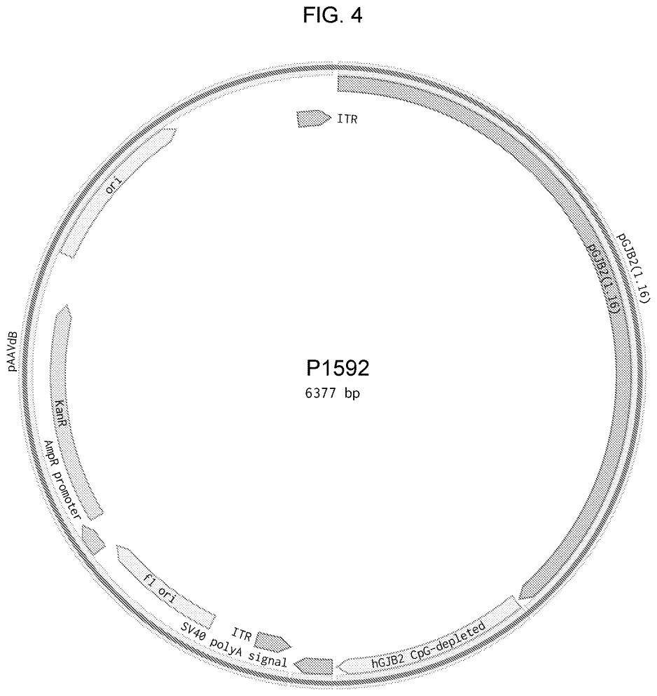

FIG. 5 is a plasmid map of plasmid P1593.

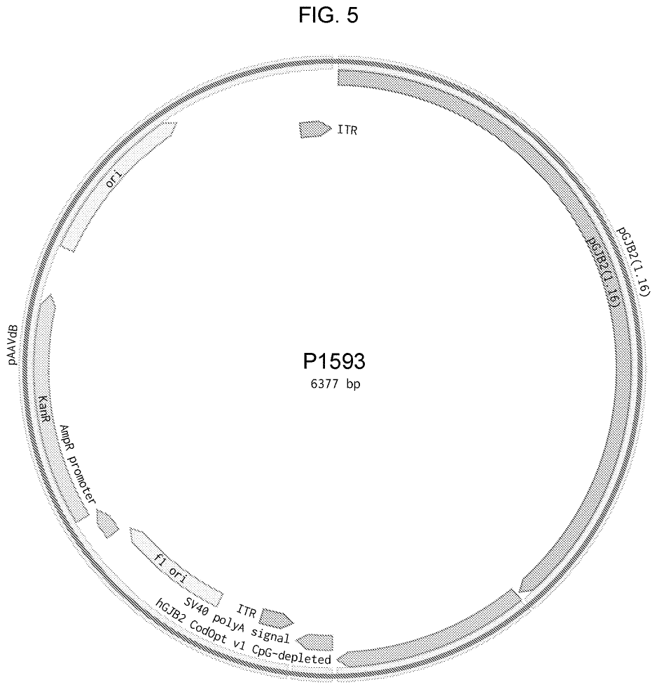

FIG. 6 is a plasmid map of plasmid P1595.

FIG. 7 is a plasmid map of plasmid P1596.

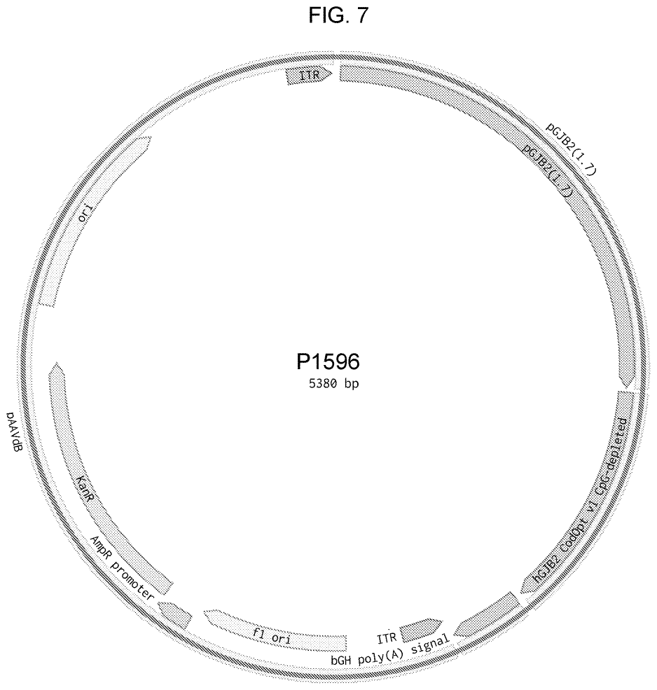

FIG. 8 is a plasmid map of plasmid P1598.

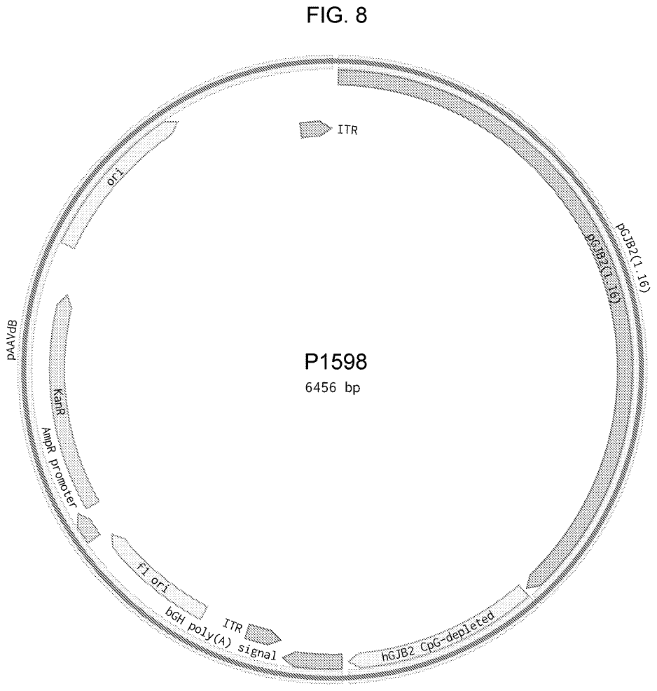

FIG. 9 is a plasmid map of plasmid P1599.

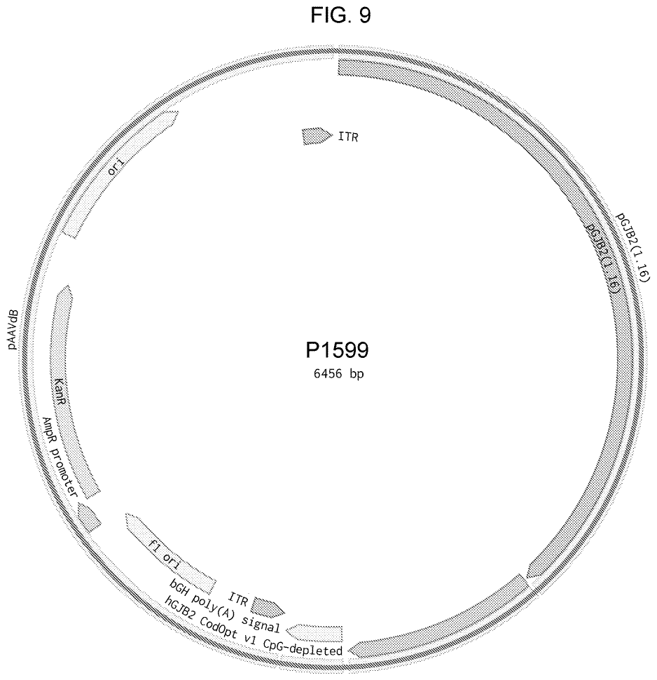

FIG. 10 is a plasmid map of plasmid P1601.

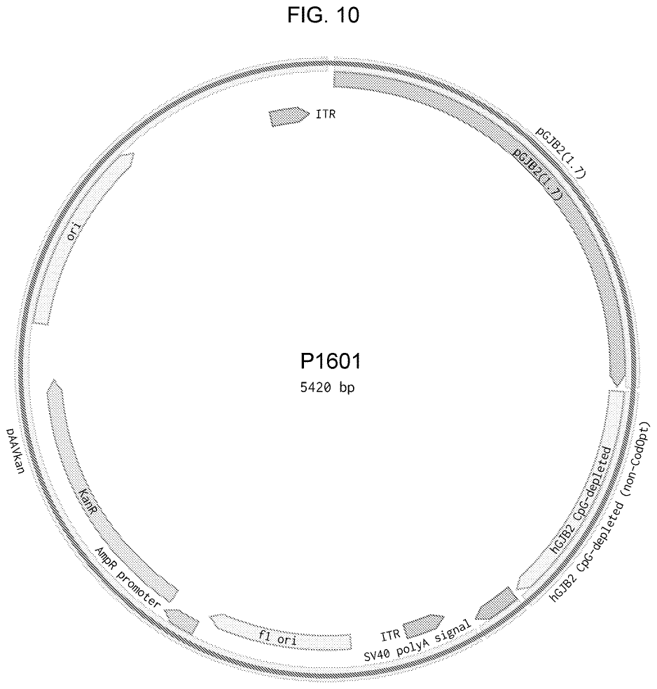

FIG. 11 is a plasmid map of plasmid P1602.

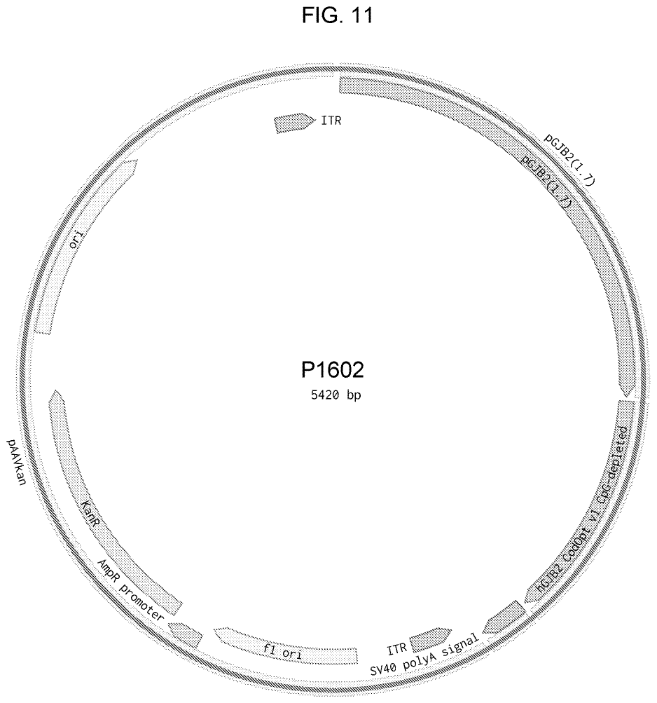

FIG. 12 is a plasmid map of plasmid P1604.

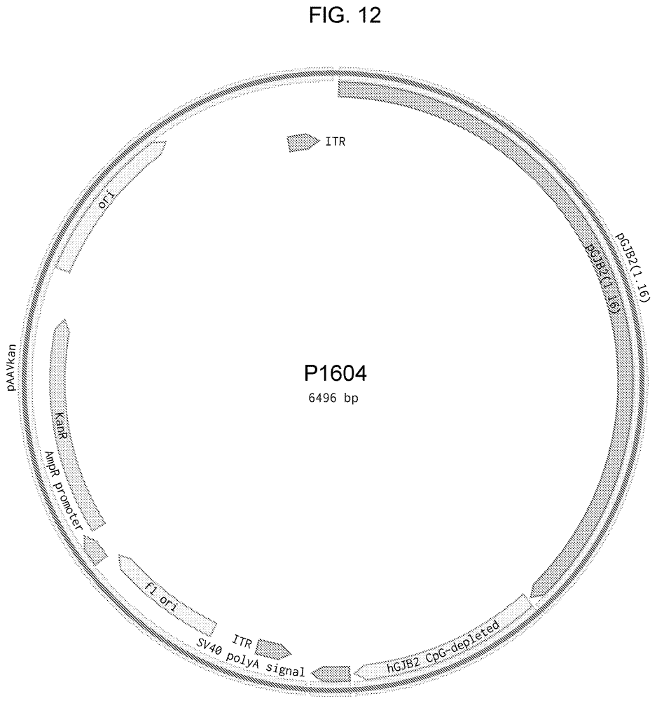

FIG. 13 is a plasmid map of plasmid P1605.

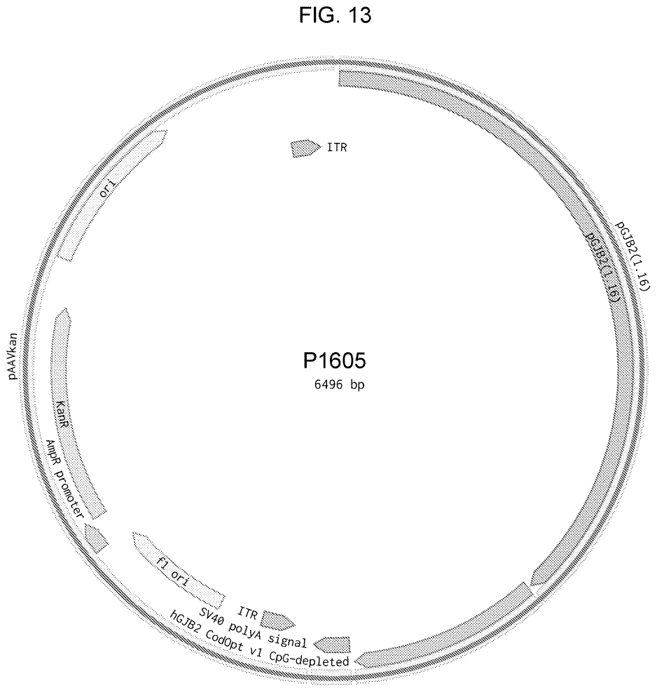

FIG. 14 is a plasmid map of plasmid P1607.

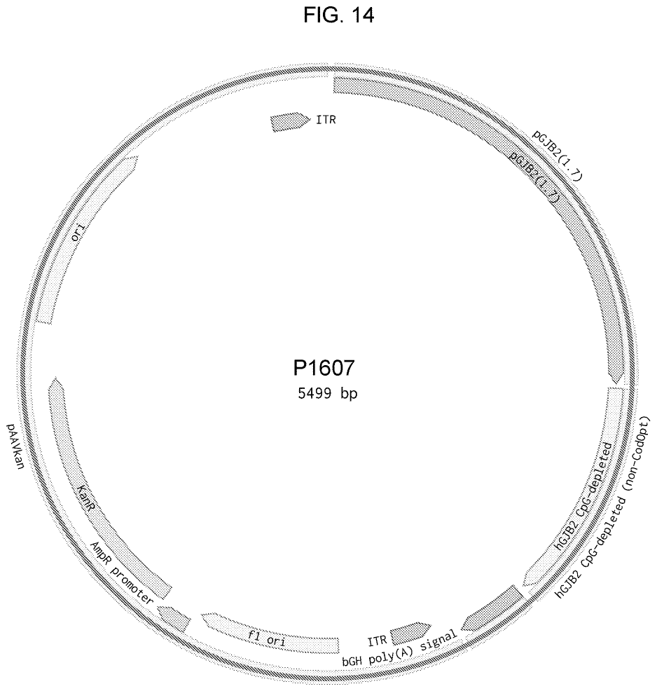

FIG. 15 is a plasmid map of plasmid P1608.

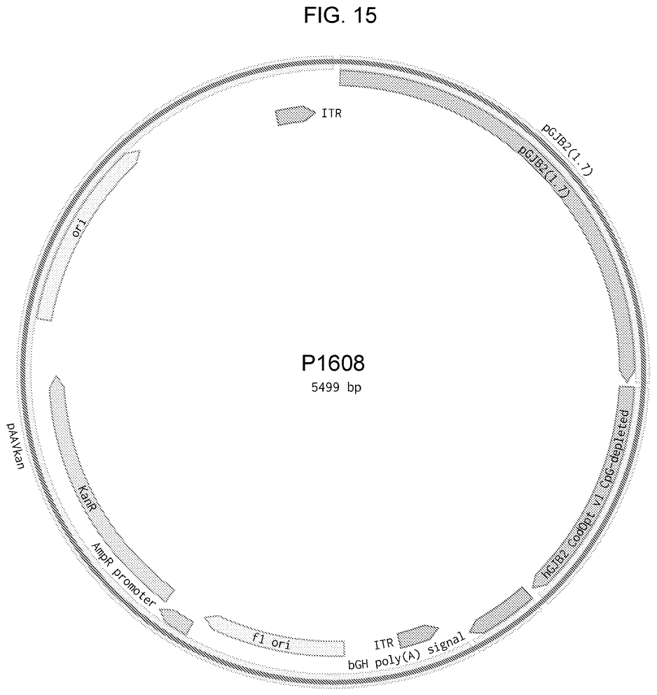

FIG. 16 is a plasmid map of plasmid P1610.

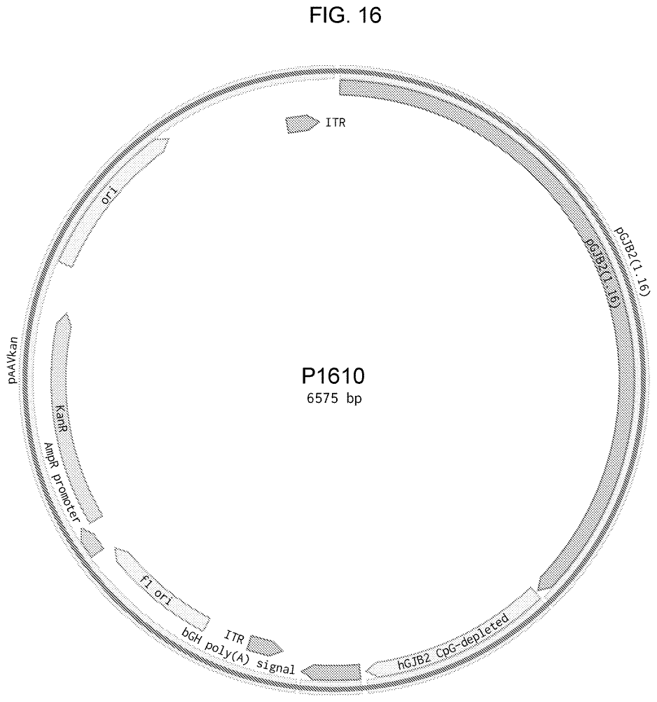

FIG. 17 is a plasmid map of plasmid P1611.

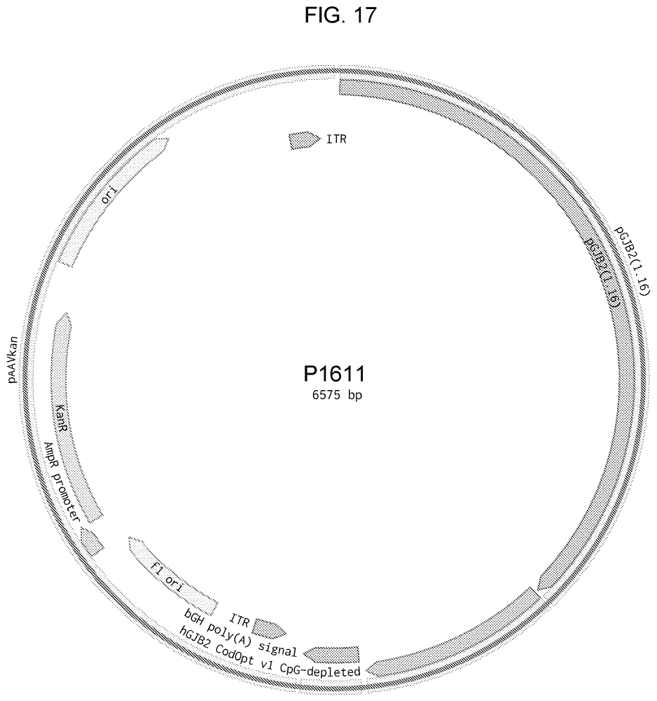

FIG. 18 is an image and a graph depicting the level of Gjb2 expression in HeLa cells transfected with different plasmids containing various FLAG-tagged versions of the human GJB2 coding sequence under control of a CMV promoter. Panel A depicts a western blot showing the level of Gjb2 expressed from each plasmid as detected by an anti-FLAG antibody. An anti-actin antibody was also used for normalization. Panel B depicts the relative intensity of anti-FLAG signal detected for each of the plasmids as compared to a plasmid harboring the wild-type GJB2 sequence, which was arbitrarily set to 1. “No GJB2” and “No Flag” are the negative control plasmid lacking a FLAG-tagged GJB2 coding sequence. “Wild-type” is a plasmid bearing the wild-type GJB2 sequence (SEQ ID NO: 5). “CpG-depleted” is a plasmid bearing a CpG-depleted GJB2 coding sequence (SEQ ID NO: 3) and a FLAG tag. “CodOpt” is a plasmid bearing a codon-optimized GJB2 coding sequence (SEQ ID NO: 28) and a FLAG tag. “CO-CpGdep” is a plasmid bearing a codon-optimized and CpG-depleted GJB2 coding sequence (SEQ ID NO: 4) and a FLAG tag.

FIG. 19 is a series of graphs depicting the ability of HeLa cells transfected with different plasmids containing various versions of the human GJB2 coding sequence under control of a CMV promoter to take up propidium iodide (“PI”) in both the presence and absence of Ca²⁺. Panel A depicts the percentage of cells that took up PI in both the presence and absence of Ca²⁺ for each transfection. Panel B depicts the percentage of cells that took up PI in the presence of Ca²⁺ for transfection with either the wild-type GJB2 coding sequence (SEQ ID NO: 5) or the CpG-depleted GJB2 coding sequence (SEQ ID NO: 3).

FIG. 20 is a series of graphs depicting the effect on hearing recovery in a GJB2 deficiency mouse model of an AAV1 vector bearing either the wild-type (SEQ ID NO: 5) or the CpG-depleted GJB2 coding sequence (SEQ ID NO: 3) under control of a GJB2 regulatory construct (SEQ ID NO: 1). Each AAV1 vector was injected into the right ear of the mice; the left ear was the negative control. The top panel depicts the effect on the auditory brainstem response (ABR) of the mice at various time points (in weeks) post-injection at various frequencies. The bottom panel depicts the effects on the distorted product otoacoustic emission (DPOAE) of the mice at various time points (in weeks) post-injection at various frequencies. The upper line in each of the four graphs depicts the results in the untreated, left ear of the mice.

FIG. 21 is a series of graphs depicting the dose-response effect of an AAV1 vector bearing the CpG-depleted GJB2 coding sequence (SEQ ID NO: 3) under control of a GJB2 regulatory construct (SEQ ID NO: 1) on hearing recovery in a GJB2 deficiency mouse model. Each AAV1 vector was injected into the right ears of the mice and measurements were taken four weeks post-administration of the vector. The “Untreated” mice were GJB2-deficient mice that were not treated with vector. The “Naïve WT” mice were non-GJB2 deficient BL6 mice that were not treated with vector. Panel A depicts the effect of various concentrations of the CpG-depleted GJB2 coding sequence on the mean auditory brainstem response (ABR) threshold of the mice as compared to the Untreated and Naïve WT mice. Panel B depicts the effect of various concentrations of the CpG-depleted GJB2 coding sequence on the mean DPOAE threshold of the mice as compared to the Untreated and Naïve WT mice.

FIG. 22 is a graph depicting the effect of a bovine growth hormone (bGH) polyadenylation signal sequence (SEQ ID NO: 6) compared to a simian virus 40 polyadenylation (SV40) polyadenylation signal sequence (SEQ ID NO: 7) on the mean expression levels of a nucleus-targeted green fluorescent protein driven by a CMV promoter (CMV.H2B-EGFP) in HEK293T cells.

FIG. 23 is a graph depicting the effect of a pAAVdB backbone (the 3,038 nucleotides spanning nucleotides 2399-5301 and 1-135 of SEQ ID NO: 12) as compared to a pAAVKan (the 3,157 nucleotides spanning nucleotides 2454-5420 and 1-190 of SEQ ID NO: 20) backbone on the mean expression levels of H2B-EGFP driven by a CMV promoter (CMV.H2B-EGFP) and having a bGH polyadenylation site (bGH_pA) in HEK293T cells. “Cassette 1” and “Cassette 2” represent two different CMV.H2B-EGFP.bGH_pA gene cassettes (sequences not shown), with different restriction enzyme cloning sites in the intragenic regions, but identical pAAVKan or pAAVdB sequences.

### DETAILED DESCRIPTION

Described herein are compositions and methods for inducing expression of a polynucleotide encoding a Gjb2 protein specifically in GJB2-expressing cells (e.g., GJB2-expressing inner ear cells, such as cochlear supporting cells). The invention features GJB2 regulatory constructs (constructs containing promoter and enhancer elements) that can induce expression of a polynucleotide encoding a Gjb2 protein (e.g., a wild-type Gjb2 protein, such as wild-type human Gjb2) in GJB2-expressing cells (e.g., cochlear supporting cells) with minimal to no expression in cochlear hair cells. The polynucleotide encoding the Gjb2 protein may be CpG depleted and/or codon optimized. The invention also features nucleic acid vectors containing the GJB2 regulatory constructs described herein operably linked to a polynucleotide encoding a Gjb2 protein. The compositions and methods described herein can be used to express a Gjb2 protein specifically in GJB2-expressing cells, and, therefore, the compositions described herein can be administered to a subject (such as a mammalian subject, for instance, a human) to treat GJB2-related hearing loss (e.g., DFNB1, DFNA3, or hearing loss associated with Bart-Pumphrey syndrome, hystrix-like ichthyosis with deafness, keratitis-ichthyosis-deafness syndrome, palmoplantar keratoderma with deafness, or Vohwinkel syndrome).

### Supporting Cells

Sensory epithelia of the inner ear contain two major cell types: hair cells and supporting cells. Hair cells are sensory cells of the auditory and vestibular systems that reside in the inner ear. Cochlear hair cells are the sensory cells of the auditory system and are made up of two main cell types: inner hair cells, which are responsible for sensing sound, and outer hair cells, which are thought to amplify low-level sound. Vestibular hair cells are located in the semicircular canal end organs and otolith organs of the inner ear and are involved in the sensation of movement that contributes to the sense of balance and spatial orientation. The development, function, and maintenance of inner ear sensory epithelia is highly dependent upon supporting cells, which are non-sensory cells that reside between hair cells. Supporting cells in the cochlea include Hensen's cells, Deiter cells, inner and outer pillar cells, Claudius cells, inner phalangeal cells, and border cells. Supporting cells are linked to each other and to hair cells by tight and adherens junctions and they communicate directly with other supporting cells by gap junctions. Gap junctions are made up of connexins that are encoded by connexin genes, such as CX26 (also known as GJB2) and CX30 (also known as GJB6). These connexin channels play an important role in recycling and regulating intracellular K+as well as pH homeostatic mechanisms and may also provide a pathway for rapid removal of ions from the region of the sensory cells during sound conduction in order to maintain sensitivity. Supporting cells have rigid cytoskeletons that maintain the structural integrity of the sensory organs during sound stimulation and head movements and, following trauma or toxicity, can eject injured hair cells from the epithelium, phagocytose hair cell debris, and, in some cases, generate new hair cells.

Gene therapy has recently emerged as an attractive therapeutic approach for treating hearing loss, particularly hearing loss caused by a mutation in a gene expressed in the inner ear. Mutations in many different genes have been found to cause hearing loss, including mutations in genes expressed in cochlear supporting cells. For example, mutations in GJB2 are the most common cause of recessive hearing loss. However, using gene therapy to treat hearing loss associated with mutations in cochlear supporting cell genes (e.g., genes expressed in cochlear supporting cells) calls for methods for inducing gene expression in cochlear supporting cells and not in cochlear hair cells, which are currently quite limited.

### GJB2

Gap junction protein beta 2 (Gjb2, also known as Connexin 26) is a protein encoded by the GJB2 gene and is a member of the connexin gene family. Connexins oligomerize into hexameric arrangements called connexons or hemichannels, which often dock with hemichannels from a contacting cell to form gap junctions. Nearly half of all hearing loss is attributed to mutations in one of four members of the connexin gene family, and GJB2 mutations are the most common. More than 100 different mutations in GJB2 have been identified that cause non-syndromic hearing loss, which is loss of hearing that is not associated with other signs and symptoms. One form of non-syndromic hearing loss that is associated with mutations in GJB2 is DFNB1, which is characterized by moderate to profound prelingual hearing loss and is inherited in an autosomal recessive pattern. DFNA3 is the other form of non-syndromic hearing loss that is associated with mutations in GJB2 and is moderate to severe prelingual or postlingual hearing loss that becomes more severe over time and is inherited in an autosomal dominant pattern. Other health conditions associated with mutations in GJB2 include Bart-Pumphrey syndrome, hystrix-like ichthyosis with deafness, keratitis-ichthyosis-deafness syndrome, palmoplantar keratoderma with deafness, and Vohwinkel syndrome, all of which are characterized by hearing loss and skin abnormalities.

The present invention is based, in part, on the discovery of regions upstream of the GJB2 coding sequence that can be used to promote expression of a polynucleotide encoding a Gjb2 protein specifically in GJB2-expressing cells (e.g., cochlear supporting cells cells). The inventors also identified CpG-depleted and/or codon-optimized polynucleotide sequences that encode wild-type human Gjb2 that can be used to reduce immune activation caused by gene therapy and/or regulate GJB2 expression levels. The compositions and methods described herein can, thus, be used to express a polynucleotide encoding a Gjb2 protein (e.g., a wild-type human Gjb2 protein) in GJB2-expressing cells (e.g., GJB2-expressing inner ear cells, such as cochlear supporting cells) to treat subjects having or at risk of developing sensorineural hearing loss (e.g., GJB2-related hearing loss). The discovery of GJB2 regulatory constructs that induce expression in GJB2-expressing cells while minimizing or eliminating off-target expression in cells that do not endogenously express GJB2 (e.g., cochlear hair cells) can improve the safety and efficacy of gene therapy by reducing toxicity associated with off-target expression.

The polynucleotides of the compositions and methods described herein include nucleic acid sequences containing GJB2 regulatory elements that are capable of expressing a transgene specifically in GJB2-expressing cells, or variants thereof, such as a nucleic acid sequences that have at least 85% sequence identity (e.g., 85%, 86%, 87%, 88%, 89%, 90%, 91%, 92%, 93%, 94%, 95%, 96%, 97%, 98%, 99%, or more sequence identity) to the nucleic acid sequences containing GJB2 regulatory elements that are capable of expressing a transgene specifically in GJB2-expressing cells. The nucleic acid sequences containing GJB2 regulatory elements that are capable of expressing a transgene specifically in GJB2-expressing cells are referred to herein as GJB2 regulatory constructs. In some embodiments, the GJB2 regulatory construct has at least 85% sequence identity (e.g., 85%, 86%, 87%, 88%, 89%, 90%, 91%, 92%, 93%, 94%, 95%, 96%, 97%, 98%, 99%, or more sequence identity) to SEQ ID NO: 1. In some embodiments, the GJB2 regulatory construct has the sequence of SEQ ID NO: 1. In some embodiments, the GJB2 regulatory construct has at least 85% sequence identity (e.g., 85%, 86%, 87%, 88%, 89%, 90%, 91%, 92%, 93%, 94%, 95%, 96%, 97%, 98%, 99%, or more sequence identity) to SEQ ID NO: 2. In some embodiments, the GJB2 regulatory construct has the sequence of SEQ ID NO: 2.

The foregoing nucleic acid sequences are provided in Table 2, below.

TABLE 2
GJB2 regulatory construct sequences

| SEQ | Description of regulatory | |
|---|---|---|
| ID NO: | construct sequence | Promoter sequence |
| 1 | GJB2 regulatory construct | AAAAACTCTTACATAATTGTAACAGATTTGAGTTTCCTCTGG |
| | pGJB2 1.7 | TTCAGGTTTTCTTGCCTCTTTGATAATCAAATGATCTGAAGA |
| | | AAGGCATAGAATTTCAAGGGAGAATCTGCATGACAGGATTA |
| | | CAATAAGGCTATTCATGGAGACTCTTTTATTAGCTTACACA |
| | | GGATCTGCATCATTCTTCGTCTCTGGCTTTGCTACAAGGCT |
| | | CCATTTAAACTTAACCCAACTTGCAGGCTTAACTGATCCAG |
| | | GAATGACTCAATACAATGAGCCAGCTACTGTAGCTCTTTTT |
| | | CCCCTTTTGATAAGGGGAGTTAACACAATGGGCTTTACAGT |
| | | TCTTAAATGAACGCGTACAGAGGACAACGACCACAGCCAT |
| | | CCCTGAACCCCGCCCACGGCACAGCGCCGGAGCCGGGGT |
| | | CTGGGGCGCCGCTTCCTGGGGGGTCCCGACTCTCAGCCG |
| | | CCCCCGCTTCACCCGGGCCGCCAAGGGGCTGGGGGAGG |
| | | CGGCGCTCGGGGTAACCGGGGGAGACTCAGGGCGCTGG |
| | | GGGCACTTGGGGAACTCATGGGGGCTCAAAGGAACTAGG |
| | | AGATCGGGACCTCGAAGGGGACTTGGGGGGTTCGGGGCT |
| | | TTCGGGGGCGGTCGGGGGTTCGCGGACCCGGGAAGCTCT |
| | | GAGGACCCAGAGGCCGGGCGCGCTCCGCCCGCGGCGCC |
| | | GCCCCCTCCGTAACTTTCCCAGTCTCCGAGGGAAGAGGCG |
| | | GGGTGTGGGGTGCGGTTAAAAGGCGCCACGGGGGGAGAC |
| | | AGGTGTTGCGGCCCCGCAGCGCCCGCGCGCTCCTCTCCC |
| | | CGACTCGGAGCCCCTCGGCGGCGCCCGGCCCAGGACCC |
| | | GCCTAGGAGCGCAGGAGCCCCAGCGCAGAGACCCCAACG |
| | | CCGAGACCCCCGCCCCGGCCCCGCCGCGCTTCCTCCCGA |
| | | CGCAGGTGAGCCCGCCGGCCCCGGACTGCCCGGCCAGG |
| | | AACCTGGCGCGGGGAGGGACCGCGAGACCCAGAGCGGTT |
| | | GCCCGGCCGCGTGGGTCTCGGGGAACCGGGGGGCTGGA |
| | | CCAACACACGTCCTTGGGCCGGGGGGGGGGGGCCGCCTT |
| | | CTGGAGCGGGCGTTTCTGTTTATGATGTGTTTAAAGATTGG |
| | | GTGAATTACTCAGGTGAACAAGCTACTTTTTATCAGAGAAC |
| | | ACCTAAAAACACGTTCAAGAGGGTTTGGGAACTATACATTT |
| | | AATCCTATGACAAACTAAGTTGGTTCTGTCTTCACCTGTTTT |
| | | GGTGAGGTTGTGTAAGAGTTGGTGTTTGCTCAGGAAGAGA |
| | | TTTAAGCATGCTTGCTTACCCAGACTCAGAGAAGTCTCCCT |
| | | GTTCTGTCCTAGCTAGTGATTCCTGTGTTGTGTGCATTCGT |
| | | CTTTTCCAGAGCAAACCGCCCAGAGTAGAAG |
| 2 | GJB2 regulatory construct | AAAAACTCTTACATAATTGTAACAGATTTGAGTTTCCTCTGG |
| | pGJB2 1.16 | TTCAGGTTTTCTTGCCTCTTTGATAATCAAATGATCTGAAGA |
| | | AAGGCATAGAATTTCAAGGGAGAATCTGCATGACAGGATTA |
| | | CAATAAGGCTATTCATGGAGACTCTTTTATTAGCTTACACA |
| | | GGATCTGCATCATTCTTCGTCTCTGGCTTTGCTACAAGGCT |
| | | CCATTTAAACTTAACCCAACTTGCAGGCTTAACTGATCCAG |
| | | GAATGACTCAATACAATGAGCCAGCTACTGTAGCTCTTTTT |
| | | CCCCTTTTGATAAGGGGAGTTAACACAATGGGCTTTACAGT |
| | | TCTTAAATGACCGTTCCTGCAGGTATTGGAAAACCTGCATC |
| | | GTATGCCTGCTGCTTTAGGAGACAACAAACTCAGCAGCCC |
| | | CTCCCCCGAAGCTGAAGTGCCAAACTTAAGGCTCCAGGTA |
| | | AACTGCCCTTCCGATGAGAGCTTTACATTTAGGCAGTAGCC |
| | | ACAGGCCTAAACAAAGTCAAATGGATGAAACTTGTGATCTC |
| | | TCCTTGGAAATATGGCCAAGAGTGAAACAACTACTTAGTTA |
| | | TTATTTTAAACGCAGGCTGCTAAATGGCTTGTGTTGGTATG |
| | | TAATGGCATCCTAGATAATGGGGGCTGCCAAGAAAATATTC |
| | | ACACTGGCAAGTCAATTAAAATACCGTTCTCTTCCTAAGAG |
| | | GCCTGGCTGGATTCCAGAGCTTCCTTTAGGGTAACGCTCA |
| | | AACCTGTCATAGTTTATTTTGTTTCTGTTTTCAGGCAACAGA |
| | | TTTGGTTATTGCTTTTATTGTATGAGTGTTGAAATGGTGTCC |
| | | TAACAGCCATCTGTACTGCCATCCCCTGGTCTCTCTGCCTT |
| | | TCCAGAAAGGAGATGAAGTTGCTGGAGCAGGCAGGTTCTC |
| | | TGAAAGGCTCCCTGAGTGTGGAAGAGCAGCTGTCACTCAT |
| | | CAGTGGTTGTCCCAATATCCAAGAAGCAGTAGAGGGTGCC |
| | | ATGCACATTCAGGTGAGTGAAGGACACTGGCGCCGCATCT |
| | | GTGGGGCCTGAGACATTGTCTCCCAGCCTCTCCGGAGGAA |
| | | CAGGGCAGGATACAGAGCAGTGGCAGGTGTGGGCATTTT |
| | | GCATCTTCTGTAATCTCCAAACAAAGTCTGCGCTAGGCCCT |
| | | TTCAGGCTATAAAGGACAGGGTACTCTGATTTCTGCTCTTG |
| | | ATGAGGGTTGATTGTGATACCAGGCGAGGGAGTAAGAGAT |
| | | ACAGGACACCATCTCTGCGGCTGCATCTTTGTCGCAGAAA |
| | | CAGACATTGTTCAGGGGACACCTGAGCCTCTGATAGTCAG |
| | | CCAGTGGCTCTGGCAGCTCACAGCAGTTATAGGGTGGCTG |
| | | TGTCCTTTTCTAAAGTCCTGACTTTTTATGGCCTGTGGTCAT |
| | | GGTGACTCCACTACGGGCTGTGTTTCTGTTGACGCGTACA |
| | | GAGGACAACGACCACAGCCATCCCTGAACCCCGCCCACG |
| | | GCACAGCGCCGGAGCCGGGGTCTGGGGCGCCGCTTCCTG |
| | | GGGGGTCCCGACTCTCAGCCGCCCCCGCTTCACCCGGGC |
| | | CGCCAAGGGGCTGGGGGAGGCGGCGCTCGGGGTAACCG |
| | | GGGGAGACTCAGGGCGCTGGGGGCACTTGGGGAACTCAT |
| | | GGGGGCTCAAAGGAACTAGGAGATCGGGACCTCGAAGGG |
| | | GACTTGGGGGGTTCGGGGCTTTCGGGGGGGGTCGGGGGT |
| | | TCGCGGACCCGGGAAGCTCTGAGGACCCAGAGGCCGGGC |
| | | GCGCTCCGCCCGCGGCGCCGCCCCCTCCGTAACTTTCCC |
| | | AGTCTCCGAGGGAAGAGGGGGGGTGTGGGGTGCGGTTAA |
| | | AAGGCGCCACGGCGGGAGACAGGTGTTGCGGCCCCGCAG |
| | | CGCCCGCGCGCTCCTCTCCCCGACTCGGAGCCCCTOGGC |
| | | GGCGCCCGGCCCAGGACCCGCCTAGGAGCGCAGGAGCC |
| | | CCAGCGCAGAGACCCCAACGCCGAGACCCCCGCCCCGGC |
| | | CCCGCCGCGCTTCCTCCCGACGCAGGTGAGCCCGCCGGC |
| | | CCCGGACTGCCCGGCCAGGAACCTGGCGCGGGGAGGGA |
| | | CCGCGAGACCCAGAGCGGTTGCCCGGCCGCGTGGGTCTC |
| | | GGGGAACCGGGGGGCTGGACCAACACACGTCCTTGGGCC |
| | | GGGGGGGGGGGGCCGCCTTCTGGAGCGGGCGTTTCTGTT |
| | | TATGATGTGTTTAAAGATTGGGTGAATTACTCAGGTGAACA |
| | | AGCTACTTTTTATCAGAGAACACCTAAAAACACGTTCAAGA |
| | | GGGTTTGGGAACTATACATTTAATCCTATGACAAACTAAGT |
| | | TGGTTCTGTCTTCACCTGTTTTGGTGAGGTTGTGTAAGAGT |
| | | TGGTGTTTGCTCAGGAAGAGATTTAAGCATGCTTGCTTACC |
| | | CAGACTCAGAGAAGTCTCCCTGTTCTGTCCTAGCTAGTGAT |
| | | TCCTGTGTTGTGTGCATTCGTCTTTTCCAGAGCAAACCGCC |
| | | CAGAGTAGAAG |

The foregoing GJB2 regulatory construct sequences can be included in a nucleic acid vector and operably linked to a polynucleotide encoding a Gjb2 protein. In some embodiments, the polynucleotide operably linked to a GJB2 regulatory construct described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more sequence identity) to SEQ ID NO: 1 or SEQ ID NO: 2) is a transgene that encodes a wild-type form of the Gjb2 protein (e.g., the wild-type human Gjb2 protein). In some embodiments, the polynucleotide operably linked to a GJB2 regulatory construct described herein is a polynucleotide encoding wild-type human Gjb2 (e.g., a polynucleotide encoding the amino acid sequence of SEQ ID NO: 29). In some embodiments, the polynucleotide sequence encoding wild-type human Gjb2 is modified relative to the wild-type GJB2 sequence (SEQ ID NO: 5). In some embodiments, the polynucleotide sequence encoding wild-type human Gjb2 is CpG depleted. An exemplary CpG depleted polynucleotide sequence that encodes wild-type human Gjb2 is the sequence of SEQ ID NO: 3. In some embodiments, the CpG depleted polynucleotide that encodes wild-type human Gjb2 has at least 90% sequence identity (e.g., 90%, 91%, 92%, 93%, 94%, 95%, 96%, 97%, 98%, 99%, or more sequence identity) to the sequence of SEQ ID NO: 3 and contains fewer CG dinucleotides than the wild-type GJB2 nucleic acid sequence. In some embodiments, the CpG depleted polynucleotide has at least 90% sequence identity (e.g., 90%, 91%, 92%, 93%, 94%, 95%, 96%, 97%, 98%, 99%, or more sequence identity) to the sequence of SEQ ID NO: 3 and contains at least 50% fewer CG dinucleotides (e.g., 50%, 60%, 70%, 75%, 80%, 85%, 90%, 95%, or fewer CG dinucleotides) than wild-type GJB2 (i.e., compared to SEQ ID NO: 5). In some embodiments, the CpG depleted polynucleotide encoding wild-type human Gjb2 has at least 90% sequence identity (e.g., 90%, 91%, 92%, 93%, 94%, 95%, 96%, 97%, 98%, 99%, or more sequence identity) to the sequence of SEQ ID NO: 3 and contains no CG dinucleotides. In some embodiments, the polynucleotide sequence encoding wild-type human Gjb2 is codon-optimized. An exemplary codon-optimized polynucleotide sequence that encodes wild-type human Gjb2 is the sequence of SEQ ID NO: 28. In some embodiments, the polynucleotide sequence encoding wild-type human Gjb2 is both CpG depleted and codon-optimized. An exemplary CpG depleted and codon optimized polynucleotide sequence that encodes wild-type human Gjb2 is the sequence of SEQ ID NO: 4. In some embodiments, the polynucleotide encoding wild-type human Gjb2 has at least 90% sequence identity (e.g., 90%, 91%, 92%, 93%, 94%, 95%, 96%, 97%, 98%, 99%, or more sequence identity) to the sequence of SEQ ID NO: 4 or SEQ ID NO: 28. In some embodiments, the polynucleotide encoding wild-type human Gjb2 that has at least 90% sequence identity (e.g., 90%, 91%, 92%, 93%, 94%, 95%, 96%, 97%, 98%, 99%, or more sequence identity) to the sequence of SEQ ID NO: 3 and contains at least 50% fewer CG dinucleotides (e.g., 50%, 60%, 70%, 75%, 80%, 85%, 90%, 95%, or fewer CG dinucleotides) than wild-type GJB2 or that has at least 90% sequence identity (e.g., 90%, 91%, 92%, 93%, 94%, 95%, 96%, 97%, 98%, 99%, or more sequence identity) to the sequence of SEQ ID NO: 4 or SEQ ID NO: 28 encodes the sequence of SEQ ID NO: 29 (e.g., due to the redundancy of the genetic code). Exemplary Gjb2 amino acid and polynucleotide sequences are listed in Table 3, below. A nucleic acid vector (e.g., an AAV vector) containing a GJB2 regulatory construct described herein operably linked to a polynucleotide encoding wild-type human Gjb2 (e.g., a polynucleotide encoding SEQ ID NO: 29, such as any one of SEQ ID NOs: 3, 4, and 28) can be administered to a subject to treat, reduce, or prevent GJB2-related hearing loss, such as hearing loss in a subject having DFNB1, DFNA3, Bart-Pumphrey syndrome, hystrix-like ichthyosis with deafness, keratitis-ichthyosis-deafness syndrome, palmoplantar keratoderma with deafness, or Vohwinkel syndrome.

TABLE 3
Gjb2 sequences

| SEQ ID | Description of | |
|---|---|---|
| NO: | sequence | Sequence |
| 3 | CpG depleted | ATGGATTGGGGCACACTGCAGACTATCCTGGGGGGTGTGAACA |
| | sequence encoding | AACACTCCACCAGCATTGGAAAGATCTGGCTCACAGTCCTCTTC |
| | human Gjb2 | ATTTTTAGGATTATGATCCTGGTTGTGGCTGCAAAGGAGGTGTG |
| | | GGGAGATGAGCAGGCTGACTTTGTCTGCAACACCCTGCAGCCA |
| | | GGCTGCAAGAATGTGTGCTATGATCACTACTTCCCCATCTCCCA |
| | | CATCAGGCTATGGGCCCTGCAGCTGATCTTTGTGTCCACTCCAG |
| | | CACTCCTAGTGGCCATGCATGTGGCCTACAGGAGACATGAGAA |
| | | GAAGAGGAAGTTCATCAAGGGGGAGATAAAGAGTGAATTTAAGG |
| | | ACATTGAGGAGATCAAAACCCAGAAGGTCAGAATTGAAGGCTCC |
| | | CTGTGGTGGACCTACACAAGCAGCATCTTCTTCAGGGTCATCTT |
| | | TGAAGCAGCCTTCATGTATGTCTTCTATGTCATGTATGATGGCTT |
| | | CTCCATGCAGAGGCTGGTGAAGTGCAATGCCTGGCCTTGTCCC |
| | | AACACTGTGGACTGCTTTGTGTCCAGGCCCACTGAGAAGACTGT |
| | | CTTCACAGTGTTCATGATTGCAGTGTCTGGAATTTGCATCCTGCT |
| | | GAATGTCACTGAATTGTGTTATTTGCTAATTAGATATTGTTCTGG |
| | | GAAGTCAAAAAAGCCAGTT |
| 4 | CpG depleted and | ATGGACTGGGGAACACTGCAAACAATCCTGGGAGGTGTGAACA |
| | codon-optimized | AGCACAGCACAAGCATTGGCAAGATCTGGCTGACAGTGCTGTTC |
| | sequence encoding | ATCTTTAGAATCATGATCCTGGTTGTGGCTGCTAAGGAAGTGTG |
| | human Gjb2 | GGGAGATGAGCAGGCTGACTTTGTTTGTAATACCCTGCAACCTG |
| | | GCTGCAAGAATGTGTGTTATGACCACTACTTCCCCATCAGCCAC |
| | | ATCAGACTGTGGGCCCTGCAACTGATCTTTGTGTCCACCCCTGC |
| | | CCTGCTGGTGGCCATGCATGTGGCCTACAGAAGGCATGAGAAG |
| | | AAGAGGAAATTCATCAAGGGAGAGATCAAGAGTGAGTTCAAGGA |
| | | CATTGAGGAAATCAAAACCCAGAAGGTGAGGATTGAAGGCTCTC |
| | | TGTGGTGGACCTACACCAGCAGCATCTTCTTCAGAGTGATCTTT |
| | | GAGGCAGCCTTTATGTATGTGTTCTATGTGATGTATGATGGCTTC |
| | | TCCATGCAGAGACTGGTGAAGTGCAATGCCTGGCCATGTCCTAA |
| | | CACAGTGGACTGCTTTGTGTCTAGACCTACAGAAAAGACAGTGT |
| | | TTACAGTCTTTATGATTGCAGTGTCTGGCATCTGCATTCTGCTGA |
| | | ATGTGACTGAGCTGTGCTACCTGCTCATTAGGTACTGTAGTGGA |
| | | AAAAGCAAGAAACCTGTG |
| 5 | Wild-type | ATGGATTGGGGCACGCTGCAGACGATCCTGGGGGGTGTGAACA |
| | (unmodified) | AACACTCCACCAGCATTGGAAAGATCTGGCTCACCGTCCTCTTC |
| | sequence encoding | ATTTTTCGCATTATGATCCTCGTTGTGGCTGCAAAGGAGGTGTG |
| | human Gjb2 | GGGAGATGAGCAGGCCGACTTTGTCTGCAACACCCTGCAGCCA |
| | | GGCTGCAAGAACGTGTGCTACGATCACTACTTCCCCATCTCCCA |
| | | CATCCGGCTATGGGCCCTGCAGCTGATCTTCGTGTCCACGCCA |
| | | GCGCTCCTAGTGGCCATGCACGTGGCCTACCGGAGACATGAGA |
| | | AGAAGAGGAAGTTCATCAAGGGGGAGATAAAGAGTGAATTTAAG |
| | | GACATCGAGGAGATCAAAACCCAGAAGGTCCGCATCGAAGGCT |
| | | CCCTGTGGTGGACCTACACAAGCAGCATCTTCTTCCGGGTCATC |
| | | TTCGAAGCCGCCTTCATGTACGTCTTCTATGTCATGTACGACGG |
| | | CTTCTCCATGCAGCGGCTGGTGAAGTGCAACGCCTGGCCTTGT |
| | | CCCAACACTGTGGACTGCTTTGTGTCCCGGCCCACGGAGAAGA |
| | | CTGTCTTCACAGTGTTCATGATTGCAGTGTCTGGAATTTGCATCC |
| | | TGCTGAATGTCACTGAATTGTGTTATTTGCTAATTAGATATTGTTC |
| | | TGGGAAGTCAAAAAAGCCAGTT |
| 28 | Codon-optimized | ATGGACTGGGGAACACTGCAAACAATCCTGGGCGGCGTGAACA |
| | sequence encoding | AGCACAGCACAAGCATCGGCAAGATCTGGCTGACCGTGCTGTT |
| | human Gjb2 | CATCTTTAGAATCATGATCCTGGTTGTGGCTGCTAAGGAAGTGT |
| | | GGGGCGATGAGCAGGCCGACTTCGTTTGTAATACCCTGCAACC |
| | | TGGCTGCAAGAACGTGTGTTACGACCACTACTTCCCCATCAGCC |
| | | ACATCAGACTGTGGGCCCTGCAACTGATCTTCGTGTCCACCCCT |
| | | GCCCTGCTGGTGGCCATGCATGTGGCCTACAGAAGGCACGAGA |
| | | AGAAGCGGAAATTCATCAAGGGCGAGATCAAGAGCGAGTTCAA |
| | | GGACATCGAGGAAATCAAAACCCAGAAGGTGCGGATCGAAGGC |
| | | TCTCTGTGGTGGACCTACACCAGCAGCATCTTCTTCAGAGTGAT |
| | | CTTCGAGGCCGCCTTTATGTACGTGTTCTACGTGATGTATGATG |
| | | GCTTCTCCATGCAGAGACTGGTGAAGTGCAACGCCTGGCCATG |
| | | TCCTAACACCGTCGACTGCTTCGTGTCTAGACCTACAGAAAAGA |
| | | CAGTGTTTACCGTCTTTATGATCGCCGTGTCTGGCATCTGCATTC |
| | | TGCTGAACGTGACCGAGCTGTGCTACCTGCTCATTCGGTACTGT |
| | | AGCGGAAAAAGCAAGAAACCTGTG |
| 29 | Human Gjb2 amino | MDWGTLQTILGGVNKHSTSIGKIWLTVLFIFRIMILVVAAKEVWGDE |
| | acid sequence | QADFVCNTLQPGCKNVCYDHYFPISHIRLWALQLIFVSTPALLVAMH |
| | (UniProt P29033) | VAYRRHEKKRKFIKGEIKSEFKDIEEIKTQKVRIEGSLWWTYTSSIFF |
| | | RVIFEAAFMYVFYVMYDGFSMQRLVKCNAWPCPNTVDCFVSRPTE |
| | | KTVFTVFMIAVSGICILLNVTELCYLLIRYCSGKSKKPV |

The disclosure also provides polynucleotides containing the codon-optimized and/or CpG depleted sequences that encode wild-type human Gjb2 shown above in Table 3 (e.g., the sequences of SEQ ID NO: 3, SEQ ID NO: 4, and SEQ ID NO: 28) and variants thereof, such as sequences having least 90% sequence identity (e.g., 90%, 91%, 92%, 93%, 94%, 95%, 96%, 97%, 98%, 99%, or more sequence identity) to the sequence of SEQ ID NO: 3 and containing at least 50% fewer CG dinucleotides (e.g., 50%, 60%, 70%, 75%, 80%, 85%, 90%, 95%, or fewer CG dinucleotides) than wild-type GJB2, and sequences having at least 90% sequence identity (e.g., 90%, 91%, 92%, 93%, 94%, 95%, 96%, 97%, 98%, 99%, or more sequence identity) to the sequence of SEQ ID NO: 4 or SEQ ID NO: 28. The codon-optimized and/or CpG depleted sequences that encode wild-type human Gjb2, or variants thereof, may be operably linked to a GJB2 promoter. GJB2 promoters that can be operably linked to a codon-optimized and/or CpG depleted sequence that encodes wild-type human Gjb2, or a variant thereof, described herein include the GJB2 promoters described in U.S. Publication No. US20210095313A1, International Publication Nos. WO2021231808A2 and WO2022056444A1, and International Application No. PCT/US2023/061953, which are incorporated herein by reference as they relate to GJB2 promoter sequences. Polynucleotides containing GJB2 promoter operably linked to a codon-optimized and/or CpG depleted sequence that encodes wild-type human Gjb2 (e.g., SEQ ID NO: 3, SEQ ID NO: 4, or SEQ ID NO: 28), or a variant thereof, described herein can be incorporated into a nucleic acid vector (e.g., an AAV vector) and administered to a subject to treat, reduce, or prevent GJB2-related hearing loss, such as hearing loss in a subject having DFNB1, DFNA3, Bart-Pumphrey syndrome, hystrix-like ichthyosis with deafness, keratitis-ichthyosis-deafness syndrome, palmoplantar keratoderma with deafness, or Vohwinkel syndrome.

### Expression of Gjb2 in Mammalian Cells

Mutations in GJB2 have been linked to sensorineural hearing loss. The compositions and methods described herein can be used to induce or increase the expression of a polynucleotide encoding Gjb2 (e.g., wild-type human Gjb2) specifically in GJB2-expressing cells (e.g., GJB2-expressing inner ear cells, such as cochlear supporting cells) by administering a nucleic acid vector that contains a GJB2 regulatory construct described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more sequence identity) to SEQ ID NO: 1 or SEQ ID NO: 2) operably linked to a polynucleotide sequence that encodes a Gjb2 protein (e.g., a polynucleotide having least 90% sequence identity (e.g., 90%, 91%, 92%, 93%, 94%, 95%, 96%, 97%, 98%, 99%, or more sequence identity) to the sequence of SEQ ID NO: 3 and containing at least 50% fewer CG dinucleotides (e.g., 50%, 60%, 70%, 75%, 80%, 85%, 90%, 95%, or fewer CG dinucleotides) than wild-type GJB2 or a polynucleotide having at least 90% sequence identity (e.g., 90%, 91%, 92%, 93%, 94%, 95%, 96%, 97%, 98%, 99%, or more sequence identity) to the sequence of SEQ ID NO: 4 or SEQ ID NO: 28). A wide array of methods has been established for the delivery of proteins to mammalian cells and for the stable expression of polynucleotides encoding proteins in mammalian cells.

### Polynucleotides Encoding Gjb2

One platform that can be used to achieve therapeutically effective intracellular concentrations of Gjb2 in mammalian cells is via the stable expression of a gene encoding Gjb2 (e.g., by integration into the nuclear or mitochondrial genome of a mammalian cell, or by episomal concatemer formation in the nucleus of a mammalian cell). The gene is a polynucleotide that encodes the primary amino acid sequence of the corresponding protein. In order to introduce exogenous genes into a mammalian cell, genes can be incorporated into a vector. Vectors can be introduced into a cell by a variety of methods, including transformation, transfection, transduction, direct uptake, projectile bombardment, and by encapsulation of the vector in a liposome. Examples of suitable methods of transfecting or transforming cells include calcium phosphate precipitation, electroporation, microinjection, infection, lipofection and direct uptake. Such methods are described in more detail, for example, in Green, et al., Molecular Cloning: A Laboratory Manual, Fourth Edition (Cold Spring Harbor University Press, New York 2014); and Ausubel, et al., Current Protocols in Molecular Biology (John Wiley & Sons, New York 2015), the disclosures of each of which are incorporated herein by reference.

Gjb2 can also be introduced into a mammalian cell by targeting a vector containing a gene encoding Gjb2 to cell membrane phospholipids. For example, vectors can be targeted to the phospholipids on the extracellular surface of the cell membrane by linking the vector molecule to a VSV-G protein, a viral protein with affinity for all cell membrane phospholipids. Such a construct can be produced using methods well known to those of skill in the field.

Recognition and binding of the polynucleotide encoding Gjb2 by mammalian RNA polymerase is important for gene expression. As such, one may include sequence elements within the polynucleotide that exhibit a high affinity for transcription factors that recruit RNA polymerase and promote the assembly of the transcription complex at the transcription initiation site. Such sequence elements include, e.g., a mammalian promoter, the sequence of which can be recognized and bound by specific transcription initiation factors and ultimately RNA polymerase. Examples of mammalian promoters have been described in Smith, et al., Mol. Sys. Biol., 3:73, online publication, the disclosure of which is incorporated herein by reference. The promoter used in the methods and compositions described herein is a GJB2 promoter contained within a GJB2 regulatory construct described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more sequence identity) to SEQ ID NO: 1 or SEQ ID NO: 2).

Once a polynucleotide encoding Gjb2 has been incorporated into a mammalian cell or stabilized in an episomal monomer or concatemer, the transcription of this polynucleotide can be induced by methods known in the art. For example, expression can be induced by exposing the mammalian cell to an external chemical reagent, such as an agent that modulates the binding of a transcription factor and/or RNA polymerase to the mammalian promoter and thus regulates gene expression. The chemical reagent can serve to facilitate the binding of RNA polymerase and/or transcription factors to the mammalian promoter, e.g., by removing a repressor protein that has bound the promoter. Alternatively, the chemical reagent can serve to enhance the affinity of the mammalian promoter for RNA polymerase and/or transcription factors such that the rate of transcription of the gene located downstream of the promoter is increased in the presence of the chemical reagent. Examples of chemical reagents that potentiate polynucleotide transcription by the above mechanisms include tetracycline and doxycycline. These reagents are commercially available and can be administered to a mammalian cell in order to promote gene expression according to established protocols.

Other DNA sequence elements that may be included in nucleic acid vectors for use in the compositions and methods described herein include enhancer sequences. Enhancers represent another class of regulatory elements that induce a conformational change in the polynucleotide containing the gene of interest such that the DNA adopts a three-dimensional orientation that is favorable for binding of transcription factors and RNA polymerase at the transcription initiation site. Thus, polynucleotides for use in the compositions and methods described herein include those that encode a Gjb2 protein and additionally include a mammalian enhancer sequence. Many enhancer sequences are now known from mammalian genes, and examples include enhancers from the genes that encode mammalian globin, elastase, albumin, α-fetoprotein, and insulin. Enhancers for use in the compositions and methods described herein also include those that are derived from the genetic material of a virus capable of infecting a eukaryotic cell. Examples include the SV40 enhancer on the late side of the replication origin, the cytomegalovirus early promoter enhancer, the polyoma enhancer on the late side of the replication origin, and adenovirus enhancers. Additional enhancer sequences that induce activation of eukaryotic gene transcription include the CMV enhancer and RSV enhancer. The GJB2 regulatory constructs described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more sequence identity) to SEQ ID NO: 1 or SEQ ID NO: 2) include one or more GJB2 enhancer sequences. An enhancer may be spliced into a vector containing a polynucleotide encoding a Gjb2 protein, for example, at a position 5′ or 3′ to this gene. In a preferred orientation, the enhancer is positioned at the 5′ side of the promoter, which in turn is located 5′ relative to the polynucleotide encoding a Gjb2 protein.

The nucleic acid vectors containing a GJB2 regulatory construct operably linked to a polynucleotide encoding a Gjb2 protein described herein may include a Woodchuck Posttranscriptional Regulatory Element (WPRE). The WPRE acts at the mRNA level, by promoting nuclear export of transcripts and/or by increasing the efficiency of polyadenylation of the nascent transcript, thus increasing the total amount of mRNA in the cell. The addition of the WPRE to a vector can result in a substantial improvement in the level of transgene expression from several different promoters, both in vitro and in vivo.

The nucleic acid vectors described herein also contain a polyadenylation (polyA) signal sequence. A polyA signal sequence is a sequence that triggers endonuclease cleavage of an mRNA and the addition of a series of adenosines to the 3′ end of the cleaved mRNA. PolyA signal sequences that can be included in the nucleic acid vectors described herein include polyA signal sequences from bovine growth hormone (bGH) mouse-β-globin, mouse-α-globin, human collagen, polyoma virus, the Herpes simplex virus thymidine kinase gene, human growth hormone (hGH), SV40, synthetic polyA, HIV-1 upstream polyA enhancer, adenovirus (L3) upstream polyA enhancer, hTHGB upstream polyA enhancer, and hC2 upstream polyA enhancer. In some embodiments, the nucleic acid vector contains a bGH polyA signal sequence having the sequence of:

(SEQ ID NO: 6)
CTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGA
GGAAATTGCATCGCATTGTCTGAGTAGGTGTCATTCTATTCTGGGGGGTG
GGGTGGGGCAGGACAGCAAGGGGGAGGATTGGGAAGACAATAGCAGGCAT
GCTGGGGA.

In some embodiments, the nucleic acid vector contains a bGH polyA signal sequence having at least 90% sequence identity (e.g., 90%, 91%, 92%, 93%, 94%, 95%, 96%, 97%, 98%, 99%, or more sequence identity) to the sequence of SEQ ID NO: 6.

In some embodiments, the nucleic acid vector contains an SV40 polyA signal sequence having the sequence of:

(SEQ ID NO: 7)
GATCCAGACATGATAAGATACATTGATGAGTTTGGACAAACCAAAACTAG
AATGCAGTGAAAAAAATGCCTTATTTGTGAAATTTGTGATGCTATTGCCT
TATTTGTAACCATTATAAGCTGCAATAAACAAGTT.

In some embodiments, the nucleic acid vector contains an SV40 polyA signal sequence having at least 90% sequence identity (e.g., 90%, 91%, 92%, 93%, 94%, 95%, 96%, 97%, 98%, 99%, or more sequence identity) to the sequence of SEQ ID NO: 7.

In some embodiments, the nucleic acid vectors containing a GJB2 regulatory construct operably linked to a polynucleotide encoding a Gjb2 protein described herein include a reporter sequence, which can be useful in verifying the expression of a gene operably linked to a GJB2 regulatory construct, for example, in cells and tissues (e.g., in GJB2-expressing cells, such as cochlear supporting cells). Reporter sequences that may be provided in a transgene include DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements that drive their expression, such as a GJB2 regulatory construct, the reporter sequences provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA), and immunohistochemistry. For example, where the marker sequence is the LacZ gene, the presence of the vector carrying the signal is detected by assays for β-galactosidase activity. Where the transgene is green fluorescent protein or luciferase, the vector carrying the signal may be measured visually by color or light production in a luminometer.

In some embodiments, the nucleic acid vectors described herein are AAV transfer plasmids. Such plasmids contain inverted terminal repeat sequences (ITRs) 5′ of the promoter (i.e., 5′ of a GJB2 regulatory construct described herein) and 3′ of the polyA signal sequence. The DNA sequence between the ITRs will be packaged into the AAV molecule, while sequence outside the ITRs will not. In some embodiments, the sequence positioned 5′ of a GJB2 regulatory construct described herein in an AAV transfer plasmid is the sequence:

(SEQ ID NO: 8)
CTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGG
GAGTGGCCAACTCCATCACTAGGGGTTCCTTGTAGTTAATGATTAACCCG
CCATGCTACTTATCTACGTAGCCATGCTCTAGGAAGATCGGAATTCTGGT
ACCTTGCTAGC.

In other embodiments, the sequence positioned 5′ of a GJB2 regulatory construct described herein in an AAV transfer plasmid is the sequence:

(SEQ ID NO: 10)
CTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGG
GAGTGGCCAACTCCATCACTAGGGGTTCCTGAGCAGAATTCTGGTACCTT
GCTAGC.

In both SEQ ID NO: 8 and SEQ ID NO: 10, shown above, nucleotides 1-130 correspond to the 5′ ITR.

In some embodiments, the sequence positioned 3′ of a polyA signal sequence in an AAV transfer plasmid is the sequence:

(SEQ ID NO: 9)
TCTAGAACTGAATTCCCGATAAGGATCTTCCTAGAGCATGGCTACGTAGA
TAAGTAGCATGGGGGTTAATCATTAACTACAAGGAACCCCTAGTGATGGA
GTTGGCCACTCCCTCTCTGCGCGCTCGCTCGCTCACTGAGGCCGGGCGAC
CAAAGGTCGCCCGACGCCCGGGCTTTGCCCGGGCGGCCTCAGTGAGCGAG
CGAGCGCGCAG.

In SEQ ID NO: 9, shown above, nucleotides 83-212 correspond to the 3′ ITR. In other embodiments, the sequence positioned 3′ of a polyA signal sequence in an AAV transfer plasmid is the sequence:

(SEQ ID NO: 11)
TCTAGAACTGAATTCACAAGGAACCCCTAGTGATGGAGTTGGCCACTCCC
TCTCTGCGCGCTCGCTCGCTCACTGAGGCCGGGCGACCAAAGGTCGCCCG
ACGCCCGGGCTTTGCCCGGGGGGCCTCAGTGAGCGAGCGAGCGCGCAG.

In SEQ ID NO: 11, shown above, nucleotides 19-148 correspond to the 3′ ITR. In some embodiments, an AAV transfer plasmid contains the 5′ sequence of SEQ ID NO: 8 and the 3′ sequence of SEQ ID NO: 9. In some embodiments, an AAV transfer plasmid contains a 5′ sequence having at least 90% sequence identity (e.g., 90%, 91%, 92%, 93%, 94%, 95%, 96%, 97%, 98%, 99%, or more sequence identity) to the sequence of SEQ ID NO: 8 and a 3′ sequence having at least 90% sequence identity (e.g., 90%, 91%, 92%, 93%, 94%, 95%, 96%, 97%, 98%, 99%, or more sequence identity) to the sequence of SEQ ID NO: 9, provided that the ITR sequence in the 5′ sequence has the sequence of nucleotides 1-130 of SEQ ID NO: 8 and the ITR sequence in the 3′ sequence has the sequence of nucleotides 83-212 of SEQ ID NO: 9. In some embodiments, an AAV transfer plasmid contains a 5′ sequence having at least 90% sequence identity (e.g., 90%, 91%, 92%, 93%, 94%, 95%, 96%, 97%, 98%, 99%, or more sequence identity) to the sequence of SEQ ID NO: 8 and a 3′ sequence having at least 90% sequence identity (e.g., 90%, 91%, 92%, 93%, 94%, 95%, 96%, 97%, 98%, 99%, or more sequence identity) to the sequence of SEQ ID NO: 9, provided that the ITR sequence in the 3′ is the reverse complement of the ITR sequence in the 5′ sequence. In some embodiments, an AAV transfer plasmid contains the 5′ sequence of SEQ ID NO: 10 and the 3′ sequence of SEQ ID NO: 11. In some embodiments, an AAV transfer plasmid contains a 5′ sequence having at least 90% sequence identity (e.g., 90%, 91%, 92%, 93%, 94%, 95%, 96%, 97%, 98%, 99%, or more sequence identity) to the sequence of SEQ ID NO: 10 and a 3′ sequence having at least 90% sequence identity (e.g., 90%, 91%, 92%, 93%, 94%, 95%, 96%, 97%, 98%, 99%, or more sequence identity) to the sequence of SEQ ID NO: 11, provided that the ITR sequence in the 5′ sequence has the sequence of nucleotides 1-130 of SEQ ID NO: 10 and the ITR sequence in the 3′ sequence has the sequence of nucleotides 19-148 of SEQ ID NO: 11. In some embodiments, an AAV transfer plasmid contains a 5′ sequence having at least 90% sequence identity (e.g., 90%, 91%, 92%, 93%, 94%, 95%, 96%, 97%, 98%, 99%, or more sequence identity) to the sequence of SEQ ID NO: 10 and a 3′ sequence having at least 90% sequence identity (e.g., 90%, 91%, 92%, 93%, 94%, 95%, 96%, 97%, 98%, 99%, or more sequence identity) to the sequence of SEQ ID NO: 11, provided that the ITR sequence in the 3′ is the reverse complement of the ITR sequence in the 5′ sequence.

In some embodiments, the 5′ flanking inverted terminal repeat has a sequence corresponding to nucleotides 1-130 of SEQ ID NO: 8 or SEQ ID NO: 10 or a sequence having at least 90% sequence identity (at least 90%, 91%, 92%, 93%, 94%, 95%, 96%, 97%, 98%, 99%, or more sequence identity) thereto; and the 3′ flanking inverted terminal repeat has a sequence corresponding to nucleotides 83-212 of SEQ ID NO: 9 or nucleotides 19-148 of SEQ ID NO: 11 or a sequence having at least 90% sequence identity (at least 90%, 91%, 92%, 93%, 94%, 95%, 96%, 97%, 98%, 99%, or more sequence identity) thereto. It will be understood by those of skill in the art that, for any given pair of inverted terminal repeat sequences in a transfer plasmid that is used to create the viral vector (typically by transfecting cells with that plasmid together with other plasmids carrying the necessary AAV genes for viral vector formation), that the corresponding sequence in the viral vector can be altered due to the ITRs adopting a “flip” or “flop” orientation during recombination. Thus, the sequence of the ITR in the transfer plasmid is not necessarily the same sequence that is found in the viral vector prepared therefrom.

A GJB2 regulatory construct described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more sequence identity) to SEQ ID NO: 1 or SEQ ID NO: 2) can be operably linked to a polynucleotide sequence that encodes a wild-type Gjb2 protein described herein (e.g., a polynucleotide having least 90% sequence identity (e.g., 90%, 91%, 92%, 93%, 94%, 95%, 96%, 97%, 98%, 99%, or more sequence identity) to the sequence of SEQ ID NO: 3 and containing at least 50% fewer CG dinucleotides (e.g., 50%, 60%, 70%, 75%, 80%, 85%, 90%, 95%, or fewer CG dinucleotides) than wild-type GJB2 or a polynucleotide having at least 90% sequence identity (e.g., 90%, 91%, 92%, 93%, 94%, 95%, 96%, 97%, 98%, 99%, or more sequence identity) to the sequence of SEQ ID NO: 4 or SEQ ID NO: 28), which can be operably linked to a polyA signal sequence described herein (e.g., a polynucleotide having at least 90% sequence identity (e.g., 90%, 91%, 92%, 93%, 94%, 95%, 96%, 97%, 98%, 99%, or more sequence identity) to the sequence of SEQ ID NO: 6 or SEQ ID NO: 7) and incorporated into a transfer plasmid that can be used to produce a nucleic acid vector (e.g., an AAV vector) for use in the compositions and methods described herein. The GJB2 regulatory sequence and the polyA signal sequence can be flanked by the and 5′ and 3′ ITR-containing sequences described herein in the transfer plasmid. The combinations of these elements that can be incorporated into such a transfer plasmid are provided in Table 4, below, in which each row represents a combination of elements for a single transfer plasmid.

TABLE 4
Sequence combinations for transfer plasmids for the production of AAV vectors

| | GJB2 regulatory | | | |
|---|---|---|---|---|
| 5′ sequence | construct | GJB2 coding | PolyA signal | 3′ sequence |
| containing ITR | sequence | sequence | sequence | containing ITR |
| SEQ ID NO: 10 | SEQ ID NO: 1 | SEQ ID NO: 3 | SEQ ID NO: 7 | SEQ ID NO: 11 |
| SEQ ID NO: 10 | SEQ ID NO: 1 | SEQ ID NO: 4 | SEQ ID NO: 7 | SEQ ID NO: 11 |
| SEQ ID NO: 10 | SEQ ID NO: 2 | SEQ ID NO: 3 | SEQ ID NO: 7 | SEQ ID NO: 11 |
| SEQ ID NO: 10 | SEQ ID NO: 2 | SEQ ID NO: 4 | SEQ ID NO: 7 | SEQ ID NO: 11 |
| SEQ ID NO: 10 | SEQ ID NO: 1 | SEQ ID NO: 3 | SEQ ID NO: 6 | SEQ ID NO: 11 |
| SEQ ID NO: 10 | SEQ ID NO: 1 | SEQ ID NO: 4 | SEQ ID NO: 6 | SEQ ID NO: 11 |
| SEQ ID NO: 10 | SEQ ID NO: 2 | SEQ ID NO: 3 | SEQ ID NO: 6 | SEQ ID NO: 11 |
| SEQ ID NO: 10 | SEQ ID NO: 2 | SEQ ID NO: 4 | SEQ ID NO: 6 | SEQ ID NO: 11 |
| SEQ ID NO: 8 | SEQ ID NO: 1 | SEQ ID NO: 3 | SEQ ID NO: 7 | SEQ ID NO: 9 |
| SEQ ID NO: 8 | SEQ ID NO: 1 | SEQ ID NO: 4 | SEQ ID NO: 7 | SEQ ID NO: 9 |
| SEQ ID NO: 8 | SEQ ID NO: 2 | SEQ ID NO: 3 | SEQ ID NO: 7 | SEQ ID NO: 9 |
| SEQ ID NO: 8 | SEQ ID NO: 2 | SEQ ID NO: 4 | SEQ ID NO: 7 | SEQ ID NO: 9 |
| SEQ ID NO: 8 | SEQ ID NO: 1 | SEQ ID NO: 3 | SEQ ID NO: 6 | SEQ ID NO: 9 |
| SEQ ID NO: 8 | SEQ ID NO: 1 | SEQ ID NO: 4 | SEQ ID NO: 6 | SEQ ID NO: 9 |
| SEQ ID NO: 8 | SEQ ID NO: 2 | SEQ ID NO: 3 | SEQ ID NO: 6 | SEQ ID NO: 9 |
| SEQ ID NO: 8 | SEQ ID NO: 2 | SEQ ID NO: 4 | SEQ ID NO: 6 | SEQ ID NO: 9 |
| SEQ ID NO: 10 | SEQ ID NO: 1 | SEQ ID NO: 28 | SEQ ID NO: 7 | SEQ ID NO: 11 |
| SEQ ID NO: 10 | SEQ ID NO: 2 | SEQ ID NO: 28 | SEQ ID NO: 7 | SEQ ID NO: 11 |
| SEQ ID NO: 10 | SEQ ID NO: 1 | SEQ ID NO: 28 | SEQ ID NO: 6 | SEQ ID NO: 11 |
| SEQ ID NO: 10 | SEQ ID NO: 2 | SEQ ID NO: 28 | SEQ ID NO: 6 | SEQ ID NO: 11 |
| SEQ ID NO: 8 | SEQ ID NO: 1 | SEQ ID NO: 28 | SEQ ID NO: 7 | SEQ ID NO: 9 |
| SEQ ID NO: 8 | SEQ ID NO: 2 | SEQ ID NO: 28 | SEQ ID NO: 7 | SEQ ID NO: 9 |
| SEQ ID NO: 8 | SEQ ID NO: 1 | SEQ ID NO: 28 | SEQ ID NO: 6 | SEQ ID NO: 9 |
| SEQ ID NO: 8 | SEQ ID NO: 2 | SEQ ID NO: 28 | SEQ ID NO: 6 | SEQ ID NO: 9 |

Transfer plasmid sequences that may be used to produce the nucleic acid vectors (e.g., AAV vectors) for use in the compositions and methods described herein are provided in Table 5. A transfer plasmid (e.g., a plasmid containing a DNA sequence to be delivered by a nucleic acid vector, e.g., to be delivered by an AAV vector) may be co-delivered into producer cells with a helper plasmid (e.g., a plasmid providing proteins necessary for AAV manufacture) and a rep/cap plasmid (e.g., a plasmid that provides AAV capsid proteins and proteins that insert the transfer plasmid DNA sequence into the capsid shell) to produce a nucleic acid vector (e.g., an AAV vector) for administration. The following transfer plasmids are designed to produce nucleic acid vectors (e.g., AAV vectors) containing a GJB2 regulatory construct described herein (e.g., a polynucleotide having the sequence of SEQ ID NO: 1 or SEQ ID NO: 2) operably linked to a polynucleotide encoding a Gjb2 protein (e.g., a polynucleotide of SEQ ID NO: 3 or SEQ ID NO: 4). The sequence spanning the 5′ ITR to the 3′ ITR (including the intervening GJB2 regulatory construct, GJB2 coding sequence, and polyA signal sequence) is packaged into the AAV vector.

TABLE 5
Transfer plasmids for the production of AAV vectors

| SEQ | | |
|---|---|---|
| ID | | |
| NO. | Description | Plasmid Sequence |
| 12 | Plasmid P1589 | CTGCGCGCTCGCTCGCTCACTGAGGCCGCCCGGGCAAAGC |
| | 5′ ITR at nucleotides | CCGGGCGTCGGGCGACCTTTGGTCGCCCGGCCTCAGTGAG |
| | 1-130 | CGAGCGAGCGCGCAGAGAGGGAGTGGCCAACTCCATCACTA |
| | GJB2 regulatory | GGGGTTCCTGAGCAGAATTCTGGTACCTTGCTAGCAAAAACT |
| | construct at nucleotides | CTTACATAATTGTAACAGATTTGAGTTTCCTCTGGTTCAGGTT |
| | 157-1541 | TTCTTGCCTCTTTGATAATCAAATGATCTGAAGAAAGGCATAG |
| | CpG depleted hGJB2 at | AATTTCAAGGGAGAATCTGCATGACAGGATTACAATAAGGCT |
| | nucleotides 1555-2232 | ATTCATGGAGACTCTTTTATTAGCTTACACAGGATCTGCATCA |
| | SV40 polyA signal | TTCTTCGTCTCTGGCTTTGCTACAAGGCTCCATTTAAACTTAA |
| | sequence at nucleotides | CCCAACTTGCAGGCTTAACTGATCCAGGAATGACTCAATACA |
| | 2249-2383 | ATGAGCCAGCTACTGTAGCTCTTTTTCCCCTTTTGATAAGGG |
| | 3′ ITR at nucleotides | GAGTTAACACAATGGGCTTTACAGTTCTTAAATGAACGCGTAC |
| | 2402-2531 | AGAGGACAACGACCACAGCCATCCCTGAACCCCGCCCACGG |
| | AmpR promoter at | CACAGCGCCGGAGCCGGGGTCTGGGGCGCCGCTTCCTGGG |
| | nucleotides 3189-3293 | GGGTCCCGACTCTCAGCCGCCCCCGCTTCACCCGGGCCGC |
| | KanR (antibiotic | CAAGGGGCTGGGGGAGGCGGCGCTCGGGGTAACCGGGGG |
| | resistance gene) at | AGACTCAGGGCGCTGGGGGCACTTGGGGAACTCATGGGGG |
| | nucleotides 3294-4103 | CTCAAAGGAACTAGGAGATCGGGACCTCGAAGGGGACTTGG |
| | Sequence to be | GGGGTTCGGGGCTTTCGGGGGCGGTCGGGGGTTCGCGGAC |
| | transferred into vector | CCGGGAAGCTCTGAGGACCCAGAGGCCGGGCGCGCTCCGC |
| | at nucleotides 1-2531 | CCGCGGCGCCGCCCCCTCCGTAACTTTCCCAGTCTCCGAGG |
| | | GAAGAGGCGGGGTGTGGGGTGCGGTTAAAAGGCGCCACGG |
| | | CGGGAGACAGGTGTTGCGGCCCCGCAGCGCCCGCGCGCTC |
| | | CTCTCCCCGACTCGGAGCCCCTCGGCGGCGCCCGGCCCAG |
| | | GACCCGCCTAGGAGCGCAGGAGCCCCAGCGCAGAGACCCC |
| | | AACGCCGAGACCCCCGCCCCGGCCCCGCCGCGCTTCCTCC |
| | | CGACGCAGGTGAGCCCGCCGGCCCCGGACTGCCCGGCCAG |
| | | GAACCTGGCGCGGGGAGGGACCGCGAGACCCAGAGCGGTT |
| | | GCCCGGCCGCGTGGGTCTCGGGGAACCGGGGGGCTGGACC |
| | | AACACACGTCCTTGGGCCGGGGGGCGGGGGCCGCCTTCTG |
| | | GAGCGGGCGTTTCTGTTTATGATGTGTTTAAAGATTGGGTGA |
| | | ATTACTCAGGTGAACAAGCTACTTTTTATCAGAGAACACCTAA |
| | | AAACACGTTCAAGAGGGTTTGGGAACTATACATTTAATCCTAT |
| | | GACAAACTAAGTTGGTTCTGTCTTCACCTGTTTTGGTGAGGTT |
| | | GTGTAAGAGTTGGTGTTTGCTCAGGAAGAGATTTAAGCATGC |
| | | TTGCTTACCCAGACTCAGAGAAGTCTCCCTGTTCTGTCCTAG |
| | | CTAGTGATTCCTGTGTTGTGTGCATTCGTCTTTTCCAGAGCAA |
| | | ACCGCCCAGAGTAGAAGCCTGCAGGCCACCATGGATTGGGG |
| | | CACACTGCAGACTATCCTGGGGGGTGTGAACAAACACTCCAC |
| | | CAGCATTGGAAAGATCTGGCTCACAGTCCTCTTCATTTTTAGG |
| | | ATTATGATCCTGGTTGTGGCTGCAAAGGAGGTGTGGGGAGAT |
| | | GAGCAGGCTGACTTTGTCTGCAACACCCTGCAGCCAGGCTG |
| | | CAAGAATGTGTGCTATGATCACTACTTCCCCATCTCCCACATC |
| | | AGGCTATGGGCCCTGCAGCTGATCTTTGTGTCCACTCCAGCA |
| | | CTCCTAGTGGCCATGCATGTGGCCTACAGGAGACATGAGAA |
| | | GAAGAGGAAGTTCATCAAGGGGGAGATAAAGAGTGAATTTAA |
| | | GGACATTGAGGAGATCAAAACCCAGAAGGTCAGAATTGAAGG |
| | | CTCCCTGTGGTGGACCTACACAAGCAGCATCTTCTTCAGGGT |
| | | CATCTTTGAAGCAGCCTTCATGTATGTCTTCTATGTCATGTAT |
| | | GATGGCTTCTCCATGCAGAGGCTGGTGAAGTGCAATGCCTG |
| | | GCCTTGTCCCAACACTGTGGACTGCTTTGTGTCCAGGCCCAC |
| | | TGAGAAGACTGTCTTCACAGTGTTCATGATTGCAGTGTCTGG |
| | | AATTTGCATCCTGCTGAATGTCACTGAATTGTGTTATTTGCTA |
| | | ATTAGATATTGTTCTGGGAAGTCAAAAAAGCCAGTTTGATAAG |
| | | CTTAGATCTGATCCAGACATGATAAGATACATTGATGAGTTTG |
| | | GACAAACCAAAACTAGAATGCAGTGAAAAAAATGCCTTATTTG |
| | | TGAAATTTGTGATGCTATTGCCTTATTTGTAACCATTATAAGCT |
| | | GCAATAAACAAGTTTCTAGAACTGAATTCACAAGGAACCCCTA |
| | | GTGATGGAGTTGGCCACTCCCTCTCTGCGCGCTCGCTCGCT |
| | | CACTGAGGCCGGGCGACCAAAGGTCGCCCGACGCCCGGGC |
| | | TTTGCCCGGGCGGCCTCAGTGAGCGAGCGAGCGCGCAGCC |
| | | TTAATTAACCTAATTCACTGGCCGTCGTTTTACAACGTCGTGA |
| | | CTGGGAAAACCCTGGCGTTACCCAACTTAATCGCCTTGCAGC |
| | | ACATCCCCCTTTCGCCAGCTGGCGTAATAGCGAAGAGGCCC |
| | | GCACCGATCGCCCTTCCCAACAGTTGCGCAGCCTGAATGGC |
| | | GAATGGGACGCGCCCTGTAGCGGCGCATTAAGCGCGGCGG |
| | | GTGTGGTGGTTACGCGCAGCGTGACCGCTACACTTGCCAGC |
| | | GCCCTAGCGCCCGCTCCTTTCGCTTTCTTCCCTTCCTTTCTC |
| | | GCCACGTTCGCCGGCTTTCCCCGTCAAGCTCTAAATCGGGG |
| | | GCTCCCTTTAGGGTTCCGATTTAGTGCTTTACGGCACCTCGA |
| | | CCCCAAAAAACTTGATTAGGGTGATGGTTCACGTAGTGGGCC |
| | | ATCGCCCTGATAGACGGTTTTTCGCCCTTTGACGTTGGAGTC |
| | | CACGTTCTTTAATAGTGGACTCTTGTTCCAAACTGGAACAACA |
| | | CTCAACCCTATCTCGGTCTATTCTTTTGATTTATAAGGGATTTT |
| | | GCCGATTTCGGCCTATTGGTTAAAAAATGAGCTGATTTAACAA |
| | | AAATTTAACGCGAATTTTAACAAAATATTAACGCTTACAATTTA |
| | | GGTGGCACTTTTCGGGGAAATGTGCGCGGAACCCCTATTTGT |
| | | TTATTTTTCTAAATACATTCAAATATGTATCCGCTCATGAGACA |
| | | ATAACCCTGATAAATGCTTCAATAATATTGAAAAAGGAAGAGT |
| | | ATGAGCCATATTCAACGGGAAACGTCGAGGCCGCGATTAAAT |
| | | TCCAACATGGATGCTGATTTATATGGGTATAAATGGGCTCGC |
| | | GATAATGTCGGGCAATCAGGTGCGACAATCTATCGCTTGTAT |
| | | GGGAAGCCCGATGCGCCAGAGTTGTTTCTGAAACATGGCAA |
| | | AGGTAGCGTTGCCAATGATGTTACAGATGAGATGGTCAGACT |
| | | AAACTGGCTGACGGAATTTATGCCTCTTCCGACCATCAAGCA |
| | | TTTTATCCGTACTCCTGATGATGCATGGTTACTCACCACTGCG |
| | | ATCCCCGGAAAAACAGCATTCCAGGTATTAGAAGAATATCCT |
| | | GATTCAGGTGAAAATATTGTTGATGCGCTGGCAGTGTTCCTG |
| | | CGCCGGTTGCATTCGATTCCTGTTTGTAATTGTCCTTTTAACA |
| | | GCGATCGCGTATTTCGTCTCGCTCAGGCGCAATCACGAATGA |
| | | ATAACGGTTTGGTTGATGCGAGTGATTTTGATGACGAGCGTA |
| | | ATGGCTGGCCTGTTGAACAAGTCTGGAAAGAAATGCATAAAC |
| | | TTTTGCCATTCTCACCGGATTCAGTCGTCACTCATGGTGATTT |
| | | CTCACTTGATAACCTTATTTTTGACGAGGGGAAATTAATAGGT |
| | | TGTATTGATGTTGGACGAGTCGGAATCGCAGACCGATACCAG |
| | | GATCTTGCCATCCTATGGAACTGCCTCGGTGAGTTTTCTCCTT |
| | | CATTACAGAAACGGCTTTTTCAAAAATATGGTATTGATAATCC |
| | | TGATATGAATAAATTGCAGTTTCATTTGATGCTCGATGAGTTT |
| | | TTCTAACTGTCAGACCAAGTTTACTCATATATACTTTAGATTGA |
| | | TTTAAAACTTCATTTTTAATTTAAAAGGATCTAGGTGAAGATCC |
| | | TTTTTGATAATCTCATGACCAAAATCCCTTAACGTGAGTTTTC |
| | | GTTCCACTGAGCGTCAGACCCCGTAGAAAAGATCAAAGGATC |
| | | TTCTTGAGATCCTTTTTTTCTGCGCGTAATCTGCTGCTTGCAA |
| | | ACAAAAAAACCACCGCTACCAGCGGTGGTTTGTTTGCCGGAT |
| | | CAAGAGCTACCAACTCTTTTTCCGAAGGTAACTGGCTTCAGC |
| | | AGAGCGCAGATACCAAATACTGTTCTTCTAGTGTAGCCGTAG |
| | | TTAGGCCACCACTTCAAGAACTCTGTAGCACCGCCTACATAC |
| | | CTCGCTCTGCTAATCCTGTTACCAGTGGCTGCTGCCAGTGGC |
| | | GATAAGTCGTGTCTTACCGGGTTGGACTCAAGACGATAGTTA |
| | | CCGGATAAGGCGCAGCGGTCGGGCTGAACGGGGGGTTCGT |
| | | GCACACAGCCCAGCTTGGAGCGAACGACCTACACCGAACTG |
| | | AGATACCTACAGCGTGAGCTATGAGAAAGCGCCACGCTTCCC |
| | | GAAGGGAGAAAGGCGGACAGGTATCCGGTAAGCGGCAGGG |
| | | TCGGAACAGGAGAGCGCACGAGGGAGCTTCCAGGGGGAAA |
| | | CGCCTGGTATCTTTATAGTCCTGTCGGGTTTCGCCACCTCTG |
| | | ACTTGAGCGTCGATTTTTGTGATGCTCGTCAGGGGGGCGGA |
| | | GCCTATGGAAAAACGCCAGCAACGCGGCCTTTTTACGGTTCC |
| | | TGGCCTTTTGCTGGCCTTTTGCTCACATGTTCTTTCCTGCGTT |
| | | ATCCCCTGATTCTGTGGATAACCGTATTACCGCCTTTGAGTG |
| | | AGCTGATACCGCTCGCCGCAGCCGAACGACCGAGCGCAGC |
| | | GAGTCAGTGAGCGAGGAAGCGGAAGAGCGCCCAATACGCAA |
| | | ACCGCCTCTCCCCGCGCGTTGGCCGATTCATTAATGCAGCTG |
| | | GCACGACAGGTTTCCCGACTGGAAAGCGGGCAGTGAGCGCA |
| | | ACGCAATTAATGTGAGTTAGCTCACTCATTAGGCACCCCAGG |
| | | CTTTACACTTTATGCTTCCGGCTCGTATGTTGTGTGGAATTGT |
| | | GAGCGGATAACAATTTCACACAGGAAACAGCTATGACCATGA |
| | | TTACGCCAGATTTAATTAAGGCCTTAATTAGG |
| 13 | Plasmid P1590 | CTGCGCGCTCGCTCGCTCACTGAGGCCGCCCGGGCAAAGC |
| | 5′ ITR at nucleotides | CCGGGCGTCGGGCGACCTTTGGTCGCCCGGCCTCAGTGAG |
| | 1-130 | CGAGCGAGCGCGCAGAGAGGGAGTGGCCAACTCCATCACTA |
| | GJB2 regulatory | GGGGTTCCTGAGCAGAATTCTGGTACCTTGCTAGCAAAAACT |
| | construct at nucleotides | CTTACATAATTGTAACAGATTTGAGTTTCCTCTGGTTCAGGTT |
| | 157-1541 | TTCTTGCCTCTTTGATAATCAAATGATCTGAAGAAAGGCATAG |
| | Codon-optimized and | AATTTCAAGGGAGAATCTGCATGACAGGATTACAATAAGGCT |
| | CpG depleted hGJB2 at | ATTCATGGAGACTCTTTTATTAGCTTACACAGGATCTGCATCA |
| | nucleotides 1555-2232 | TTCTTCGTCTCTGGCTTTGCTACAAGGCTCCATTTAAACTTAA |
| | SV40 polyA signal | CCCAACTTGCAGGCTTAACTGATCCAGGAATGACTCAATACA |
| | sequence at nucleotides | ATGAGCCAGCTACTGTAGCTCTTTTTCCCCTTTTGATAAGGG |
| | 2249-2383 | GAGTTAACACAATGGGCTTTACAGTTCTTAAATGAACGCGTAC |
| | 3′ ITR at nucleotides | AGAGGACAACGACCACAGCCATCCCTGAACCCCGCCCACGG |
| | 2402-2531 | CACAGCGCCGGAGCCGGGGTCTGGGGCGCCGCTTCCTGGG |
| | AmpR promoter at | GGGTCCCGACTCTCAGCCGCCCCCGCTTCACCCGGGCCGC |
| | nucleotides 3189-3293 | CAAGGGGCTGGGGGAGGCGGCGCTCGGGGTAACCGGGGG |
| | KanR at nucleotides | AGACTCAGGGCGCTGGGGGCACTTGGGGAACTCATGGGGG |
| | 3294-4103 | CTCAAAGGAACTAGGAGATCGGGACCTCGAAGGGGACTTGG |
| | Sequence to be | GGGGTTCGGGGCTTTCGGGGGCGGTCGGGGGTTCGCGGAC |
| | transferred into vector | CCGGGAAGCTCTGAGGACCCAGAGGCCGGGCGCGCTCCGC |
| | at nucleotides 1-2531 | CCGCGGCGCCGCCCCCTCCGTAACTTTCCCAGTCTCCGAGG |
| | | GAAGAGGCGGGGTGTGGGGTGCGGTTAAAAGGCGCCACGG |
| | | CGGGAGACAGGTGTTGCGGCCCCGCAGCGCCCGCGCGCTC |
| | | CTCTCCCCGACTCGGAGCCCCTCGGCGGCGCCCGGCCCAG |
| | | GACCCGCCTAGGAGCGCAGGAGCCCCAGCGCAGAGACCCC |
| | | AACGCCGAGACCCCCGCCCCGGCCCCGCCGCGCTTCCTCC |
| | | CGACGCAGGTGAGCCCGCCGGCCCCGGACTGCCCGGCCAG |
| | | GAACCTGGCGCGGGGAGGGACCGCGAGACCCAGAGCGGTT |
| | | GCCCGGCCGCGTGGGTCTCGGGGAACCGGGGGGCTGGACC |
| | | AACACACGTCCTTGGGCCGGGGGGCGGGGGCCGCCTTCTG |
| | | GAGCGGGCGTTTCTGTTTATGATGTGTTTAAAGATTGGGTGA |
| | | ATTACTCAGGTGAACAAGCTACTTTTTATCAGAGAACACCTAA |
| | | AAACACGTTCAAGAGGGTTTGGGAACTATACATTTAATCCTAT |
| | | GACAAACTAAGTTGGTTCTGTCTTCACCTGTTTTGGTGAGGTT |
| | | GTGTAAGAGTTGGTGTTTGCTCAGGAAGAGATTTAAGCATGC |
| | | TTGCTTACCCAGACTCAGAGAAGTCTCCCTGTTCTGTCCTAG |
| | | CTAGTGATTCCTGTGTTGTGTGCATTCGTCTTTTCCAGAGCAA |
| | | ACCGCCCAGAGTAGAAGCCTGCAGGCCACCATGGACTGGGG |
| | | AACACTGCAAACAATCCTGGGAGGTGTGAACAAGCACAGCAC |
| | | AAGCATTGGCAAGATCTGGCTGACAGTGCTGTTCATCTTTAG |
| | | AATCATGATCCTGGTTGTGGCTGCTAAGGAAGTGTGGGGAGA |
| | | TGAGCAGGCTGACTTTGTTTGTAATACCCTGCAACCTGGCTG |
| | | CAAGAATGTGTGTTATGACCACTACTTCCCCATCAGCCACAT |
| | | CAGACTGTGGGCCCTGCAACTGATCTTTGTGTCCACCCCTGC |
| | | CCTGCTGGTGGCCATGCATGTGGCCTACAGAAGGCATGAGA |
| | | AGAAGAGGAAATTCATCAAGGGAGAGATCAAGAGTGAGTTCA |
| | | AGGACATTGAGGAAATCAAAACCCAGAAGGTGAGGATTGAAG |
| | | GCTCTCTGTGGTGGACCTACACCAGCAGCATCTTCTTCAGAG |
| | | TGATCTTTGAGGCAGCCTTTATGTATGTGTTCTATGTGATGTA |
| | | TGATGGCTTCTCCATGCAGAGACTGGTGAAGTGCAATGCCTG |
| | | GCCATGTCCTAACACAGTGGACTGCTTTGTGTCTAGACCTAC |
| | | AGAAAAGACAGTGTTTACAGTCTTTATGATTGCAGTGTCTGGC |
| | | ATCTGCATTCTGCTGAATGTGACTGAGCTGTGCTACCTGCTC |
| | | ATTAGGTACTGTAGTGGAAAAAGCAAGAAACCTGTGTGATAA |
| | | GCTTAGATCTGATCCAGACATGATAAGATACATTGATGAGTTT |
| | | GGACAAACCAAAACTAGAATGCAGTGAAAAAAATGCCTTATTT |
| | | GTGAAATTTGTGATGCTATTGCCTTATTTGTAACCATTATAAG |
| | | CTGCAATAAACAAGTTTCTAGAACTGAATTCACAAGGAACCCC |
| | | TAGTGATGGAGTTGGCCACTCCCTCTCTGCGCGCTCGCTCG |
| | | CTCACTGAGGCCGGGCGACCAAAGGTCGCCCGACGCCCGG |
| | | GCTTTGCCCGGGCGGCCTCAGTGAGCGAGCGAGCGCGCAG |
| | | CCTTAATTAACCTAATTCACTGGCCGTCGTTTTACAACGTCGT |
| | | GACTGGGAAAACCCTGGCGTTACCCAACTTAATCGCCTTGCA |
| | | GCACATCCCCCTTTCGCCAGCTGGCGTAATAGCGAAGAGGC |
| | | CCGCACCGATCGCCCTTCCCAACAGTTGCGCAGCCTGAATG |
| | | GCGAATGGGACGCGCCCTGTAGCGGCGCATTAAGCGCGGC |
| | | GGGTGTGGTGGTTACGCGCAGCGTGACCGCTACACTTGCCA |
| | | GCGCCCTAGCGCCCGCTCCTTTCGCTTTCTTCCCTTCCTTTC |
| | | TCGCCACGTTCGCCGGCTTTCCCCGTCAAGCTCTAAATCGGG |
| | | GGCTCCCTTTAGGGTTCCGATTTAGTGCTTTACGGCACCTCG |
| | | ACCCCAAAAAACTTGATTAGGGTGATGGTTCACGTAGTGGGC |
| | | CATCGCCCTGATAGACGGTTTTTCGCCCTTTGACGTTGGAGT |
| | | CCACGTTCTTTAATAGTGGACTCTTGTTCCAAACTGGAACAAC |
| | | ACTCAACCCTATCTCGGTCTATTCTTTTGATTTATAAGGGATTT |
| | | TGCCGATTTCGGCCTATTGGTTAAAAAATGAGCTGATTTAACA |
| | | AAAATTTAACGCGAATTTTAACAAAATATTAACGCTTACAATTT |
| | | AGGTGGCACTTTTCGGGGAAATGTGCGCGGAACCCCTATTTG |
| | | TTTATTTTTCTAAATACATTCAAATATGTATCCGCTCATGAGAC |
| | | AATAACCCTGATAAATGCTTCAATAATATTGAAAAAGGAAGAG |
| | | TATGAGCCATATTCAACGGGAAACGTCGAGGCCGCGATTAAA |
| | | TTCCAACATGGATGCTGATTTATATGGGTATAAATGGGCTCG |
| | | CGATAATGTCGGGCAATCAGGTGCGACAATCTATCGCTTGTA |
| | | TGGGAAGCCCGATGCGCCAGAGTTGTTTCTGAAACATGGCAA |
| | | AGGTAGCGTTGCCAATGATGTTACAGATGAGATGGTCAGACT |
| | | AAACTGGCTGACGGAATTTATGCCTCTTCCGACCATCAAGCA |
| | | TTTTATCCGTACTCCTGATGATGCATGGTTACTCACCACTGCG |
| | | ATCCCCGGAAAAACAGCATTCCAGGTATTAGAAGAATATCCT |
| | | GATTCAGGTGAAAATATTGTTGATGCGCTGGCAGTGTTCCTG |
| | | CGCCGGTTGCATTCGATTCCTGTTTGTAATTGTCCTTTTAACA |
| | | GCGATCGCGTATTTCGTCTCGCTCAGGCGCAATCACGAATGA |
| | | ATAACGGTTTGGTTGATGCGAGTGATTTTGATGACGAGCGTA |
| | | ATGGCTGGCCTGTTGAACAAGTCTGGAAAGAAATGCATAAAC |
| | | TTTTGCCATTCTCACCGGATTCAGTCGTCACTCATGGTGATTT |
| | | CTCACTTGATAACCTTATTTTTGACGAGGGGAAATTAATAGGT |
| | | TGTATTGATGTTGGACGAGTCGGAATCGCAGACCGATACCAG |
| | | GATCTTGCCATCCTATGGAACTGCCTCGGTGAGTTTTCTCCTT |
| | | CATTACAGAAACGGCTTTTTCAAAAATATGGTATTGATAATCC |
| | | TGATATGAATAAATTGCAGTTTCATTTGATGCTCGATGAGTTT |
| | | TTCTAACTGTCAGACCAAGTTTACTCATATATACTTTAGATTGA |
| | | TTTAAAACTTCATTTTTAATTTAAAAGGATCTAGGTGAAGATCC |
| | | TTTTTGATAATCTCATGACCAAAATCCCTTAACGTGAGTTTTC |
| | | GTTCCACTGAGCGTCAGACCCCGTAGAAAAGATCAAAGGATC |
| | | TTCTTGAGATCCTTTTTTTCTGCGCGTAATCTGCTGCTTGCAA |
| | | ACAAAAAAACCACCGCTACCAGCGGTGGTTTGTTTGCCGGAT |
| | | CAAGAGCTACCAACTCTTTTTCCGAAGGTAACTGGCTTCAGC |
| | | AGAGCGCAGATACCAAATACTGTTCTTCTAGTGTAGCCGTAG |
| | | TTAGGCCACCACTTCAAGAACTCTGTAGCACCGCCTACATAC |
| | | CTCGCTCTGCTAATCCTGTTACCAGTGGCTGCTGCCAGTGGC |
| | | GATAAGTCGTGTCTTACCGGGTTGGACTCAAGACGATAGTTA |
| | | CCGGATAAGGCGCAGCGGTCGGGCTGAACGGGGGGTTCGT |
| | | GCACACAGCCCAGCTTGGAGCGAACGACCTACACCGAACTG |
| | | AGATACCTACAGCGTGAGCTATGAGAAAGCGCCACGCTTCCC |
| | | GAAGGGAGAAAGGCGGACAGGTATCCGGTAAGCGGCAGGG |
| | | TCGGAACAGGAGAGCGCACGAGGGAGCTTCCAGGGGGAAA |
| | | CGCCTGGTATCTTTATAGTCCTGTCGGGTTTCGCCACCTCTG |
| | | ACTTGAGCGTCGATTTTTGTGATGCTCGTCAGGGGGGCGGA |
| | | GCCTATGGAAAAACGCCAGCAACGCGGCCTTTTTACGGTTCC |
| | | TGGCCTTTTGCTGGCCTTTTGCTCACATGTTCTTTCCTGCGTT |
| | | ATCCCCTGATTCTGTGGATAACCGTATTACCGCCTTTGAGTG |
| | | AGCTGATACCGCTCGCCGCAGCCGAACGACCGAGCGCAGC |
| | | GAGTCAGTGAGCGAGGAAGCGGAAGAGCGCCCAATACGCAA |
| | | ACCGCCTCTCCCCGCGCGTTGGCCGATTCATTAATGCAGCTG |
| | | GCACGACAGGTTTCCCGACTGGAAAGCGGGCAGTGAGCGCA |
| | | ACGCAATTAATGTGAGTTAGCTCACTCATTAGGCACCCCAGG |
| | | CTTTACACTTTATGCTTCCGGCTCGTATGTTGTGTGGAATTGT |
| | | GAGCGGATAACAATTTCACACAGGAAACAGCTATGACCATGA |
| | | TTACGCCAGATTTAATTAAGGCCTTAATTAGG |
| 14 | Plasmid P1592 | CTGCGCGCTCGCTCGCTCACTGAGGCCGCCCGGGCAAAGC |
| | 5′ ITR at nucleotides | CCGGGCGTCGGGCGACCTTTGGTCGCCCGGCCTCAGTGAG |
| | 1-130 | CGAGCGAGCGCGCAGAGAGGGAGTGGCCAACTCCATCACTA |
| | GJB2 regulatory | GGGGTTCCTGAGCAGAATTCTGGTACCTTGCTAGCAAAAACT |
| | construct at nucleotides | CTTACATAATTGTAACAGATTTGAGTTTCCTCTGGTTCAGGTT |
| | 157-2617 | TTCTTGCCTCTTTGATAATCAAATGATCTGAAGAAAGGCATAG |
| | CpG depleted hGJB2 at | AATTTCAAGGGAGAATCTGCATGACAGGATTACAATAAGGCT |
| | nucleotides 2631-3308 | ATTCATGGAGACTCTTTTATTAGCTTACACAGGATCTGCATCA |
| | SV40 polyA signal | TTCTTCGTCTCTGGCTTTGCTACAAGGCTCCATTTAAACTTAA |
| | sequence at nucleotides | CCCAACTTGCAGGCTTAACTGATCCAGGAATGACTCAATACA |
| | 3325-3459 | ATGAGCCAGCTACTGTAGCTCTTTTTCCCCTTTTGATAAGGG |
| | 3′ ITR at nucleotides | GAGTTAACACAATGGGCTTTACAGTTCTTAAATGACCGTTCCT |
| | 3478-3607 | GCAGGTATTGGAAAACCTGCATCGTATGCCTGCTGCTTTAGG |
| | AmpR promoter at | AGACAACAAACTCAGCAGCCCCTCCCCCGAAGCTGAAGTGC |
| | nucleotides 4265-4369 | CAAACTTAAGGCTCCAGGTAAACTGCCCTTCCGATGAGAGCT |
| | KanR at nucleotides | TTACATTTAGGCAGTAGCCACAGGCCTAAACAAAGTCAAATG |
| | 4370-5179 | GATGAAACTTGTGATCTCTCCTTGGAAATATGGCCAAGAGTG |
| | Sequence to be | AAACAACTACTTAGTTATTATTTTAAACGCAGGCTGCTAAATG |
| | transferred into vector | GCTTGTGTTGGTATGTAATGGCATCCTAGATAATGGGGGCTG |
| | at nucleotides 1-3607 | CCAAGAAAATATTCACACTGGCAAGTCAATTAAAATACCGTTC |
| | | TCTTCCTAAGAGGCCTGGCTGGATTCCAGAGCTTCCTTTAGG |
| | | GTAACGCTCAAACCTGTCATAGTTTATTTTGTTTCTGTTTTCAG |
| | | GCAACAGATTTGGTTATTGCTTTTATTGTATGAGTGTTGAAAT |
| | | GGTGTCCTAACAGCCATCTGTACTGCCATCCCCTGGTCTCTC |
| | | TGCCTTTCCAGAAAGGAGATGAAGTTGCTGGAGCAGGCAGG |
| | | TTCTCTGAAAGGCTCCCTGAGTGTGGAAGAGCAGCTGTCACT |
| | | CATCAGTGGTTGTCCCAATATCCAAGAAGCAGTAGAGGGTGC |
| | | CATGCACATTCAGGTGAGTGAAGGACACTGGCGCCGCATCT |
| | | GTGGGGCCTGAGACATTGTCTCCCAGCCTCTCCGGAGGAAC |
| | | AGGGCAGGATACAGAGCAGTGGCAGGTGTGGGCATTTTGCA |
| | | TCTTCTGTAATCTCCAAACAAAGTCTGCGCTAGGCCCTTTCAG |
| | | GCTATAAAGGACAGGGTACTCTGATTTCTGCTCTTGATGAGG |
| | | GTTGATTGTGATACCAGGCGAGGGAGTAAGAGATACAGGAC |
| | | ACCATCTCTGCGGCTGCATCTTTGTCGCAGAAACAGACATTG |
| | | TTCAGGGGACACCTGAGCCTCTGATAGTCAGCCAGTGGCTCT |
| | | GGCAGCTCACAGCAGTTATAGGGTGGCTGTGTCCTTTTCTAA |
| | | AGTCCTGACTTTTTATGGCCTGTGGTCATGGTGACTCCACTA |
| | | CGGGCTGTGTTTCTGTTGACGCGTACAGAGGACAACGACCA |
| | | CAGCCATCCCTGAACCCCGCCCACGGCACAGCGCCGGAGC |
| | | CGGGGTCTGGGGCGCCGCTTCCTGGGGGGTCCCGACTCTC |
| | | AGCCGCCCCCGCTTCACCCGGGCCGCCAAGGGGCTGGGGG |
| | | AGGCGGCGCTCGGGGTAACCGGGGGAGACTCAGGGCGCTG |
| | | GGGGCACTTGGGGAACTCATGGGGGCTCAAAGGAACTAGGA |
| | | GATCGGGACCTCGAAGGGGACTTGGGGGGTTCGGGGCTTTC |
| | | GGGGGCGGTCGGGGGTTCGCGGACCCGGGAAGCTCTGAGG |
| | | ACCCAGAGGCCGGGCGCGCTCCGCCCGCGGCGCCGCCCCC |
| | | TCCGTAACTTTCCCAGTCTCCGAGGGAAGAGGCGGGGTGTG |
| | | GGGTGCGGTTAAAAGGCGCCACGGCGGGAGACAGGTGTTG |
| | | CGGCCCCGCAGCGCCCGCGCGCTCCTCTCCCCGACTCGGA |
| | | GCCCCTCGGCGGCGCCCGGCCCAGGACCCGCCTAGGAGCG |
| | | CAGGAGCCCCAGCGCAGAGACCCCAACGCCGAGACCCCCG |
| | | CCCCGGCCCCGCCGCGCTTCCTCCCGACGCAGGTGAGCCC |
| | | GCCGGCCCCGGACTGCCCGGCCAGGAACCTGGCGCGGGGA |
| | | GGGACCGCGAGACCCAGAGCGGTTGCCCGGCCGCGTGGGT |
| | | CTCGGGGAACCGGGGGGCTGGACCAACACACGTCCTTGGG |
| | | CCGGGGGGCGGGGGCCGCCTTCTGGAGCGGGCGTTTCTGT |
| | | TTATGATGTGTTTAAAGATTGGGTGAATTACTCAGGTGAACAA |
| | | GCTACTTTTTATCAGAGAACACCTAAAAACACGTTCAAGAGG |
| | | GTTTGGGAACTATACATTTAATCCTATGACAAACTAAGTTGGT |
| | | TCTGTCTTCACCTGTTTTGGTGAGGTTGTGTAAGAGTTGGTGT |
| | | TTGCTCAGGAAGAGATTTAAGCATGCTTGCTTACCCAGACTC |
| | | AGAGAAGTCTCCCTGTTCTGTCCTAGCTAGTGATTCCTGTGTT |
| | | GTGTGCATTCGTCTTTTCCAGAGCAAACCGCCCAGAGTAGAA |
| | | GCCTGCAGGCCACCATGGATTGGGGCACACTGCAGACTATC |
| | | CTGGGGGGTGTGAACAAACACTCCACCAGCATTGGAAAGAT |
| | | CTGGCTCACAGTCCTCTTCATTTTTAGGATTATGATCCTGGTT |
| | | GTGGCTGCAAAGGAGGTGTGGGGAGATGAGCAGGCTGACTT |
| | | TGTCTGCAACACCCTGCAGCCAGGCTGCAAGAATGTGTGCTA |
| | | TGATCACTACTTCCCCATCTCCCACATCAGGCTATGGGCCCT |
| | | GCAGCTGATCTTTGTGTCCACTCCAGCACTCCTAGTGGCCAT |
| | | GCATGTGGCCTACAGGAGACATGAGAAGAAGAGGAAGTTCA |
| | | TCAAGGGGGAGATAAAGAGTGAATTTAAGGACATTGAGGAGA |
| | | TCAAAACCCAGAAGGTCAGAATTGAAGGCTCCCTGTGGTGGA |
| | | CCTACACAAGCAGCATCTTCTTCAGGGTCATCTTTGAAGCAG |
| | | CCTTCATGTATGTCTTCTATGTCATGTATGATGGCTTCTCCAT |
| | | GCAGAGGCTGGTGAAGTGCAATGCCTGGCCTTGTCCCAACA |
| | | CTGTGGACTGCTTTGTGTCCAGGCCCACTGAGAAGACTGTCT |
| | | TCACAGTGTTCATGATTGCAGTGTCTGGAATTTGCATCCTGCT |
| | | GAATGTCACTGAATTGTGTTATTTGCTAATTAGATATTGTTCTG |
| | | GGAAGTCAAAAAAGCCAGTTTGATAAGCTTAGATCTGATCCA |
| | | GACATGATAAGATACATTGATGAGTTTGGACAAACCAAAACTA |
| | | GAATGCAGTGAAAAAAATGCCTTATTTGTGAAATTTGTGATGC |
| | | TATTGCCTTATTTGTAACCATTATAAGCTGCAATAAACAAGTTT |
| | | CTAGAACTGAATTCACAAGGAACCCCTAGTGATGGAGTTGGC |
| | | CACTCCCTCTCTGCGCGCTCGCTCGCTCACTGAGGCCGGGC |
| | | GACCAAAGGTCGCCCGACGCCCGGGCTTTGCCCGGGCGGC |
| | | CTCAGTGAGCGAGCGAGCGCGCAGCCTTAATTAACCTAATTC |
| | | ACTGGCCGTCGTTTTACAACGTCGTGACTGGGAAAACCCTGG |
| | | CGTTACCCAACTTAATCGCCTTGCAGCACATCCCCCTTTCGC |
| | | CAGCTGGCGTAATAGCGAAGAGGCCCGCACCGATCGCCCTT |
| | | CCCAACAGTTGCGCAGCCTGAATGGCGAATGGGACGCGCCC |
| | | TGTAGCGGCGCATTAAGCGCGGCGGGTGTGGTGGTTACGCG |
| | | CAGCGTGACCGCTACACTTGCCAGCGCCCTAGCGCCCGCTC |
| | | CTTTCGCTTTCTTCCCTTCCTTTCTCGCCACGTTCGCCGGCTT |
| | | TCCCCGTCAAGCTCTAAATCGGGGGCTCCCTTTAGGGTTCCG |
| | | ATTTAGTGCTTTACGGCACCTCGACCCCAAAAAACTTGATTAG |
| | | GGTGATGGTTCACGTAGTGGGCCATCGCCCTGATAGACGGT |
| | | TTTTCGCCCTTTGACGTTGGAGTCCACGTTCTTTAATAGTGGA |
| | | CTCTTGTTCCAAACTGGAACAACACTCAACCCTATCTCGGTCT |
| | | ATTCTTTTGATTTATAAGGGATTTTGCCGATTTCGGCCTATTG |
| | | GTTAAAAAATGAGCTGATTTAACAAAAATTTAACGCGAATTTTA |
| | | ACAAAATATTAACGCTTACAATTTAGGTGGCACTTTTCGGGGA |
| | | AATGTGCGCGGAACCCCTATTTGTTTATTTTTCTAAATACATT |
| | | CAAATATGTATCCGCTCATGAGACAATAACCCTGATAAATGCT |
| | | TCAATAATATTGAAAAAGGAAGAGTATGAGCCATATTCAACGG |
| | | GAAACGTCGAGGCCGCGATTAAATTCCAACATGGATGCTGAT |
| | | TTATATGGGTATAAATGGGCTCGCGATAATGTCGGGCAATCA |
| | | GGTGCGACAATCTATCGCTTGTATGGGAAGCCCGATGCGCC |
| | | AGAGTTGTTTCTGAAACATGGCAAAGGTAGCGTTGCCAATGA |
| | | TGTTACAGATGAGATGGTCAGACTAAACTGGCTGACGGAATT |
| | | TATGCCTCTTCCGACCATCAAGCATTTTATCCGTACTCCTGAT |
| | | GATGCATGGTTACTCACCACTGCGATCCCCGGAAAAACAGCA |
| | | TTCCAGGTATTAGAAGAATATCCTGATTCAGGTGAAAATATTG |
| | | TTGATGCGCTGGCAGTGTTCCTGCGCCGGTTGCATTCGATTC |
| | | CTGTTTGTAATTGTCCTTTTAACAGCGATCGCGTATTTCGTCT |
| | | CGCTCAGGCGCAATCACGAATGAATAACGGTTTGGTTGATGC |
| | | GAGTGATTTTGATGACGAGCGTAATGGCTGGCCTGTTGAACA |
| | | AGTCTGGAAAGAAATGCATAAACTTTTGCCATTCTCACCGGAT |
| | | TCAGTCGTCACTCATGGTGATTTCTCACTTGATAACCTTATTT |
| | | TTGACGAGGGGAAATTAATAGGTTGTATTGATGTTGGACGAG |
| | | TCGGAATCGCAGACCGATACCAGGATCTTGCCATCCTATGGA |
| | | ACTGCCTCGGTGAGTTTTCTCCTTCATTACAGAAACGGCTTTT |
| | | TCAAAAATATGGTATTGATAATCCTGATATGAATAAATTGCAG |
| | | TTTCATTTGATGCTCGATGAGTTTTTCTAACTGTCAGACCAAG |
| | | TTTACTCATATATACTTTAGATTGATTTAAAACTTCATTTTTAAT |
| | | TTAAAAGGATCTAGGTGAAGATCCTTTTTGATAATCTCATGAC |
| | | CAAAATCCCTTAACGTGAGTTTTCGTTCCACTGAGCGTCAGA |
| | | CCCCGTAGAAAAGATCAAAGGATCTTCTTGAGATCCTTTTTTT |
| | | CTGCGCGTAATCTGCTGCTTGCAAACAAAAAAACCACCGCTA |
| | | CCAGCGGTGGTTTGTTTGCCGGATCAAGAGCTACCAACTCTT |
| | | TTTCCGAAGGTAACTGGCTTCAGCAGAGCGCAGATACCAAAT |
| | | ACTGTTCTTCTAGTGTAGCCGTAGTTAGGCCACCACTTCAAG |
| | | AACTCTGTAGCACCGCCTACATACCTCGCTCTGCTAATCCTG |
| | | TTACCAGTGGCTGCTGCCAGTGGCGATAAGTCGTGTCTTACC |
| | | GGGTTGGACTCAAGACGATAGTTACCGGATAAGGCGCAGCG |
| | | GTCGGGCTGAACGGGGGGTTCGTGCACACAGCCCAGCTTGG |
| | | AGCGAACGACCTACACCGAACTGAGATACCTACAGCGTGAG |
| | | CTATGAGAAAGCGCCACGCTTCCCGAAGGGAGAAAGGCGGA |
| | | CAGGTATCCGGTAAGCGGCAGGGTCGGAACAGGAGAGCGC |
| | | ACGAGGGAGCTTCCAGGGGGAAACGCCTGGTATCTTTATAGT |
| | | CCTGTCGGGTTTCGCCACCTCTGACTTGAGCGTCGATTTTTG |
| | | TGATGCTCGTCAGGGGGGCGGAGCCTATGGAAAAACGCCAG |
| | | CAACGCGGCCTTTTTACGGTTCCTGGCCTTTTGCTGGCCTTT |
| | | TGCTCACATGTTCTTTCCTGCGTTATCCCCTGATTCTGTGGAT |
| | | AACCGTATTACCGCCTTTGAGTGAGCTGATACCGCTCGCCGC |
| | | AGCCGAACGACCGAGCGCAGCGAGTCAGTGAGCGAGGAAG |
| | | CGGAAGAGCGCCCAATACGCAAACCGCCTCTCCCCGCGCGT |
| | | TGGCCGATTCATTAATGCAGCTGGCACGACAGGTTTCCCGAC |
| | | TGGAAAGCGGGCAGTGAGCGCAACGCAATTAATGTGAGTTA |
| | | GCTCACTCATTAGGCACCCCAGGCTTTACACTTTATGCTTCC |
| | | GGCTCGTATGTTGTGTGGAATTGTGAGCGGATAACAATTTCA |
| | | CACAGGAAACAGCTATGACCATGATTACGCCAGATTTAATTAA |
| | | GGCCTTAATTAGG |
| 15 | Plasmid P1593 | CTGCGCGCTCGCTCGCTCACTGAGGCCGCCCGGGCAAAGC |
| | 5′ ITR at nucleotides | CCGGGCGTCGGGCGACCTTTGGTCGCCCGGCCTCAGTGAG |
| | 1-130 | CGAGCGAGCGCGCAGAGAGGGAGTGGCCAACTCCATCACTA |
| | GJB2 regulatory | GGGGTTCCTGAGCAGAATTCTGGTACCTTGCTAGCAAAAACT |
| | construct at nucleotides | CTTACATAATTGTAACAGATTTGAGTTTCCTCTGGTTCAGGTT |
| | 157-2617 | TTCTTGCCTCTTTGATAATCAAATGATCTGAAGAAAGGCATAG |
| | Codon-optimized and | AATTTCAAGGGAGAATCTGCATGACAGGATTACAATAAGGCT |
| | CpG depleted hGJB2 at | ATTCATGGAGACTCTTTTATTAGCTTACACAGGATCTGCATCA |
| | nucleotides 2631-3308 | TTCTTCGTCTCTGGCTTTGCTACAAGGCTCCATTTAAACTTAA |
| | SV40 polyA signal | CCCAACTTGCAGGCTTAACTGATCCAGGAATGACTCAATACA |
| | sequence at nucleotides | ATGAGCCAGCTACTGTAGCTCTTTTTCCCCTTTTGATAAGGG |
| | 3325-3459 | GAGTTAACACAATGGGCTTTACAGTTCTTAAATGACCGTTCCT |
| | 3′ ITR at nucleotides | GCAGGTATTGGAAAACCTGCATCGTATGCCTGCTGCTTTAGG |
| | 3478-3607 | AGACAACAAACTCAGCAGCCCCTCCCCCGAAGCTGAAGTGC |
| | AmpR promoter at | CAAACTTAAGGCTCCAGGTAAACTGCCCTTCCGATGAGAGCT |
| | nucleotides 4265-4369 | TTACATTTAGGCAGTAGCCACAGGCCTAAACAAAGTCAAATG |
| | KanR at nucleotides | GATGAAACTTGTGATCTCTCCTTGGAAATATGGCCAAGAGTG |
| | 4370-5179 | AAACAACTACTTAGTTATTATTTTAAACGCAGGCTGCTAAATG |
| | Sequence to be | GCTTGTGTTGGTATGTAATGGCATCCTAGATAATGGGGGCTG |
| | transferred into vector | CCAAGAAAATATTCACACTGGCAAGTCAATTAAAATACCGTTC |
| | at nucleotides 1-3607 | TCTTCCTAAGAGGCCTGGCTGGATTCCAGAGCTTCCTTTAGG |
| | | GTAACGCTCAAACCTGTCATAGTTTATTTTGTTTCTGTTTTCAG |
| | | GCAACAGATTTGGTTATTGCTTTTATTGTATGAGTGTTGAAAT |
| | | GGTGTCCTAACAGCCATCTGTACTGCCATCCCCTGGTCTCTC |
| | | TGCCTTTCCAGAAAGGAGATGAAGTTGCTGGAGCAGGCAGG |
| | | TTCTCTGAAAGGCTCCCTGAGTGTGGAAGAGCAGCTGTCACT |
| | | CATCAGTGGTTGTCCCAATATCCAAGAAGCAGTAGAGGGTGC |
| | | CATGCACATTCAGGTGAGTGAAGGACACTGGCGCCGCATCT |
| | | GTGGGGCCTGAGACATTGTCTCCCAGCCTCTCCGGAGGAAC |
| | | AGGGCAGGATACAGAGCAGTGGCAGGTGTGGGCATTTTGCA |
| | | TCTTCTGTAATCTCCAAACAAAGTCTGCGCTAGGCCCTTTCAG |
| | | GCTATAAAGGACAGGGTACTCTGATTTCTGCTCTTGATGAGG |
| | | GTTGATTGTGATACCAGGCGAGGGAGTAAGAGATACAGGAC |
| | | ACCATCTCTGCGGCTGCATCTTTGTCGCAGAAACAGACATTG |
| | | TTCAGGGGACACCTGAGCCTCTGATAGTCAGCCAGTGGCTCT |
| | | GGCAGCTCACAGCAGTTATAGGGTGGCTGTGTCCTTTTCTAA |
| | | AGTCCTGACTTTTTATGGCCTGTGGTCATGGTGACTCCACTA |
| | | CGGGCTGTGTTTCTGTTGACGCGTACAGAGGACAACGACCA |
| | | CAGCCATCCCTGAACCCCGCCCACGGCACAGCGCCGGAGC |
| | | CGGGGTCTGGGGCGCCGCTTCCTGGGGGGTCCCGACTCTC |
| | | AGCCGCCCCCGCTTCACCCGGGCCGCCAAGGGGCTGGGGG |
| | | AGGCGGCGCTCGGGGTAACCGGGGGAGACTCAGGGCGCTG |
| | | GGGGCACTTGGGGAACTCATGGGGGCTCAAAGGAACTAGGA |
| | | GATCGGGACCTCGAAGGGGACTTGGGGGGTTCGGGGCTTTC |
| | | GGGGGCGGTCGGGGGTTCGCGGACCCGGGAAGCTCTGAGG |
| | | ACCCAGAGGCCGGGCGCGCTCCGCCCGCGGCGCCGCCCCC |
| | | TCCGTAACTTTCCCAGTCTCCGAGGGAAGAGGCGGGGTGTG |
| | | GGGTGCGGTTAAAAGGCGCCACGGCGGGAGACAGGTGTTG |
| | | CGGCCCCGCAGCGCCCGCGCGCTCCTCTCCCCGACTCGGA |
| | | GCCCCTCGGCGGCGCCCGGCCCAGGACCCGCCTAGGAGCG |
| | | CAGGAGCCCCAGCGCAGAGACCCCAACGCCGAGACCCCCG |
| | | CCCCGGCCCCGCCGCGCTTCCTCCCGACGCAGGTGAGCCC |
| | | GCCGGCCCCGGACTGCCCGGCCAGGAACCTGGCGCGGGGA |
| | | GGGACCGCGAGACCCAGAGCGGTTGCCCGGCCGCGTGGGT |
| | | CTCGGGGAACCGGGGGGCTGGACCAACACACGTCCTTGGG |
| | | CCGGGGGGCGGGGGCCGCCTTCTGGAGCGGGCGTTTCTGT |
| | | TTATGATGTGTTTAAAGATTGGGTGAATTACTCAGGTGAACAA |
| | | GCTACTTTTTATCAGAGAACACCTAAAAACACGTTCAAGAGG |
| | | GTTTGGGAACTATACATTTAATCCTATGACAAACTAAGTTGGT |
| | | TCTGTCTTCACCTGTTTTGGTGAGGTTGTGTAAGAGTTGGTGT |
| | | TTGCTCAGGAAGAGATTTAAGCATGCTTGCTTACCCAGACTC |
| | | AGAGAAGTCTCCCTGTTCTGTCCTAGCTAGTGATTCCTGTGTT |
| | | GTGTGCATTCGTCTTTTCCAGAGCAAACCGCCCAGAGTAGAA |
| | | GCCTGCAGGCCACCATGGATTGGGGCACACTGCAGACTATC |
| | | CTGGGGGGTGTGAACAAACACTCCACCAGCATTGGAAAGAT |
| | | CTGGCTCACAGTCCTCTTCATTTTTAGGATTATGATCCTGGTT |
| | | GTGGCTGCAAAGGAGGTGTGGGGAGATGAGCAGGCTGACTT |
| | | TGTCTGCAACACCCTGCAGCCAGGCTGCAAGAATGTGTGCTA |
| | | TGATCACTACTTCCCCATCTCCCACATCAGGCTATGGGCCCT |
| | | GCAGCTGATCTTTGTGTCCACTCCAGCACTCCTAGTGGCCAT |
| | | GCATGTGGCCTACAGGAGACATGAGAAGAAGAGGAAGTTCA |
| | | TCAAGGGGGAGATAAAGAGTGAATTTAAGGACATTGAGGAGA |
| | | TCAAAACCCAGAAGGTCAGAATTGAAGGCTCCCTGTGGTGGA |
| | | CCTACACAAGCAGCATCTTCTTCAGGGTCATCTTTGAAGCAG |
| | | CCTTCATGTATGTCTTCTATGTCATGTATGATGGCTTCTCCAT |
| | | GCAGAGGCTGGTGAAGTGCAATGCCTGGCCTTGTCCCAACA |
| | | CTGTGGACTGCTTTGTGTCCAGGCCCACTGAGAAGACTGTCT |
| | | TCACAGTGTTCATGATTGCAGTGTCTGGAATTTGCATCCTGCT |
| | | GAATGTCACTGAATTGTGTTATTTGCTAATTAGATATTGTTCTG |
| | | GGAAGTCAAAAAAGCCAGTTTGATAAGCTTAGATCTGATCCA |
| | | GACATGATAAGATACATTGATGAGTTTGGACAAACCAAAACTA |
| | | GAATGCAGTGAAAAAAATGCCTTATTTGTGAAATTTGTGATGC |
| | | TATTGCCTTATTTGTAACCATTATAAGCTGCAATAAACAAGTTT |
| | | CTAGAACTGAATTCACAAGGAACCCCTAGTGATGGAGTTGGC |
| | | CACTCCCTCTCTGCGCGCTCGCTCGCTCACTGAGGCCGGGC |
| | | GACCAAAGGTCGCCCGACGCCCGGGCTTTGCCCGGGCGGC |
| | | CTCAGTGAGCGAGCGAGCGCGCAGCCTTAATTAACCTAATTC |
| | | ACTGGCCGTCGTTTTACAACGTCGTGACTGGGAAAACCCTGG |
| | | CGTTACCCAACTTAATCGCCTTGCAGCACATCCCCCTTTCGC |
| | | CAGCTGGCGTAATAGCGAAGAGGCCCGCACCGATCGCCCTT |
| | | CCCAACAGTTGCGCAGCCTGAATGGCGAATGGGACGCGCCC |
| | | TGTAGCGGCGCATTAAGCGCGGCGGGTGTGGTGGTTACGCG |
| | | CAGCGTGACCGCTACACTTGCCAGCGCCCTAGCGCCCGCTC |
| | | CTTTCGCTTTCTTCCCTTCCTTTCTCGCCACGTTCGCCGGCTT |
| | | TCCCCGTCAAGCTCTAAATCGGGGGCTCCCTTTAGGGTTCCG |
| | | ATTTAGTGCTTTACGGCACCTCGACCCCAAAAAACTTGATTAG |
| | | GGTGATGGTTCACGTAGTGGGCCATCGCCCTGATAGACGGT |
| | | TTTTCGCCCTTTGACGTTGGAGTCCACGTTCTTTAATAGTGGA |
| | | CTCTTGTTCCAAACTGGAACAACACTCAACCCTATCTCGGTCT |
| | | ATTCTTTTGATTTATAAGGGATTTTGCCGATTTCGGCCTATTG |
| | | GTTAAAAAATGAGCTGATTTAACAAAAATTTAACGCGAATTTTA |
| | | ACAAAATATTAACGCTTACAATTTAGGTGGCACTTTTCGGGGA |
| | | AATGTGCGCGGAACCCCTATTTGTTTATTTTTCTAAATACATT |
| | | CAAATATGTATCCGCTCATGAGACAATAACCCTGATAAATGCT |
| | | TCAATAATATTGAAAAAGGAAGAGTATGAGCCATATTCAACGG |
| | | GAAACGTCGAGGCCGCGATTAAATTCCAACATGGATGCTGAT |
| | | TTATATGGGTATAAATGGGCTCGCGATAATGTCGGGCAATCA |
| | | GGTGCGACAATCTATCGCTTGTATGGGAAGCCCGATGCGCC |
| | | AGAGTTGTTTCTGAAACATGGCAAAGGTAGCGTTGCCAATGA |
| | | TGTTACAGATGAGATGGTCAGACTAAACTGGCTGACGGAATT |
| | | TATGCCTCTTCCGACCATCAAGCATTTTATCCGTACTCCTGAT |
| | | GATGCATGGTTACTCACCACTGCGATCCCCGGAAAAACAGCA |
| | | TTCCAGGTATTAGAAGAATATCCTGATTCAGGTGAAAATATTG |
| | | TTGATGCGCTGGCAGTGTTCCTGCGCCGGTTGCATTCGATTC |
| | | CTGTTTGTAATTGTCCTTTTAACAGCGATCGCGTATTTCGTCT |
| | | CGCTCAGGCGCAATCACGAATGAATAACGGTTTGGTTGATGC |
| | | GAGTGATTTTGATGACGAGCGTAATGGCTGGCCTGTTGAACA |
| | | AGTCTGGAAAGAAATGCATAAACTTTTGCCATTCTCACCGGAT |
| | | TCAGTCGTCACTCATGGTGATTTCTCACTTGATAACCTTATTT |
| | | TTGACGAGGGGAAATTAATAGGTTGTATTGATGTTGGACGAG |
| | | TCGGAATCGCAGACCGATACCAGGATCTTGCCATCCTATGGA |
| | | ACTGCCTCGGTGAGTTTTCTCCTTCATTACAGAAACGGCTTTT |
| | | TCAAAAATATGGTATTGATAATCCTGATATGAATAAATTGCAG |
| | | TTTCATTTGATGCTCGATGAGTTTTTCTAACTGTCAGACCAAG |
| | | TTTACTCATATATACTTTAGATTGATTTAAAACTTCATTTTTAAT |
| | | TTAAAAGGATCTAGGTGAAGATCCTTTTTGATAATCTCATGAC |
| | | CAAAATCCCTTAACGTGAGTTTTCGTTCCACTGAGCGTCAGA |
| | | CCCCGTAGAAAAGATCAAAGGATCTTCTTGAGATCCTTTTTTT |
| | | CTGCGCGTAATCTGCTGCTTGCAAACAAAAAAACCACCGCTA |
| | | CCAGCGGTGGTTTGTTTGCCGGATCAAGAGCTACCAACTCTT |
| | | TTTCCGAAGGTAACTGGCTTCAGCAGAGCGCAGATACCAAAT |
| | | ACTGTTCTTCTAGTGTAGCCGTAGTTAGGCCACCACTTCAAG |
| | | AACTCTGTAGCACCGCCTACATACCTCGCTCTGCTAATCCTG |
| | | TTACCAGTGGCTGCTGCCAGTGGCGATAAGTCGTGTCTTACC |
| | | GGGTTGGACTCAAGACGATAGTTACCGGATAAGGCGCAGCG |
| | | GTCGGGCTGAACGGGGGGTTCGTGCACACAGCCCAGCTTGG |
| | | AGCGAACGACCTACACCGAACTGAGATACCTACAGCGTGAG |
| | | CTATGAGAAAGCGCCACGCTTCCCGAAGGGAGAAAGGCGGA |
| | | CAGGTATCCGGTAAGCGGCAGGGTCGGAACAGGAGAGCGC |
| | | ACGAGGGAGCTTCCAGGGGGAAACGCCTGGTATCTTTATAGT |
| | | CCTGTCGGGTTTCGCCACCTCTGACTTGAGCGTCGATTTTTG |
| | | TGATGCTCGTCAGGGGGGCGGAGCCTATGGAAAAACGCCAG |
| | | CAACGCGGCCTTTTTACGGTTCCTGGCCTTTTGCTGGCCTTT |
| | | TGCTCACATGTTCTTTCCTGCGTTATCCCCTGATTCTGTGGAT |
| | | AACCGTATTACCGCCTTTGAGTGAGCTGATACCGCTCGCCGC |
| | | AGCCGAACGACCGAGCGCAGCGAGTCAGTGAGCGAGGAAG |
| | | CGGAAGAGCGCCCAATACGCAAACCGCCTCTCCCCGCGCGT |
| | | TGGCCGATTCATTAATGCAGCTGGCACGACAGGTTTCCCGAC |
| | | TGGAAAGCGGGCAGTGAGCGCAACGCAATTAATGTGAGTTA |
| | | GCTCACTCATTAGGCACCCCAGGCTTTACACTTTATGCTTCC |
| | | GGCTCGTATGTTGTGTGGAATTGTGAGCGGATAACAATTTCA |
| | | CACAGGAAACAGCTATGACCATGATTACGCCAGATTTAATTAA |
| | | GGCCTTAATTAGG |
| 16 | Plasmid P1595 | CTGCGCGCTCGCTCGCTCACTGAGGCCGCCCGGGCAAAGC |
| | 5′ ITR at nucleotides | CCGGGCGTCGGGCGACCTTTGGTCGCCCGGCCTCAGTGAG |
| | 1-130 | CGAGCGAGCGCGCAGAGAGGGAGTGGCCAACTCCATCACTA |
| | GJB2 regulatory | GGGGTTCCTGAGCAGAATTCTGGTACCTTGCTAGCAAAAACT |
| | construct at nucleotides | CTTACATAATTGTAACAGATTTGAGTTTCCTCTGGTTCAGGTT |
| | 157-1541 | TTCTTGCCTCTTTGATAATCAAATGATCTGAAGAAAGGCATAG |
| | CpG depleted hGJB2 at | AATTTCAAGGGAGAATCTGCATGACAGGATTACAATAAGGCT |
| | nucleotides 1555-2232 | ATTCATGGAGACTCTTTTATTAGCTTACACAGGATCTGCATCA |
| | bGH polyA signal | TTCTTCGTCTCTGGCTTTGCTACAAGGCTCCATTTAAACTTAA |
| | sequence at nucleotides | CCCAACTTGCAGGCTTAACTGATCCAGGAATGACTCAATACA |
| | 2255-2462 | ATGAGCCAGCTACTGTAGCTCTTTTTCCCCTTTTGATAAGGG |
| | 3′ ITR at nucleotides | GAGTTAACACAATGGGCTTTACAGTTCTTAAATGAACGCGTAC |
| | 2481-2610 | AGAGGACAACGACCACAGCCATCCCTGAACCCCGCCCACGG |
| | AmpR promoter at | CACAGCGCCGGAGCCGGGGTCTGGGGCGCCGCTTCCTGGG |
| | nucleotides 3268-3372 | GGGTCCCGACTCTCAGCCGCCCCCGCTTCACCCGGGCCGC |
| | KanR at nucleotides | CAAGGGGCTGGGGGAGGCGGCGCTCGGGGTAACCGGGGG |
| | 3373-4182 | AGACTCAGGGCGCTGGGGGCACTTGGGGAACTCATGGGGG |
| | Sequence to be | CTCAAAGGAACTAGGAGATCGGGACCTCGAAGGGGACTTGG |
| | transferred into vector | GGGGTTCGGGGCTTTCGGGGGCGGTCGGGGGTTCGCGGAC |
| | at nucleotides 1-2610 | CCGGGAAGCTCTGAGGACCCAGAGGCCGGGCGCGCTCCGC |
| | | CCGCGGCGCCGCCCCCTCCGTAACTTTCCCAGTCTCCGAGG |
| | | GAAGAGGCGGGGTGTGGGGTGCGGTTAAAAGGCGCCACGG |
| | | CGGGAGACAGGTGTTGCGGCCCCGCAGCGCCCGCGCGCTC |
| | | CTCTCCCCGACTCGGAGCCCCTCGGCGGCGCCCGGCCCAG |
| | | GACCCGCCTAGGAGCGCAGGAGCCCCAGCGCAGAGACCCC |
| | | AACGCCGAGACCCCCGCCCCGGCCCCGCCGCGCTTCCTCC |
| | | CGACGCAGGTGAGCCCGCCGGCCCCGGACTGCCCGGCCAG |
| | | GAACCTGGCGCGGGGAGGGACCGCGAGACCCAGAGCGGTT |
| | | GCCCGGCCGCGTGGGTCTCGGGGAACCGGGGGGCTGGACC |
| | | AACACACGTCCTTGGGCCGGGGGGCGGGGGCCGCCTTCTG |
| | | GAGCGGGCGTTTCTGTTTATGATGTGTTTAAAGATTGGGTGA |
| | | ATTACTCAGGTGAACAAGCTACTTTTTATCAGAGAACACCTAA |
| | | AAACACGTTCAAGAGGGTTTGGGAACTATACATTTAATCCTAT |
| | | GACAAACTAAGTTGGTTCTGTCTTCACCTGTTTTGGTGAGGTT |
| | | GTGTAAGAGTTGGTGTTTGCTCAGGAAGAGATTTAAGCATGC |
| | | TTGCTTACCCAGACTCAGAGAAGTCTCCCTGTTCTGTCCTAG |
| | | CTAGTGATTCCTGTGTTGTGTGCATTCGTCTTTTCCAGAGCAA |
| | | ACCGCCCAGAGTAGAAGCCTGCAGGCCACCATGGATTGGGG |
| | | CACACTGCAGACTATCCTGGGGGGTGTGAACAAACACTCCAC |
| | | CAGCATTGGAAAGATCTGGCTCACAGTCCTCTTCATTTTTAGG |
| | | ATTATGATCCTGGTTGTGGCTGCAAAGGAGGTGTGGGGAGAT |
| | | GAGCAGGCTGACTTTGTCTGCAACACCCTGCAGCCAGGCTG |
| | | CAAGAATGTGTGCTATGATCACTACTTCCCCATCTCCCACATC |
| | | AGGCTATGGGCCCTGCAGCTGATCTTTGTGTCCACTCCAGCA |
| | | CTCCTAGTGGCCATGCATGTGGCCTACAGGAGACATGAGAA |
| | | GAAGAGGAAGTTCATCAAGGGGGAGATAAAGAGTGAATTTAA |
| | | GGACATTGAGGAGATCAAAACCCAGAAGGTCAGAATTGAAGG |
| | | CTCCCTGTGGTGGACCTACACAAGCAGCATCTTCTTCAGGGT |
| | | CATCTTTGAAGCAGCCTTCATGTATGTCTTCTATGTCATGTAT |
| | | GATGGCTTCTCCATGCAGAGGCTGGTGAAGTGCAATGCCTG |
| | | GCCTTGTCCCAACACTGTGGACTGCTTTGTGTCCAGGCCCAC |
| | | TGAGAAGACTGTCTTCACAGTGTTCATGATTGCAGTGTCTGG |
| | | AATTTGCATCCTGCTGAATGTCACTGAATTGTGTTATTTGCTA |
| | | ATTAGATATTGTTCTGGGAAGTCAAAAAAGCCAGTTTGATAAG |
| | | CTTAGATCTGCCTGACTGTGCCTTCTAGTTGCCAGCCATCTG |
| | | TTGTTTGCCCCTCCCCCGTGCCTTCCTTGACCCTGGAAGGTG |
| | | CCACTCCCACTGTCCTTTCCTAATAAAATGAGGAAATTGCATC |
| | | GCATTGTCTGAGTAGGTGTCATTCTATTCTGGGGGGTGGGGT |
| | | GGGGCAGGACAGCAAGGGGGAGGATTGGGAAGACAATAGC |
| | | AGGCATGCTGGGGATCTAGAACTGAATTCACAAGGAACCCCT |
| | | AGTGATGGAGTTGGCCACTCCCTCTCTGCGCGCTCGCTCGC |
| | | TCACTGAGGCCGGGCGACCAAAGGTCGCCCGACGCCCGGG |
| | | CTTTGCCCGGGCGGCCTCAGTGAGCGAGCGAGCGCGCAGC |
| | | CTTAATTAACCTAATTCACTGGCCGTCGTTTTACAACGTCGTG |
| | | ACTGGGAAAACCCTGGCGTTACCCAACTTAATCGCCTTGCAG |
| | | CACATCCCCCTTTCGCCAGCTGGCGTAATAGCGAAGAGGCC |
| | | CGCACCGATCGCCCTTCCCAACAGTTGCGCAGCCTGAATGG |
| | | CGAATGGGACGCGCCCTGTAGCGGCGCATTAAGCGCGGCG |
| | | GGTGTGGTGGTTACGCGCAGCGTGACCGCTACACTTGCCAG |
| | | CGCCCTAGCGCCCGCTCCTTTCGCTTTCTTCCCTTCCTTTCT |
| | | CGCCACGTTCGCCGGCTTTCCCCGTCAAGCTCTAAATCGGG |
| | | GGCTCCCTTTAGGGTTCCGATTTAGTGCTTTACGGCACCTCG |
| | | ACCCCAAAAAACTTGATTAGGGTGATGGTTCACGTAGTGGGC |
| | | CATCGCCCTGATAGACGGTTTTTCGCCCTTTGACGTTGGAGT |
| | | CCACGTTCTTTAATAGTGGACTCTTGTTCCAAACTGGAACAAC |
| | | ACTCAACCCTATCTCGGTCTATTCTTTTGATTTATAAGGGATTT |
| | | TGCCGATTTCGGCCTATTGGTTAAAAAATGAGCTGATTTAACA |
| | | AAAATTTAACGCGAATTTTAACAAAATATTAACGCTTACAATTT |
| | | AGGTGGCACTTTTCGGGGAAATGTGCGCGGAACCCCTATTTG |
| | | TTTATTTTTCTAAATACATTCAAATATGTATCCGCTCATGAGAC |
| | | AATAACCCTGATAAATGCTTCAATAATATTGAAAAAGGAAGAG |
| | | TATGAGCCATATTCAACGGGAAACGTCGAGGCCGCGATTAAA |
| | | TTCCAACATGGATGCTGATTTATATGGGTATAAATGGGCTCG |
| | | CGATAATGTCGGGCAATCAGGTGCGACAATCTATCGCTTGTA |
| | | TGGGAAGCCCGATGCGCCAGAGTTGTTTCTGAAACATGGCAA |
| | | AGGTAGCGTTGCCAATGATGTTACAGATGAGATGGTCAGACT |
| | | AAACTGGCTGACGGAATTTATGCCTCTTCCGACCATCAAGCA |
| | | TTTTATCCGTACTCCTGATGATGCATGGTTACTCACCACTGCG |
| | | ATCCCCGGAAAAACAGCATTCCAGGTATTAGAAGAATATCCT |
| | | GATTCAGGTGAAAATATTGTTGATGCGCTGGCAGTGTTCCTG |
| | | CGCCGGTTGCATTCGATTCCTGTTTGTAATTGTCCTTTTAACA |
| | | GCGATCGCGTATTTCGTCTCGCTCAGGCGCAATCACGAATGA |
| | | ATAACGGTTTGGTTGATGCGAGTGATTTTGATGACGAGCGTA |
| | | ATGGCTGGCCTGTTGAACAAGTCTGGAAAGAAATGCATAAAC |
| | | TTTTGCCATTCTCACCGGATTCAGTCGTCACTCATGGTGATTT |
| | | CTCACTTGATAACCTTATTTTTGACGAGGGGAAATTAATAGGT |
| | | TGTATTGATGTTGGACGAGTCGGAATCGCAGACCGATACCAG |
| | | GATCTTGCCATCCTATGGAACTGCCTCGGTGAGTTTTCTCCTT |
| | | CATTACAGAAACGGCTTTTTCAAAAATATGGTATTGATAATCC |
| | | TGATATGAATAAATTGCAGTTTCATTTGATGCTCGATGAGTTT |
| | | TTCTAACTGTCAGACCAAGTTTACTCATATATACTTTAGATTGA |
| | | TTTAAAACTTCATTTTTAATTTAAAAGGATCTAGGTGAAGATCC |
| | | TTTTTGATAATCTCATGACCAAAATCCCTTAACGTGAGTTTTC |
| | | GTTCCACTGAGCGTCAGACCCCGTAGAAAAGATCAAAGGATC |
| | | TTCTTGAGATCCTTTTTTTCTGCGCGTAATCTGCTGCTTGCAA |
| | | ACAAAAAAACCACCGCTACCAGCGGTGGTTTGTTTGCCGGAT |
| | | CAAGAGCTACCAACTCTTTTTCCGAAGGTAACTGGCTTCAGC |
| | | AGAGCGCAGATACCAAATACTGTTCTTCTAGTGTAGCCGTAG |
| | | TTAGGCCACCACTTCAAGAACTCTGTAGCACCGCCTACATAC |
| | | CTCGCTCTGCTAATCCTGTTACCAGTGGCTGCTGCCAGTGGC |
| | | GATAAGTCGTGTCTTACCGGGTTGGACTCAAGACGATAGTTA |
| | | CCGGATAAGGCGCAGCGGTCGGGCTGAACGGGGGGTTCGT |
| | | GCACACAGCCCAGCTTGGAGCGAACGACCTACACCGAACTG |
| | | AGATACCTACAGCGTGAGCTATGAGAAAGCGCCACGCTTCCC |
| | | GAAGGGAGAAAGGCGGACAGGTATCCGGTAAGCGGCAGGG |
| | | TCGGAACAGGAGAGCGCACGAGGGAGCTTCCAGGGGGAAA |
| | | CGCCTGGTATCTTTATAGTCCTGTCGGGTTTCGCCACCTCTG |
| | | ACTTGAGCGTCGATTTTTGTGATGCTCGTCAGGGGGGCGGA |
| | | GCCTATGGAAAAACGCCAGCAACGCGGCCTTTTTACGGTTCC |
| | | TGGCCTTTTGCTGGCCTTTTGCTCACATGTTCTTTCCTGCGTT |
| | | ATCCCCTGATTCTGTGGATAACCGTATTACCGCCTTTGAGTG |
| | | AGCTGATACCGCTCGCCGCAGCCGAACGACCGAGCGCAGC |
| | | GAGTCAGTGAGCGAGGAAGCGGAAGAGCGCCCAATACGCAA |
| | | ACCGCCTCTCCCCGCGCGTTGGCCGATTCATTAATGCAGCTG |
| | | GCACGACAGGTTTCCCGACTGGAAAGCGGGCAGTGAGCGCA |
| | | ACGCAATTAATGTGAGTTAGCTCACTCATTAGGCACCCCAGG |
| | | CTTTACACTTTATGCTTCCGGCTCGTATGTTGTGTGGAATTGT |
| | | GAGCGGATAACAATTTCACACAGGAAACAGCTATGACCATGA |
| | | TTACGCCAGATTTAATTAAGGCCTTAATTAGG |
| 17 | Plasmid P1596 | CTGCGCGCTCGCTCGCTCACTGAGGCCGCCCGGGCAAAGC |
| | 5′ ITR at nucleotides | CCGGGCGTCGGGCGACCTTTGGTCGCCCGGCCTCAGTGAG |
| | 1-130 | CGAGCGAGCGCGCAGAGAGGGAGTGGCCAACTCCATCACTA |
| | GJB2 regulatory | GGGGTTCCTGAGCAGAATTCTGGTACCTTGCTAGCAAAAACT |
| | construct at nucleotides | CTTACATAATTGTAACAGATTTGAGTTTCCTCTGGTTCAGGTT |
| | 157-1541 | TTCTTGCCTCTTTGATAATCAAATGATCTGAAGAAAGGCATAG |
| | Codon-optimized and | AATTTCAAGGGAGAATCTGCATGACAGGATTACAATAAGGCT |
| | CpG depleted hGJB2 at | ATTCATGGAGACTCTTTTATTAGCTTACACAGGATCTGCATCA |
| | nucleotides 1555-2232 | TTCTTCGTCTCTGGCTTTGCTACAAGGCTCCATTTAAACTTAA |
| | bGH polyA signal | CCCAACTTGCAGGCTTAACTGATCCAGGAATGACTCAATACA |
| | sequence at nucleotides | ATGAGCCAGCTACTGTAGCTCTTTTTCCCCTTTTGATAAGGG |
| | 2255-2462 | GAGTTAACACAATGGGCTTTACAGTTCTTAAATGAACGCGTAC |
| | 3′ ITR at nucleotides | AGAGGACAACGACCACAGCCATCCCTGAACCCCGCCCACGG |
| | 2481-2610 | CACAGCGCCGGAGCCGGGGTCTGGGGCGCCGCTTCCTGGG |
| | AmpR promoter at | GGGTCCCGACTCTCAGCCGCCCCCGCTTCACCCGGGCCGC |
| | nucleotides 3268-3372 | CAAGGGGCTGGGGGAGGCGGCGCTCGGGGTAACCGGGGG |
| | KanR at nucleotides | AGACTCAGGGCGCTGGGGGCACTTGGGGAACTCATGGGGG |
| | 3373-4182 | CTCAAAGGAACTAGGAGATCGGGACCTCGAAGGGGACTTGG |
| | Sequence to be | GGGGTTCGGGGCTTTCGGGGGCGGTCGGGGGTTCGCGGAC |
| | transferred into vector | CCGGGAAGCTCTGAGGACCCAGAGGCCGGGCGCGCTCCGC |
| | at nucleotides 1-2610 | CCGCGGCGCCGCCCCCTCCGTAACTTTCCCAGTCTCCGAGG |
| | | GAAGAGGCGGGGTGTGGGGTGCGGTTAAAAGGCGCCACGG |
| | | CGGGAGACAGGTGTTGCGGCCCCGCAGCGCCCGCGCGCTC |
| | | CTCTCCCCGACTCGGAGCCCCTCGGCGGCGCCCGGCCCAG |
| | | GACCCGCCTAGGAGCGCAGGAGCCCCAGCGCAGAGACCCC |
| | | AACGCCGAGACCCCCGCCCCGGCCCCGCCGCGCTTCCTCC |
| | | CGACGCAGGTGAGCCCGCCGGCCCCGGACTGCCCGGCCAG |
| | | GAACCTGGCGCGGGGAGGGACCGCGAGACCCAGAGCGGTT |
| | | GCCCGGCCGCGTGGGTCTCGGGGAACCGGGGGGCTGGACC |
| | | AACACACGTCCTTGGGCCGGGGGGCGGGGGCCGCCTTCTG |
| | | GAGCGGGCGTTTCTGTTTATGATGTGTTTAAAGATTGGGTGA |
| | | ATTACTCAGGTGAACAAGCTACTTTTTATCAGAGAACACCTAA |
| | | AAACACGTTCAAGAGGGTTTGGGAACTATACATTTAATCCTAT |
| | | GACAAACTAAGTTGGTTCTGTCTTCACCTGTTTTGGTGAGGTT |
| | | GTGTAAGAGTTGGTGTTTGCTCAGGAAGAGATTTAAGCATGC |
| | | TTGCTTACCCAGACTCAGAGAAGTCTCCCTGTTCTGTCCTAG |
| | | CTAGTGATTCCTGTGTTGTGTGCATTCGTCTTTTCCAGAGCAA |
| | | ACCGCCCAGAGTAGAAGCCTGCAGGCCACCATGGACTGGGG |
| | | AACACTGCAAACAATCCTGGGAGGTGTGAACAAGCACAGCAC |
| | | AAGCATTGGCAAGATCTGGCTGACAGTGCTGTTCATCTTTAG |
| | | AATCATGATCCTGGTTGTGGCTGCTAAGGAAGTGTGGGGAGA |
| | | TGAGCAGGCTGACTTTGTTTGTAATACCCTGCAACCTGGCTG |
| | | CAAGAATGTGTGTTATGACCACTACTTCCCCATCAGCCACAT |
| | | CAGACTGTGGGCCCTGCAACTGATCTTTGTGTCCACCCCTGC |
| | | CCTGCTGGTGGCCATGCATGTGGCCTACAGAAGGCATGAGA |
| | | AGAAGAGGAAATTCATCAAGGGAGAGATCAAGAGTGAGTTCA |
| | | AGGACATTGAGGAAATCAAAACCCAGAAGGTGAGGATTGAAG |
| | | GCTCTCTGTGGTGGACCTACACCAGCAGCATCTTCTTCAGAG |
| | | TGATCTTTGAGGCAGCCTTTATGTATGTGTTCTATGTGATGTA |
| | | TGATGGCTTCTCCATGCAGAGACTGGTGAAGTGCAATGCCTG |
| | | GCCATGTCCTAACACAGTGGACTGCTTTGTGTCTAGACCTAC |
| | | AGAAAAGACAGTGTTTACAGTCTTTATGATTGCAGTGTCTGGC |
| | | ATCTGCATTCTGCTGAATGTGACTGAGCTGTGCTACCTGCTC |
| | | ATTAGGTACTGTAGTGGAAAAAGCAAGAAACCTGTGTGATAA |
| | | GCTTAGATCTGCCTGACTGTGCCTTCTAGTTGCCAGCCATCT |
| | | GTTGTTTGCCCCTCCCCCGTGCCTTCCTTGACCCTGGAAGGT |
| | | GCCACTCCCACTGTCCTTTCCTAATAAAATGAGGAAATTGCAT |
| | | CGCATTGTCTGAGTAGGTGTCATTCTATTCTGGGGGGTGGGG |
| | | TGGGGCAGGACAGCAAGGGGGAGGATTGGGAAGACAATAG |
| | | CAGGCATGCTGGGGATCTAGAACTGAATTCACAAGGAACCCC |
| | | TAGTGATGGAGTTGGCCACTCCCTCTCTGCGCGCTCGCTCG |
| | | CTCACTGAGGCCGGGCGACCAAAGGTCGCCCGACGCCCGG |
| | | GCTTTGCCCGGGCGGCCTCAGTGAGCGAGCGAGCGCGCAG |
| | | CCTTAATTAACCTAATTCACTGGCCGTCGTTTTACAACGTCGT |
| | | GACTGGGAAAACCCTGGCGTTACCCAACTTAATCGCCTTGCA |
| | | GCACATCCCCCTTTCGCCAGCTGGCGTAATAGCGAAGAGGC |
| | | CCGCACCGATCGCCCTTCCCAACAGTTGCGCAGCCTGAATG |
| | | GCGAATGGGACGCGCCCTGTAGCGGCGCATTAAGCGCGGC |
| | | GGGTGTGGTGGTTACGCGCAGCGTGACCGCTACACTTGCCA |
| | | GCGCCCTAGCGCCCGCTCCTTTCGCTTTCTTCCCTTCCTTTC |
| | | TCGCCACGTTCGCCGGCTTTCCCCGTCAAGCTCTAAATCGGG |
| | | GGCTCCCTTTAGGGTTCCGATTTAGTGCTTTACGGCACCTCG |
| | | ACCCCAAAAAACTTGATTAGGGTGATGGTTCACGTAGTGGGC |
| | | CATCGCCCTGATAGACGGTTTTTCGCCCTTTGACGTTGGAGT |
| | | CCACGTTCTTTAATAGTGGACTCTTGTTCCAAACTGGAACAAC |
| | | ACTCAACCCTATCTCGGTCTATTCTTTTGATTTATAAGGGATTT |
| | | TGCCGATTTCGGCCTATTGGTTAAAAAATGAGCTGATTTAACA |
| | | AAAATTTAACGCGAATTTTAACAAAATATTAACGCTTACAATTT |
| | | AGGTGGCACTTTTCGGGGAAATGTGCGCGGAACCCCTATTTG |
| | | TTTATTTTTCTAAATACATTCAAATATGTATCCGCTCATGAGAC |
| | | AATAACCCTGATAAATGCTTCAATAATATTGAAAAAGGAAGAG |
| | | TATGAGCCATATTCAACGGGAAACGTCGAGGCCGCGATTAAA |
| | | TTCCAACATGGATGCTGATTTATATGGGTATAAATGGGCTCG |
| | | CGATAATGTCGGGCAATCAGGTGCGACAATCTATCGCTTGTA |
| | | TGGGAAGCCCGATGCGCCAGAGTTGTTTCTGAAACATGGCAA |
| | | AGGTAGCGTTGCCAATGATGTTACAGATGAGATGGTCAGACT |
| | | AAACTGGCTGACGGAATTTATGCCTCTTCCGACCATCAAGCA |
| | | TTTTATCCGTACTCCTGATGATGCATGGTTACTCACCACTGCG |
| | | ATCCCCGGAAAAACAGCATTCCAGGTATTAGAAGAATATCCT |
| | | GATTCAGGTGAAAATATTGTTGATGCGCTGGCAGTGTTCCTG |
| | | CGCCGGTTGCATTCGATTCCTGTTTGTAATTGTCCTTTTAACA |
| | | GCGATCGCGTATTTCGTCTCGCTCAGGCGCAATCACGAATGA |
| | | ATAACGGTTTGGTTGATGCGAGTGATTTTGATGACGAGCGTA |
| | | ATGGCTGGCCTGTTGAACAAGTCTGGAAAGAAATGCATAAAC |
| | | TTTTGCCATTCTCACCGGATTCAGTCGTCACTCATGGTGATTT |
| | | CTCACTTGATAACCTTATTTTTGACGAGGGGAAATTAATAGGT |
| | | TGTATTGATGTTGGACGAGTCGGAATCGCAGACCGATACCAG |
| | | GATCTTGCCATCCTATGGAACTGCCTCGGTGAGTTTTCTCCTT |
| | | CATTACAGAAACGGCTTTTTCAAAAATATGGTATTGATAATCC |
| | | TGATATGAATAAATTGCAGTTTCATTTGATGCTCGATGAGTTT |
| | | TTCTAACTGTCAGACCAAGTTTACTCATATATACTTTAGATTGA |
| | | TTTAAAACTTCATTTTTAATTTAAAAGGATCTAGGTGAAGATCC |
| | | TTTTTGATAATCTCATGACCAAAATCCCTTAACGTGAGTTTTC |
| | | GTTCCACTGAGCGTCAGACCCCGTAGAAAAGATCAAAGGATC |
| | | TTCTTGAGATCCTTTTTTTCTGCGCGTAATCTGCTGCTTGCAA |
| | | ACAAAAAAACCACCGCTACCAGCGGTGGTTTGTTTGCCGGAT |
| | | CAAGAGCTACCAACTCTTTTTCCGAAGGTAACTGGCTTCAGC |
| | | AGAGCGCAGATACCAAATACTGTTCTTCTAGTGTAGCCGTAG |
| | | TTAGGCCACCACTTCAAGAACTCTGTAGCACCGCCTACATAC |
| | | CTCGCTCTGCTAATCCTGTTACCAGTGGCTGCTGCCAGTGGC |
| | | GATAAGTCGTGTCTTACCGGGTTGGACTCAAGACGATAGTTA |
| | | CCGGATAAGGCGCAGCGGTCGGGCTGAACGGGGGGTTCGT |
| | | GCACACAGCCCAGCTTGGAGCGAACGACCTACACCGAACTG |
| | | AGATACCTACAGCGTGAGCTATGAGAAAGCGCCACGCTTCCC |
| | | GAAGGGAGAAAGGCGGACAGGTATCCGGTAAGCGGCAGGG |
| | | TCGGAACAGGAGAGCGCACGAGGGAGCTTCCAGGGGGAAA |
| | | CGCCTGGTATCTTTATAGTCCTGTCGGGTTTCGCCACCTCTG |
| | | ACTTGAGCGTCGATTTTTGTGATGCTCGTCAGGGGGGCGGA |
| | | GCCTATGGAAAAACGCCAGCAACGCGGCCTTTTTACGGTTCC |
| | | TGGCCTTTTGCTGGCCTTTTGCTCACATGTTCTTTCCTGCGTT |
| | | ATCCCCTGATTCTGTGGATAACCGTATTACCGCCTTTGAGTG |
| | | AGCTGATACCGCTCGCCGCAGCCGAACGACCGAGCGCAGC |
| | | GAGTCAGTGAGCGAGGAAGCGGAAGAGCGCCCAATACGCAA |
| | | ACCGCCTCTCCCCGCGCGTTGGCCGATTCATTAATGCAGCTG |
| | | GCACGACAGGTTTCCCGACTGGAAAGCGGGCAGTGAGCGCA |
| | | ACGCAATTAATGTGAGTTAGCTCACTCATTAGGCACCCCAGG |
| | | CTTTACACTTTATGCTTCCGGCTCGTATGTTGTGTGGAATTGT |
| | | GAGCGGATAACAATTTCACACAGGAAACAGCTATGACCATGA |
| | | TTACGCCAGATTTAATTAAGGCCTTAATTAGG |
| 18 | Plasmid P1598 | CTGCGCGCTCGCTCGCTCACTGAGGCCGCCCGGGCAAAGC |
| | 5′ ITR at nucleotides | CCGGGCGTCGGGCGACCTTTGGTCGCCCGGCCTCAGTGAG |
| | 1-130 | CGAGCGAGCGCGCAGAGAGGGAGTGGCCAACTCCATCACTA |
| | GJB2 regulatory | GGGGTTCCTGAGCAGAATTCTGGTACCTTGCTAGCAAAAACT |
| | construct at nucleotides | CTTACATAATTGTAACAGATTTGAGTTTCCTCTGGTTCAGGTT |
| | 157-2617 | TTCTTGCCTCTTTGATAATCAAATGATCTGAAGAAAGGCATAG |
| | CpG depleted hGJB2 at | AATTTCAAGGGAGAATCTGCATGACAGGATTACAATAAGGCT |
| | nucleotides 2631-3308 | ATTCATGGAGACTCTTTTATTAGCTTACACAGGATCTGCATCA |
| | bGH polyA signal | TTCTTCGTCTCTGGCTTTGCTACAAGGCTCCATTTAAACTTAA |
| | sequence at nucleotides | CCCAACTTGCAGGCTTAACTGATCCAGGAATGACTCAATACA |
| | 3331-3538 | ATGAGCCAGCTACTGTAGCTCTTTTTCCCCTTTTGATAAGGG |
| | 3′ ITR at nucleotides | GAGTTAACACAATGGGCTTTACAGTTCTTAAATGACCGTTCCT |
| | 3557-3686 | GCAGGTATTGGAAAACCTGCATCGTATGCCTGCTGCTTTAGG |
| | AmpR promoter at | AGACAACAAACTCAGCAGCCCCTCCCCCGAAGCTGAAGTGC |
| | nucleotides 4344-4448 | CAAACTTAAGGCTCCAGGTAAACTGCCCTTCCGATGAGAGCT |
| | KanR at nucleotides | TTACATTTAGGCAGTAGCCACAGGCCTAAACAAAGTCAAATG |
| | 4449-5258 | GATGAAACTTGTGATCTCTCCTTGGAAATATGGCCAAGAGTG |
| | Sequence to be | AAACAACTACTTAGTTATTATTTTAAACGCAGGCTGCTAAATG |
| | transferred into vector | GCTTGTGTTGGTATGTAATGGCATCCTAGATAATGGGGGCTG |
| | at nucleotides 1-3686 | CCAAGAAAATATTCACACTGGCAAGTCAATTAAAATACCGTTC |
| | | TCTTCCTAAGAGGCCTGGCTGGATTCCAGAGCTTCCTTTAGG |
| | | GTAACGCTCAAACCTGTCATAGTTTATTTTGTTTCTGTTTTCAG |
| | | GCAACAGATTTGGTTATTGCTTTTATTGTATGAGTGTTGAAAT |
| | | GGTGTCCTAACAGCCATCTGTACTGCCATCCCCTGGTCTCTC |
| | | TGCCTTTCCAGAAAGGAGATGAAGTTGCTGGAGCAGGCAGG |
| | | TTCTCTGAAAGGCTCCCTGAGTGTGGAAGAGCAGCTGTCACT |
| | | CATCAGTGGTTGTCCCAATATCCAAGAAGCAGTAGAGGGTGC |
| | | CATGCACATTCAGGTGAGTGAAGGACACTGGCGCCGCATCT |
| | | GTGGGGCCTGAGACATTGTCTCCCAGCCTCTCCGGAGGAAC |
| | | AGGGCAGGATACAGAGCAGTGGCAGGTGTGGGCATTTTGCA |
| | | TCTTCTGTAATCTCCAAACAAAGTCTGCGCTAGGCCCTTTCAG |
| | | GCTATAAAGGACAGGGTACTCTGATTTCTGCTCTTGATGAGG |
| | | GTTGATTGTGATACCAGGCGAGGGAGTAAGAGATACAGGAC |
| | | ACCATCTCTGCGGCTGCATCTTTGTCGCAGAAACAGACATTG |
| | | TTCAGGGGACACCTGAGCCTCTGATAGTCAGCCAGTGGCTCT |
| | | GGCAGCTCACAGCAGTTATAGGGTGGCTGTGTCCTTTTCTAA |
| | | AGTCCTGACTTTTTATGGCCTGTGGTCATGGTGACTCCACTA |
| | | CGGGCTGTGTTTCTGTTGACGCGTACAGAGGACAACGACCA |
| | | CAGCCATCCCTGAACCCCGCCCACGGCACAGCGCCGGAGC |
| | | CGGGGTCTGGGGCGCCGCTTCCTGGGGGGTCCCGACTCTC |
| | | AGCCGCCCCCGCTTCACCCGGGCCGCCAAGGGGCTGGGGG |
| | | AGGCGGCGCTCGGGGTAACCGGGGGAGACTCAGGGCGCTG |
| | | GGGGCACTTGGGGAACTCATGGGGGCTCAAAGGAACTAGGA |
| | | GATCGGGACCTCGAAGGGGACTTGGGGGGTTCGGGGCTTTC |
| | | GGGGGGGGTCGGGGGTTCGCGGACCCGGGAAGCTCTGAGG |
| | | ACCCAGAGGCCGGGCGCGCTCCGCCCGCGGCGCCGCCCCC |
| | | TCCGTAACTTTCCCAGTCTCCGAGGGAAGAGGCGGGGTGTG |
| | | GGGTGCGGTTAAAAGGCGCCACGGCGGGAGACAGGTGTTG |
| | | CGGCCCCGCAGCGCCCGCGCGCTCCTCTCCCCGACTCGGA |
| | | GCCCCTCGGCGGCGCCCGGCCCAGGACCCGCCTAGGAGCG |
| | | CAGGAGCCCCAGCGCAGAGACCCCAACGCCGAGACCCCCG |
| | | CCCCGGCCCCGCCGCGCTTCCTCCCGACGCAGGTGAGCCC |
| | | GCCGGCCCCGGACTGCCCGGCCAGGAACCTGGCGCGGGGA |
| | | GGGACCGCGAGACCCAGAGCGGTTGCCCGGCCGCGTGGGT |
| | | CTCGGGGAACCGGGGGGCTGGACCAACACACGTCCTTGGG |
| | | CCGGGGGGCGGGGGCCGCCTTCTGGAGCGGGCGTTTCTGT |
| | | TTATGATGTGTTTAAAGATTGGGTGAATTACTCAGGTGAACAA |
| | | GCTACTTTTTATCAGAGAACACCTAAAAACACGTTCAAGAGG |
| | | GTTTGGGAACTATACATTTAATCCTATGACAAACTAAGTTGGT |
| | | TCTGTCTTCACCTGTTTTGGTGAGGTTGTGTAAGAGTTGGTGT |
| | | TTGCTCAGGAAGAGATTTAAGCATGCTTGCTTACCCAGACTC |
| | | AGAGAAGTCTCCCTGTTCTGTCCTAGCTAGTGATTCCTGTGTT |
| | | GTGTGCATTCGTCTTTTCCAGAGCAAACCGCCCAGAGTAGAA |
| | | GCCTGCAGGCCACCATGGATTGGGGCACACTGCAGACTATC |
| | | CTGGGGGGTGTGAACAAACACTCCACCAGCATTGGAAAGAT |
| | | CTGGCTCACAGTCCTCTTCATTTTTAGGATTATGATCCTGGTT |
| | | GTGGCTGCAAAGGAGGTGTGGGGAGATGAGCAGGCTGACTT |
| | | TGTCTGCAACACCCTGCAGCCAGGCTGCAAGAATGTGTGCTA |
| | | TGATCACTACTTCCCCATCTCCCACATCAGGCTATGGGCCCT |
| | | GCAGCTGATCTTTGTGTCCACTCCAGCACTCCTAGTGGCCAT |
| | | GCATGTGGCCTACAGGAGACATGAGAAGAAGAGGAAGTTCA |
| | | TCAAGGGGGAGATAAAGAGTGAATTTAAGGACATTGAGGAGA |
| | | TCAAAACCCAGAAGGTCAGAATTGAAGGCTCCCTGTGGTGGA |
| | | CCTACACAAGCAGCATCTTCTTCAGGGTCATCTTTGAAGCAG |
| | | CCTTCATGTATGTCTTCTATGTCATGTATGATGGCTTCTCCAT |
| | | GCAGAGGCTGGTGAAGTGCAATGCCTGGCCTTGTCCCAACA |
| | | CTGTGGACTGCTTTGTGTCCAGGCCCACTGAGAAGACTGTCT |
| | | TCACAGTGTTCATGATTGCAGTGTCTGGAATTTGCATCCTGCT |
| | | GAATGTCACTGAATTGTGTTATTTGCTAATTAGATATTGTTCTG |
| | | GGAAGTCAAAAAAGCCAGTTTGATAAGCTTAGATCTGCCTGA |
| | | CTGTGCCTTCTAGTTGCCAGCCATCTGTTGTTTGCCCCTCCC |
| | | CCGTGCCTTCCTTGACCCTGGAAGGTGCCACTCCCACTGTCC |
| | | TTTCCTAATAAAATGAGGAAATTGCATCGCATTGTCTGAGTAG |
| | | GTGTCATTCTATTCTGGGGGGTGGGGTGGGGCAGGACAGCA |
| | | AGGGGGAGGATTGGGAAGACAATAGCAGGCATGCTGGGGAT |
| | | CTAGAACTGAATTCACAAGGAACCCCTAGTGATGGAGTTGGC |
| | | CACTCCCTCTCTGCGCGCTCGCTCGCTCACTGAGGCCGGGC |
| | | GACCAAAGGTCGCCCGACGCCCGGGCTTTGCCCGGGCGGC |
| | | CTCAGTGAGCGAGCGAGCGCGCAGCCTTAATTAACCTAATTC |
| | | ACTGGCCGTCGTTTTACAACGTCGTGACTGGGAAAACCCTGG |
| | | CGTTACCCAACTTAATCGCCTTGCAGCACATCCCCCTTTCGC |
| | | CAGCTGGCGTAATAGCGAAGAGGCCCGCACCGATCGCCCTT |
| | | CCCAACAGTTGCGCAGCCTGAATGGCGAATGGGACGCGCCC |
| | | TGTAGCGGCGCATTAAGCGCGGCGGGTGTGGTGGTTACGCG |
| | | CAGCGTGACCGCTACACTTGCCAGCGCCCTAGCGCCCGCTC |
| | | CTTTCGCTTTCTTCCCTTCCTTTCTCGCCACGTTCGCCGGCTT |
| | | TCCCCGTCAAGCTCTAAATCGGGGGCTCCCTTTAGGGTTCCG |
| | | ATTTAGTGCTTTACGGCACCTCGACCCCAAAAAACTTGATTAG |
| | | GGTGATGGTTCACGTAGTGGGCCATCGCCCTGATAGACGGT |
| | | TTTTCGCCCTTTGACGTTGGAGTCCACGTTCTTTAATAGTGGA |
| | | CTCTTGTTCCAAACTGGAACAACACTCAACCCTATCTCGGTCT |
| | | ATTCTTTTGATTTATAAGGGATTTTGCCGATTTCGGCCTATTG |
| | | GTTAAAAAATGAGCTGATTTAACAAAAATTTAACGCGAATTTTA |
| | | ACAAAATATTAACGCTTACAATTTAGGTGGCACTTTTCGGGGA |
| | | AATGTGCGCGGAACCCCTATTTGTTTATTTTTCTAAATACATT |
| | | CAAATATGTATCCGCTCATGAGACAATAACCCTGATAAATGCT |
| | | TCAATAATATTGAAAAAGGAAGAGTATGAGCCATATTCAACGG |
| | | GAAACGTCGAGGCCGCGATTAAATTCCAACATGGATGCTGAT |
| | | TTATATGGGTATAAATGGGCTCGCGATAATGTCGGGCAATCA |
| | | GGTGCGACAATCTATCGCTTGTATGGGAAGCCCGATGCGCC |
| | | AGAGTTGTTTCTGAAACATGGCAAAGGTAGCGTTGCCAATGA |
| | | TGTTACAGATGAGATGGTCAGACTAAACTGGCTGACGGAATT |
| | | TATGCCTCTTCCGACCATCAAGCATTTTATCCGTACTCCTGAT |
| | | GATGCATGGTTACTCACCACTGCGATCCCCGGAAAAACAGCA |
| | | TTCCAGGTATTAGAAGAATATCCTGATTCAGGTGAAAATATTG |
| | | TTGATGCGCTGGCAGTGTTCCTGCGCCGGTTGCATTCGATTC |
| | | CTGTTTGTAATTGTCCTTTTAACAGCGATCGCGTATTTCGTCT |
| | | CGCTCAGGCGCAATCACGAATGAATAACGGTTTGGTTGATGC |
| | | GAGTGATTTTGATGACGAGCGTAATGGCTGGCCTGTTGAACA |
| | | AGTCTGGAAAGAAATGCATAAACTTTTGCCATTCTCACCGGAT |
| | | TCAGTCGTCACTCATGGTGATTTCTCACTTGATAACCTTATTT |
| | | TTGACGAGGGGAAATTAATAGGTTGTATTGATGTTGGACGAG |
| | | TCGGAATCGCAGACCGATACCAGGATCTTGCCATCCTATGGA |
| | | ACTGCCTCGGTGAGTTTTCTCCTTCATTACAGAAACGGCTTTT |
| | | TCAAAAATATGGTATTGATAATCCTGATATGAATAAATTGCAG |
| | | TTTCATTTGATGCTCGATGAGTTTTTCTAACTGTCAGACCAAG |
| | | TTTACTCATATATACTTTAGATTGATTTAAAACTTCATTTTTAAT |
| | | TTAAAAGGATCTAGGTGAAGATCCTTTTTGATAATCTCATGAC |
| | | CAAAATCCCTTAACGTGAGTTTTCGTTCCACTGAGCGTCAGA |
| | | CCCCGTAGAAAAGATCAAAGGATCTTCTTGAGATCCTTTTTTT |
| | | CTGCGCGTAATCTGCTGCTTGCAAACAAAAAAACCACCGCTA |
| | | CCAGCGGTGGTTTGTTTGCCGGATCAAGAGCTACCAACTCTT |
| | | TTTCCGAAGGTAACTGGCTTCAGCAGAGCGCAGATACCAAAT |
| | | ACTGTTCTTCTAGTGTAGCCGTAGTTAGGCCACCACTTCAAG |
| | | AACTCTGTAGCACCGCCTACATACCTCGCTCTGCTAATCCTG |
| | | TTACCAGTGGCTGCTGCCAGTGGCGATAAGTCGTGTCTTACC |
| | | GGGTTGGACTCAAGACGATAGTTACCGGATAAGGCGCAGCG |
| | | GTCGGGCTGAACGGGGGGTTCGTGCACACAGCCCAGCTTGG |
| | | AGCGAACGACCTACACCGAACTGAGATACCTACAGCGTGAG |
| | | CTATGAGAAAGCGCCACGCTTCCCGAAGGGAGAAAGGCGGA |
| | | CAGGTATCCGGTAAGCGGCAGGGTCGGAACAGGAGAGCGC |
| | | ACGAGGGAGCTTCCAGGGGGAAACGCCTGGTATCTTTATAGT |
| | | CCTGTCGGGTTTCGCCACCTCTGACTTGAGCGTCGATTTTTG |
| | | TGATGCTCGTCAGGGGGGCGGAGCCTATGGAAAAACGCCAG |
| | | CAACGCGGCCTTTTTACGGTTCCTGGCCTTTTGCTGGCCTTT |
| | | TGCTCACATGTTCTTTCCTGCGTTATCCCCTGATTCTGTGGAT |
| | | AACCGTATTACCGCCTTTGAGTGAGCTGATACCGCTCGCCGC |
| | | AGCCGAACGACCGAGCGCAGCGAGTCAGTGAGCGAGGAAG |
| | | CGGAAGAGCGCCCAATACGCAAACCGCCTCTCCCCGCGCGT |
| | | TGGCCGATTCATTAATGCAGCTGGCACGACAGGTTTCCCGAC |
| | | TGGAAAGCGGGCAGTGAGCGCAACGCAATTAATGTGAGTTA |
| | | GCTCACTCATTAGGCACCCCAGGCTTTACACTTTATGCTTCC |
| | | GGCTCGTATGTTGTGTGGAATTGTGAGCGGATAACAATTTCA |
| | | CACAGGAAACAGCTATGACCATGATTACGCCAGATTTAATTAA |
| | | GGCCTTAATTAGG |
| 19 | Plasmid P1599 | CTGCGCGCTCGCTCGCTCACTGAGGCCGCCCGGGCAAAGC |
| | 5′ ITR at nucleotides | CCGGGCGTCGGGCGACCTTTGGTCGCCCGGCCTCAGTGAG |
| | 1-130 | CGAGCGAGCGCGCAGAGAGGGAGTGGCCAACTCCATCACTA |
| | GJB2 regulatory | GGGGTTCCTGAGCAGAATTCTGGTACCTTGCTAGCAAAAACT |
| | construct at nucleotides | CTTACATAATTGTAACAGATTTGAGTTTCCTCTGGTTCAGGTT |
| | 157-2617 | TTCTTGCCTCTTTGATAATCAAATGATCTGAAGAAAGGCATAG |
| | Codon-optimized and | AATTTCAAGGGAGAATCTGCATGACAGGATTACAATAAGGCT |
| | CpG depleted hGJB2 at | ATTCATGGAGACTCTTTTATTAGCTTACACAGGATCTGCATCA |
| | nucleotides 2631-3308 | TTCTTCGTCTCTGGCTTTGCTACAAGGCTCCATTTAAACTTAA |
| | bGH polyA signal | CCCAACTTGCAGGCTTAACTGATCCAGGAATGACTCAATACA |
| | sequence at nucleotides | ATGAGCCAGCTACTGTAGCTCTTTTTCCCCTTTTGATAAGGG |
| | 3331-3538 | GAGTTAACACAATGGGCTTTACAGTTCTTAAATGACCGTTCCT |
| | 3′ ITR at nucleotides | GCAGGTATTGGAAAACCTGCATCGTATGCCTGCTGCTTTAGG |
| | 3557-3686 | AGACAACAAACTCAGCAGCCCCTCCCCCGAAGCTGAAGTGC |
| | AmpR promoter at | CAAACTTAAGGCTCCAGGTAAACTGCCCTTCCGATGAGAGCT |
| | nucleotides 4344-4448 | TTACATTTAGGCAGTAGCCACAGGCCTAAACAAAGTCAAATG |
| | KanR at nucleotides | GATGAAACTTGTGATCTCTCCTTGGAAATATGGCCAAGAGTG |
| | 4449-5258 | AAACAACTACTTAGTTATTATTTTAAACGCAGGCTGCTAAATG |
| | Sequence to be | GCTTGTGTTGGTATGTAATGGCATCCTAGATAATGGGGGCTG |
| | transferred into vector | CCAAGAAAATATTCACACTGGCAAGTCAATTAAAATACCGTTC |
| | at nucleotides 1-3686 | TCTTCCTAAGAGGCCTGGCTGGATTCCAGAGCTTCCTTTAGG |
| | | GTAACGCTCAAACCTGTCATAGTTTATTTTGTTTCTGTTTTCAG |
| | | GCAACAGATTTGGTTATTGCTTTTATTGTATGAGTGTTGAAAT |
| | | GGTGTCCTAACAGCCATCTGTACTGCCATCCCCTGGTCTCTC |
| | | TGCCTTTCCAGAAAGGAGATGAAGTTGCTGGAGCAGGCAGG |
| | | TTCTCTGAAAGGCTCCCTGAGTGTGGAAGAGCAGCTGTCACT |
| | | CATCAGTGGTTGTCCCAATATCCAAGAAGCAGTAGAGGGTGC |
| | | CATGCACATTCAGGTGAGTGAAGGACACTGGCGCCGCATCT |
| | | GTGGGGCCTGAGACATTGTCTCCCAGCCTCTCCGGAGGAAC |
| | | AGGGCAGGATACAGAGCAGTGGCAGGTGTGGGCATTTTGCA |
| | | TCTTCTGTAATCTCCAAACAAAGTCTGCGCTAGGCCCTTTCAG |
| | | GCTATAAAGGACAGGGTACTCTGATTTCTGCTCTTGATGAGG |
| | | GTTGATTGTGATACCAGGCGAGGGAGTAAGAGATACAGGAC |
| | | ACCATCTCTGCGGCTGCATCTTTGTCGCAGAAACAGACATTG |
| | | TTCAGGGGACACCTGAGCCTCTGATAGTCAGCCAGTGGCTCT |
| | | GGCAGCTCACAGCAGTTATAGGGTGGCTGTGTCCTTTTCTAA |
| | | AGTCCTGACTTTTTATGGCCTGTGGTCATGGTGACTCCACTA |
| | | CGGGCTGTGTTTCTGTTGACGCGTACAGAGGACAACGACCA |
| | | CAGCCATCCCTGAACCCCGCCCACGGCACAGCGCCGGAGC |
| | | CGGGGTCTGGGGCGCCGCTTCCTGGGGGGTCCCGACTCTC |
| | | AGCCGCCCCCGCTTCACCCGGGCCGCCAAGGGGCTGGGGG |
| | | AGGCGGCGCTCGGGGTAACCGGGGGAGACTCAGGGCGCTG |
| | | GGGGCACTTGGGGAACTCATGGGGGCTCAAAGGAACTAGGA |
| | | GATCGGGACCTCGAAGGGGACTTGGGGGGTTCGGGGCTTTC |
| | | GGGGGGGGTCGGGGGTTCGCGGACCCGGGAAGCTCTGAGG |
| | | ACCCAGAGGCCGGGCGCGCTCCGCCCGCGGCGCCGCCCCC |
| | | TCCGTAACTTTCCCAGTCTCCGAGGGAAGAGGCGGGGTGTG |
| | | GGGTGCGGTTAAAAGGCGCCACGGCGGGAGACAGGTGTTG |
| | | CGGCCCCGCAGCGCCCGCGCGCTCCTCTCCCCGACTCGGA |
| | | GCCCCTCGGCGGCGCCCGGCCCAGGACCCGCCTAGGAGCG |
| | | CAGGAGCCCCAGCGCAGAGACCCCAACGCCGAGACCCCCG |
| | | CCCCGGCCCCGCCGCGCTTCCTCCCGACGCAGGTGAGCCC |
| | | GCCGGCCCCGGACTGCCCGGCCAGGAACCTGGCGCGGGGA |
| | | GGGACCGCGAGACCCAGAGCGGTTGCCCGGCCGCGTGGGT |
| | | CTCGGGGAACCGGGGGGCTGGACCAACACACGTCCTTGGG |
| | | CCGGGGGGCGGGGGCCGCCTTCTGGAGCGGGCGTTTCTGT |
| | | TTATGATGTGTTTAAAGATTGGGTGAATTACTCAGGTGAACAA |
| | | GCTACTTTTTATCAGAGAACACCTAAAAACACGTTCAAGAGG |
| | | GTTTGGGAACTATACATTTAATCCTATGACAAACTAAGTTGGT |
| | | TCTGTCTTCACCTGTTTTGGTGAGGTTGTGTAAGAGTTGGTGT |
| | | TTGCTCAGGAAGAGATTTAAGCATGCTTGCTTACCCAGACTC |
| | | AGAGAAGTCTCCCTGTTCTGTCCTAGCTAGTGATTCCTGTGTT |
| | | GTGTGCATTCGTCTTTTCCAGAGCAAACCGCCCAGAGTAGAA |
| | | GCCTGCAGGCCACCATGGACTGGGGAACACTGCAAACAATC |
| | | CTGGGAGGTGTGAACAAGCACAGCACAAGCATTGGCAAGAT |
| | | CTGGCTGACAGTGCTGTTCATCTTTAGAATCATGATCCTGGTT |
| | | GTGGCTGCTAAGGAAGTGTGGGGAGATGAGCAGGCTGACTT |
| | | TGTTTGTAATACCCTGCAACCTGGCTGCAAGAATGTGTGTTAT |
| | | GACCACTACTTCCCCATCAGCCACATCAGACTGTGGGCCCTG |
| | | CAACTGATCTTTGTGTCCACCCCTGCCCTGCTGGTGGCCATG |
| | | CATGTGGCCTACAGAAGGCATGAGAAGAAGAGGAAATTCATC |
| | | AAGGGAGAGATCAAGAGTGAGTTCAAGGACATTGAGGAAATC |
| | | AAAACCCAGAAGGTGAGGATTGAAGGCTCTCTGTGGTGGAC |
| | | CTACACCAGCAGCATCTTCTTCAGAGTGATCTTTGAGGCAGC |
| | | CTTTATGTATGTGTTCTATGTGATGTATGATGGCTTCTCCATG |
| | | CAGAGACTGGTGAAGTGCAATGCCTGGCCATGTCCTAACACA |
| | | GTGGACTGCTTTGTGTCTAGACCTACAGAAAAGACAGTGTTT |
| | | ACAGTCTTTATGATTGCAGTGTCTGGCATCTGCATTCTGCTGA |
| | | ATGTGACTGAGCTGTGCTACCTGCTCATTAGGTACTGTAGTG |
| | | GAAAAAGCAAGAAACCTGTGTGATAAGCTTAGATCTGCCTGA |
| | | CTGTGCCTTCTAGTTGCCAGCCATCTGTTGTTTGCCCCTCCC |
| | | CCGTGCCTTCCTTGACCCTGGAAGGTGCCACTCCCACTGTCC |
| | | TTTCCTAATAAAATGAGGAAATTGCATCGCATTGTCTGAGTAG |
| | | GTGTCATTCTATTCTGGGGGGTGGGGTGGGGCAGGACAGCA |
| | | AGGGGGAGGATTGGGAAGACAATAGCAGGCATGCTGGGGAT |
| | | CTAGAACTGAATTCACAAGGAACCCCTAGTGATGGAGTTGGC |
| | | CACTCCCTCTCTGCGCGCTCGCTCGCTCACTGAGGCCGGGC |
| | | GACCAAAGGTCGCCCGACGCCCGGGCTTTGCCCGGGCGGC |
| | | CTCAGTGAGCGAGCGAGCGCGCAGCCTTAATTAACCTAATTC |
| | | ACTGGCCGTCGTTTTACAACGTCGTGACTGGGAAAACCCTGG |
| | | CGTTACCCAACTTAATCGCCTTGCAGCACATCCCCCTTTCGC |
| | | CAGCTGGCGTAATAGCGAAGAGGCCCGCACCGATCGCCCTT |
| | | CCCAACAGTTGCGCAGCCTGAATGGCGAATGGGACGCGCCC |
| | | TGTAGCGGCGCATTAAGCGCGGCGGGTGTGGTGGTTACGCG |
| | | CAGCGTGACCGCTACACTTGCCAGCGCCCTAGCGCCCGCTC |
| | | CTTTCGCTTTCTTCCCTTCCTTTCTCGCCACGTTCGCCGGCTT |
| | | TCCCCGTCAAGCTCTAAATCGGGGGCTCCCTTTAGGGTTCCG |
| | | ATTTAGTGCTTTACGGCACCTCGACCCCAAAAAACTTGATTAG |
| | | GGTGATGGTTCACGTAGTGGGCCATCGCCCTGATAGACGGT |
| | | TTTTCGCCCTTTGACGTTGGAGTCCACGTTCTTTAATAGTGGA |
| | | CTCTTGTTCCAAACTGGAACAACACTCAACCCTATCTCGGTCT |
| | | ATTCTTTTGATTTATAAGGGATTTTGCCGATTTCGGCCTATTG |
| | | GTTAAAAAATGAGCTGATTTAACAAAAATTTAACGCGAATTTTA |
| | | ACAAAATATTAACGCTTACAATTTAGGTGGCACTTTTCGGGGA |
| | | AATGTGCGCGGAACCCCTATTTGTTTATTTTTCTAAATACATT |
| | | CAAATATGTATCCGCTCATGAGACAATAACCCTGATAAATGCT |
| | | TCAATAATATTGAAAAAGGAAGAGTATGAGCCATATTCAACGG |
| | | GAAACGTCGAGGCCGCGATTAAATTCCAACATGGATGCTGAT |
| | | TTATATGGGTATAAATGGGCTCGCGATAATGTCGGGCAATCA |
| | | GGTGCGACAATCTATCGCTTGTATGGGAAGCCCGATGCGCC |
| | | AGAGTTGTTTCTGAAACATGGCAAAGGTAGCGTTGCCAATGA |
| | | TGTTACAGATGAGATGGTCAGACTAAACTGGCTGACGGAATT |
| | | TATGCCTCTTCCGACCATCAAGCATTTTATCCGTACTCCTGAT |
| | | GATGCATGGTTACTCACCACTGCGATCCCCGGAAAAACAGCA |
| | | TTCCAGGTATTAGAAGAATATCCTGATTCAGGTGAAAATATTG |
| | | TTGATGCGCTGGCAGTGTTCCTGCGCCGGTTGCATTCGATTC |
| | | CTGTTTGTAATTGTCCTTTTAACAGCGATCGCGTATTTCGTCT |
| | | CGCTCAGGCGCAATCACGAATGAATAACGGTTTGGTTGATGC |
| | | GAGTGATTTTGATGACGAGCGTAATGGCTGGCCTGTTGAACA |
| | | AGTCTGGAAAGAAATGCATAAACTTTTGCCATTCTCACCGGAT |
| | | TCAGTCGTCACTCATGGTGATTTCTCACTTGATAACCTTATTT |
| | | TTGACGAGGGGAAATTAATAGGTTGTATTGATGTTGGACGAG |
| | | TCGGAATCGCAGACCGATACCAGGATCTTGCCATCCTATGGA |
| | | ACTGCCTCGGTGAGTTTTCTCCTTCATTACAGAAACGGCTTTT |
| | | TCAAAAATATGGTATTGATAATCCTGATATGAATAAATTGCAG |
| | | TTTCATTTGATGCTCGATGAGTTTTTCTAACTGTCAGACCAAG |
| | | TTTACTCATATATACTTTAGATTGATTTAAAACTTCATTTTTAAT |
| | | TTAAAAGGATCTAGGTGAAGATCCTTTTTGATAATCTCATGAC |
| | | CAAAATCCCTTAACGTGAGTTTTCGTTCCACTGAGCGTCAGA |
| | | CCCCGTAGAAAAGATCAAAGGATCTTCTTGAGATCCTTTTTTT |
| | | CTGCGCGTAATCTGCTGCTTGCAAACAAAAAAACCACCGCTA |
| | | CCAGCGGTGGTTTGTTTGCCGGATCAAGAGCTACCAACTCTT |
| | | TTTCCGAAGGTAACTGGCTTCAGCAGAGCGCAGATACCAAAT |
| | | ACTGTTCTTCTAGTGTAGCCGTAGTTAGGCCACCACTTCAAG |
| | | AACTCTGTAGCACCGCCTACATACCTCGCTCTGCTAATCCTG |
| | | TTACCAGTGGCTGCTGCCAGTGGCGATAAGTCGTGTCTTACC |
| | | GGGTTGGACTCAAGACGATAGTTACCGGATAAGGCGCAGCG |
| | | GTCGGGCTGAACGGGGGGTTCGTGCACACAGCCCAGCTTGG |
| | | AGCGAACGACCTACACCGAACTGAGATACCTACAGCGTGAG |
| | | CTATGAGAAAGCGCCACGCTTCCCGAAGGGAGAAAGGCGGA |
| | | CAGGTATCCGGTAAGCGGCAGGGTCGGAACAGGAGAGCGC |
| | | ACGAGGGAGCTTCCAGGGGGAAACGCCTGGTATCTTTATAGT |
| | | CCTGTCGGGTTTCGCCACCTCTGACTTGAGCGTCGATTTTTG |
| | | TGATGCTCGTCAGGGGGGCGGAGCCTATGGAAAAACGCCAG |
| | | CAACGCGGCCTTTTTACGGTTCCTGGCCTTTTGCTGGCCTTT |
| | | TGCTCACATGTTCTTTCCTGCGTTATCCCCTGATTCTGTGGAT |
| | | AACCGTATTACCGCCTTTGAGTGAGCTGATACCGCTCGCCGC |
| | | AGCCGAACGACCGAGCGCAGCGAGTCAGTGAGCGAGGAAG |
| | | CGGAAGAGCGCCCAATACGCAAACCGCCTCTCCCCGCGCGT |
| | | TGGCCGATTCATTAATGCAGCTGGCACGACAGGTTTCCCGAC |
| | | TGGAAAGCGGGCAGTGAGCGCAACGCAATTAATGTGAGTTA |
| | | GCTCACTCATTAGGCACCCCAGGCTTTACACTTTATGCTTCC |
| | | GGCTCGTATGTTGTGTGGAATTGTGAGCGGATAACAATTTCA |
| | | CACAGGAAACAGCTATGACCATGATTACGCCAGATTTAATTAA |
| | | GGCCTTAATTAGG |
| 20 | Plasmid P1601 | CTGCGCGCTCGCTCGCTCACTGAGGCCGCCCGGGCAAAGC |
| | 5′ ITR at nucleotides | CCGGGCGTCGGGCGACCTTTGGTCGCCCGGCCTCAGTGAG |
| | 1-130 | CGAGCGAGCGCGCAGAGAGGGAGTGGCCAACTCCATCACTA |
| | GJB2 regulatory | GGGGTTCCTTGTAGTTAATGATTAACCCGCCATGCTACTTATC |
| | construct at nucleotides | TACGTAGCCATGCTCTAGGAAGATCGGAATTCTGGTACCTTG |
| | 212-1596 | CTAGCAAAAACTCTTACATAATTGTAACAGATTTGAGTTTCCT |
| | CpG depleted hGJB2 at | CTGGTTCAGGTTTTCTTGCCTCTTTGATAATCAAATGATCTGA |
| | nucleotides 1610-2287 | AGAAAGGCATAGAATTTCAAGGGAGAATCTGCATGACAGGAT |
| | SV40 polyA signal | TACAATAAGGCTATTCATGGAGACTCTTTTATTAGCTTACACA |
| | sequence at nucleotides | GGATCTGCATCATTCTTCGTCTCTGGCTTTGCTACAAGGCTC |
| | 2304-2438 | CATTTAAACTTAACCCAACTTGCAGGCTTAACTGATCCAGGAA |
| | 3′ ITR at nucleotides | TGACTCAATACAATGAGCCAGCTACTGTAGCTCTTTTTCCCCT |
| | 2521-2650 | TTTGATAAGGGGAGTTAACACAATGGGCTTTACAGTTCTTAAA |
| | AmpR promoter at | TGAACGCGTACAGAGGACAACGACCACAGCCATCCCTGAAC |
| | nucleotides 3308-3412 | CCCGCCCACGGCACAGCGCCGGAGCCGGGGTCTGGGGCGC |
| | KanR at nucleotides | CGCTTCCTGGGGGGTCCCGACTCTCAGCCGCCCCCGCTTCA |
| | 3413-4222 | CCCGGGCCGCCAAGGGGCTGGGGGAGGCGGCGCTCGGGG |
| | Sequence to be | TAACCGGGGGAGACTCAGGGCGCTGGGGGCACTTGGGGAA |
| | transferred into vector | CTCATGGGGGCTCAAAGGAACTAGGAGATCGGGACCTCGAA |
| | at nucleotides 1-2650 | GGGGACTTGGGGGGTTCGGGGCTTTCGGGGGCGGGTCGGGG |
| | | GTTCGCGGACCCGGGAAGCTCTGAGGACCCAGAGGCCGGG |
| | | CGCGCTCCGCCCGCGGCGCCGCCCCCTCCGTAACTTTCCCA |
| | | GTCTCCGAGGGAAGAGGGGGGGTGTGGGGTGCGGTTAAAA |
| | | GGCGCCACGGCGGGAGACAGGTGTTGCGGCCCCGCAGCGC |
| | | CCGCGCGCTCCTCTCCCCGACTCGGAGCCCCTCGGCGGCG |
| | | CCCGGCCCAGGACCCGCCTAGGAGCGCAGGAGCCCCAGCG |
| | | CAGAGACCCCAACGCCGAGACCCCCGCCCCGGCCCCGCCG |
| | | CGCTTCCTCCCGACGCAGGTGAGCCCGCCGGCCCCGGACT |
| | | GCCCGGCCAGGAACCTGGCGCGGGGAGGGACCGCGAGACC |
| | | CAGAGCGGTTGCCCGGCCGCGTGGGTCTCGGGGAACCGGG |
| | | GGGCTGGACCAACACACGTCCTTGGGCCGGGGGGCGGGGG |
| | | CCGCCTTCTGGAGCGGGCGTTTCTGTTTATGATGTGTTTAAA |
| | | GATTGGGTGAATTACTCAGGTGAACAAGCTACTTTTTATCAGA |
| | | GAACACCTAAAAACACGTTCAAGAGGGTTTGGGAACTATACA |
| | | TTTAATCCTATGACAAACTAAGTTGGTTCTGTCTTCACCTGTTT |
| | | TGGTGAGGTTGTGTAAGAGTTGGTGTTTGCTCAGGAAGAGAT |
| | | TTAAGCATGCTTGCTTACCCAGACTCAGAGAAGTCTCCCTGT |
| | | TCTGTCCTAGCTAGTGATTCCTGTGTTGTGTGCATTCGTCTTT |
| | | TCCAGAGCAAACCGCCCAGAGTAGAAGCCTGCAGGCCACCA |
| | | TGGATTGGGGCACACTGCAGACTATCCTGGGGGGTGTGAAC |
| | | AAACACTCCACCAGCATTGGAAAGATCTGGCTCACAGTCCTC |
| | | TTCATTTTTAGGATTATGATCCTGGTTGTGGCTGCAAAGGAG |
| | | GTGTGGGGAGATGAGCAGGCTGACTTTGTCTGCAACACCCT |
| | | GCAGCCAGGCTGCAAGAATGTGTGCTATGATCACTACTTCCC |
| | | CATCTCCCACATCAGGCTATGGGCCCTGCAGCTGATCTTTGT |
| | | GTCCACTCCAGCACTCCTAGTGGCCATGCATGTGGCCTACAG |
| | | GAGACATGAGAAGAAGAGGAAGTTCATCAAGGGGGAGATAA |
| | | AGAGTGAATTTAAGGACATTGAGGAGATCAAAACCCAGAAGG |
| | | TCAGAATTGAAGGCTCCCTGTGGTGGACCTACACAAGCAGCA |
| | | TCTTCTTCAGGGTCATCTTTGAAGCAGCCTTCATGTATGTCTT |
| | | CTATGTCATGTATGATGGCTTCTCCATGCAGAGGCTGGTGAA |
| | | GTGCAATGCCTGGCCTTGTCCCAACACTGTGGACTGCTTTGT |
| | | GTCCAGGCCCACTGAGAAGACTGTCTTCACAGTGTTCATGAT |
| | | TGCAGTGTCTGGAATTTGCATCCTGCTGAATGTCACTGAATT |
| | | GTGTTATTTGCTAATTAGATATTGTTCTGGGAAGTCAAAAAAG |
| | | CCAGTTTGATAAGCTTAGATCTGATCCAGACATGATAAGATAC |
| | | ATTGATGAGTTTGGACAAACCAAAACTAGAATGCAGTGAAAAA |
| | | AATGCCTTATTTGTGAAATTTGTGATGCTATTGCCTTATTTGTA |
| | | ACCATTATAAGCTGCAATAAACAAGTTTCTAGAACTGAATTCC |
| | | CGATAAGGATCTTCCTAGAGCATGGCTACGTAGATAAGTAGC |
| | | ATGGCGGGTTAATCATTAACTACAAGGAACCCCTAGTGATGG |
| | | AGTTGGCCACTCCCTCTCTGCGCGCTCGCTCGCTCACTGAG |
| | | GCCGGGCGACCAAAGGTCGCCCGACGCCCGGGCTTTGCCC |
| | | GGGCGGCCTCAGTGAGCGAGCGAGCGCGCAGCCTTAATTAA |
| | | CCTAATTCACTGGCCGTCGTTTTACAACGTCGTGACTGGGAA |
| | | AACCCTGGCGTTACCCAACTTAATCGCCTTGCAGCACATCCC |
| | | CCTTTCGCCAGCTGGCGTAATAGCGAAGAGGCCCGCACCGA |
| | | TCGCCCTTCCCAACAGTTGCGCAGCCTGAATGGCGAATGGG |
| | | ACGCGCCCTGTAGCGGCGCATTAAGCGCGGCGGGTGTGGT |
| | | GGTTACGCGCAGCGTGACCGCTACACTTGCCAGCGCCCTAG |
| | | CGCCCGCTCCTTTCGCTTTCTTCCCTTCCTTTCTCGCCACGTT |
| | | CGCCGGCTTTCCCCGTCAAGCTCTAAATCGGGGGCTCCCTTT |
| | | AGGGTTCCGATTTAGTGCTTTACGGCACCTCGACCCCAAAAA |
| | | ACTTGATTAGGGTGATGGTTCACGTAGTGGGCCATCGCCCTG |
| | | ATAGACGGTTTTTCGCCCTTTGACGTTGGAGTCCACGTTCTTT |
| | | AATAGTGGACTCTTGTTCCAAACTGGAACAACACTCAACCCTA |
| | | TCTCGGTCTATTCTTTTGATTTATAAGGGATTTTGCCGATTTC |
| | | GGCCTATTGGTTAAAAAATGAGCTGATTTAACAAAAATTTAAC |
| | | GCGAATTTTAACAAAATATTAACGCTTACAATTTAGGTGGCAC |
| | | TTTTCGGGGAAATGTGCGCGGAACCCCTATTTGTTTATTTTTC |
| | | TAAATACATTCAAATATGTATCCGCTCATGAGACAATAACCCT |
| | | GATAAATGCTTCAATAATATTGAAAAAGGAAGAGTATGAGCCA |
| | | TATTCAACGGGAAACGTCGAGGCCGCGATTAAATTCCAACAT |
| | | GGATGCTGATTTATATGGGTATAAATGGGCTCGCGATAATGT |
| | | CGGGCAATCAGGTGCGACAATCTATCGCTTGTATGGGAAGC |
| | | CCGATGCGCCAGAGTTGTTTCTGAAACATGGCAAAGGTAGCG |
| | | TTGCCAATGATGTTACAGATGAGATGGTCAGACTAAACTGGC |
| | | TGACGGAATTTATGCCTCTTCCGACCATCAAGCATTTTATCCG |
| | | TACTCCTGATGATGCATGGTTACTCACCACTGCGATCCCCGG |
| | | AAAAACAGCATTCCAGGTATTAGAAGAATATCCTGATTCAGGT |
| | | GAAAATATTGTTGATGCGCTGGCAGTGTTCCTGCGCCGGTTG |
| | | CATTCGATTCCTGTTTGTAATTGTCCTTTTAACAGCGATCGCG |
| | | TATTTCGTCTTGCTCAGGCGCAATCACGAATGAATAACGGTTT |
| | | GGTTGATGCGAGTGATTTTGATGACGAGCGTAATGGCTGGCC |
| | | TGTTGAACAAGTCTGGAAAGAAATGCATAAACTTTTGCCATTC |
| | | TCACCGGATTCAGTCGTCACTCATGGTGATTTCTCACTTGATA |
| | | ACCTTATTTTTGACGAGGGGAAATTAATAGGTTGTATTGATGT |
| | | TGGACGAGTCGGAATCGCAGACCGATACCAGGATCTTGCCA |
| | | TCCTATGGAACTGCCTCGGTGAGTTTTCTCCTTCATTACAGAA |
| | | ACGGCTTTTTCAAAAATATGGTATTGATAATCCTGATATGAAT |
| | | AAATTGCAGTTTCATTTGATGCTCGATGAGTTTTTCTAACTGT |
| | | CAGACCAAGTTTACTCATATATACTTTAGATTGATTTAAAACTT |
| | | CATTTTTAATTTAAAAGGATCTAGGTGAAGATCCTTTTTGATAA |
| | | TCTCATGACCAAAATCCCTTAACGTGAGTTTTCGTTCCACTGA |
| | | GCGTCAGACCCCGTAGAAAAGATCAAAGGATCTTCTTGAGAT |
| | | CCTTTTTTTCTGCGCGTAATCTGCTGCTTGCAAACAAAAAAAC |
| | | CACCGCTACCAGCGGTGGTTTGTTTGCCGGATCAAGAGCTAC |
| | | CAACTCTTTTTCCGAAGGTAACTGGCTTCAGCAGAGCGCAGA |
| | | TACCAAATACTGTTCTTCTAGTGTAGCCGTAGTTAGGCCACCA |
| | | CTTCAAGAACTCTGTAGCACCGCCTACATACCTCGCTCTGCT |
| | | AATCCTGTTACCAGTGGCTGCTGCCAGTGGCGATAAGTCGTG |
| | | TCTTACCGGGTTGGACTCAAGACGATAGTTACCGGATAAGGC |
| | | GCAGCGGTCGGGCTGAACGGGGGGTTCGTGCACACAGCCC |
| | | AGCTTGGAGCGAACGACCTACACCGAACTGAGATACCTACAG |
| | | CGTGAGCTATGAGAAAGCGCCACGCTTCCCGAAGGGAGAAA |
| | | GGCGGACAGGTATCCGGTAAGCGGCAGGGTCGGAACAGGA |
| | | GAGCGCACGAGGGAGCTTCCAGGGGGAAACGCCTGGTATCT |
| | | TTATAGTCCTGTCGGGTTTCGCCACCTCTGACTTGAGCGTCG |
| | | ATTTTTGTGATGCTCGTCAGGGGGGCGGAGCCTATGGAAAAA |
| | | CGCCAGCAACGCGGCCTTTTTACGGTTCCTGGCCTTTTGCTG |
| | | GCCTTTTGCTCACATGTTCTTTCCTGCGTTATCCCCTGATTCT |
| | | GTGGATAACCGTATTACCGCCTTTGAGTGAGCTGATACCGCT |
| | | CGCCGCAGCCGAACGACCGAGCGCAGCGAGTCAGTGAGCG |
| | | AGGAAGCGGAAGAGCGCCCAATACGCAAACCGCCTCTCCCC |
| | | GCGCGTTGGCCGATTCATTAATGCAGCTGGCACGACAGGTTT |
| | | CCCGACTGGAAAGCGGGCAGTGAGCGCAACGCAATTAATGT |
| | | GAGTTAGCTCACTCATTAGGCACCCCAGGCTTTACACTTTAT |
| | | GCTTCCGGCTCGTATGTTGTGTGGAATTGTGAGCGGATAACA |
| | | ATTTCACACAGGAAACAGCTATGACCATGATTACGCCAGATTT |
| | | AATTAAGGCCTTAATTAGG |
| 21 | Plasmid P1602 | CTGCGCGCTCGCTCGCTCACTGAGGCCGCCCGGGCAAAGC |
| | 5′ ITR at nucleotides | CCGGGCGTCGGGCGACCTTTGGTCGCCCGGCCTCAGTGAG |
| | 1-130 | CGAGCGAGCGCGCAGAGAGGGAGTGGCCAACTCCATCACTA |
| | GJB2 regulatory | GGGGTTCCTTGTAGTTAATGATTAACCCGCCATGCTACTTATC |
| | construct at nucleotides | TACGTAGCCATGCTCTAGGAAGATCGGAATTCTGGTACCTTG |
| | 212-1596 | CTAGCAAAAACTCTTACATAATTGTAACAGATTTGAGTTTCCT |
| | Codon-optimized and | CTGGTTCAGGTTTTCTTGCCTCTTTGATAATCAAATGATCTGA |
| | CpG depleted hGJB2 at | AGAAAGGCATAGAATTTCAAGGGAGAATCTGCATGACAGGAT |
| | nucleotides 1610-2287 | TACAATAAGGCTATTCATGGAGACTCTTTTATTAGCTTACACA |
| | SV40 polyA signal | GGATCTGCATCATTCTTCGTCTCTGGCTTTGCTACAAGGCTC |
| | sequence at nucleotides | CATTTAAACTTAACCCAACTTGCAGGCTTAACTGATCCAGGAA |
| | 2304-2438 | TGACTCAATACAATGAGCCAGCTACTGTAGCTCTTTTTCCCCT |
| | 3′ ITR at nucleotides | TTTGATAAGGGGAGTTAACACAATGGGCTTTACAGTTCTTAAA |
| | 2521-2650 | TGAACGCGTACAGAGGACAACGACCACAGCCATCCCTGAAC |
| | AmpR promoter at | CCCGCCCACGGCACAGCGCCGGAGCCGGGGTCTGGGGCGC |
| | nucleotides 3308-3412 | CGCTTCCTGGGGGGTCCCGACTCTCAGCCGCCCCCGCTTCA |
| | KanR at nucleotides | CCCGGGCCGCCAAGGGGCTGGGGGAGGCGGCGCTCGGGG |
| | 3413-4222 | TAACCGGGGGAGACTCAGGGCGCTGGGGGCACTTGGGGAA |
| | Sequence to be | CTCATGGGGGCTCAAAGGAACTAGGAGATCGGGACCTCGAA |
| | transferred into vector | GGGGACTTGGGGGGTTCGGGGCTTTCGGGGGCGGTCGGGG |
| | at nucleotides 1-2650 | GTTCGCGGACCCGGGAAGCTCTGAGGACCCAGAGGCCGGG |
| | | CGCGCTCCGCCCGCGGCGCCGCCCCCTCCGTAACTTTCCCA |
| | | GTCTCCGAGGGAAGAGGCGGGGTGTGGGGTGCGGTTAAAA |
| | | GGCGCCACGGCGGGAGACAGGTGTTGCGGCCCCGCAGCGC |
| | | CCGCGCGCTCCTCTCCCCGACTCGGAGCCCCTCGGCGGCG |
| | | CCCGGCCCAGGACCCGCCTAGGAGCGCAGGAGCCCCAGCG |
| | | CAGAGACCCCAACGCCGAGACCCCCGCCCCGGCCCCGCCG |
| | | CGCTTCCTCCCGACGCAGGTGAGCCCGCCGGCCCCGGACT |
| | | GCCCGGCCAGGAACCTGGCGCGGGGAGGGACCGCGAGACC |
| | | CAGAGCGGTTGCCCGGCCGCGTGGGTCTCGGGGAACCGGG |
| | | GGGCTGGACCAACACACGTCCTTGGGCCGGGGGGCGGGGG |
| | | CCGCCTTCTGGAGGGGGCGTTTCTGTTTATGATGTGTTTAAA |
| | | GATTGGGTGAATTACTCAGGTGAACAAGCTACTTTTTATCAGA |
| | | GAACACCTAAAAACACGTTCAAGAGGGTTTGGGAACTATACA |
| | | TTTAATCCTATGACAAACTAAGTTGGTTCTGTCTTCACCTGTTT |
| | | TGGTGAGGTTGTGTAAGAGTTGGTGTTTGCTCAGGAAGAGAT |
| | | TTAAGCATGCTTGCTTACCCAGACTCAGAGAAGTCTCCCTGT |
| | | TCTGTCCTAGCTAGTGATTCCTGTGTTGTGTGCATTCGTCTTT |
| | | TCCAGAGCAAACCGCCCAGAGTAGAAGCCTGCAGGCCACCA |
| | | TGGATTGGGGCACACTGCAGACTATCCTGGGGGGTGTGAAC |
| | | AAACACTCCACCAGCATTGGAAAGATCTGGCTCACAGTCCTC |
| | | TTCATTTTTAGGATTATGATCCTGGTTGTGGCTGCAAAGGAG |
| | | GTGTGGGGAGATGAGCAGGCTGACTTTGTCTGCAACACCCT |
| | | GCAGCCAGGCTGCAAGAATGTGTGCTATGATCACTACTTCCC |
| | | CATCTCCCACATCAGGCTATGGGCCCTGCAGCTGATCTTTGT |
| | | GTCCACTCCAGCACTCCTAGTGGCCATGCATGTGGCCTACAG |
| | | GAGACATGAGAAGAAGAGGAAGTTCATCAAGGGGGAGATAA |
| | | AGAGTGAATTTAAGGACATTGAGGAGATCAAAACCCAGAAGG |
| | | TCAGAATTGAAGGCTCCCTGTGGTGGACCTACACAAGCAGCA |
| | | TCTTCTTCAGGGTCATCTTTGAAGCAGCCTTCATGTATGTCTT |
| | | CTATGTCATGTATGATGGCTTCTCCATGCAGAGGCTGGTGAA |
| | | GTGCAATGCCTGGCCTTGTCCCAACACTGTGGACTGCTTTGT |
| | | GTCCAGGCCCACTGAGAAGACTGTCTTCACAGTGTTCATGAT |
| | | TGCAGTGTCTGGAATTTGCATCCTGCTGAATGTCACTGAATT |
| | | GTGTTATTTGCTAATTAGATATTGTTCTGGGAAGTCAAAAAAG |
| | | CCAGTTTGATAAGCTTAGATCTGATCCAGACATGATAAGATAC |
| | | ATTGATGAGTTTGGACAAACCAAAACTAGAATGCAGTGAAAAA |
| | | AATGCCTTATTTGTGAAATTTGTGATGCTATTGCCTTATTTGTA |
| | | ACCATTATAAGCTGCAATAAACAAGTTTCTAGAACTGAATTCC |
| | | CGATAAGGATCTTCCTAGAGCATGGCTACGTAGATAAGTAGC |
| | | ATGGCGGGTTAATCATTAACTACAAGGAACCCCTAGTGATGG |
| | | AGTTGGCCACTCCCTCTCTGCGCGCTCGCTCGCTCACTGAG |
| | | GCCGGGCGACCAAAGGTCGCCCGACGCCCGGGCTTTGCCC |
| | | GGGCGGCCTCAGTGAGCGAGCGAGCGCGCAGCCTTAATTAA |
| | | CCTAATTCACTGGCCGTCGTTTTACAACGTCGTGACTGGGAA |
| | | AACCCTGGCGTTACCCAACTTAATCGCCTTGCAGCACATCCC |
| | | CCTTTCGCCAGCTGGCGTAATAGCGAAGAGGCCCGCACCGA |
| | | TCGCCCTTCCCAACAGTTGCGCAGCCTGAATGGCGAATGGG |
| | | ACGCGCCCTGTAGCGGCGCATTAAGCGCGGCGGGTGTGGT |
| | | GGTTACGCGCAGCGTGACCGCTACACTTGCCAGCGCCCTAG |
| | | CGCCCGCTCCTTTCGCTTTCTTCCCTTCCTTTCTCGCCACGTT |
| | | CGCCGGCTTTCCCCGTCAAGCTCTAAATCGGGGGCTCCCTTT |
| | | AGGGTTCCGATTTAGTGCTTTACGGCACCTCGACCCCAAAAA |
| | | ACTTGATTAGGGTGATGGTTCACGTAGTGGGCCATCGCCCTG |
| | | ATAGACGGTTTTTCGCCCTTTGACGTTGGAGTCCACGTTCTTT |
| | | AATAGTGGACTCTTGTTCCAAACTGGAACAACACTCAACCCTA |
| | | TCTCGGTCTATTCTTTTGATTTATAAGGGATTTTGCCGATTTC |
| | | GGCCTATTGGTTAAAAAATGAGCTGATTTAACAAAAATTTAAC |
| | | GCGAATTTTAACAAAATATTAACGCTTACAATTTAGGTGGCAC |
| | | TTTTCGGGGAAATGTGCGCGGAACCCCTATTTGTTTATTTTTC |
| | | TAAATACATTCAAATATGTATCCGCTCATGAGACAATAACCCT |
| | | GATAAATGCTTCAATAATATTGAAAAAGGAAGAGTATGAGCCA |
| | | TATTCAACGGGAAACGTCGAGGCCGCGATTAAATTCCAACAT |
| | | GGATGCTGATTTATATGGGTATAAATGGGCTCGCGATAATGT |
| | | CGGGCAATCAGGTGCGACAATCTATCGCTTGTATGGGAAGC |
| | | CCGATGCGCCAGAGTTGTTTCTGAAACATGGCAAAGGTAGCG |
| | | TTGCCAATGATGTTACAGATGAGATGGTCAGACTAAACTGGC |
| | | TGACGGAATTTATGCCTCTTCCGACCATCAAGCATTTTATCCG |
| | | TACTCCTGATGATGCATGGTTACTCACCACTGCGATCCCCGG |
| | | AAAAACAGCATTCCAGGTATTAGAAGAATATCCTGATTCAGGT |
| | | GAAAATATTGTTGATGCGCTGGCAGTGTTCCTGCGCCGGTTG |
| | | CATTCGATTCCTGTTTGTAATTGTCCTTTTAACAGCGATCGCG |
| | | TATTTCGTCTTGCTCAGGCGCAATCACGAATGAATAACGGTTT |
| | | GGTTGATGCGAGTGATTTTGATGACGAGCGTAATGGCTGGCC |
| | | TGTTGAACAAGTCTGGAAAGAAATGCATAAACTTTTGCCATTC |
| | | TCACCGGATTCAGTCGTCACTCATGGTGATTTCTCACTTGATA |
| | | ACCTTATTTTTGACGAGGGGAAATTAATAGGTTGTATTGATGT |
| | | TGGACGAGTCGGAATCGCAGACCGATACCAGGATCTTGCCA |
| | | TCCTATGGAACTGCCTCGGTGAGTTTTCTCCTTCATTACAGAA |
| | | ACGGCTTTTTCAAAAATATGGTATTGATAATCCTGATATGAAT |
| | | AAATTGCAGTTTCATTTGATGCTCGATGAGTTTTTCTAACTGT |
| | | CAGACCAAGTTTACTCATATATACTTTAGATTGATTTAAAACTT |
| | | CATTTTTAATTTAAAAGGATCTAGGTGAAGATCCTTTTTGATAA |
| | | TCTCATGACCAAAATCCCTTAACGTGAGTTTTCGTTCCACTGA |
| | | GCGTCAGACCCCGTAGAAAAGATCAAAGGATCTTCTTGAGAT |
| | | CCTTTTTTTCTGCGCGTAATCTGCTGCTTGCAAACAAAAAAAC |
| | | CACCGCTACCAGCGGTGGTTTGTTTGCCGGATCAAGAGCTAC |
| | | CAACTCTTTTTCCGAAGGTAACTGGCTTCAGCAGAGCGCAGA |
| | | TACCAAATACTGTTCTTCTAGTGTAGCCGTAGTTAGGCCACCA |
| | | CTTCAAGAACTCTGTAGCACCGCCTACATACCTCGCTCTGCT |
| | | AATCCTGTTACCAGTGGCTGCTGCCAGTGGCGATAAGTCGTG |
| | | TCTTACCGGGTTGGACTCAAGACGATAGTTACCGGATAAGGC |
| | | GCAGCGGTCGGGCTGAACGGGGGGTTCGTGCACACAGCCC |
| | | AGCTTGGAGCGAACGACCTACACCGAACTGAGATACCTACAG |
| | | CGTGAGCTATGAGAAAGCGCCACGCTTCCCGAAGGGAGAAA |
| | | GGCGGACAGGTATCCGGTAAGCGGCAGGGTCGGAACAGGA |
| | | GAGCGCACGAGGGAGCTTCCAGGGGGAAACGCCTGGTATCT |
| | | TTATAGTCCTGTCGGGTTTCGCCACCTCTGACTTGAGCGTCG |
| | | ATTTTTGTGATGCTCGTCAGGGGGGCGGAGCCTATGGAAAAA |
| | | CGCCAGCAACGCGGCCTTTTTACGGTTCCTGGCCTTTTGCTG |
| | | GCCTTTTGCTCACATGTTCTTTCCTGCGTTATCCCCTGATTCT |
| | | GTGGATAACCGTATTACCGCCTTTGAGTGAGCTGATACCGCT |
| | | CGCCGCAGCCGAACGACCGAGCGCAGCGAGTCAGTGAGCG |
| | | AGGAAGCGGAAGAGCGCCCAATACGCAAACCGCCTCTCCCC |
| | | GCGCGTTGGCCGATTCATTAATGCAGCTGGCACGACAGGTTT |
| | | CCCGACTGGAAAGCGGGCAGTGAGCGCAACGCAATTAATGT |
| | | GAGTTAGCTCACTCATTAGGCACCCCAGGCTTTACACTTTAT |
| | | GCTTCCGGCTCGTATGTTGTGTGGAATTGTGAGCGGATAACA |
| | | ATTTCACACAGGAAACAGCTATGACCATGATTACGCCAGATTT |
| | | AATTAAGGCCTTAATTAGG |
| 22 | Plasmid P1604 | CTGCGCGCTCGCTCGCTCACTGAGGCCGCCCGGGCAAAGC |
| | 5′ ITR at nucleotides | CCGGGCGTCGGGCGACCTTTGGTCGCCCGGCCTCAGTGAG |
| | 1-130 | CGAGCGAGCGCGCAGAGAGGGAGTGGCCAACTCCATCACTA |
| | GJB2 regulatory | GGGGTTCCTTGTAGTTAATGATTAACCCGCCATGCTACTTATC |
| | construct at nucleotides | TACGTAGCCATGCTCTAGGAAGATCGGAATTCTGGTACCTTG |
| | 212-2672 | CTAGCAAAAACTCTTACATAATTGTAACAGATTTGAGTTTCCT |
| | CpG depleted hGJB2 at | CTGGTTCAGGTTTTCTTGCCTCTTTGATAATCAAATGATCTGA |
| | nucleotides 2686-3363 | AGAAAGGCATAGAATTTCAAGGGAGAATCTGCATGACAGGAT |
| | SV40 polyA signal | TACAATAAGGCTATTCATGGAGACTCTTTTATTAGCTTACACA |
| | sequence at nucleotides | GGATCTGCATCATTCTTCGTCTCTGGCTTTGCTACAAGGCTC |
| | 3380-3514 | CATTTAAACTTAACCCAACTTGCAGGCTTAACTGATCCAGGAA |
| | 3′ ITR at nucleotides | TGACTCAATACAATGAGCCAGCTACTGTAGCTCTTTTTCCCCT |
| | 3597-3726 | TTTGATAAGGGGAGTTAACACAATGGGCTTTACAGTTCTTAAA |
| | AmpR promoter at | TGACCGTTCCTGCAGGTATTGGAAAACCTGCATCGTATGCCT |
| | nucleotides 4384-4488 | GCTGCTTTAGGAGACAACAAACTCAGCAGCCCCTCCCCCGAA |
| | KanR at nucleotides | GCTGAAGTGCCAAACTTAAGGCTCCAGGTAAACTGCCCTTCC |
| | 4489-5298 | GATGAGAGCTTTACATTTAGGCAGTAGCCACAGGCCTAAACA |
| | Sequence to be | AAGTCAAATGGATGAAACTTGTGATCTCTCCTTGGAAATATGG |
| | transferred into vector | CCAAGAGTGAAACAACTACTTAGTTATTATTTTAAACGCAGGC |
| | at nucleotides 1-3726 | TGCTAAATGGCTTGTGTTGGTATGTAATGGCATCCTAGATAAT |
| | | GGGGGCTGCCAAGAAAATATTCACACTGGCAAGTCAATTAAA |
| | | ATACCGTTCTCTTCCTAAGAGGCCTGGCTGGATTCCAGAGCT |
| | | TCCTTTAGGGTAACGCTCAAACCTGTCATAGTTTATTTTGTTT |
| | | CTGTTTTCAGGCAACAGATTTGGTTATTGCTTTTATTGTATGA |
| | | GTGTTGAAATGGTGTCCTAACAGCCATCTGTACTGCCATCCC |
| | | CTGGTCTCTCTGCCTTTCCAGAAAGGAGATGAAGTTGCTGGA |
| | | GCAGGCAGGTTCTCTGAAAGGCTCCCTGAGTGTGGAAGAGC |
| | | AGCTGTCACTCATCAGTGGTTGTCCCAATATCCAAGAAGCAG |
| | | TAGAGGGTGCCATGCACATTCAGGTGAGTGAAGGACACTGG |
| | | CGCCGCATCTGTGGGGCCTGAGACATTGTCTCCCAGCCTCT |
| | | CCGGAGGAACAGGGCAGGATACAGAGCAGTGGCAGGTGTG |
| | | GGCATTTTGCATCTTCTGTAATCTCCAAACAAAGTCTGCGCTA |
| | | GGCCCTTTCAGGCTATAAAGGACAGGGTACTCTGATTTCTGC |
| | | TCTTGATGAGGGTTGATTGTGATACCAGGCGAGGGAGTAAGA |
| | | GATACAGGACACCATCTCTGCGGCTGCATCTTTGTCGCAGAA |
| | | ACAGACATTGTTCAGGGGACACCTGAGCCTCTGATAGTCAGC |
| | | CAGTGGCTCTGGCAGCTCACAGCAGTTATAGGGTGGCTGTG |
| | | TCCTTTTCTAAAGTCCTGACTTTTTATGGCCTGTGGTCATGGT |
| | | GACTCCACTACGGGCTGTGTTTCTGTTGACGCGTACAGAGGA |
| | | CAACGACCACAGCCATCCCTGAACCCCGCCCACGGCACAGC |
| | | GCCGGAGCCGGGGTCTGGGGCGCCGCTTCCTGGGGGGTCC |
| | | CGACTCTCAGCCGCCCCCGCTTCACCCGGGCCGCCAAGGG |
| | | GCTGGGGGAGGCGGCGCTCGGGGTAACCGGGGGAGACTCA |
| | | GGGCGCTGGGGGCACTTGGGGAACTCATGGGGGCTCAAAG |
| | | GAACTAGGAGATCGGGACCTCGAAGGGGACTTGGGGGGTTC |
| | | GGGGCTTTCGGGGGCGGTCGGGGGTTCGCGGACCCGGGAA |
| | | GCTCTGAGGACCCAGAGGCCGGGCGCGCTCCGCCCGCGGC |
| | | GCCGCCCCCTCCGTAACTTTCCCAGTCTCCGAGGGAAGAGG |
| | | CGGGGTGTGGGGTGCGGTTAAAAGGCGCCACGGCGGGAGA |
| | | CAGGTGTTGCGGCCCCGCAGCGCCCGCGCGCTCCTCTCCC |
| | | CGACTCGGAGCCCCTCGGCGGCGCCCGGCCCAGGACCCGC |
| | | CTAGGAGCGCAGGAGCCCCAGCGCAGAGACCCCAACGCCG |
| | | AGACCCCCGCCCCGGCCCCGCCGCGCTTCCTCCCGACGCA |
| | | GGTGAGCCCGCCGGCCCCGGACTGCCCGGCCAGGAACCTG |
| | | GCGCGGGGAGGGACCGCGAGACCCAGAGCGGTTGCCCGGC |
| | | CGCGTGGGTCTCGGGGAACCGGGGGGCTGGACCAACACAC |
| | | GTCCTTGGGCCGGGGGGCGGGGGCCGCCTTCTGGAGCGGG |
| | | CGTTTCTGTTTATGATGTGTTTAAAGATTGGGTGAATTACTCA |
| | | GGTGAACAAGCTACTTTTTATCAGAGAACACCTAAAAACACGT |
| | | TCAAGAGGGTTTGGGAACTATACATTTAATCCTATGACAAACT |
| | | AAGTTGGTTCTGTCTTCACCTGTTTTGGTGAGGTTGTGTAAGA |
| | | GTTGGTGTTTGCTCAGGAAGAGATTTAAGCATGCTTGCTTAC |
| | | CCAGACTCAGAGAAGTCTCCCTGTTCTGTCCTAGCTAGTGAT |
| | | TCCTGTGTTGTGTGCATTCGTCTTTTCCAGAGCAAACCGCCC |
| | | AGAGTAGAAGCCTGCAGGCCACCATGGATTGGGGCACACTG |
| | | CAGACTATCCTGGGGGGTGTGAACAAACACTCCACCAGCATT |
| | | GGAAAGATCTGGCTCACAGTCCTCTTCATTTTTAGGATTATGA |
| | | TCCTGGTTGTGGCTGCAAAGGAGGTGTGGGGAGATGAGCAG |
| | | GCTGACTTTGTCTGCAACACCCTGCAGCCAGGCTGCAAGAAT |
| | | GTGTGCTATGATCACTACTTCCCCATCTCCCACATCAGGCTAT |
| | | GGGCCCTGCAGCTGATCTTTGTGTCCACTCCAGCACTCCTAG |
| | | TGGCCATGCATGTGGCCTACAGGAGACATGAGAAGAAGAGG |
| | | AAGTTCATCAAGGGGGAGATAAAGAGTGAATTTAAGGACATT |
| | | GAGGAGATCAAAACCCAGAAGGTCAGAATTGAAGGCTCCCT |
| | | GTGGTGGACCTACACAAGCAGCATCTTCTTCAGGGTCATCTT |
| | | TGAAGCAGCCTTCATGTATGTCTTCTATGTCATGTATGATGGC |
| | | TTCTCCATGCAGAGGCTGGTGAAGTGCAATGCCTGGCCTTGT |
| | | CCCAACACTGTGGACTGCTTTGTGTCCAGGCCCACTGAGAAG |
| | | ACTGTCTTCACAGTGTTCATGATTGCAGTGTCTGGAATTTGCA |
| | | TCCTGCTGAATGTCACTGAATTGTGTTATTTGCTAATTAGATA |
| | | TTGTTCTGGGAAGTCAAAAAAGCCAGTTTGATAAGCTTAGATC |
| | | TGATCCAGACATGATAAGATACATTGATGAGTTTGGACAAACC |
| | | AAAACTAGAATGCAGTGAAAAAAATGCCTTATTTGTGAAATTT |
| | | GTGATGCTATTGCCTTATTTGTAACCATTATAAGCTGCAATAA |
| | | ACAAGTTTCTAGAACTGAATTCCCGATAAGGATCTTCCTAGAG |
| | | CATGGCTACGTAGATAAGTAGCATGGCGGGTTAATCATTAAC |
| | | TACAAGGAACCCCTAGTGATGGAGTTGGCCACTCCCTCTCTG |
| | | CGCGCTCGCTCGCTCACTGAGGCCGGGCGACCAAAGGTCG |
| | | CCCGACGCCCGGGCTTTGCCCGGGCGGCCTCAGTGAGCGA |
| | | GCGAGCGCGCAGCCTTAATTAACCTAATTCACTGGCCGTCGT |
| | | TTTACAACGTCGTGACTGGGAAAACCCTGGCGTTACCCAACT |
| | | TAATCGCCTTGCAGCACATCCCCCTTTCGCCAGCTGGCGTAA |
| | | TAGCGAAGAGGCCCGCACCGATCGCCCTTCCCAACAGTTGC |
| | | GCAGCCTGAATGGCGAATGGGACGCGCCCTGTAGCGGCGC |
| | | ATTAAGCGCGGCGGGTGTGGTGGTTACGCGCAGCGTGACCG |
| | | CTACACTTGCCAGCGCCCTAGCGCCCGCTCCTTTCGCTTTCT |
| | | TCCCTTCCTTTCTCGCCACGTTCGCCGGCTTTCCCCGTCAAG |
| | | CTCTAAATCGGGGGCTCCCTTTAGGGTTCCGATTTAGTGCTT |
| | | TACGGCACCTCGACCCCAAAAAACTTGATTAGGGTGATGGTT |
| | | CACGTAGTGGGCCATCGCCCTGATAGACGGTTTTTCGCCCTT |
| | | TGACGTTGGAGTCCACGTTCTTTAATAGTGGACTCTTGTTCCA |
| | | AACTGGAACAACACTCAACCCTATCTCGGTCTATTCTTTTGAT |
| | | TTATAAGGGATTTTGCCGATTTCGGCCTATTGGTTAAAAAATG |
| | | AGCTGATTTAACAAAAATTTAACGCGAATTTTAACAAAATATTA |
| | | ACGCTTACAATTTAGGTGGCACTTTTCGGGGAAATGTGCGCG |
| | | GAACCCCTATTTGTTTATTTTTCTAAATACATTCAAATATGTAT |
| | | CCGCTCATGAGACAATAACCCTGATAAATGCTTCAATAATATT |
| | | GAAAAAGGAAGAGTATGAGCCATATTCAACGGGAAACGTCGA |
| | | GGCCGCGATTAAATTCCAACATGGATGCTGATTTATATGGGT |
| | | ATAAATGGGCTCGCGATAATGTCGGGCAATCAGGTGCGACAA |
| | | TCTATCGCTTGTATGGGAAGCCCGATGCGCCAGAGTTGTTTC |
| | | TGAAACATGGCAAAGGTAGCGTTGCCAATGATGTTACAGATG |
| | | AGATGGTCAGACTAAACTGGCTGACGGAATTTATGCCTCTTC |
| | | CGACCATCAAGCATTTTATCCGTACTCCTGATGATGCATGGTT |
| | | ACTCACCACTGCGATCCCCGGAAAAACAGCATTCCAGGTATT |
| | | AGAAGAATATCCTGATTCAGGTGAAAATATTGTTGATGCGCTG |
| | | GCAGTGTTCCTGCGCCGGTTGCATTCGATTCCTGTTTGTAAT |
| | | TGTCCTTTTAACAGCGATCGCGTATTTCGTCTTGCTCAGGCG |
| | | CAATCACGAATGAATAACGGTTTGGTTGATGCGAGTGATTTT |
| | | GATGACGAGCGTAATGGCTGGCCTGTTGAACAAGTCTGGAAA |
| | | GAAATGCATAAACTTTTGCCATTCTCACCGGATTCAGTCGTCA |
| | | CTCATGGTGATTTCTCACTTGATAACCTTATTTTTGACGAGGG |
| | | GAAATTAATAGGTTGTATTGATGTTGGACGAGTCGGAATCGC |
| | | AGACCGATACCAGGATCTTGCCATCCTATGGAACTGCCTCGG |
| | | TGAGTTTTCTCCTTCATTACAGAAACGGCTTTTTCAAAAATAT |
| | | GGTATTGATAATCCTGATATGAATAAATTGCAGTTTCATTTGAT |
| | | GCTCGATGAGTTTTTCTAACTGTCAGACCAAGTTTACTCATAT |
| | | ATACTTTAGATTGATTTAAAACTTCATTTTTAATTTAAAAGGAT |
| | | CTAGGTGAAGATCCTTTTTGATAATCTCATGACCAAAATCCCT |
| | | TAACGTGAGTTTTCGTTCCACTGAGCGTCAGACCCCGTAGAA |
| | | AAGATCAAAGGATCTTCTTGAGATCCTTTTTTTCTGCGCGTAA |
| | | TCTGCTGCTTGCAAACAAAAAAACCACCGCTACCAGCGGTGG |
| | | TTTGTTTGCCGGATCAAGAGCTACCAACTCTTTTTCCGAAGGT |
| | | AACTGGCTTCAGCAGAGCGCAGATACCAAATACTGTTCTTCT |
| | | AGTGTAGCCGTAGTTAGGCCACCACTTCAAGAACTCTGTAGC |
| | | ACCGCCTACATACCTCGCTCTGCTAATCCTGTTACCAGTGGC |
| | | TGCTGCCAGTGGCGATAAGTCGTGTCTTACCGGGTTGGACTC |
| | | AAGACGATAGTTACCGGATAAGGCGCAGCGGTCGGGCTGAA |
| | | CGGGGGGTTCGTGCACACAGCCCAGCTTGGAGCGAACGACC |
| | | TACACCGAACTGAGATACCTACAGCGTGAGCTATGAGAAAGC |
| | | GCCACGCTTCCCGAAGGGAGAAAGGCGGACAGGTATCCGGT |
| | | AAGCGGCAGGGTCGGAACAGGAGAGCGCACGAGGGAGCTT |
| | | CCAGGGGGAAACGCCTGGTATCTTTATAGTCCTGTCGGGTTT |
| | | CGCCACCTCTGACTTGAGCGTCGATTTTTGTGATGCTCGTCA |
| | | GGGGGGCGGAGCCTATGGAAAAACGCCAGCAACGCGGCCT |
| | | TTTTACGGTTCCTGGCCTTTTGCTGGCCTTTTGCTCACATGTT |
| | | CTTTCCTGCGTTATCCCCTGATTCTGTGGATAACCGTATTACC |
| | | GCCTTTGAGTGAGCTGATACCGCTCGCCGCAGCCGAACGAC |
| | | CGAGCGCAGCGAGTCAGTGAGCGAGGAAGCGGAAGAGCGC |
| | | CCAATACGCAAACCGCCTCTCCCCGCGCGTTGGCCGATTCAT |
| | | TAATGCAGCTGGCACGACAGGTTTCCCGACTGGAAAGCGGG |
| | | CAGTGAGCGCAACGCAATTAATGTGAGTTAGCTCACTCATTA |
| | | GGCACCCCAGGCTTTACACTTTATGCTTCCGGCTCGTATGTT |
| | | GTGTGGAATTGTGAGCGGATAACAATTTCACACAGGAAACAG |
| | | CTATGACCATGATTACGCCAGATTTAATTAAGGCCTTAATTAG |
| | | G |
| 23 | Plasmid P1605 | CTGCGCGCTCGCTCGCTCACTGAGGCCGCCCGGGCAAAGC |
| | 5′ ITR at nucleotides | CCGGGCGTCGGGCGACCTTTGGTCGCCCGGCCTCAGTGAG |
| | 1-130 | CGAGCGAGCGCGCAGAGAGGGAGTGGCCAACTCCATCACTA |
| | GJB2 regulatory | GGGGTTCCTTGTAGTTAATGATTAACCCGCCATGCTACTTATC |
| | construct at nucleotides | TACGTAGCCATGCTCTAGGAAGATCGGAATTCTGGTACCTTG |
| | 212-2672 | CTAGCAAAAACTCTTACATAATTGTAACAGATTTGAGTTTCCT |
| | Codon-optimized and | CTGGTTCAGGTTTTCTTGCCTCTTTGATAATCAAATGATCTGA |
| | CpG depleted hGJB2 at | AGAAAGGCATAGAATTTCAAGGGAGAATCTGCATGACAGGAT |
| | nucleotides 2686-3363 | TACAATAAGGCTATTCATGGAGACTCTTTTATTAGCTTACACA |
| | SV40 polyA signal | GGATCTGCATCATTCTTCGTCTCTGGCTTTGCTACAAGGCTC |
| | sequence at nucleotides | CATTTAAACTTAACCCAACTTGCAGGCTTAACTGATCCAGGAA |
| | 3380-3514 | TGACTCAATACAATGAGCCAGCTACTGTAGCTCTTTTTCCCCT |
| | 3′ ITR at nucleotides | TTTGATAAGGGGAGTTAACACAATGGGCTTTACAGTTCTTAAA |
| | 3597-3726 | TGACCGTTCCTGCAGGTATTGGAAAACCTGCATCGTATGCCT |
| | AmpR promoter at | GCTGCTTTAGGAGACAACAAACTCAGCAGCCCCTCCCCCGAA |
| | nucleotides 4384-4488 | GCTGAAGTGCCAAACTTAAGGCTCCAGGTAAACTGCCCTTCC |
| | KanR at nucleotides | GATGAGAGCTTTACATTTAGGCAGTAGCCACAGGCCTAAACA |
| | 4489-5298 | AAGTCAAATGGATGAAACTTGTGATCTCTCCTTGGAAATATGG |
| | Sequence to be | CCAAGAGTGAAACAACTACTTAGTTATTATTTTAAACGCAGGC |
| | transferred into vector | TGCTAAATGGCTTGTGTTGGTATGTAATGGCATCCTAGATAAT |
| | at nucleotides 1-3726 | GGGGGCTGCCAAGAAAATATTCACACTGGCAAGTCAATTAAA |
| | | ATACCGTTCTCTTCCTAAGAGGCCTGGCTGGATTCCAGAGCT |
| | | TCCTTTAGGGTAACGCTCAAACCTGTCATAGTTTATTTTGTTT |
| | | CTGTTTTCAGGCAACAGATTTGGTTATTGCTTTTATTGTATGA |
| | | GTGTTGAAATGGTGTCCTAACAGCCATCTGTACTGCCATCCC |
| | | CTGGTCTCTCTGCCTTTCCAGAAAGGAGATGAAGTTGCTGGA |
| | | GCAGGCAGGTTCTCTGAAAGGCTCCCTGAGTGTGGAAGAGC |
| | | AGCTGTCACTCATCAGTGGTTGTCCCAATATCCAAGAAGCAG |
| | | TAGAGGGTGCCATGCACATTCAGGTGAGTGAAGGACACTGG |
| | | CGCCGCATCTGTGGGGCCTGAGACATTGTCTCCCAGCCTCT |
| | | CCGGAGGAACAGGGCAGGATACAGAGCAGTGGCAGGTGTG |
| | | GGCATTTTGCATCTTCTGTAATCTCCAAACAAAGTCTGCGCTA |
| | | GGCCCTTTCAGGCTATAAAGGACAGGGTACTCTGATTTCTGC |
| | | TCTTGATGAGGGTTGATTGTGATACCAGGCGAGGGAGTAAGA |
| | | GATACAGGACACCATCTCTGCGGCTGCATCTTTGTCGCAGAA |
| | | ACAGACATTGTTCAGGGGACACCTGAGCCTCTGATAGTCAGC |
| | | CAGTGGCTCTGGCAGCTCACAGCAGTTATAGGGTGGCTGTG |
| | | TCCTTTTCTAAAGTCCTGACTTTTTATGGCCTGTGGTCATGGT |
| | | GACTCCACTACGGGCTGTGTTTCTGTTGACGCGTACAGAGGA |
| | | CAACGACCACAGCCATCCCTGAACCCCGCCCACGGCACAGC |
| | | GCCGGAGCCGGGGTCTGGGGCGCCGCTTCCTGGGGGGTCC |
| | | CGACTCTCAGCCGCCCCCGCTTCACCCGGGCCGCCAAGGG |
| | | GCTGGGGGAGGCGGCGCTCGGGGTAACCGGGGGAGACTCA |
| | | GGGCGCTGGGGGCACTTGGGGAACTCATGGGGGCTCAAAG |
| | | GAACTAGGAGATCGGGACCTCGAAGGGGACTTGGGGGGTTC |
| | | GGGGCTTTCGGGGGCGGTCGGGGGTTCGCGGACCCGGGAA |
| | | GCTCTGAGGACCCAGAGGCCGGGCGCGCTCCGCCCGCGGC |
| | | GCCGCCCCCTCCGTAACTTTCCCAGTCTCCGAGGGAAGAGG |
| | | CGGGGTGTGGGGTGCGGTTAAAAGGCGCCACGGCGGGAGA |
| | | CAGGTGTTGCGGCCCCGCAGCGCCCGCGCGCTCCTCTCCC |
| | | CGACTCGGAGCCCCTCGGCGGCGCCCGGCCCAGGACCCGC |
| | | CTAGGAGCGCAGGAGCCCCAGCGCAGAGACCCCAACGCCG |
| | | AGACCCCCGCCCCGGCCCCGCCGCGCTTCCTCCCGACGCA |
| | | GGTGAGCCCGCCGGCCCCGGACTGCCCGGCCAGGAACCTG |
| | | GCGCGGGGAGGGACCGCGAGACCCAGAGCGGTTGCCCGGC |
| | | CGCGTGGGTCTCGGGGAACCGGGGGGCTGGACCAACACAC |
| | | GTCCTTGGGCCGGGGGGCGGGGGCCGCCTTCTGGAGCGGG |
| | | CGTTTCTGTTTATGATGTGTTTAAAGATTGGGTGAATTACTCA |
| | | GGTGAACAAGCTACTTTTTATCAGAGAACACCTAAAAACACGT |
| | | TCAAGAGGGTTTGGGAACTATACATTTAATCCTATGACAAACT |
| | | AAGTTGGTTCTGTCTTCACCTGTTTTGGTGAGGTTGTGTAAGA |
| | | GTTGGTGTTTGCTCAGGAAGAGATTTAAGCATGCTTGCTTAC |
| | | CCAGACTCAGAGAAGTCTCCCTGTTCTGTCCTAGCTAGTGAT |
| | | TCCTGTGTTGTGTGCATTCGTCTTTTCCAGAGCAAACCGCCC |
| | | AGAGTAGAAGCCTGCAGGCCACCATGGACTGGGGAACACTG |
| | | CAAACAATCCTGGGAGGTGTGAACAAGCACAGCACAAGCATT |
| | | GGCAAGATCTGGCTGACAGTGCTGTTCATCTTTAGAATCATG |
| | | ATCCTGGTTGTGGCTGCTAAGGAAGTGTGGGGAGATGAGCA |
| | | GGCTGACTTTGTTTGTAATACCCTGCAACCTGGCTGCAAGAA |
| | | TGTGTGTTATGACCACTACTTCCCCATCAGCCACATCAGACT |
| | | GTGGGCCCTGCAACTGATCTTTGTGTCCACCCCTGCCCTGCT |
| | | GGTGGCCATGCATGTGGCCTACAGAAGGCATGAGAAGAAGA |
| | | GGAAATTCATCAAGGGAGAGATCAAGAGTGAGTTCAAGGACA |
| | | TTGAGGAAATCAAAACCCAGAAGGTGAGGATTGAAGGCTCTC |
| | | TGTGGTGGACCTACACCAGCAGCATCTTCTTCAGAGTGATCT |
| | | TTGAGGCAGCCTTTATGTATGTGTTCTATGTGATGTATGATGG |
| | | CTTCTCCATGCAGAGACTGGTGAAGTGCAATGCCTGGCCATG |
| | | TCCTAACACAGTGGACTGCTTTGTGTCTAGACCTACAGAAAA |
| | | GACAGTGTTTACAGTCTTTATGATTGCAGTGTCTGGCATCTGC |
| | | ATTCTGCTGAATGTGACTGAGCTGTGCTACCTGCTCATTAGG |
| | | TACTGTAGTGGAAAAAGCAAGAAACCTGTGTGATAAGCTTAG |
| | | ATCTGATCCAGACATGATAAGATACATTGATGAGTTTGGACAA |
| | | ACCAAAACTAGAATGCAGTGAAAAAAATGCCTTATTTGTGAAA |
| | | TTTGTGATGCTATTGCCTTATTTGTAACCATTATAAGCTGCAAT |
| | | AAACAAGTTTCTAGAACTGAATTCCCGATAAGGATCTTCCTAG |
| | | AGCATGGCTACGTAGATAAGTAGCATGGCGGGTTAATCATTA |
| | | ACTACAAGGAACCCCTAGTGATGGAGTTGGCCACTCCCTCTC |
| | | TGCGCGCTCGCTCGCTCACTGAGGCCGGGCGACCAAAGGTC |
| | | GCCCGACGCCCGGGCTTTGCCCGGGCGGCCTCAGTGAGCG |
| | | AGCGAGCGCGCAGCCTTAATTAACCTAATTCACTGGCCGTCG |
| | | TTTTACAACGTCGTGACTGGGAAAACCCTGGCGTTACCCAAC |
| | | TTAATCGCCTTGCAGCACATCCCCCTTTCGCCAGCTGGCGTA |
| | | ATAGCGAAGAGGCCCGCACCGATCGCCCTTCCCAACAGTTG |
| | | CGCAGCCTGAATGGCGAATGGGACGCGCCCTGTAGCGGCG |
| | | CATTAAGCGCGGCGGGTGTGGTGGTTACGCGCAGCGTGACC |
| | | GCTACACTTGCCAGCGCCCTAGCGCCCGCTCCTTTCGCTTTC |
| | | TTCCCTTCCTTTCTCGCCACGTTCGCCGGCTTTCCCCGTCAA |
| | | GCTCTAAATCGGGGGCTCCCTTTAGGGTTCCGATTTAGTGCT |
| | | TTACGGCACCTCGACCCCAAAAAACTTGATTAGGGTGATGGT |
| | | TCACGTAGTGGGCCATCGCCCTGATAGACGGTTTTTCGCCCT |
| | | TTGACGTTGGAGTCCACGTTCTTTAATAGTGGACTCTTGTTCC |
| | | AAACTGGAACAACACTCAACCCTATCTCGGTCTATTCTTTTGA |
| | | TTTATAAGGGATTTTGCCGATTTCGGCCTATTGGTTAAAAAAT |
| | | GAGCTGATTTAACAAAAATTTAACGCGAATTTTAACAAAATATT |
| | | AACGCTTACAATTTAGGTGGCACTTTTCGGGGAAATGTGCGC |
| | | GGAACCCCTATTTGTTTATTTTTCTAAATACATTCAAATATGTA |
| | | TCCGCTCATGAGACAATAACCCTGATAAATGCTTCAATAATAT |
| | | TGAAAAAGGAAGAGTATGAGCCATATTCAACGGGAAACGTCG |
| | | AGGCCGCGATTAAATTCCAACATGGATGCTGATTTATATGGG |
| | | TATAAATGGGCTCGCGATAATGTCGGGCAATCAGGTGCGACA |
| | | ATCTATCGCTTGTATGGGAAGCCCGATGCGCCAGAGTTGTTT |
| | | CTGAAACATGGCAAAGGTAGCGTTGCCAATGATGTTACAGAT |
| | | GAGATGGTCAGACTAAACTGGCTGACGGAATTTATGCCTCTT |
| | | CCGACCATCAAGCATTTTATCCGTACTCCTGATGATGCATGG |
| | | TTACTCACCACTGCGATCCCCGGAAAAACAGCATTCCAGGTA |
| | | TTAGAAGAATATCCTGATTCAGGTGAAAATATTGTTGATGCGC |
| | | TGGCAGTGTTCCTGCGCCGGTTGCATTCGATTCCTGTTTGTA |
| | | ATTGTCCTTTTAACAGCGATCGCGTATTTCGTCTTGCTCAGGC |
| | | GCAATCACGAATGAATAACGGTTTGGTTGATGCGAGTGATTT |
| | | TGATGACGAGCGTAATGGCTGGCCTGTTGAACAAGTCTGGAA |
| | | AGAAATGCATAAACTTTTGCCATTCTCACCGGATTCAGTCGTC |
| | | ACTCATGGTGATTTCTCACTTGATAACCTTATTTTTGACGAGG |
| | | GGAAATTAATAGGTTGTATTGATGTTGGACGAGTCGGAATCG |
| | | CAGACCGATACCAGGATCTTGCCATCCTATGGAACTGCCTCG |
| | | GTGAGTTTTCTCCTTCATTACAGAAACGGCTTTTTCAAAAATA |
| | | TGGTATTGATAATCCTGATATGAATAAATTGCAGTTTCATTTGA |
| | | TGCTCGATGAGTTTTTCTAACTGTCAGACCAAGTTTACTCATA |
| | | TATACTTTAGATTGATTTAAAACTTCATTTTTAATTTAAAAGGAT |
| | | CTAGGTGAAGATCCTTTTTGATAATCTCATGACCAAAATCCCT |
| | | TAACGTGAGTTTTCGTTCCACTGAGCGTCAGACCCCGTAGAA |
| | | AAGATCAAAGGATCTTCTTGAGATCCTTTTTTTCTGCGCGTAA |
| | | TCTGCTGCTTGCAAACAAAAAAACCACCGCTACCAGCGGTGG |
| | | TTTGTTTGCCGGATCAAGAGCTACCAACTCTTTTTCCGAAGGT |
| | | AACTGGCTTCAGCAGAGCGCAGATACCAAATACTGTTCTTCT |
| | | AGTGTAGCCGTAGTTAGGCCACCACTTCAAGAACTCTGTAGC |
| | | ACCGCCTACATACCTCGCTCTGCTAATCCTGTTACCAGTGGC |
| | | TGCTGCCAGTGGCGATAAGTCGTGTCTTACCGGGTTGGACTC |
| | | AAGACGATAGTTACCGGATAAGGCGCAGCGGTCGGGCTGAA |
| | | CGGGGGGTTCGTGCACACAGCCCAGCTTGGAGCGAACGACC |
| | | TACACCGAACTGAGATACCTACAGCGTGAGCTATGAGAAAGC |
| | | GCCACGCTTCCCGAAGGGAGAAAGGCGGACAGGTATCCGGT |
| | | AAGCGGCAGGGTCGGAACAGGAGAGCGCACGAGGGAGCTT |
| | | CCAGGGGGAAACGCCTGGTATCTTTATAGTCCTGTCGGGTTT |
| | | CGCCACCTCTGACTTGAGCGTCGATTTTTGTGATGCTCGTCA |
| | | GGGGGGCGGAGCCTATGGAAAAACGCCAGCAACGCGGCCT |
| | | TTTTACGGTTCCTGGCCTTTTGCTGGCCTTTTGCTCACATGTT |
| | | CTTTCCTGCGTTATCCCCTGATTCTGTGGATAACCGTATTACC |
| | | GCCTTTGAGTGAGCTGATACCGCTCGCCGCAGCCGAACGAC |
| | | CGAGCGCAGCGAGTCAGTGAGCGAGGAAGCGGAAGAGCGC |
| | | CCAATACGCAAACCGCCTCTCCCCGCGCGTTGGCCGATTCAT |
| | | TAATGCAGCTGGCACGACAGGTTTCCCGACTGGAAAGCGGG |
| | | CAGTGAGCGCAACGCAATTAATGTGAGTTAGCTCACTCATTA |
| | | GGCACCCCAGGCTTTACACTTTATGCTTCCGGCTCGTATGTT |
| | | GTGTGGAATTGTGAGCGGATAACAATTTCACACAGGAAACAG |
| | | CTATGACCATGATTACGCCAGATTTAATTAAGGCCTTAATTAG |
| | | G |
| 24 | Plasmid P1607 | CTGCGCGCTCGCTCGCTCACTGAGGCCGCCCGGGCAAAGC |
| | 5′ ITR at nucleotides | CCGGGCGTCGGGCGACCTTTGGTCGCCCGGCCTCAGTGAG |
| | 1-130 | CGAGCGAGCGCGCAGAGAGGGAGTGGCCAACTCCATCACTA |
| | GJB2 regulatory | GGGGTTCCTTGTAGTTAATGATTAACCCGCCATGCTACTTATC |
| | construct at nucleotides | TACGTAGCCATGCTCTAGGAAGATCGGAATTCTGGTACCTTG |
| | 212-1596 | CTAGCAAAAACTCTTACATAATTGTAACAGATTTGAGTTTCCT |
| | CpG depleted hGJB2 at | CTGGTTCAGGTTTTCTTGCCTCTTTGATAATCAAATGATCTGA |
| | nucleotides 1610-2287 | AGAAAGGCATAGAATTTCAAGGGAGAATCTGCATGACAGGAT |
| | bGH polyA signal | TACAATAAGGCTATTCATGGAGACTCTTTTATTAGCTTACACA |
| | sequence at nucleotides | GGATCTGCATCATTCTTCGTCTCTGGCTTTGCTACAAGGCTC |
| | 2310-2517 | CATTTAAACTTAACCCAACTTGCAGGCTTAACTGATCCAGGAA |
| | 3′ ITR at nucleotides | TGACTCAATACAATGAGCCAGCTACTGTAGCTCTTTTTCCCCT |
| | 2600-2729 | TTTGATAAGGGGAGTTAACACAATGGGCTTTACAGTTCTTAAA |
| | AmpR promoter at | TGAACGCGTACAGAGGACAACGACCACAGCCATCCCTGAAC |
| | nucleotides 3387-3491 | CCCGCCCACGGCACAGCGCCGGAGCCGGGGTCTGGGGCGC |
| | KanR at nucleotides | CGCTTCCTGGGGGGTCCCGACTCTCAGCCGCCCCCGCTTCA |
| | 3492-4301 | CCCGGGCCGCCAAGGGGCTGGGGGAGGCGGCGCTCGGGG |
| | Sequence to be | TAACCGGGGGAGACTCAGGGCGCTGGGGGCACTTGGGGAA |
| | transferred into vector | CTCATGGGGGCTCAAAGGAACTAGGAGATCGGGACCTCGAA |
| | at nucleotides 1-2729 | GGGGACTTGGGGGGTTCGGGGCTTTCGGGGGCGGTCGGGG |
| | | GTTCGCGGACCCGGGAAGCTCTGAGGACCCAGAGGCCGGG |
| | | CGCGCTCCGCCCGCGGCGCCGCCCCCTCCGTAACTTTCCCA |
| | | GTCTCCGAGGGAAGAGGCGGGGTGTGGGGTGCGGTTAAAA |
| | | GGCGCCACGGCGGGAGACAGGTGTTGCGGCCCCGCAGCGC |
| | | CCGCGCGCTCCTCTCCCCGACTCGGAGCCCCTCGGCGGCG |
| | | CCCGGCCCAGGACCCGCCTAGGAGCGCAGGAGCCCCAGCG |
| | | CAGAGACCCCAACGCCGAGACCCCCGCCCCGGCCCCGCCG |
| | | CGCTTCCTCCCGACGCAGGTGAGCCCGCCGGCCCCGGACT |
| | | GCCCGGCCAGGAACCTGGCGCGGGGAGGGACCGCGAGACC |
| | | CAGAGCGGTTGCCCGGCCGCGTGGGTCTCGGGGAACCGGG |
| | | GGGCTGGACCAACACACGTCCTTGGGCCGGGGGGCGGGGG |
| | | CCGCCTTCTGGAGCGGGCGTTTCTGTTTATGATGTGTTTAAA |
| | | GATTGGGTGAATTACTCAGGTGAACAAGCTACTTTTTATCAGA |
| | | GAACACCTAAAAACACGTTCAAGAGGGTTTGGGAACTATACA |
| | | TTTAATCCTATGACAAACTAAGTTGGTTCTGTCTTCACCTGTTT |
| | | TGGTGAGGTTGTGTAAGAGTTGGTGTTTGCTCAGGAAGAGAT |
| | | TTAAGCATGCTTGCTTACCCAGACTCAGAGAAGTCTCCCTGT |
| | | TCTGTCCTAGCTAGTGATTCCTGTGTTGTGTGCATTCGTCTTT |
| | | TCCAGAGCAAACCGCCCAGAGTAGAAGCCTGCAGGCCACCA |
| | | TGGATTGGGGCACACTGCAGACTATCCTGGGGGGTGTGAAC |
| | | AAACACTCCACCAGCATTGGAAAGATCTGGCTCACAGTCCTC |
| | | TTCATTTTTAGGATTATGATCCTGGTTGTGGCTGCAAAGGAG |
| | | GTGTGGGGAGATGAGCAGGCTGACTTTGTCTGCAACACCCT |
| | | GCAGCCAGGCTGCAAGAATGTGTGCTATGATCACTACTTCCC |
| | | CATCTCCCACATCAGGCTATGGGCCCTGCAGCTGATCTTTGT |
| | | GTCCACTCCAGCACTCCTAGTGGCCATGCATGTGGCCTACAG |
| | | GAGACATGAGAAGAAGAGGAAGTTCATCAAGGGGGAGATAA |
| | | AGAGTGAATTTAAGGACATTGAGGAGATCAAAACCCAGAAGG |
| | | TCAGAATTGAAGGCTCCCTGTGGTGGACCTACACAAGCAGCA |
| | | TCTTCTTCAGGGTCATCTTTGAAGCAGCCTTCATGTATGTCTT |
| | | CTATGTCATGTATGATGGCTTCTCCATGCAGAGGCTGGTGAA |
| | | GTGCAATGCCTGGCCTTGTCCCAACACTGTGGACTGCTTTGT |
| | | GTCCAGGCCCACTGAGAAGACTGTCTTCACAGTGTTCATGAT |
| | | TGCAGTGTCTGGAATTTGCATCCTGCTGAATGTCACTGAATT |
| | | GTGTTATTTGCTAATTAGATATTGTTCTGGGAAGTCAAAAAAG |
| | | CCAGTTTGATAAGCTTAGATCTGCCTGACTGTGCCTTCTAGTT |
| | | GCCAGCCATCTGTTGTTTGCCCCTCCCCCGTGCCTTCCTTGA |
| | | CCCTGGAAGGTGCCACTCCCACTGTCCTTTCCTAATAAAATG |
| | | AGGAAATTGCATCGCATTGTCTGAGTAGGTGTCATTCTATTCT |
| | | GGGGGGTGGGGTGGGGCAGGACAGCAAGGGGGAGGATTG |
| | | GGAAGACAATAGCAGGCATGCTGGGGATCTAGAACTGAATTC |
| | | CCGATAAGGATCTTCCTAGAGCATGGCTACGTAGATAAGTAG |
| | | CATGGCGGGTTAATCATTAACTACAAGGAACCCCTAGTGATG |
| | | GAGTTGGCCACTCCCTCTCTGCGCGCTCGCTCGCTCACTGA |
| | | GGCCGGGCGACCAAAGGTCGCCCGACGCCCGGGCTTTGCC |
| | | CGGGCGGCCTCAGTGAGCGAGCGAGCGCGCAGCCTTAATTA |
| | | ACCTAATTCACTGGCCGTCGTTTTACAACGTCGTGACTGGGA |
| | | AAACCCTGGCGTTACCCAACTTAATCGCCTTGCAGCACATCC |
| | | CCCTTTCGCCAGCTGGCGTAATAGCGAAGAGGCCCGCACCG |
| | | ATCGCCCTTCCCAACAGTTGCGCAGCCTGAATGGCGAATGG |
| | | GACGCGCCCTGTAGCGGCGCATTAAGCGCGGCGGGTGTGG |
| | | TGGTTACGCGCAGCGTGACCGCTACACTTGCCAGCGCCCTA |
| | | GCGCCCGCTCCTTTCGCTTTCTTCCCTTCCTTTCTCGCCACG |
| | | TTCGCCGGCTTTCCCCGTCAAGCTCTAAATCGGGGGCTCCCT |
| | | TTAGGGTTCCGATTTAGTGCTTTACGGCACCTCGACCCCAAA |
| | | AAACTTGATTAGGGTGATGGTTCACGTAGTGGGCCATCGCCC |
| | | TGATAGACGGTTTTTCGCCCTTTGACGTTGGAGTCCACGTTC |
| | | TTTAATAGTGGACTCTTGTTCCAAACTGGAACAACACTCAACC |
| | | CTATCTCGGTCTATTCTTTTGATTTATAAGGGATTTTGCCGATT |
| | | TCGGCCTATTGGTTAAAAAATGAGCTGATTTAACAAAAATTTA |
| | | ACGCGAATTTTAACAAAATATTAACGCTTACAATTTAGGTGGC |
| | | ACTTTTCGGGGAAATGTGCGCGGAACCCCTATTTGTTTATTTT |
| | | TCTAAATACATTCAAATATGTATCCGCTCATGAGACAATAACC |
| | | CTGATAAATGCTTCAATAATATTGAAAAAGGAAGAGTATGAGC |
| | | CATATTCAACGGGAAACGTCGAGGCCGCGATTAAATTCCAAC |
| | | ATGGATGCTGATTTATATGGGTATAAATGGGCTCGCGATAAT |
| | | GTCGGGCAATCAGGTGCGACAATCTATCGCTTGTATGGGAAG |
| | | CCCGATGCGCCAGAGTTGTTTCTGAAACATGGCAAAGGTAGC |
| | | GTTGCCAATGATGTTACAGATGAGATGGTCAGACTAAACTGG |
| | | CTGACGGAATTTATGCCTCTTCCGACCATCAAGCATTTTATCC |
| | | GTACTCCTGATGATGCATGGTTACTCACCACTGCGATCCCCG |
| | | GAAAAACAGCATTCCAGGTATTAGAAGAATATCCTGATTCAG |
| | | GTGAAAATATTGTTGATGCGCTGGCAGTGTTCCTGCGCCGGT |
| | | TGCATTCGATTCCTGTTTGTAATTGTCCTTTTAACAGCGATCG |
| | | CGTATTTCGTCTTGCTCAGGCGCAATCACGAATGAATAACGG |
| | | TTTGGTTGATGCGAGTGATTTTGATGACGAGCGTAATGGCTG |
| | | GCCTGTTGAACAAGTCTGGAAAGAAATGCATAAACTTTTGCC |
| | | ATTCTCACCGGATTCAGTCGTCACTCATGGTGATTTCTCACTT |
| | | GATAACCTTATTTTTGACGAGGGGAAATTAATAGGTTGTATTG |
| | | ATGTTGGACGAGTCGGAATCGCAGACCGATACCAGGATCTTG |
| | | CCATCCTATGGAACTGCCTCGGTGAGTTTTCTCCTTCATTACA |
| | | GAAACGGCTTTTTCAAAAATATGGTATTGATAATCCTGATATG |
| | | AATAAATTGCAGTTTCATTTGATGCTCGATGAGTTTTTCTAACT |
| | | GTCAGACCAAGTTTACTCATATATACTTTAGATTGATTTAAAAC |
| | | TTCATTTTTAATTTAAAAGGATCTAGGTGAAGATCCTTTTTGAT |
| | | AATCTCATGACCAAAATCCCTTAACGTGAGTTTTCGTTCCACT |
| | | GAGCGTCAGACCCCGTAGAAAAGATCAAAGGATCTTCTTGAG |
| | | ATCCTTTTTTTCTGCGCGTAATCTGCTGCTTGCAAACAAAAAA |
| | | ACCACCGCTACCAGCGGTGGTTTGTTTGCCGGATCAAGAGCT |
| | | ACCAACTCTTTTTCCGAAGGTAACTGGCTTCAGCAGAGCGCA |
| | | GATACCAAATACTGTTCTTCTAGTGTAGCCGTAGTTAGGCCA |
| | | CCACTTCAAGAACTCTGTAGCACCGCCTACATACCTCGCTCT |
| | | GCTAATCCTGTTACCAGTGGCTGCTGCCAGTGGCGATAAGTC |
| | | GTGTCTTACCGGGTTGGACTCAAGACGATAGTTACCGGATAA |
| | | GGCGCAGCGGTCGGGCTGAACGGGGGGTTCGTGCACACAG |
| | | CCCAGCTTGGAGCGAACGACCTACACCGAACTGAGATACCTA |
| | | CAGCGTGAGCTATGAGAAAGCGCCACGCTTCCCGAAGGGAG |
| | | AAAGGCGGACAGGTATCCGGTAAGCGGCAGGGTCGGAACAG |
| | | GAGAGCGCACGAGGGAGCTTCCAGGGGGAAACGCCTGGTAT |
| | | CTTTATAGTCCTGTCGGGTTTCGCCACCTCTGACTTGAGCGT |
| | | CGATTTTTGTGATGCTCGTCAGGGGGGCGGAGCCTATGGAA |
| | | AAACGCCAGCAACGCGGCCTTTTTACGGTTCCTGGCCTTTTG |
| | | CTGGCCTTTTGCTCACATGTTCTTTCCTGCGTTATCCCCTGAT |
| | | TCTGTGGATAACCGTATTACCGCCTTTGAGTGAGCTGATACC |
| | | GCTCGCCGCAGCCGAACGACCGAGCGCAGCGAGTCAGTGA |
| | | GCGAGGAAGCGGAAGAGCGCCCAATACGCAAACCGCCTCTC |
| | | CCCGCGCGTTGGCCGATTCATTAATGCAGCTGGCACGACAG |
| | | GTTTCCCGACTGGAAAGCGGGCAGTGAGCGCAACGCAATTA |
| | | ATGTGAGTTAGCTCACTCATTAGGCACCCCAGGCTTTACACT |
| | | TTATGCTTCCGGCTCGTATGTTGTGTGGAATTGTGAGCGGAT |
| | | AACAATTTCACACAGGAAACAGCTATGACCATGATTACGCCA |
| | | GATTTAATTAAGGCCTTAATTAGG |
| 25 | Plasmid P1608 | CTGCGCGCTCGCTCGCTCACTGAGGCCGCCCGGGCAAAGC |
| | 5′ ITR at nucleotides | CCGGGCGTCGGGCGACCTTTGGTCGCCCGGCCTCAGTGAG |
| | 1-130 | CGAGCGAGCGCGCAGAGAGGGAGTGGCCAACTCCATCACTA |
| | GJB2 regulatory | GGGGTTCCTTGTAGTTAATGATTAACCCGCCATGCTACTTATC |
| | construct at nucleotides | TACGTAGCCATGCTCTAGGAAGATCGGAATTCTGGTACCTTG |
| | 212-1596 | CTAGCAAAAACTCTTACATAATTGTAACAGATTTGAGTTTCCT |
| | Codon-optimized and | CTGGTTCAGGTTTTCTTGCCTCTTTGATAATCAAATGATCTGA |
| | CpG depleted hGJB2 at | AGAAAGGCATAGAATTTCAAGGGAGAATCTGCATGACAGGAT |
| | nucleotides 1610-2287 | TACAATAAGGCTATTCATGGAGACTCTTTTATTAGCTTACACA |
| | bGH polyA signal | GGATCTGCATCATTCTTCGTCTCTGGCTTTGCTACAAGGCTC |
| | sequence at nucleotides | CATTTAAACTTAACCCAACTTGCAGGCTTAACTGATCCAGGAA |
| | 2310-2517 | TGACTCAATACAATGAGCCAGCTACTGTAGCTCTTTTTCCCCT |
| | 3′ ITR at nucleotides | TTTGATAAGGGGAGTTAACACAATGGGCTTTACAGTTCTTAAA |
| | 2600-2729 | TGAACGCGTACAGAGGACAACGACCACAGCCATCCCTGAAC |
| | AmpR promoter at | CCCGCCCACGGCACAGCGCCGGAGCCGGGGTCTGGGGCGC |
| | nucleotides 3387-3491 | CGCTTCCTGGGGGGTCCCGACTCTCAGCCGCCCCCGCTTCA |
| | KanR at nucleotides | CCCGGGCCGCCAAGGGGCTGGGGGAGGCGGCGCTCGGGG |
| | 3492-4301 | TAACCGGGGGAGACTCAGGGCGCTGGGGGCACTTGGGGAA |
| | Sequence to be | CTCATGGGGGCTCAAAGGAACTAGGAGATCGGGACCTCGAA |
| | transferred into vector | GGGGACTTGGGGGGTTCGGGGCTTTCGGGGGCGGTCGGGG |
| | at nucleotides 1-2729 | GTTCGCGGACCCGGGAAGCTCTGAGGACCCAGAGGCCGGG |
| | | CGCGCTCCGCCCGCGGCGCCGCCCCCTCCGTAACTTTCCCA |
| | | GTCTCCGAGGGAAGAGGCGGGGTGTGGGGTGCGGTTAAAA |
| | | GGCGCCACGGCGGGAGACAGGTGTTGCGGCCCCGCAGCGC |
| | | CCGCGCGCTCCTCTCCCCGACTCGGAGCCCCTCGGCGGCG |
| | | CCCGGCCCAGGACCCGCCTAGGAGCGCAGGAGCCCCAGCG |
| | | CAGAGACCCCAACGCCGAGACCCCCGCCCCGGCCCCGCCG |
| | | CGCTTCCTCCCGACGCAGGTGAGCCCGCCGGCCCCGGACT |
| | | GCCCGGCCAGGAACCTGGCGCGGGGAGGGACCGCGAGACC |
| | | CAGAGCGGTTGCCCGGCCGCGTGGGTCTCGGGGAACCGGG |
| | | GGGCTGGACCAACACACGTCCTTGGGCCGGGGGGCGGGGG |
| | | CCGCCTTCTGGAGCGGGCGTTTCTGTTTATGATGTGTTTAAA |
| | | GATTGGGTGAATTACTCAGGTGAACAAGCTACTTTTTATCAGA |
| | | GAACACCTAAAAACACGTTCAAGAGGGTTTGGGAACTATACA |
| | | TTTAATCCTATGACAAACTAAGTTGGTTCTGTCTTCACCTGTTT |
| | | TGGTGAGGTTGTGTAAGAGTTGGTGTTTGCTCAGGAAGAGAT |
| | | TTAAGCATGCTTGCTTACCCAGACTCAGAGAAGTCTCCCTGT |
| | | TCTGTCCTAGCTAGTGATTCCTGTGTTGTGTGCATTCGTCTTT |
| | | TCCAGAGCAAACCGCCCAGAGTAGAAGCCTGCAGGCCACCA |
| | | TGGACTGGGGAACACTGCAAACAATCCTGGGAGGTGTGAAC |
| | | AAGCACAGCACAAGCATTGGCAAGATCTGGCTGACAGTGCT |
| | | GTTCATCTTTAGAATCATGATCCTGGTTGTGGCTGCTAAGGAA |
| | | GTGTGGGGAGATGAGCAGGCTGACTTTGTTTGTAATACCCTG |
| | | CAACCTGGCTGCAAGAATGTGTGTTATGACCACTACTTCCCC |
| | | ATCAGCCACATCAGACTGTGGGCCCTGCAACTGATCTTTGTG |
| | | TCCACCCCTGCCCTGCTGGTGGCCATGCATGTGGCCTACAG |
| | | AAGGCATGAGAAGAAGAGGAAATTCATCAAGGGAGAGATCAA |
| | | GAGTGAGTTCAAGGACATTGAGGAAATCAAAACCCAGAAGGT |
| | | GAGGATTGAAGGCTCTCTGTGGTGGACCTACACCAGCAGCA |
| | | TCTTCTTCAGAGTGATCTTTGAGGCAGCCTTTATGTATGTGTT |
| | | CTATGTGATGTATGATGGCTTCTCCATGCAGAGACTGGTGAA |
| | | GTGCAATGCCTGGCCATGTCCTAACACAGTGGACTGCTTTGT |
| | | GTCTAGACCTACAGAAAAGACAGTGTTTACAGTCTTTATGATT |
| | | GCAGTGTCTGGCATCTGCATTCTGCTGAATGTGACTGAGCTG |
| | | TGCTACCTGCTCATTAGGTACTGTAGTGGAAAAAGCAAGAAA |
| | | CCTGTGTGATAAGCTTAGATCTGCCTGACTGTGCCTTCTAGTT |
| | | GCCAGCCATCTGTTGTTTGCCCCTCCCCCGTGCCTTCCTTGA |
| | | CCCTGGAAGGTGCCACTCCCACTGTCCTTTCCTAATAAAATG |
| | | AGGAAATTGCATCGCATTGTCTGAGTAGGTGTCATTCTATTCT |
| | | GGGGGGTGGGGTGGGGCAGGACAGCAAGGGGGAGGATTG |
| | | GGAAGACAATAGCAGGCATGCTGGGGATCTAGAACTGAATTC |
| | | CCGATAAGGATCTTCCTAGAGCATGGCTACGTAGATAAGTAG |
| | | CATGGCGGGTTAATCATTAACTACAAGGAACCCCTAGTGATG |
| | | GAGTTGGCCACTCCCTCTCTGCGCGCTCGCTCGCTCACTGA |
| | | GGCCGGGCGACCAAAGGTCGCCCGACGCCCGGGCTTTGCC |
| | | CGGGCGGCCTCAGTGAGCGAGCGAGCGCGCAGCCTTAATTA |
| | | ACCTAATTCACTGGCCGTCGTTTTACAACGTCGTGACTGGGA |
| | | AAACCCTGGCGTTACCCAACTTAATCGCCTTGCAGCACATCC |
| | | CCCTTTCGCCAGCTGGCGTAATAGCGAAGAGGCCCGCACCG |
| | | ATCGCCCTTCCCAACAGTTGCGCAGCCTGAATGGCGAATGG |
| | | GACGCGCCCTGTAGCGGCGCATTAAGCGCGGCGGGTGTGG |
| | | TGGTTACGCGCAGCGTGACCGCTACACTTGCCAGCGCCCTA |
| | | GCGCCCGCTCCTTTCGCTTTCTTCCCTTCCTTTCTCGCCACG |
| | | TTCGCCGGCTTTCCCCGTCAAGCTCTAAATCGGGGGCTCCCT |
| | | TTAGGGTTCCGATTTAGTGCTTTACGGCACCTCGACCCCAAA |
| | | AAACTTGATTAGGGTGATGGTTCACGTAGTGGGCCATCGCCC |
| | | TGATAGACGGTTTTTCGCCCTTTGACGTTGGAGTCCACGTTC |
| | | TTTAATAGTGGACTCTTGTTCCAAACTGGAACAACACTCAACC |
| | | CTATCTCGGTCTATTCTTTTGATTTATAAGGGATTTTGCCGATT |
| | | TCGGCCTATTGGTTAAAAAATGAGCTGATTTAACAAAAATTTA |
| | | ACGCGAATTTTAACAAAATATTAACGCTTACAATTTAGGTGGC |
| | | ACTTTTCGGGGAAATGTGCGCGGAACCCCTATTTGTTTATTTT |
| | | TCTAAATACATTCAAATATGTATCCGCTCATGAGACAATAACC |
| | | CTGATAAATGCTTCAATAATATTGAAAAAGGAAGAGTATGAGC |
| | | CATATTCAACGGGAAACGTCGAGGCCGCGATTAAATTCCAAC |
| | | ATGGATGCTGATTTATATGGGTATAAATGGGCTCGCGATAAT |
| | | GTCGGGCAATCAGGTGCGACAATCTATCGCTTGTATGGGAAG |
| | | CCCGATGCGCCAGAGTTGTTTCTGAAACATGGCAAAGGTAGC |
| | | GTTGCCAATGATGTTACAGATGAGATGGTCAGACTAAACTGG |
| | | CTGACGGAATTTATGCCTCTTCCGACCATCAAGCATTTTATCC |
| | | GTACTCCTGATGATGCATGGTTACTCACCACTGCGATCCCCG |
| | | GAAAAACAGCATTCCAGGTATTAGAAGAATATCCTGATTCAG |
| | | GTGAAAATATTGTTGATGCGCTGGCAGTGTTCCTGCGCCGGT |
| | | TGCATTCGATTCCTGTTTGTAATTGTCCTTTTAACAGCGATCG |
| | | CGTATTTCGTCTTGCTCAGGCGCAATCACGAATGAATAACGG |
| | | TTTGGTTGATGCGAGTGATTTTGATGACGAGCGTAATGGCTG |
| | | GCCTGTTGAACAAGTCTGGAAAGAAATGCATAAACTTTTGCC |
| | | ATTCTCACCGGATTCAGTCGTCACTCATGGTGATTTCTCACTT |
| | | GATAACCTTATTTTTGACGAGGGGAAATTAATAGGTTGTATTG |
| | | ATGTTGGACGAGTCGGAATCGCAGACCGATACCAGGATCTTG |
| | | CCATCCTATGGAACTGCCTCGGTGAGTTTTCTCCTTCATTACA |
| | | GAAACGGCTTTTTCAAAAATATGGTATTGATAATCCTGATATG |
| | | AATAAATTGCAGTTTCATTTGATGCTCGATGAGTTTTTCTAACT |
| | | GTCAGACCAAGTTTACTCATATATACTTTAGATTGATTTAAAAC |
| | | TTCATTTTTAATTTAAAAGGATCTAGGTGAAGATCCTTTTTGAT |
| | | AATCTCATGACCAAAATCCCTTAACGTGAGTTTTCGTTCCACT |
| | | GAGCGTCAGACCCCGTAGAAAAGATCAAAGGATCTTCTTGAG |
| | | ATCCTTTTTTTCTGCGCGTAATCTGCTGCTTGCAAACAAAAAA |
| | | ACCACCGCTACCAGCGGTGGTTTGTTTGCCGGATCAAGAGCT |
| | | ACCAACTCTTTTTCCGAAGGTAACTGGCTTCAGCAGAGCGCA |
| | | GATACCAAATACTGTTCTTCTAGTGTAGCCGTAGTTAGGCCA |
| | | CCACTTCAAGAACTCTGTAGCACCGCCTACATACCTCGCTCT |
| | | GCTAATCCTGTTACCAGTGGCTGCTGCCAGTGGCGATAAGTC |
| | | GTGTCTTACCGGGTTGGACTCAAGACGATAGTTACCGGATAA |
| | | GGCGCAGCGGTCGGGCTGAACGGGGGGTTCGTGCACACAG |
| | | CCCAGCTTGGAGCGAACGACCTACACCGAACTGAGATACCTA |
| | | CAGCGTGAGCTATGAGAAAGCGCCACGCTTCCCGAAGGGAG |
| | | AAAGGCGGACAGGTATCCGGTAAGCGGCAGGGTCGGAACAG |
| | | GAGAGCGCACGAGGGAGCTTCCAGGGGGAAACGCCTGGTAT |
| | | CTTTATAGTCCTGTCGGGTTTCGCCACCTCTGACTTGAGCGT |
| | | CGATTTTTGTGATGCTCGTCAGGGGGGCGGAGCCTATGGAA |
| | | AAACGCCAGCAACGCGGCCTTTTTACGGTTCCTGGCCTTTTG |
| | | CTGGCCTTTTGCTCACATGTTCTTTCCTGCGTTATCCCCTGAT |
| | | TCTGTGGATAACCGTATTACCGCCTTTGAGTGAGCTGATACC |
| | | GCTCGCCGCAGCCGAACGACCGAGCGCAGCGAGTCAGTGA |
| | | GCGAGGAAGCGGAAGAGCGCCCAATACGCAAACCGCCTCTC |
| | | CCCGCGCGTTGGCCGATTCATTAATGCAGCTGGCACGACAG |
| | | GTTTCCCGACTGGAAAGCGGGCAGTGAGCGCAACGCAATTA |
| | | ATGTGAGTTAGCTCACTCATTAGGCACCCCAGGCTTTACACT |
| | | TTATGCTTCCGGCTCGTATGTTGTGTGGAATTGTGAGCGGAT |
| | | AACAATTTCACACAGGAAACAGCTATGACCATGATTACGCCA |
| | | GATTTAATTAAGGCCTTAATTAGG |
| 26 | Plasmid P1610 | CTGCGCGCTCGCTCGCTCACTGAGGCCGCCCGGGCAAAGC |
| | 5′ ITR at nucleotides | CCGGGCGTCGGGCGACCTTTGGTCGCCCGGCCTCAGTGAG |
| | 1-130 | CGAGCGAGCGCGCAGAGAGGGAGTGGCCAACTCCATCACTA |
| | GJB2 regulatory | GGGGTTCCTTGTAGTTAATGATTAACCCGCCATGCTACTTATC |
| | construct at nucleotides | TACGTAGCCATGCTCTAGGAAGATCGGAATTCTGGTACCTTG |
| | 212-2672 | CTAGCAAAAACTCTTACATAATTGTAACAGATTTGAGTTTCCT |
| | CpG depleted hGJB2 at | CTGGTTCAGGTTTTCTTGCCTCTTTGATAATCAAATGATCTGA |
| | nucleotides 2686-3363 | AGAAAGGCATAGAATTTCAAGGGAGAATCTGCATGACAGGAT |
| | bGH polyA signal | TACAATAAGGCTATTCATGGAGACTCTTTTATTAGCTTACACA |
| | sequence at nucleotides | GGATCTGCATCATTCTTCGTCTCTGGCTTTGCTACAAGGCTC |
| | 3386-3593 | CATTTAAACTTAACCCAACTTGCAGGCTTAACTGATCCAGGAA |
| | 3′ ITR at nucleotides | TGACTCAATACAATGAGCCAGCTACTGTAGCTCTTTTTCCCCT |
| | 3676-3805 | TTTGATAAGGGGAGTTAACACAATGGGCTTTACAGTTCTTAAA |
| | AmpR promoter at | TGACCGTTCCTGCAGGTATTGGAAAACCTGCATCGTATGCCT |
| | nucleotides 4463-4567 | GCTGCTTTAGGAGACAACAAACTCAGCAGCCCCTCCCCCGAA |
| | KanR at nucleotides | GCTGAAGTGCCAAACTTAAGGCTCCAGGTAAACTGCCCTTCC |
| | 4568-5377 | GATGAGAGCTTTACATTTAGGCAGTAGCCACAGGCCTAAACA |
| | Sequence to be | AAGTCAAATGGATGAAACTTGTGATCTCTCCTTGGAAATATGG |
| | transferred into vector | CCAAGAGTGAAACAACTACTTAGTTATTATTTTAAACGCAGGC |
| | at nucleotides 1-3805 | TGCTAAATGGCTTGTGTTGGTATGTAATGGCATCCTAGATAAT |
| | | GGGGGCTGCCAAGAAAATATTCACACTGGCAAGTCAATTAAA |
| | | ATACCGTTCTCTTCCTAAGAGGCCTGGCTGGATTCCAGAGCT |
| | | TCCTTTAGGGTAACGCTCAAACCTGTCATAGTTTATTTTGTTT |
| | | CTGTTTTCAGGCAACAGATTTGGTTATTGCTTTTATTGTATGA |
| | | GTGTTGAAATGGTGTCCTAACAGCCATCTGTACTGCCATCCC |
| | | CTGGTCTCTCTGCCTTTCCAGAAAGGAGATGAAGTTGCTGGA |
| | | GCAGGCAGGTTCTCTGAAAGGCTCCCTGAGTGTGGAAGAGC |
| | | AGCTGTCACTCATCAGTGGTTGTCCCAATATCCAAGAAGCAG |
| | | TAGAGGGTGCCATGCACATTCAGGTGAGTGAAGGACACTGG |
| | | CGCCGCATCTGTGGGGCCTGAGACATTGTCTCCCAGCCTCT |
| | | CCGGAGGAACAGGGCAGGATACAGAGCAGTGGCAGGTGTG |
| | | GGCATTTTGCATCTTCTGTAATCTCCAAACAAAGTCTGCGCTA |
| | | GGCCCTTTCAGGCTATAAAGGACAGGGTACTCTGATTTCTGC |
| | | TCTTGATGAGGGTTGATTGTGATACCAGGCGAGGGAGTAAGA |
| | | GATACAGGACACCATCTCTGCGGCTGCATCTTTGTCGCAGAA |
| | | ACAGACATTGTTCAGGGGACACCTGAGCCTCTGATAGTCAGC |
| | | CAGTGGCTCTGGCAGCTCACAGCAGTTATAGGGTGGCTGTG |
| | | TCCTTTTCTAAAGTCCTGACTTTTTATGGCCTGTGGTCATGGT |
| | | GACTCCACTACGGGCTGTGTTTCTGTTGACGCGTACAGAGGA |
| | | CAACGACCACAGCCATCCCTGAACCCCGCCCACGGCACAGC |
| | | GCCGGAGCCGGGGTCTGGGGCGCCGCTTCCTGGGGGGTCC |
| | | CGACTCTCAGCCGCCCCCGCTTCACCCGGGCCGCCAAGGG |
| | | GCTGGGGGAGGCGGCGCTCGGGGTAACCGGGGGAGACTCA |
| | | GGGCGCTGGGGGCACTTGGGGAACTCATGGGGGCTCAAAG |
| | | GAACTAGGAGATCGGGACCTCGAAGGGGACTTGGGGGGTTC |
| | | GGGGCTTTCGGGGGCGGTCGGGGGTTCGCGGACCCGGGAA |
| | | GCTCTGAGGACCCAGAGGCCGGGCGCGCTCCGCCCGCGGC |
| | | GCCGCCCCCTCCGTAACTTTCCCAGTCTCCGAGGGAAGAGG |
| | | CGGGGTGTGGGGTGCGGTTAAAAGGCGCCACGGCGGGAGA |
| | | CAGGTGTTGCGGCCCCGCAGCGCCCGCGCGCTCCTCTCCC |
| | | CGACTCGGAGCCCCTCGGCGGCGCCCGGCCCAGGACCCGC |
| | | CTAGGAGCGCAGGAGCCCCAGCGCAGAGACCCCAACGCCG |
| | | AGACCCCCGCCCCGGCCCCGCCGCGCTTCCTCCCGACGCA |
| | | GGTGAGCCCGCCGGCCCCGGACTGCCCGGCCAGGAACCTG |
| | | GCGCGGGGAGGGACCGCGAGACCCAGAGCGGTTGCCCGGC |
| | | CGCGTGGGTCTCGGGGAACCGGGGGGCTGGACCAACACAC |
| | | GTCCTTGGGCCGGGGGGCGGGGGCCGCCTTCTGGAGCGGG |
| | | CGTTTCTGTTTATGATGTGTTTAAAGATTGGGTGAATTACTCA |
| | | GGTGAACAAGCTACTTTTTATCAGAGAACACCTAAAAACACGT |
| | | TCAAGAGGGTTTGGGAACTATACATTTAATCCTATGACAAACT |
| | | AAGTTGGTTCTGTCTTCACCTGTTTTGGTGAGGTTGTGTAAGA |
| | | GTTGGTGTTTGCTCAGGAAGAGATTTAAGCATGCTTGCTTAC |
| | | CCAGACTCAGAGAAGTCTCCCTGTTCTGTCCTAGCTAGTGAT |
| | | TCCTGTGTTGTGTGCATTCGTCTTTTCCAGAGCAAACCGCCC |
| | | AGAGTAGAAGCCTGCAGGCCACCATGGATTGGGGCACACTG |
| | | CAGACTATCCTGGGGGGTGTGAACAAACACTCCACCAGCATT |
| | | GGAAAGATCTGGCTCACAGTCCTCTTCATTTTTAGGATTATGA |
| | | TCCTGGTTGTGGCTGCAAAGGAGGTGTGGGGAGATGAGCAG |
| | | GCTGACTTTGTCTGCAACACCCTGCAGCCAGGCTGCAAGAAT |
| | | GTGTGCTATGATCACTACTTCCCCATCTCCCACATCAGGCTAT |
| | | GGGCCCTGCAGCTGATCTTTGTGTCCACTCCAGCACTCCTAG |
| | | TGGCCATGCATGTGGCCTACAGGAGACATGAGAAGAAGAGG |
| | | AAGTTCATCAAGGGGGAGATAAAGAGTGAATTTAAGGACATT |
| | | GAGGAGATCAAAACCCAGAAGGTCAGAATTGAAGGCTCCCT |
| | | GTGGTGGACCTACACAAGCAGCATCTTCTTCAGGGTCATCTT |
| | | TGAAGCAGCCTTCATGTATGTCTTCTATGTCATGTATGATGGC |
| | | TTCTCCATGCAGAGGCTGGTGAAGTGCAATGCCTGGCCTTGT |
| | | CCCAACACTGTGGACTGCTTTGTGTCCAGGCCCACTGAGAAG |
| | | ACTGTCTTCACAGTGTTCATGATTGCAGTGTCTGGAATTTGCA |
| | | TCCTGCTGAATGTCACTGAATTGTGTTATTTGCTAATTAGATA |
| | | TTGTTCTGGGAAGTCAAAAAAGCCAGTTTGATAAGCTTAGATC |
| | | TGCCTGACTGTGCCTTCTAGTTGCCAGCCATCTGTTGTTTGC |
| | | CCCTCCCCCGTGCCTTCCTTGACCCTGGAAGGTGCCACTCC |
| | | CACTGTCCTTTCCTAATAAAATGAGGAAATTGCATCGCATTGT |
| | | CTGAGTAGGTGTCATTCTATTCTGGGGGGTGGGGTGGGGCA |
| | | GGACAGCAAGGGGGAGGATTGGGAAGACAATAGCAGGCATG |
| | | CTGGGGATCTAGAACTGAATTCCCGATAAGGATCTTCCTAGA |
| | | GCATGGCTACGTAGATAAGTAGCATGGCGGGTTAATCATTAA |
| | | CTACAAGGAACCCCTAGTGATGGAGTTGGCCACTCCCTCTCT |
| | | GCGCGCTCGCTCGCTCACTGAGGCCGGGCGACCAAAGGTC |
| | | GCCCGACGCCCGGGCTTTGCCCGGGCGGCCTCAGTGAGCG |
| | | AGCGAGCGCGCAGCCTTAATTAACCTAATTCACTGGCCGTCG |
| | | TTTTACAACGTCGTGACTGGGAAAACCCTGGCGTTACCCAAC |
| | | TTAATCGCCTTGCAGCACATCCCCCTTTCGCCAGCTGGCGTA |
| | | ATAGCGAAGAGGCCCGCACCGATCGCCCTTCCCAACAGTTG |
| | | CGCAGCCTGAATGGCGAATGGGACGCGCCCTGTAGCGGCG |
| | | CATTAAGCGCGGCGGGTGTGGTGGTTACGCGCAGCGTGACC |
| | | GCTACACTTGCCAGCGCCCTAGCGCCCGCTCCTTTCGCTTTC |
| | | TTCCCTTCCTTTCTCGCCACGTTCGCCGGCTTTCCCCGTCAA |
| | | GCTCTAAATCGGGGGCTCCCTTTAGGGTTCCGATTTAGTGCT |
| | | TTACGGCACCTCGACCCCAAAAAACTTGATTAGGGTGATGGT |
| | | TCACGTAGTGGGCCATCGCCCTGATAGACGGTTTTTCGCCCT |
| | | TTGACGTTGGAGTCCACGTTCTTTAATAGTGGACTCTTGTTCC |
| | | AAACTGGAACAACACTCAACCCTATCTCGGTCTATTCTTTTGA |
| | | TTTATAAGGGATTTTGCCGATTTCGGCCTATTGGTTAAAAAAT |
| | | GAGCTGATTTAACAAAAATTTAACGCGAATTTTAACAAAATATT |
| | | AACGCTTACAATTTAGGTGGCACTTTTCGGGGAAATGTGCGC |
| | | GGAACCCCTATTTGTTTATTTTTCTAAATACATTCAAATATGTA |
| | | TCCGCTCATGAGACAATAACCCTGATAAATGCTTCAATAATAT |
| | | TGAAAAAGGAAGAGTATGAGCCATATTCAACGGGAAACGTCG |
| | | AGGCCGCGATTAAATTCCAACATGGATGCTGATTTATATGGG |
| | | TATAAATGGGCTCGCGATAATGTCGGGCAATCAGGTGCGACA |
| | | ATCTATCGCTTGTATGGGAAGCCCGATGCGCCAGAGTTGTTT |
| | | CTGAAACATGGCAAAGGTAGCGTTGCCAATGATGTTACAGAT |
| | | GAGATGGTCAGACTAAACTGGCTGACGGAATTTATGCCTCTT |
| | | CCGACCATCAAGCATTTTATCCGTACTCCTGATGATGCATGG |
| | | TTACTCACCACTGCGATCCCCGGAAAAACAGCATTCCAGGTA |
| | | TTAGAAGAATATCCTGATTCAGGTGAAAATATTGTTGATGCGC |
| | | TGGCAGTGTTCCTGCGCCGGTTGCATTCGATTCCTGTTTGTA |
| | | ATTGTCCTTTTAACAGCGATCGCGTATTTCGTCTTGCTCAGGC |
| | | GCAATCACGAATGAATAACGGTTTGGTTGATGCGAGTGATTT |
| | | TGATGACGAGCGTAATGGCTGGCCTGTTGAACAAGTCTGGAA |
| | | AGAAATGCATAAACTTTTGCCATTCTCACCGGATTCAGTCGTC |
| | | ACTCATGGTGATTTCTCACTTGATAACCTTATTTTTGACGAGG |
| | | GGAAATTAATAGGTTGTATTGATGTTGGACGAGTCGGAATCG |
| | | CAGACCGATACCAGGATCTTGCCATCCTATGGAACTGCCTCG |
| | | GTGAGTTTTCTCCTTCATTACAGAAACGGCTTTTTCAAAAATA |
| | | TGGTATTGATAATCCTGATATGAATAAATTGCAGTTTCATTTGA |
| | | TGCTCGATGAGTTTTTCTAACTGTCAGACCAAGTTTACTCATA |
| | | TATACTTTAGATTGATTTAAAACTTCATTTTTAATTTAAAAGGAT |
| | | CTAGGTGAAGATCCTTTTTGATAATCTCATGACCAAAATCCCT |
| | | TAACGTGAGTTTTCGTTCCACTGAGCGTCAGACCCCGTAGAA |
| | | AAGATCAAAGGATCTTCTTGAGATCCTTTTTTTCTGCGCGTAA |
| | | TCTGCTGCTTGCAAACAAAAAAACCACCGCTACCAGCGGTGG |
| | | TTTGTTTGCCGGATCAAGAGCTACCAACTCTTTTTCCGAAGGT |
| | | AACTGGCTTCAGCAGAGCGCAGATACCAAATACTGTTCTTCT |
| | | AGTGTAGCCGTAGTTAGGCCACCACTTCAAGAACTCTGTAGC |
| | | ACCGCCTACATACCTCGCTCTGCTAATCCTGTTACCAGTGGC |
| | | TGCTGCCAGTGGCGATAAGTCGTGTCTTACCGGGTTGGACTC |
| | | AAGACGATAGTTACCGGATAAGGCGCAGCGGTCGGGCTGAA |
| | | CGGGGGGTTCGTGCACACAGCCCAGCTTGGAGCGAACGACC |
| | | TACACCGAACTGAGATACCTACAGCGTGAGCTATGAGAAAGC |
| | | GCCACGCTTCCCGAAGGGAGAAAGGCGGACAGGTATCCGGT |
| | | AAGCGGCAGGGTCGGAACAGGAGAGCGCACGAGGGAGCTT |
| | | CCAGGGGGAAACGCCTGGTATCTTTATAGTCCTGTCGGGTTT |
| | | CGCCACCTCTGACTTGAGCGTCGATTTTTGTGATGCTCGTCA |
| | | GGGGGGCGGAGCCTATGGAAAAACGCCAGCAACGCGGCCT |
| | | TTTTACGGTTCCTGGCCTTTTGCTGGCCTTTTGCTCACATGTT |
| | | CTTTCCTGCGTTATCCCCTGATTCTGTGGATAACCGTATTACC |
| | | GCCTTTGAGTGAGCTGATACCGCTCGCCGCAGCCGAACGAC |
| | | CGAGCGCAGCGAGTCAGTGAGCGAGGAAGCGGAAGAGCGC |
| | | CCAATACGCAAACCGCCTCTCCCCGCGCGTTGGCCGATTCAT |
| | | TAATGCAGCTGGCACGACAGGTTTCCCGACTGGAAAGCGGG |
| | | CAGTGAGCGCAACGCAATTAATGTGAGTTAGCTCACTCATTA |
| | | GGCACCCCAGGCTTTACACTTTATGCTTCCGGCTCGTATGTT |
| | | GTGTGGAATTGTGAGCGGATAACAATTTCACACAGGAAACAG |
| | | CTATGACCATGATTACGCCAGATTTAATTAAGGCCTTAATTAG |
| | | G |
| 27 | Plasmid P1611 | CTGCGCGCTCGCTCGCTCACTGAGGCCGCCCGGGCAAAGC |
| | 5′ ITR at nucleotides | CCGGGCGTCGGGCGACCTTTGGTCGCCCGGCCTCAGTGAG |
| | 1-130 | CGAGCGAGCGCGCAGAGAGGGAGTGGCCAACTCCATCACTA |
| | GJB2 regulatory | GGGGTTCCTTGTAGTTAATGATTAACCCGCCATGCTACTTATC |
| | construct at nucleotides | TACGTAGCCATGCTCTAGGAAGATCGGAATTCTGGTACCTTG |
| | 212-2672 | CTAGCAAAAACTCTTACATAATTGTAACAGATTTGAGTTTCCT |
| | Codon-optimized and | CTGGTTCAGGTTTTCTTGCCTCTTTGATAATCAAATGATCTGA |
| | CpG depleted hGJB2 at | AGAAAGGCATAGAATTTCAAGGGAGAATCTGCATGACAGGAT |
| | nucleotides 2686-3363 | TACAATAAGGCTATTCATGGAGACTCTTTTATTAGCTTACACA |
| | bGH polyA signal | GGATCTGCATCATTCTTCGTCTCTGGCTTTGCTACAAGGCTC |
| | sequence at nucleotides | CATTTAAACTTAACCCAACTTGCAGGCTTAACTGATCCAGGAA |
| | 3386-3593 | TGACTCAATACAATGAGCCAGCTACTGTAGCTCTTTTTCCCCT |
| | 3′ ITR at nucleotides | TTTGATAAGGGGAGTTAACACAATGGGCTTTACAGTTCTTAAA |
| | 3676-3805 | TGACCGTTCCTGCAGGTATTGGAAAACCTGCATCGTATGCCT |
| | AmpR promoter at | GCTGCTTTAGGAGACAACAAACTCAGCAGCCCCTCCCCCGAA |
| | nucleotides 4463-4567 | GCTGAAGTGCCAAACTTAAGGCTCCAGGTAAACTGCCCTTCC |
| | KanR at nucleotides | GATGAGAGCTTTACATTTAGGCAGTAGCCACAGGCCTAAACA |
| | 4568-5377 | AAGTCAAATGGATGAAACTTGTGATCTCTCCTTGGAAATATGG |
| | Sequence to be | CCAAGAGTGAAACAACTACTTAGTTATTATTTTAAACGCAGGC |
| | transferred into vector | TGCTAAATGGCTTGTGTTGGTATGTAATGGCATCCTAGATAAT |
| | at nucleotides 1-3805 | GGGGGCTGCCAAGAAAATATTCACACTGGCAAGTCAATTAAA |
| | | ATACCGTTCTCTTCCTAAGAGGCCTGGCTGGATTCCAGAGCT |
| | | TCCTTTAGGGTAACGCTCAAACCTGTCATAGTTTATTTTGTTT |
| | | CTGTTTTCAGGCAACAGATTTGGTTATTGCTTTTATTGTATGA |
| | | GTGTTGAAATGGTGTCCTAACAGCCATCTGTACTGCCATCCC |
| | | CTGGTCTCTCTGCCTTTCCAGAAAGGAGATGAAGTTGCTGGA |
| | | GCAGGCAGGTTCTCTGAAAGGCTCCCTGAGTGTGGAAGAGC |
| | | AGCTGTCACTCATCAGTGGTTGTCCCAATATCCAAGAAGCAG |
| | | TAGAGGGTGCCATGCACATTCAGGTGAGTGAAGGACACTGG |
| | | CGCCGCATCTGTGGGGCCTGAGACATTGTCTCCCAGCCTCT |
| | | CCGGAGGAACAGGGCAGGATACAGAGCAGTGGCAGGTGTG |
| | | GGCATTTTGCATCTTCTGTAATCTCCAAACAAAGTCTGCGCTA |
| | | GGCCCTTTCAGGCTATAAAGGACAGGGTACTCTGATTTCTGC |
| | | TCTTGATGAGGGTTGATTGTGATACCAGGCGAGGGAGTAAGA |
| | | GATACAGGACACCATCTCTGCGGCTGCATCTTTGTCGCAGAA |
| | | ACAGACATTGTTCAGGGGACACCTGAGCCTCTGATAGTCAGC |
| | | CAGTGGCTCTGGCAGCTCACAGCAGTTATAGGGTGGCTGTG |
| | | TCCTTTTCTAAAGTCCTGACTTTTTATGGCCTGTGGTCATGGT |
| | | GACTCCACTACGGGCTGTGTTTCTGTTGACGCGTACAGAGGA |
| | | CAACGACCACAGCCATCCCTGAACCCCGCCCACGGCACAGC |
| | | GCCGGAGCCGGGGTCTGGGGCGCCGCTTCCTGGGGGGTCC |
| | | CGACTCTCAGCCGCCCCCGCTTCACCCGGGCCGCCAAGGG |
| | | GCTGGGGGAGGCGGCGCTCGGGGTAACCGGGGGAGACTCA |
| | | GGGCGCTGGGGGCACTTGGGGAACTCATGGGGGCTCAAAG |
| | | GAACTAGGAGATCGGGACCTCGAAGGGGACTTGGGGGGTTC |
| | | GGGGCTTTCGGGGGCGGTCGGGGGTTCGCGGACCCGGGAA |
| | | GCTCTGAGGACCCAGAGGCCGGGCGCGCTCCGCCCGCGGC |
| | | GCCGCCCCCTCCGTAACTTTCCCAGTCTCCGAGGGAAGAGG |
| | | CGGGGTGTGGGGTGCGGTTAAAAGGCGCCACGGCGGGAGA |
| | | CAGGTGTTGCGGCCCCGCAGCGCCCGCGCGCTCCTCTCCC |
| | | CGACTCGGAGCCCCTCGGCGGCGCCCGGCCCAGGACCCGC |
| | | CTAGGAGCGCAGGAGCCCCAGCGCAGAGACCCCAACGCCG |
| | | AGACCCCCGCCCCGGCCCCGCCGCGCTTCCTCCCGACGCA |
| | | GGTGAGCCCGCCGGCCCCGGACTGCCCGGCCAGGAACCTG |
| | | GCGCGGGGAGGGACCGCGAGACCCAGAGCGGTTGCCCGGC |
| | | CGCGTGGGTCTCGGGGAACCGGGGGGCTGGACCAACACAC |
| | | GTCCTTGGGCCGGGGGGCGGGGGCCGCCTTCTGGAGCGGG |
| | | CGTTTCTGTTTATGATGTGTTTAAAGATTGGGTGAATTACTCA |
| | | GGTGAACAAGCTACTTTTTATCAGAGAACACCTAAAAACACGT |
| | | TCAAGAGGGTTTGGGAACTATACATTTAATCCTATGACAAACT |
| | | AAGTTGGTTCTGTCTTCACCTGTTTTGGTGAGGTTGTGTAAGA |
| | | GTTGGTGTTTGCTCAGGAAGAGATTTAAGCATGCTTGCTTAC |
| | | CCAGACTCAGAGAAGTCTCCCTGTTCTGTCCTAGCTAGTGAT |
| | | TCCTGTGTTGTGTGCATTCGTCTTTTCCAGAGCAAACCGCCC |
| | | AGAGTAGAAGCCTGCAGGCCACCATGGACTGGGGAACACTG |
| | | CAAACAATCCTGGGAGGTGTGAACAAGCACAGCACAAGCATT |
| | | GGCAAGATCTGGCTGACAGTGCTGTTCATCTTTAGAATCATG |
| | | ATCCTGGTTGTGGCTGCTAAGGAAGTGTGGGGAGATGAGCA |
| | | GGCTGACTTTGTTTGTAATACCCTGCAACCTGGCTGCAAGAA |
| | | TGTGTGTTATGACCACTACTTCCCCATCAGCCACATCAGACT |
| | | GTGGGCCCTGCAACTGATCTTTGTGTCCACCCCTGCCCTGCT |
| | | GGTGGCCATGCATGTGGCCTACAGAAGGCATGAGAAGAAGA |
| | | GGAAATTCATCAAGGGAGAGATCAAGAGTGAGTTCAAGGACA |
| | | TTGAGGAAATCAAAACCCAGAAGGTGAGGATTGAAGGCTCTC |
| | | TGTGGTGGACCTACACCAGCAGCATCTTCTTCAGAGTGATCT |
| | | TTGAGGCAGCCTTTATGTATGTGTTCTATGTGATGTATGATGG |
| | | CTTCTCCATGCAGAGACTGGTGAAGTGCAATGCCTGGCCATG |
| | | TCCTAACACAGTGGACTGCTTTGTGTCTAGACCTACAGAAAA |
| | | GACAGTGTTTACAGTCTTTATGATTGCAGTGTCTGGCATCTGC |
| | | ATTCTGCTGAATGTGACTGAGCTGTGCTACCTGCTCATTAGG |
| | | TACTGTAGTGGAAAAAGCAAGAAACCTGTGTGATAAGCTTAG |
| | | ATCTGCCTGACTGTGCCTTCTAGTTGCCAGCCATCTGTTGTTT |
| | | GCCCCTCCCCCGTGCCTTCCTTGACCCTGGAAGGTGCCACT |
| | | CCCACTGTCCTTTCCTAATAAAATGAGGAAATTGCATCGCATT |
| | | GTCTGAGTAGGTGTCATTCTATTCTGGGGGGTGGGGTGGGG |
| | | CAGGACAGCAAGGGGGAGGATTGGGAAGACAATAGCAGGCA |
| | | TGCTGGGGATCTAGAACTGAATTCCCGATAAGGATCTTCCTA |
| | | GAGCATGGCTACGTAGATAAGTAGCATGGCGGGTTAATCATT |
| | | AACTACAAGGAACCCCTAGTGATGGAGTTGGCCACTCCCTCT |
| | | CTGCGCGCTCGCTCGCTCACTGAGGCCGGGCGACCAAAGGT |
| | | CGCCCGACGCCCGGGCTTTGCCCGGGCGGCCTCAGTGAGC |
| | | GAGCGAGCGCGCAGCCTTAATTAACCTAATTCACTGGCCGTC |
| | | GTTTTACAACGTCGTGACTGGGAAAACCCTGGCGTTACCCAA |
| | | CTTAATCGCCTTGCAGCACATCCCCCTTTCGCCAGCTGGCGT |
| | | AATAGCGAAGAGGCCCGCACCGATCGCCCTTCCCAACAGTT |
| | | GCGCAGCCTGAATGGCGAATGGGACGCGCCCTGTAGCGGC |
| | | GCATTAAGCGCGGCGGGTGTGGTGGTTACGCGCAGCGTGAC |
| | | CGCTACACTTGCCAGCGCCCTAGCGCCCGCTCCTTTCGCTTT |
| | | CTTCCCTTCCTTTCTCGCCACGTTCGCCGGCTTTCCCCGTCA |
| | | AGCTCTAAATCGGGGGCTCCCTTTAGGGTTCCGATTTAGTGC |
| | | TTTACGGCACCTCGACCCCAAAAAACTTGATTAGGGTGATGG |
| | | TTCACGTAGTGGGCCATCGCCCTGATAGACGGTTTTTCGCCC |
| | | TTTGACGTTGGAGTCCACGTTCTTTAATAGTGGACTCTTGTTC |
| | | CAAACTGGAACAACACTCAACCCTATCTCGGTCTATTCTTTTG |
| | | ATTTATAAGGGATTTTGCCGATTTCGGCCTATTGGTTAAAAAA |
| | | TGAGCTGATTTAACAAAAATTTAACGCGAATTTTAACAAAATAT |
| | | TAACGCTTACAATTTAGGTGGCACTTTTCGGGGAAATGTGCG |
| | | CGGAACCCCTATTTGTTTATTTTTCTAAATACATTCAAATATGT |
| | | ATCCGCTCATGAGACAATAACCCTGATAAATGCTTCAATAATA |
| | | TTGAAAAAGGAAGAGTATGAGCCATATTCAACGGGAAACGTC |
| | | GAGGCCGCGATTAAATTCCAACATGGATGCTGATTTATATGG |
| | | GTATAAATGGGCTCGCGATAATGTCGGGCAATCAGGTGCGA |
| | | CAATCTATCGCTTGTATGGGAAGCCCGATGCGCCAGAGTTGT |
| | | TTCTGAAACATGGCAAAGGTAGCGTTGCCAATGATGTTACAG |
| | | ATGAGATGGTCAGACTAAACTGGCTGACGGAATTTATGCCTC |
| | | TTCCGACCATCAAGCATTTTATCCGTACTCCTGATGATGCATG |
| | | GTTACTCACCACTGCGATCCCCGGAAAAACAGCATTCCAGGT |
| | | ATTAGAAGAATATCCTGATTCAGGTGAAAATATTGTTGATGCG |
| | | CTGGCAGTGTTCCTGCGCCGGTTGCATTCGATTCCTGTTTGT |
| | | AATTGTCCTTTTAACAGCGATCGCGTATTTCGTCTTGCTCAGG |
| | | CGCAATCACGAATGAATAACGGTTTGGTTGATGCGAGTGATT |
| | | TTGATGACGAGCGTAATGGCTGGCCTGTTGAACAAGTCTGGA |
| | | AAGAAATGCATAAACTTTTGCCATTCTCACCGGATTCAGTCGT |
| | | CACTCATGGTGATTTCTCACTTGATAACCTTATTTTTGACGAG |
| | | GGGAAATTAATAGGTTGTATTGATGTTGGACGAGTCGGAATC |
| | | GCAGACCGATACCAGGATCTTGCCATCCTATGGAACTGCCTC |
| | | GGTGAGTTTTCTCCTTCATTACAGAAACGGCTTTTTCAAAAAT |
| | | ATGGTATTGATAATCCTGATATGAATAAATTGCAGTTTCATTTG |
| | | ATGCTCGATGAGTTTTTCTAACTGTCAGACCAAGTTTACTCAT |
| | | ATATACTTTAGATTGATTTAAAACTTCATTTTTAATTTAAAAGG |
| | | ATCTAGGTGAAGATCCTTTTTGATAATCTCATGACCAAAATCC |
| | | CTTAACGTGAGTTTTCGTTCCACTGAGCGTCAGACCCCGTAG |
| | | AAAAGATCAAAGGATCTTCTTGAGATCCTTTTTTTCTGCGCGT |
| | | AATCTGCTGCTTGCAAACAAAAAAACCACCGCTACCAGCGGT |
| | | GGTTTGTTTGCCGGATCAAGAGCTACCAACTCTTTTTCCGAA |
| | | GGTAACTGGCTTCAGCAGAGCGCAGATACCAAATACTGTTCT |
| | | TCTAGTGTAGCCGTAGTTAGGCCACCACTTCAAGAACTCTGT |
| | | AGCACCGCCTACATACCTCGCTCTGCTAATCCTGTTACCAGT |
| | | GGCTGCTGCCAGTGGCGATAAGTCGTGTCTTACCGGGTTGG |
| | | ACTCAAGACGATAGTTACCGGATAAGGCGCAGCGGTCGGGC |
| | | TGAACGGGGGGTTCGTGCACACAGCCCAGCTTGGAGCGAAC |
| | | GACCTACACCGAACTGAGATACCTACAGCGTGAGCTATGAGA |
| | | AAGCGCCACGCTTCCCGAAGGGAGAAAGGCGGACAGGTATC |
| | | CGGTAAGCGGCAGGGTCGGAACAGGAGAGCGCACGAGGGA |
| | | GCTTCCAGGGGGAAACGCCTGGTATCTTTATAGTCCTGTCGG |
| | | GTTTCGCCACCTCTGACTTGAGCGTCGATTTTTGTGATGCTC |
| | | GTCAGGGGGGCGGAGCCTATGGAAAAACGCCAGCAACGCG |
| | | GCCTTTTTACGGTTCCTGGCCTTTTGCTGGCCTTTTGCTCACA |
| | | TGTTCTTTCCTGCGTTATCCCCTGATTCTGTGGATAACCGTAT |
| | | TACCGCCTTTGAGTGAGCTGATACCGCTCGCCGCAGCCGAA |
| | | CGACCGAGCGCAGCGAGTCAGTGAGCGAGGAAGCGGAAGA |
| | | GCGCCCAATACGCAAACCGCCTCTCCCCGCGCGTTGGCCGA |
| | | TTCATTAATGCAGCTGGCACGACAGGTTTCCCGACTGGAAAG |
| | | CGGGCAGTGAGCGCAACGCAATTAATGTGAGTTAGCTCACTC |
| | | ATTAGGCACCCCAGGCTTTACACTTTATGCTTCCGGCTCGTA |
| | | TGTTGTGTGGAATTGTGAGCGGATAACAATTTCACACAGGAA |
| | | ACAGCTATGACCATGATTACGCCAGATTTAATTAAGGCCTTAA |
| | | TTAGG |

### Methods for the Delivery of a Polynucleotide Encoding Gjb2 to Target Cells

Techniques that can be used to introduce a polynucleotide encoding Gjb2 (e.g., wild-type human Gjb2) that is operably linked to a GJB2 regulatory construct described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more sequence identity) to SEQ ID NO: 1 or SEQ ID NO: 2) into a target cell (e.g., a mammalian cell) are well known in the art. For instance, electroporation can be used to permeabilize mammalian cells (e.g., human target cells) by the application of an electrostatic potential to the cell of interest. Mammalian cells, such as human cells, subjected to an external electric field in this manner are subsequently predisposed to the uptake of exogenous polynucleotides. Electroporation of mammalian cells is described in detail, e.g., in Chu et al., Nucleic Acids Research 15:1311 (1987), the disclosure of which is incorporated herein by reference. A similar technique, Nucleofection™, utilizes an applied electric field in order to stimulate the uptake of exogenous polynucleotides into the nucleus of a eukaryotic cell. Nucleofection™ and protocols useful for performing this technique are described in detail, e.g., in Distler et al., Experimental Dermatology 14:315 (2005), as well as in US 2010/0317114, the disclosures of each of which are incorporated herein by reference.

Additional techniques useful for the transfection of target cells include the squeeze-poration methodology. This technique induces the rapid mechanical deformation of cells in order to stimulate the uptake of exogenous DNA through membranous pores that form in response to the applied stress. This technology is advantageous in that a vector is not required for delivery of polynucleotides into a cell, such as a human target cell. Squeeze-poration is described in detail, e.g., in Sharei et al., Journal of Visualized Experiments 81:e50980 (2013), the disclosure of which is incorporated herein by reference.

Lipofection represents another technique useful for transfection of target cells. This method involves the loading of polynucleotides into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous polynucleotides, for instance, by direct fusion of the liposome with the cell membrane or by endocytosis of the complex. Lipofection is described in detail, for instance, in U.S. Pat. No. 7,442,386, the disclosure of which is incorporated herein by reference. Similar techniques that exploit ionic interactions with the cell membrane to provoke the uptake of foreign polynucleotides include contacting a cell with a cationic polymer-polynucleotide complex. Exemplary cationic molecules that associate with polynucleotides so as to impart a positive charge favorable for interaction with the cell membrane include activated dendrimers (described, e.g., in Dennig, Topics in Current Chemistry 228:227 (2003), the disclosure of which is incorporated herein by reference) polyethylenimine, and diethylaminoethyl (DEAE)-dextran, the use of which as a transfection agent is described in detail, for instance, in Gulick et al., Current Protocols in Molecular Biology 40:1:9.2:9.2.1 (1997), the disclosure of which is incorporated herein by reference. Magnetic beads are another tool that can be used to transfect target cells in a mild and efficient manner, as this methodology utilizes an applied magnetic field in order to direct the uptake of polynucleotides. This technology is described in detail, for instance, in US 2010/0227406, the disclosure of which is incorporated herein by reference.

Another useful tool for inducing the uptake of exogenous polynucleotides by target cells is laserfection, also called optical transfection, a technique that involves exposing a cell to electromagnetic radiation of a particular wavelength in order to gently permeabilize the cells and allow polynucleotides to penetrate the cell membrane. The bioactivity of this technique is similar to, and in some cases found superior to, electroporation.

Impalefection is another technique that can be used to deliver genetic material to target cells. It relies on the use of nanomaterials, such as carbon nanofibers, carbon nanotubes, and nanowires. Needle-like nanostructures are synthesized perpendicular to the surface of a substrate. DNA containing the gene, intended for intracellular delivery, is attached to the nanostructure surface. A chip with arrays of these needles is then pressed against cells or tissue. Cells that are impaled by nanostructures can express the delivered gene(s). An example of this technique is described in Shalek et al., PNAS 107: 1870 (2010), the disclosure of which is incorporated herein by reference.

Magnetofection can also be used to deliver polynucleotides to target cells. The magnetofection principle is to associate polynucleotides with cationic magnetic nanoparticles. The magnetic nanoparticles are made of iron oxide, which is fully biodegradable, and coated with specific cationic proprietary molecules varying upon the applications. Their association with the gene vectors (DNA, siRNA, viral vector, etc.) is achieved by salt-induced colloidal aggregation and electrostatic interaction. The magnetic particles are then concentrated on the target cells by the influence of an external magnetic field generated by magnets. This technique is described in detail in Scherer et al., Gene Therapy 9:102 (2002), the disclosure of which is incorporated herein by reference.

Another useful tool for inducing the uptake of exogenous polynucleotides by target cells is sonoporation, a technique that involves the use of sound (typically ultrasonic frequencies) for modifying the permeability of the cell plasma membrane to permeabilize the cells and allow polynucleotides to penetrate the cell membrane. This technique is described in detail, e.g., in Rhodes et al., Methods in Cell Biology 82:309 (2007), the disclosure of which is incorporated herein by reference.

Microvesicles represent another potential vehicle that can be used to modify the genome of a target cell according to the methods described herein. For instance, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene or regulatory sequence. The use of such vesicles, also referred to as Gesicles, for the genetic modification of eukaryotic cells is described in detail, e.g., in Quinn et al., Genetic Modification of Target Cells by Direct Delivery of Active Protein [abstract]. In: Methylation changes in early embryonic genes in cancer [abstract], in: Proceedings of the 18th Annual Meeting of the American Society of Gene and Cell Therapy; 2015 May 13, Abstract No. 122.

### Vectors for the Expression of GJB2

In addition to achieving high rates of transcription and translation, stable expression of an exogenous polynucleotide in a mammalian cell can be achieved by integration of the polynucleotide into the nuclear genome of the mammalian cell. A variety of vectors for the delivery and integration of polynucleotides encoding exogenous proteins into the nuclear DNA of a mammalian cell have been developed. Examples of expression vectors are described in, e.g., Gellissen, Production of Recombinant Proteins: Novel Microbial and Eukaryotic Expression Systems (John Wiley & Sons, Marblehead, MA, 2006). Expression vectors for use in the compositions and methods described herein contain a GJB2 regulatory construct described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more sequence identity) to SEQ ID NO: 1 or SEQ ID NO: 2) operably linked to a polynucleotide encoding a Gjb2 protein (e.g., wild-type human Gjb2, such as a polynucleotide having least 90% sequence identity (e.g., 90%, 91%, 92%, 93%, 94%, 95%, 96%, 97%, 98%, 99%, or more sequence identity) to the sequence of SEQ ID NO: 3 and containing at least 50% fewer CG dinucleotides (e.g., 50%, 60%, 70%, 75%, 80%, 85%, 90%, 95%, or fewer CG dinucleotides) than wild-type GJB2 or a polynucleotide having at least 90% sequence identity (e.g., 90%, 91%, 92%, 93%, 94%, 95%, 96%, 97%, 98%, 99%, or more sequence identity) to the sequence of SEQ ID NO: 4 or SEQ ID NO: 28), as well as, e.g., additional sequence elements used for the expression of these agents and/or the integration of these polynucleotide sequences into the genome of a mammalian cell. Vectors that can contain a GJB2 regulatory construct operably linked to polynucleotide encoding Gjb2 include plasmids (e.g., circular DNA molecules that can autonomously replicate inside a cell), cosmids (e.g., pWE or sCos vectors), artificial chromosomes (e.g., a human artificial chromosome (HAC), a yeast artificial chromosome (YAC), a bacterial artificial chromosome (BAC), or a P1-derived artificial chromosome (PAC)), and viral vectors. Certain vectors that can be used for the expression of a polynucleotide encoding a Gjb2 protein include plasmids that contain regulatory sequences, such as enhancer regions, which direct gene transcription. Other useful vectors for expression of a polynucleotide encoding a Gjb2 protein contain polynucleotide sequences that enhance the rate of translation of these genes or improve the stability or nuclear export of the mRNA that results from gene transcription. These sequence elements include, e.g., 5′ and 3′ untranslated regions and a polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

### Viral Vectors for Polynucleotide Delivery

Viral genomes provide a rich source of vectors that can be used for the efficient delivery of a gene of interest into the genome of a target cell (e.g., a mammalian cell, such as a human cell). Viral genomes are particularly useful vectors for gene delivery because the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors include a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology, Third Edition (Lippincott-Raven, Philadelphia, 1996)). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, U.S. Pat. No. 5,801,030, the disclosure of which is incorporated herein by reference as it pertains to viral vectors for use in gene therapy.

### AAV Vectors for Polynucleotide Delivery

In some embodiments, polynucleotides of the compositions and methods described herein are incorporated into rAAV vectors and/or virions in order to facilitate their introduction into a cell. In some embodiments, rAAV vectors useful in the compositions and methods described herein are recombinant polynucleotide constructs that include (1) a GJB2 regulatory construct described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more sequence identity) to SEQ ID NO: 1 or SEQ ID NO: 2), (2) a sequence to be expressed (e.g., a polynucleotide encoding Gjb2, such as a polynucleotide having least 90% sequence identity (e.g., 90%, 91%, 92%, 93%, 94%, 95%, 96%, 97%, 98%, 99%, or more sequence identity) to the sequence of SEQ ID NO: 3 and containing at least 50% fewer CG dinucleotides (e.g., 50%, 60%, 70%, 75%, 80%, 85%, 90%, 95%, or fewer CG dinucleotides) than wild-type GJB2 or a polynucleotide having at least 90% sequence identity (e.g., 90%, 91%, 92%, 93%, 94%, 95%, 96%, 97%, 98%, 99%, or more sequence identity) to the sequence of SEQ ID NO: 4 or SEQ ID NO: 28), and (3) viral sequences that facilitate integration and expression of the sequence to be expressed. The viral sequences may include those sequences of AAV that are required in cis for replication and packaging (e.g., functional ITRs) of the DNA into a virion. Such rAAV vectors may also contain marker or reporter genes. Useful rAAV vectors have one or more of the AAV WT genes deleted in whole or in part but retain functional flanking ITR sequences. The AAV ITRs may be of any serotype suitable for a particular application. For use in the methods and compositions described herein, the ITRs can be AAV2 ITRs. Methods for using rAAV vectors are described, for example, in Tal et al., J. Biomed. Sci. 7:279 (2000), and Monahan and Samulski, Gene Delivery 7:24 (2000), the disclosures of each of which are incorporated herein by reference as they pertain to AAV vectors for gene delivery.

The polynucleotides and vectors described herein (e.g., a GJB2 regulatory construct operably linked to a polynucleotide encoding Gjb2) can be incorporated into a rAAV virion in order to facilitate introduction of the polynucleotide or vector into a cell. The capsid proteins of AAV compose the exterior, non-nucleic acid portion of the virion and are encoded by the AAV cap gene. The cap gene encodes three viral coat proteins, VP1, VP2 and VP3, which are required for virion assembly. The construction of rAAV virions has been described, for instance, in U.S. Pat. Nos. 5,173,414; 5,139,941; 5,863,541; 5,869,305; 6,057,152; and 6,376,237; as well as in Rabinowitz et al., J. Virol. 76:791 (2002) and Bowles et al., J. Virol. 77:423 (2003), the disclosures of each of which are incorporated herein by reference as they pertain to AAV vectors for gene delivery.

rAAV virions useful in conjunction with the compositions and methods described herein include those derived from a variety of AAV serotypes including AAV 1, 2, 3, 4, 5, 6, 7, 8, 9, 10, 11, rh10, rh39, rh43, rh74, Anc80, Anc80L65, DJ, DJ/8, DJ/9, 7m8, PHP.B, PHP.eb, and PHP.S. For targeting GJB2-expressing cells, AAV1, AAV2, AAV2quad(Y-F), AAV6, AAV8, AAV9, Anc80, Anc80L65, AAV-DJ, AAV-DJ/9, 7m8, and PHP.B may be particularly useful. Serotypes evolved for transduction of the retina may also be used in the methods and compositions described herein. Construction and use of AAV vectors and AAV proteins of different serotypes are described, for instance, in Chao et al., Mol. Ther. 2:619 (2000); Davidson et al., Proc. Natl. Acad. Sci. USA 97:3428 (2000); Xiao et al., J. Virol. 72:2224 (1998); Halbert et al., J. Virol. 74:1524 (2000); Halbert et al., J. Virol. 75:6615 (2001); and Auricchio et al., Hum. Molec. Genet. 10:3075 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype (e.g., AAV9) pseudotyped with a capsid gene derived from a serotype other than the given serotype (e.g., AAV1, AAV2, AAV2quad(Y-F), AAV3, AAV4, AAV5, AAV6, AAV7, AAV8, etc.). Techniques involving the construction and use of pseudotyped rAAV virions are known in the art and are described, for instance, in Duan et al., J. Virol. 75:7662 (2001); Halbert et al., J. Virol. 74:1524 (2000); Zolotukhin et al., Methods, 28:158 (2002); and Auricchio et al., Hum. Molec. Genet. 10:3075 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635 (2000). Other rAAV virions that can be used in methods described herein include those capsid hybrids that are generated by molecular breeding of viruses as well as by exon shuffling. See, e.g., Soong et al., Nat. Genet., 25:436 (2000) and Kolman and Stemmer, Nat. Biotechnol. 19:423 (2001).

### Pharmaceutical Compositions

The nucleic acid vectors described herein (e.g., a vector containing a GJB2 regulatory construct described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more sequence identity) to SEQ ID NO: 1 or SEQ ID NO: 2) operably linked to a polynucleotide encoding Gjb2 (e.g., wild-type human Gjb2, such as a polynucleotide having least 90% sequence identity (e.g., 90%, 91%, 92%, 93%, 94%, 95%, 96%, 97%, 98%, 99%, or more sequence identity) to the sequence of SEQ ID NO: 3 and containing at least 50% fewer CG dinucleotides (e.g., 50%, 60%, 70%, 75%, 80%, 85%, 90%, 95%, or fewer CG dinucleotides) than wild-type GJB2 or a polynucleotide having at least 90% sequence identity (e.g., 90%, 91%, 92%, 93%, 94%, 95%, 96%, 97%, 98%, 99%, or more sequence identity) to the sequence of SEQ ID NO: 4 or SEQ ID NO: 28) may be incorporated into a vehicle for administration into a patient, such as a human patient suffering from sensorineural hearing loss (e.g., GJB2-related hearing loss). Pharmaceutical compositions containing vectors, such as viral vectors, that contain a GJB2 regulatory construct described herein operably linked to a polynucleotide encoding Gjb2 can be prepared using methods known in the art. For example, such compositions can be prepared using, e.g., physiologically acceptable carriers, excipients, or stabilizers (Remington: The Science and Practice of Pharmacology 22nd edition, Allen, L. Ed. (2013); incorporated herein by reference), and in a desired form, e.g., in the form of lyophilized formulations or aqueous solutions.

Mixtures of nucleic acid vectors (e.g., viral vectors) containing a GJB2 regulatory construct described herein operably linked to a polynucleotide encoding Gjb2 may be prepared in water suitably mixed with one or more excipients, carriers, or diluents. Dispersions may also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (described in U.S. Pat. No. 5,466,468, the disclosure of which is incorporated herein by reference). In any case the formulation may be sterile and may be fluid to the extent that easy syringability exists. Formulations may be stable under the conditions of manufacture and storage and may be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For example, a solution containing a pharmaceutical composition described herein may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the subject being treated. For local administration to the inner ear, the composition may be formulated to contain a synthetic perilymph solution. An exemplary synthetic perilymph solution includes 20-200 mM NaCl, 1-5 mM KCl, 0.1-10 mM CaCl₂), 1-10 mM glucose, and 2-50 mM HEPEs, with a pH between about 6 and 9 and an osmolality of about 300 mOsm/kg. The person responsible for administration will, in any event, determine the appropriate dose for the individual subject. Moreover, for human administration, preparations may meet sterility, pyrogenicity, general safety, and purity standards as required by FDA Office of Biologics standards.

### Methods of Treatment

The compositions described herein may be administered to a subject having or at risk of developing sensorineural hearing loss (e.g., GJB2-related hearing loss) by a variety of routes, such as local administration to the middle or inner ear (e.g., administration into the perilymph or endolymph, such as to or through the oval window, round window, or semicircular canal (e.g., the horizontal canal), or by transtympanic or intratympanic injection, e.g., administration to a GJB2-expressing inner ear cell), intravenous, parenteral, intradermal, transdermal, intramuscular, intranasal, subcutaneous, percutaneous, intratracheal, intraperitoneal, intraarterial, intravascular, inhalation, perfusion, lavage, and oral administration. The most suitable route for administration in any given case will depend on the particular composition administered, the patient, pharmaceutical formulation methods, administration methods (e.g., administration time and administration route), the patient's age, body weight, sex, severity of the disease being treated, the patient's diet, and the patient's excretion rate. Compositions may be administered once, or more than once (e.g., once annually, twice annually, three times annually, bi-monthly, monthly, or bi-weekly).

Subjects that may be treated as described herein are subjects having or at risk of developing sensorineural hearing loss. In some embodiments, the compositions described herein are used to treat GJB2-related hearing loss (e.g., DFNB1 or DFNA3, or hearing loss associated with Bart-Pumphrey syndrome, hystrix-like ichthyosis with deafness, keratitis-ichthyosis-deafness syndrome, palmoplantar keratoderma with deafness, or Vohwinkel syndrome). GJB2-related hearing loss, such as DFNB1 or DFNA3, can be treated by administration of a nucleic acid vector containing a GJB2 regulatory construct described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more sequence identity) to SEQ ID NO: 1 or SEQ ID NO: 2) operably linked to a polynucleotide encoding Gjb2 (e.g., a polynucleotide encoding wild-type human Gjb2, such as a polynucleotide having least 90% sequence identity (e.g., 90%, 91%, 92%, 93%, 94%, 95%, 96%, 97%, 98%, 99%, or more sequence identity) to the sequence of SEQ ID NO: 3 and containing at least 50% fewer CG dinucleotides (e.g., 50%, 60%, 70%, 75%, 80%, 85%, 90%, 95%, or fewer CG dinucleotides) than wild-type GJB2 or a polynucleotide having at least 90% sequence identity (e.g., 90%, 91%, 92%, 93%, 94%, 95%, 96%, 97%, 98%, 99%, or more sequence identity) to the sequence of SEQ ID NO: 4 or SEQ ID NO: 28). The subject may have or be identified as having a mutation in GJB2 and/or GJB6 (e.g., a mutation associated with GJB2-related hearing loss, such as a mutation that disrupts GJB2 expression or Gjb2 function) and may have severe, moderate, or mild hearing loss when treatment is initiated or may be treated prior to symptom onset (e.g., preventative treatment).

The methods described herein may include a step of screening a subject for one or more mutations in genes known to be associated with GJB2-related hearing loss (e.g., one or more mutations in GJB2 and/or GJB6) prior to treatment with or administration of the compositions described herein. A subject can be screened for a genetic mutation using standard methods known to those of skill in the art (e.g., genetic testing). The methods described herein may also include a step of assessing hearing in a subject prior to treatment with or administration of the compositions described herein. Hearing can be assessed using standard tests, such as audiometry, auditory brainstem response (ABR), electrocochleography (ECOG), and otoacoustic emissions (DPOAE). These tests can also be used to assess hearing in a subject after treatment with or administration of the compositions described herein.

Treatment may include administration of a composition containing a nucleic acid vector (e.g., an AAV vector) containing a GJB2 regulatory construct operably linked to a polynucleotide encoding Gjb2 described herein in various unit doses. Each unit dose will ordinarily contain a predetermined quantity of the therapeutic composition. The quantity to be administered, and the particular route of administration and formulation, are within the skill of those in the clinical arts. A unit dose need not be administered as a single injection but may comprise continuous infusion over a set period of time. Dosing may be performed using a syringe pump to control infusion rate in order to minimize damage to the inner ear (e.g., the cochlea and/or vestibular system). In cases in which the nucleic acid vectors are AAV vectors (e.g., AAV1, AAV2, AAV2quad(Y-F), AAV3, AAV4, AAV5, AAV6, AAV7, AAV8, AAV9, AAV10, rh10, rh39, rh43, rh74, Anc80, Anc80L65, DJ, DJ/8, DJ/9, 7m8, PHP.B, PHP.eb, or PHP.S vectors), the viral vectors may be administered to the patient at a dose of, for example, from about 1×10⁹ vector genomes (VG)/mL to about 1×10¹⁶ VG/mL (e.g., 1×10⁹ VG/mL, 2×10⁹ VG/mL, 3×10⁹ VG/mL, 4×10⁹ VG/mL, 5×10⁹ VG/mL, 6×10⁹ VG/mL, 7×10⁹ VG/mL, 8×10⁹ VG/mL, 9×10⁹ VG/mL, 1×10¹⁰ VG/mL, 2×10¹⁰ VG/mL, 3×10¹⁰ VG/mL, 4×10¹⁰ VG/mL, 5×10¹⁰ VG/mL, 6×10¹⁰ VG/mL, 7×10¹⁰ VG/mL, 8×10¹⁰ VG/mL, 9×10¹⁰ VG/mL, 1×10¹¹ VG/mL, 2×10¹¹ VG/mL, 3×10¹¹ VG/mL, 4×10¹¹ VG/mL, 5×10¹¹ VG/mL, 6×10¹¹ VG/mL, 7×10¹¹ VG/mL, 8×10¹¹ VG/mL, 9×10¹¹ VG/mL, 1×10¹² VG/mL, 2×10¹² VG/mL, 3×10¹² VG/mL, 4×10¹² VG/mL, 5×10¹² VG/mL, 6×10¹² VG/mL, 7×10¹² VG/mL, 8×10¹² VG/mL, 9×10¹² VG/mL, 1×10¹³ VG/mL, 2×10¹³ VG/mL, 3×10¹³ VG/mL, 4×10¹³ VG/mL, 5×10¹³ VG/mL, 6×10¹³ VG/mL, 7×10¹³ VG/mL, 8×10¹³ VG/mL, 9×10¹³ VG/mL, 1×10¹⁴ VG/mL, 2×10¹⁴ VG/mL, 3×10¹⁴ VG/mL, 4×10¹⁴ VG/mL, 5×10¹⁴ VG/mL, 6×10¹⁴ VG/mL, 7×10¹⁴ VG/mL, 8×10¹⁴ VG/mL, 9×10¹⁴ VG/mL, 1×10¹⁵ VG/mL, 2×10¹⁵ VG/mL, 3×10¹⁵ VG/mL, 4×10¹⁵ VG/mL, 5×10¹⁵ VG/mL, 6×10¹⁵ VG/mL, 7×10¹⁵ VG/mL, 8×10¹⁵ VG/mL, 9×10¹⁵ VG/mL, or 1×10¹⁶ VG/mL) in a volume of 1 μL to 200 μL (e.g., 1, 2, 3, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or 200 μL). The AAV vectors may be administered to the subject at a dose of about 1×10⁷ VG/ear to about 2×10¹⁵ VG/ear (e.g., 1×10⁷ VG/ear, 2×10⁷ VG/ear, 3×10⁷ VG/ear, 4×10⁷ VG/ear, 5×10⁷ VG/ear, 6×10⁷ VG/ear, 7×10⁷ VG/ear, 8×10⁷ VG/ear, 9×10⁷ VG/ear, 1×10⁸ VG/ear, 2×10⁸ VG/ear, 3×10⁸ VG/ear, 4×10⁸ VG/ear, 5×10⁸ VG/ear, 6×10⁸ VG/ear, 7×10⁸ VG/ear, 8×10⁸ VG/ear, 9×10⁸ VG/ear, 1×10⁹ VG/ear, 2×10⁹ VG/ear, 3×10⁹ VG/ear, 4×10⁹ VG/ear, 5×10⁹ VG/ear, 6×10⁹ VG/ear, 7×10⁹ VG/ear, 8×10⁹ VG/ear, 9×10⁹ VG/ear, 1×10¹⁰ VG/ear, 2×10¹⁰ VG/ear, 3×10¹⁰ VG/ear, 4×10¹⁰ VG/ear, 5×10¹⁰ VG/ear, 6×10¹⁰ VG/ear, 7×10¹⁰ VG/ear, 8×10¹⁰ VG/ear, 9×10¹⁰ VG/ear, 1×10¹¹ VG/ear, 2×10¹¹ VG/ear, 3×10¹ VG/ear, 4×10¹ VG/ear, 5×10¹ VG/ear, 6×10¹¹ VG/ear, 7×10¹ VG/ear, 8×10¹¹ VG/ear, 9×10¹ VG/ear, 1×10¹² VG/ear, 2×10¹² VG/ear, 3×10¹² VG/ear, 4×10¹² VG/ear, 5×10¹² VG/ear, 6×10¹² VG/ear, 7×10¹² VG/ear, 8×10¹² VG/ear, 9×10¹² VG/ear, 1×10¹³ VG/ear, 2×10¹³ VG/ear, 3×10¹³ VG/ear, 4×10¹³ VG/ear, 5×10¹³ VG/ear, 6×10¹³ VG/ear, 7×10¹³ VG/ear, 8×10¹³ VG/ear, 9×10¹³ VG/ear, 1×10¹⁴ VG/ear, 2×10¹⁴ VG/ear, 3×10¹⁴ VG/ear, 4×10¹⁴ VG/ear, 5×10¹⁴ VG/ear, 6×10¹⁴ VG/ear, 7×10¹⁴ VG/ear, 8×10¹⁴ VG/ear, 9×10¹⁴ VG/ear, 1×10¹⁵ VG/ear, or 2×10¹⁵ VG/ear).

The compositions described herein are administered in an amount sufficient to improve hearing, increase or induce expression of wild-type Gjb2 in GJB2-expressing cells (e.g., cochlear supporting cells), increase or improve Gjb2 function, promote or increase cochlear supporting cell survival, or improve cochlear supporting cell function and/or structure. Hearing may be evaluated using standard hearing tests (e.g., audiometry, ABR, electrocochleography (ECOG), and otoacoustic emissions) and may be improved by 5% or more (e.g., 5%, 10%, 15%, 20%, 30%, 40%, 50%, 60%, 70%, 80%, 90%, 100%, 125%, 150%, 200% or more) compared to hearing measurements obtained prior to treatment. In some embodiments, the compositions are administered in an amount sufficient to improve the subject's ability to understand speech. The compositions described herein may also be administered in an amount sufficient to slow or prevent the development or progression of sensorineural hearing loss (e.g., in subjects who carry a genetic mutation associated with GJB2-related hearing loss but do not exhibit hearing impairment, or in subjects exhibiting only mild to moderate hearing loss at the time of treatment initiation). GJB2 expression may be evaluated using immunohistochemistry, Western blot analysis, quantitative real-time PCR, or other methods known in the art for detection protein or mRNA, and may be increased by 5% or more (e.g., 5%, 10%, 15%, 20%, 30%, 40%, 50%, 60%, 70%, 80%, 90%, 100%, 125%, 150%, 200% or more) compared to expression prior to administration of the compositions described herein. Cochlear supporting cell function and/or Gjb2 function may be evaluated indirectly based on hearing tests, and may be increased by 5% or more (e.g., 5%, 10%, 15%, 20%, 30%, 40%, 50%, 60%, 70%, 80%, 90%, 100%, 125%, 150%, 200% or more) compared to cochlear supporting cell function and/or Gjb2 function prior to administration of the compositions described herein. The compositions and methods described herein may also reduce the toxicity associated with administration of a nucleic acid vector compared to the toxicity observed after the administration of a nucleic acid vector that does not contain a GJB2 regulatory construct described herein (e.g., administration of a nucleic acid vector in which a polynucleotide encoding Gjb2 is expressed using a ubiquitous promoter). These effects may occur, for example, within 1 week, 2 weeks, 3 weeks, 4 weeks, 5 weeks, 6 weeks, 7 weeks, 8 weeks, 9 weeks, 10 weeks, 15 weeks, 20 weeks, 25 weeks, or more, following administration of the compositions described herein. The patient may be evaluated 1 month, 2 months, 3 months, 4 months, 5 months, 6 months, or more following administration of the composition depending on the dose and route of administration used for treatment. Depending on the outcome of the evaluation, the patient may receive additional treatments.

### Kits

The compositions described herein can be provided in a kit for use in treating sensorineural hearing loss (e.g., GJB2-related hearing loss). Compositions may include a polynucleotide containing a GJB2 regulatory construct described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more sequence identity) to SEQ ID NO: 1 or SEQ ID NO: 2) operably linked to a polynucleotide encoding Gjb2 (e.g., a polynucleotide encoding wild-type human Gjb2, such as a polynucleotide having least 90% sequence identity (e.g., 90%, 91%, 92%, 93%, 94%, 95%, 96%, 97%, 98%, 99%, or more sequence identity) to the sequence of SEQ ID NO: 3 and containing at least 50% fewer CG dinucleotides (e.g., 50%, 60%, 70%, 75%, 80%, 85%, 90%, 95%, or fewer CG dinucleotides) than wild-type GJB2 or a polynucleotide having at least 90% sequence identity (e.g., 90%, 91%, 92%, 93%, 94%, 95%, 96%, 97%, 98%, 99%, or more sequence identity) to the sequence of SEQ ID NO: 4 or SEQ ID NO: 28) or a nucleic acid vector containing such a polynucleotide. The nucleic acid vector may be packaged in an AAV virus capsid (e.g., AAV1, AAV2, AAV2quad(Y-F), AAV6, AAV8, AAV9, Anc80, Anc80L65, AAV-DJ, DJ/9, 7m8, or PHP.B). The kit can further include a package insert that instructs a user of the kit, such as a physician, to perform the methods described herein. The kit may optionally include a syringe or other device for administering the composition.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1. Western Blot for Expression of Flag-Tagged hGJB2 Codon Variants

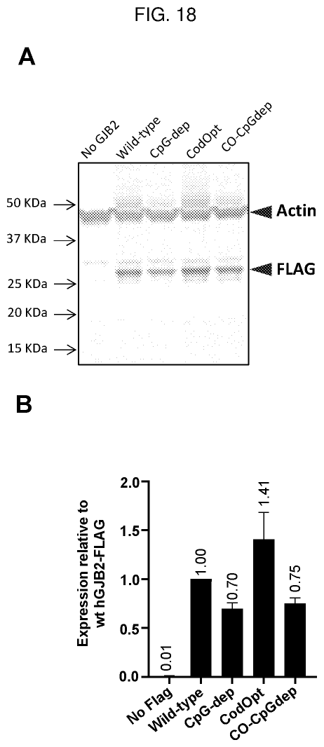

Coding sequence variants for human GJB2 (hGJB2), including wild-type (SEQ ID NO: 5), CpG-depleted (CpG-dep; SEQ ID NO: 3), codon-optimized (CodOpt; SEQ ID NO: 28), and CpG-depleted codon-optimized (CO-CpG-dep; SEQ ID NO: 4) were cloned into plasmids with the same C-terminal 3×FLAG tag sequence, under control of a CMV promoter. One microgram of each plasmid was transfected into a different well of a 6-well plate with 6×10⁵ HeLa cells using Lipofectamine 3000 (Invitrogen L3000-015). Transfected cells were incubated at 37° C. for 72 hours. Cells were then harvested, and cell lysates were run on a western blot. An anti-Flag antibody was used to detect the hGJB2-3×Flag proteins, and an anti-Actin antibody was used as an internal control (FIG. 18, panel A). Band intensity for hGJB2-3×Flag was measured and normalized to the Actin band intensity (FIG. 18, panel B). These results demonstrated modest reduction in hGJB2 levels in vitro with CpG depletion in both the wild-type and codon-optimized contexts.

### Example 2. In Vitro Propidium Iodide Uptake by Cells Expressing Wild-Type or Codon-Variant hGJB2

To compare Gjb2 function from proteins produced by different hGJB2 coding sequences, in vitro functional assays were performed. When expressed in HeLa cells, human Gjb2 forms hemichannels. These channels will remain closed in the presence of calcium ions but will open in the absence of calcium. Uptake of propidium iodide (PI) through the open hemichannels can be used to measure the functional activity of Gjb2 in vitro.

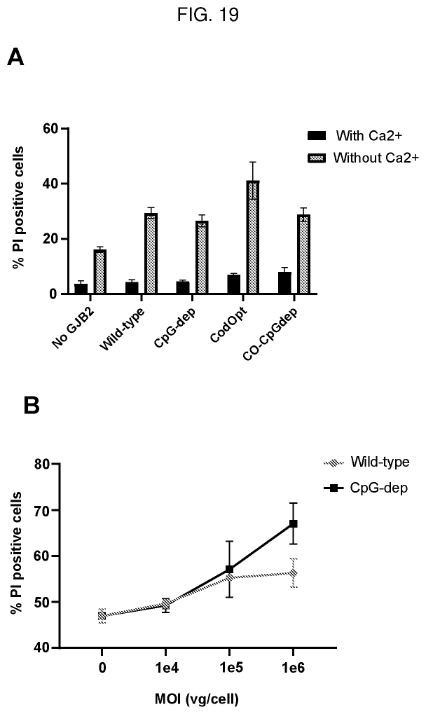

Coding sequence variants for hGJB2, including wild-type (SEQ ID NO: 5), CpG-depleted (CpG-dep; SEQ ID NO: 3), codon optimized (CodOpt; SEQ ID NO: 28), and CpG-depleted codon optimized (CO-CpG-dep; SEQ ID NO: 4) were cloned into plasmids under control of a CMV promoter. Two hundred nanograms of each plasmid was transfected into a different well of a 24-well plate with 1×10⁵ HeLa cells using Lipofectamine 3000 (Invitrogen L3000-015). Transfected cells were incubated at 37° C. for 72 hours. Media was then aspirated and replaced by PI buffered in Hanks' balanced salt solution (HBSS) with or without Ca²⁺. Final solutions were prepared as follows: Buffer with Ca²⁺: 1×HBSS (Thermo 14065056) and 0.1 mg/mL PI (Sigma P4864); Buffer without Ca²⁺: 1×HBSS without calcium (Thermo 14185052), 200 mM MgCl₂ (VWR 97062-850), 10 mM EGTA (Fisher 50-255-956), and 0.1 mg/mL PI. Cells were incubated in PI buffer for 40 minutes at room temperature. Buffer was then aspirated, and cells were harvested using TrypLE Express (Gibco 12605-010) and run on the Sony SH800 flow cytometer in analyzer mode using the 561 nm laser and the PE (600/60) filter. The percentage of cells that took up PI were graphed for each of the coding sequence variants (FIG. 19, panel A). All variants led to higher uptake of PI than the “no GJB2” condition. The two CpG-depleted versions were functionally similar to wild-type hGJB2, while the codon optimized version without CpG depletion resulted in increased PI uptake.

Wild-type (SEQ ID NO: 5) and CpG-depleted (SEQ ID NO: 3) hGJB2 genes driven by CMV were packaged into AAV1 vectors. HeLa cells were transduced with these vectors at different multiplicities of infection (MOIs): 1×10⁴, 1×10⁵, and 1×10⁶ viral genomes (vg) per cell into 2.5×10⁵ cells total. Transduced cells were incubated at 37° C. for 72 hours, then media was replaced with PI buffer without Ca²⁺ as previously described. Cells were incubated for 40 minutes at room temperature, then harvested and run on the SH800 flow cytometer as described above. Percentage of PI-positive cells were graphed at each MOI (FIG. 19, panel B). Both wild-type and CpG-dep vectors show an increase in PI-uptake at higher MOIs, with higher uptake by the CpG-dep version at MOI=1×10⁶ vg/cell.

### Example 3. Hearing Recovery Driven by CpG-Depleted and Wild-Type hGJB2 Transgenes in a GJB2 Deficiency Mouse Model

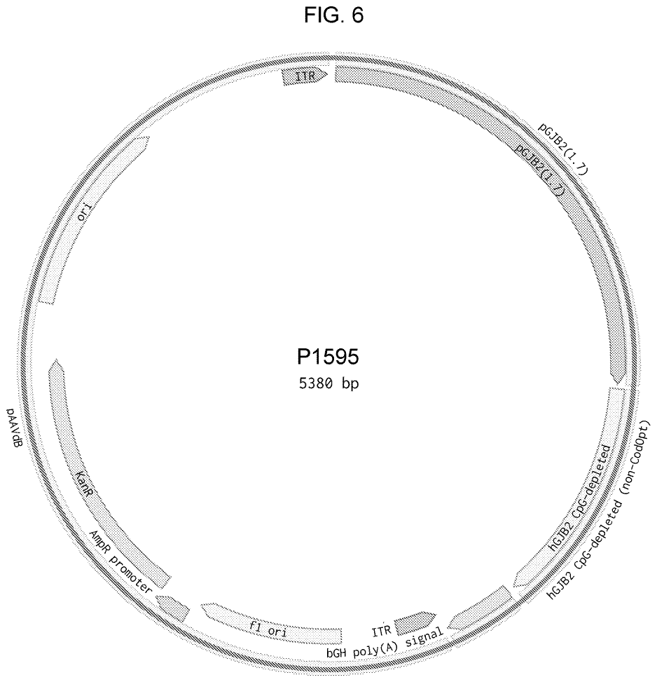

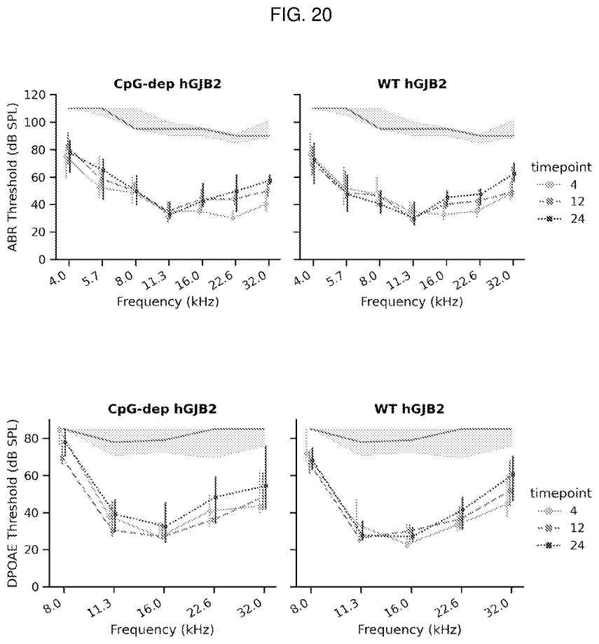

Neonatal GJB6-LacZ mice were injected unilaterally one to three days after birth via fenestration in the posterior semicircular canal with one microliter of AAV1 vector created from transgene plasmid P1595 (FIG. 6; SEQ ID NO: 16) containing CpG-dep hGJB2 driven by a GJB2 regulatory construct, or a corresponding transgene plasmid containing wild-type hGJB2 driven by the same GJB2 regulatory construct. To test for hearing recovery, animals were anesthetized with ketamine and xylazine and the auditory brainstem response (ABR) and distortion product otoacoustic emission (DPOAE) were measured in both ears (injected and uninjected). Hearing recovery was tested at multiple time points after treatment, from 4 weeks to 24 weeks. Level of hearing recovery, as well as durability, were comparable between both treatment groups (FIG. 20), indicating that efficacy driven by wild-type and CpG-depleted transcripts is equivalent.

### Example 4. Hearing Recovery Using a CpG-Depleted hGJB2 Transgene Follows a Clear Dose Response

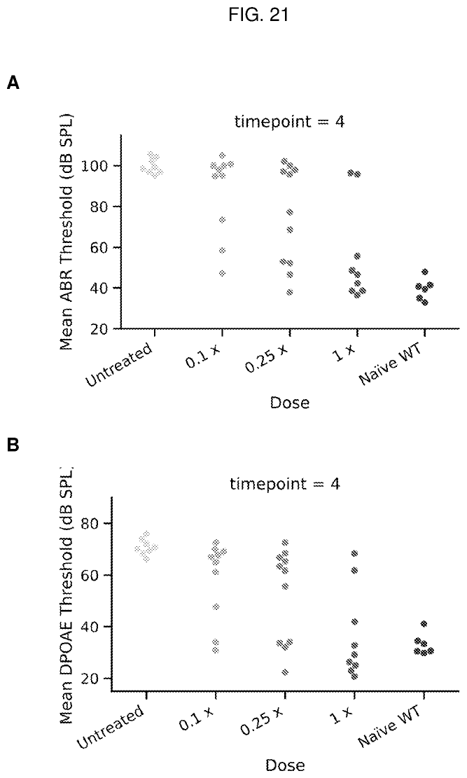

Neonatal GJB6-LacZ mice were injected unilaterally with one microliter of 1×, 0.25×, or 0.1× dose of an AAV1 vector created from transgene plasmid P1595 (FIG. 6; SEQ ID NO: 16). Surgical injection route and timing was as described in Example 3. Similar to Example 3, auditory brainstem response and distortion product otoacoustic emission were measured four weeks after treatment. Results indicated a clear dose response, with the number of responders and magnitude of hearing recovery increasing with dose level (FIG. 21).

### Example 5. The bGH polyA Signal Sequence Yields Higher Gene Expression than the SV40 polyA Signal Sequence In Vitro

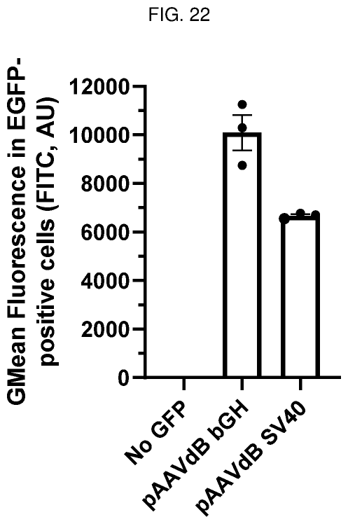

Either the bovine growth hormone polyadenylation (bGH_pA) signal (SEQ ID NO: 6) or the simian virus 40 polyadenylation (SV40_pA) signal (SEQ ID NO: 7) was inserted into plasmids downstream of a H2B-EGFP sequence driven by a CMV promoter (CMV.H2B-EGFP). Five hundred nanograms of each plasmid was transfected into a different well of a 24-well plate with 1×10⁵ HEK293T cells using Lipofectamine 3000 (Invitrogen L3000-015). Transfected cells were incubated at 37° C. for 48 hours. Cells were then harvested using TrypLE Express (Gibco 12605-010) and run on the Sony SH800 flow cytometer in analyzer mode using the 488 nm laser and the FITC (525/50) filter. The geometric mean of fluorescence in the EGFP-positive cells was measured and graphed (FIG. 22). Using the bGH_pA signal sequence resulted in a higher mean expression of EGFP driven by CMV than the SV40_pA signal sequence in vitro.

### Example 6. PAAVdB Backbone Functions Similarly to pAAVKan In Vitro

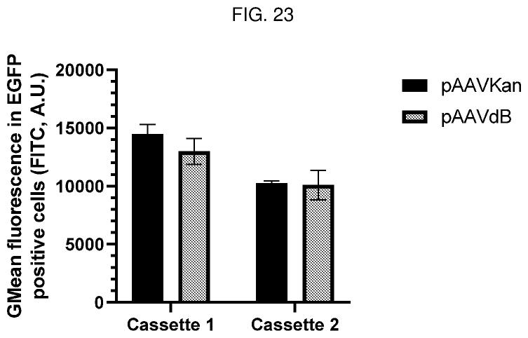

Two different CMV.H2B-EGFP.bGH_pA gene cassettes (sequences not shown), with different restriction enzyme cloning sites in the intragenic regions, were separately cloned into either the pAAVKan (the 3,157 nucleotides spanning nucleotides 2454-5420 and 1-190 of SEQ ID NO: 20) or the pAAVdB (the 3,038 nucleotides spanning nucleotides 2399-5301 and 1-135 of SEQ ID NO: 12) ITR-containing plasmid backbones. Five hundred nanograms of each plasmid was transfected into a different well of a 24-well plate with 1×10⁵ HEK293T cells using Lipofectamine 3000 (Invitrogen L3000-015). Transfected cells were incubated at 37° C. for 48 hours. Cells were then harvested using TrypLE Express (Gibco 12605-010) and run on the Sony SH800 flow cytometer in analyzer mode using the 488 nm laser and the FITC (525/50) filter. The geometric mean of fluorescence in the EGFP-positive cells was measured and graphed (FIG. 23). While there was some variation between the GFP cassettes, the expression did not vary significantly between the two plasmid backbones.

### Example 7. Administration of a Composition Containing a Nucleic Acid Vector Described Herein to a Subject with GJB2-Related Hearing Loss

According to the methods disclosed herein, a physician of skill in the art can treat a patient, such as a human patient, with GJB2-related hearing loss so as to improve or restore hearing, or to slow or stop the progression of hearing loss. To this end, a physician of skill in the art can administer to the human patient a composition containing an AAV vector (e.g., an AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ, DJ/8, DJ/9, 7m8, PHP.B, PHP.eB, or PHP.S vector) containing a GJB2 regulatory construct described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more sequence identity) to SEQ ID NO: 1 or SEQ ID NO: 2) operably linked to a polynucleotide encoding a wild-type form of Gjb2 (e.g., a polynucleotide encoding the amino acid sequence of SEQ ID NO: 38, such as a polynucleotide having least 90% sequence identity (e.g., 90%, 91%, 92%, 93%, 94%, 95%, 96%, 97%, 98%, 99%, or more sequence identity) to the sequence of SEQ ID NO: 3 and containing at least 50% fewer CG dinucleotides (e.g., 50%, 60%, 70%, 75%, 80%, 85%, 90%, 95%, or fewer CG dinucleotides) than wild-type GJB2 or a polynucleotide having at least 90% sequence identity (e.g., 90%, 91%, 92%, 93%, 94%, 95%, 96%, 97%, 98%, 99%, or more sequence identity) to the sequence of SEQ ID NO: 4 or SEQ ID NO: 28). The composition containing the AAV vector may be administered to the patient, for example, by local administration to the inner ear (e.g., injection into the perilymph or through the round window membrane) to treat sensorineural hearing loss.

Following administration of the composition to a patient, a practitioner of skill in the art can monitor the patient's improvement in response to the therapy by a variety of methods. For example, a physician can monitor the patient's hearing by performing standard tests, such as audiometry, ABR, electrocochleography (ECOG), and otoacoustic emissions (DPOAE) following administration of the composition. A finding that the patient exhibits improved hearing in one or more of the tests following administration of the composition compared to hearing test results prior to administration of the composition indicates that the patient is responding favorably to the treatment. Subsequent doses can be determined and administered as needed.

Exemplary embodiments of the invention are described in the enumerated paragraphs below.
E1. A nucleic acid vector comprising, in 5′-to-3′ order:
   (a) a GJB2 regulatory construct having at least 90% sequence identity (e.g., 90%, 91%, 92%, 93%, 94%, 95%, 96%, 97%, 98%, 99%, or more sequence identity) to the sequence of SEQ ID NO: 1 or SEQ ID NO: 2 operably linked to:
   (b) a human GJB2 coding sequence selected from the group consisting of SEQ ID NO: 3, SEQ ID NO: 4, SEQ ID NO: 28, a sequence having at least 90% sequence identity (e.g., 90%, 91%, 92%, 93%, 94%, 95%, 96%, 97%, 98%, 99%, or more sequence identity) to SEQ ID NO: 3 and comprising at least 50% fewer CG dinucleotides (e.g., 50%, 60%, 70%, 75%, 80%, 85%, 90%, 95%, or fewer CG dinucleotides) than wild-type GJB2, and a sequence having at least 90% sequence identity (e.g., 90%, 91%, 92%, 93%, 94%, 95%, 96%, 97%, 98%, 99%, or more sequence identity) to SEQ ID NO: 4 or SEQ ID NO: 28 operably linked to:
   (c) a polyadenylation (polyA) signal sequence.
E2. The nucleic acid vector of E1, wherein the GJB2 regulatory construct has at least 90% sequence identity (e.g., 90%, 91%, 92%, 93%, 94%, 95%, 96%, 97%, 98%, 99%, or more sequence identity) to the sequence of SEQ ID NO: 1.
E3. The nucleic acid vector of E2, wherein the GJB2 regulatory construct has the sequence of SEQ ID NO: 1.
E4. The nucleic acid vector of E1, wherein the GJB2 regulatory construct has at least 90% sequence identity (e.g., 90%, 91%, 92%, 93%, 94%, 95%, 96%, 97%, 98%, 99%, or more sequence identity) to the sequence of SEQ ID NO: 2.
E5. The nucleic acid vector of E4, wherein the GJB2 regulatory construct has the sequence of SEQ ID NO: 2.
E6. The nucleic acid vector of any one of E1-E5, wherein the human GJB2 coding sequence has at least 90% sequence identity (e.g., 90%, 91%, 92%, 93%, 94%, 95%, 96%, 97%, 98%, 99%, or more sequence identity) to the sequence of SEQ ID NO: 3 and comprises at least 50% fewer CG dinucleotides (e.g., 50%, 60%, 70%, 75%, 80%, 85%, 90%, 95%, or fewer CG dinucleotides) than wild-type GJB2.
E7. The nucleic acid vector of E6, wherein the human GJB2 coding sequence has at least 90% sequence identity (e.g., 90%, 91%, 92%, 93%, 94%, 95%, 96%, 97%, 98%, 99%, or more sequence identity) to the sequence of SEQ ID NO: 3 and comprises no CG dinucleotides.
E8. The nucleic acid vector of E6 or E7, wherein the human GJB2 coding sequence has the sequence of SEQ ID NO: 3.
E9. The nucleic acid vector of any one of E1-E5, wherein the human GJB2 coding sequence has at least 90% sequence identity (e.g., 90%, 91%, 92%, 93%, 94%, 95%, 96%, 97%, 98%, 99%, or more sequence identity) to the sequence of SEQ ID NO: 4.
E10. The nucleic acid vector of E9, wherein the human GJB2 coding sequence has the sequence of SEQ ID NO: 4.
E11. The nucleic acid vector of any one of E1-E5, wherein the human GJB2 coding sequence has at least 90% sequence identity (e.g., 90%, 91%, 92%, 93%, 94%, 95%, 96%, 97%, 98%, 99%, or more sequence identity) to the sequence of SEQ ID NO: 28.
E12. The nucleic acid vector of E11, wherein the human GJB2 coding sequence has the sequence of SEQ ID NO: 28.
E13. The nucleic acid vector of any one of E1-E3 and E6-E8, wherein the GJB2 regulatory construct has the sequence of SEQ ID NO: 1 and the human GJB2 coding sequence has the sequence of SEQ ID NO: 3.
E14. The nucleic acid vector of any one of E1-E3, E9, and E10, wherein the GJB2 regulatory construct has the sequence of SEQ ID NO: 1 and the human GJB2 coding sequence has the sequence of SEQ ID NO: 4.
E15. The nucleic acid vector of any one of E1-E3, E11, and E12, wherein the GJB2 regulatory construct has the sequence of SEQ ID NO: 1 and the human GJB2 coding sequence has the sequence of SEQ ID NO: 28.
E16. The nucleic acid vector of any one of E1 and E4-E8, wherein the GJB2 regulatory construct has the sequence of SEQ ID NO: 2 and the human GJB2 coding sequence has the sequence of SEQ ID NO: 3.
E17. The nucleic acid vector of any one of E1, E4, E5, E9, and E10, wherein the GJB2 regulatory construct has the sequence of SEQ ID NO: 2 and the human GJB2 coding sequence has the sequence of SEQ ID NO: 4.
E18. The nucleic acid vector of any one of E1, E4, E5, E11, and E12, wherein the GJB2 regulatory construct has the sequence of SEQ ID NO: 2 and the human GJB2 coding sequence has the sequence of SEQ ID NO: 28.
E19. The nucleic acid vector of any one of E1-E18, wherein the polyA signal sequence has at least 90% sequence identity (e.g., 90%, 91%, 92%, 93%, 94%, 95%, 96%, 97%, 98%, 99%, or more sequence identity) to the sequence of SEQ ID NO: 6 or SEQ ID NO: 7.
E20. The nucleic acid vector of E19, wherein the polyA signal sequence has at least 90% sequence identity (e.g., 90%, 91%, 92%, 93%, 94%, 95%, 96%, 97%, 98%, 99%, or more sequence identity) to the sequence of SEQ ID NO: 6.
E21. The nucleic acid vector of E20, wherein the polyA signal sequence has the sequence of SEQ ID NO: 6.
E22. The nucleic acid vector of E19, wherein the polyA signal sequence has at least 90% sequence identity (e.g., 90%, 91%, 92%, 93%, 94%, 95%, 96%, 97%, 98%, 99%, or more sequence identity) to the sequence of SEQ ID NO: 7.
E23. The nucleic acid vector of E22, wherein the polyA signal sequence has the sequence of SEQ ID NO: 7.
E24. The nucleic acid vector of any one of E1-E23, further comprising a first polynucleotide having at least 90% sequence identity (e.g., 90%, 91%, 92%, 93%, 94%, 95%, 96%, 97%, 98%, 99%, or more sequence identity) to SEQ ID NO: 8 that comprises a first member of an ITR pair and is positioned 5′ of the GJB2 regulatory construct sequence and a second polynucleotide having at least 90% sequence identity (e.g., 90%, 91%, 92%, 93%, 94%, 95%, 96%, 97%, 98%, 99%, or more sequence identity) to SEQ ID NO: 9 that comprises a second member of an ITR pair and is positioned 3′ of the polyA signal sequence.
E25. The nucleic acid vector of E24, wherein the first polynucleotide has the sequence of SEQ ID NO: 8 and the second polynucleotide has the sequence of SEQ ID NO: 9.
E26. The nucleic acid vector of any one of E1-E23, further comprising a first polynucleotide having at least 90% sequence identity (e.g., 90%, 91%, 92%, 93%, 94%, 95%, 96%, 97%, 98%, 99%, or more sequence identity) to SEQ ID NO: 10 that comprises a first member of an ITR pair and is positioned 5′ of the GJB2 regulatory construct sequence and a second polynucleotide having at least 90% sequence identity (e.g., 90%, 91%, 92%, 93%, 94%, 95%, 96%, 97%, 98%, 99%, or more sequence identity) to SEQ ID NO: 11 that comprises a second member of an ITR pair and is positioned 3′ of the polyA signal sequence.
E27. The nucleic acid vector of E26, wherein the first polynucleotide has the sequence of SEQ ID NO: 8 and the second polynucleotide has the sequence of SEQ ID NO: 9.
E28. The nucleic acid vector of any one of E1-E27, wherein a stop codon is positioned 3′ of the GJB2 coding sequence (e.g., the stop codon is directly linked to the 3′ end of the GJB2 coding sequence).
E29. The nucleic acid vector of E1, wherein the vector comprises a polynucleotide sequence comprising nucleotides 157 to 2383 of SEQ ID NO: 12.
E30. The nucleic acid vector of E1 or E29, wherein the vector comprises a polynucleotide sequence comprising nucleotides 1 to 2531 of SEQ ID NO: 12.
E31. The nucleic acid vector of E1, wherein the vector comprises a polynucleotide sequence comprising nucleotides 157 to 2383 of SEQ ID NO: 13.
E32. The nucleic acid vector of E1 or E31, wherein the vector comprises a polynucleotide sequence comprising nucleotides 1 to 2531 of SEQ ID NO: 13.
E33. The nucleic acid vector of E1, wherein the vector comprises a polynucleotide sequence comprising nucleotides 157 to 3459 of SEQ ID NO: 14.
E34. The nucleic acid vector of E1 or E33, wherein the vector comprises a polynucleotide sequence comprising nucleotides 1 to 3607 of SEQ ID NO: 14.
E35. The nucleic acid vector of E1, wherein the vector comprises a polynucleotide sequence comprising nucleotides 157 to 3459 of SEQ ID NO: 15.
E36. The nucleic acid vector of E1 or E35, wherein the vector comprises a polynucleotide sequence comprising nucleotides 1 to 3607 of SEQ ID NO: 15.
E37. The nucleic acid vector of E1, wherein the vector comprises a polynucleotide sequence comprising nucleotides 157 to 2462 of SEQ ID NO: 16.
E38. The nucleic acid vector of E1 or E37, wherein the vector comprises a polynucleotide sequence comprising nucleotides 1 to 2610 of SEQ ID NO: 16.
E39. The nucleic acid vector of E1, wherein the vector comprises a polynucleotide sequence comprising nucleotides 157 to 2462 of SEQ ID NO: 17.
E40. The nucleic acid vector of E1 or E39, wherein the vector comprises a polynucleotide sequence comprising nucleotides 1 to 2610 of SEQ ID NO: 17.
E41. The nucleic acid vector of E1, wherein the vector comprises a polynucleotide sequence comprising nucleotides 157 to 3538 of SEQ ID NO: 18.
E42. The nucleic acid vector of E1 or E41, wherein the vector comprises a polynucleotide sequence comprising nucleotides 1 to 3686 of SEQ ID NO: 18.
E43. The nucleic acid vector of E1, wherein the vector comprises a polynucleotide sequence comprising nucleotides 157 to 3538 of SEQ ID NO: 19.
E44. The nucleic acid vector of E1 or E43, wherein the vector comprises a polynucleotide sequence comprising nucleotides 1 to 3686 of SEQ ID NO: 19.
E45. The nucleic acid vector of E1, wherein the vector comprises a polynucleotide sequence comprising nucleotides 212 to 2438 of SEQ ID NO: 20.
E46. The nucleic acid vector of E1 or E45, wherein the vector comprises a polynucleotide sequence comprising nucleotides 1 to 2650 of SEQ ID NO: 20.
E47. The nucleic acid vector of E1, wherein the vector comprises a polynucleotide sequence comprising nucleotides 212 to 2438 of SEQ ID NO: 21.
E48. The nucleic acid vector of E1 or E47, wherein the vector comprises a polynucleotide sequence comprising nucleotides 1 to 2650 of SEQ ID NO: 21.
E49. The nucleic acid vector of E1, wherein the vector comprises a polynucleotide sequence comprising nucleotides 212 to 3514 of SEQ ID NO: 22.
E50. The nucleic acid vector of E1 or E49, wherein the vector comprises a polynucleotide sequence comprising nucleotides 1 to 3726 of SEQ ID NO: 22.
E51. The nucleic acid vector of E1, wherein the vector comprises a polynucleotide sequence comprising nucleotides 212 to 3514 of SEQ ID NO: 23.
E52. The nucleic acid vector of E1 or E51, wherein the vector comprises a polynucleotide sequence comprising nucleotides 1 to 3726 of SEQ ID NO: 23.
E53. The nucleic acid vector of E1, wherein the vector comprises a polynucleotide sequence comprising nucleotides 212 to 2517 of SEQ ID NO: 24.
E54. The nucleic acid vector of E1 or E53, wherein the vector comprises a polynucleotide sequence comprising nucleotides 1 to 2729 of SEQ ID NO: 24.
E55. The nucleic acid vector of E1, wherein the vector comprises a polynucleotide sequence comprising nucleotides 212 to 2517 of SEQ ID NO: 25.
E56. The nucleic acid vector of E1 or E55, wherein the vector comprises a polynucleotide sequence comprising nucleotides 1 to 2729 of SEQ ID NO: 25.
E57. The nucleic acid vector of E1, wherein the vector comprises a polynucleotide sequence comprising nucleotides 212 to 3593 of SEQ ID NO: 26.
E58. The nucleic acid vector of E1 or E57, wherein the vector comprises a polynucleotide sequence comprising nucleotides 1 to 3805 of SEQ ID NO: 26.
E59. The nucleic acid vector of E1, wherein the vector comprises a polynucleotide sequence comprising nucleotides 212 to 3593 of SEQ ID NO: 27.
E60. The nucleic acid vector of E1 or E59, wherein the vector comprises a polynucleotide sequence comprising nucleotides 1 to 3805 of SEQ ID NO: 27.
E61. A polynucleotide comprising a human GJB2 coding sequence selected from the group consisting of SEQ ID NO: 3, SEQ ID NO: 4, SEQ ID NO: 28, a sequence having at least 90% sequence identity (e.g., 90%, 91%, 92%, 93%, 94%, 95%, 96%, 97%, 98%, 99%, or more sequence identity) to SEQ ID NO: 3 and comprising at least 50% fewer CG dinucleotides (e.g., 50%, 60%, 70%, 75%, 80%, 85%, 90%, 95%, or fewer CG dinucleotides) than wild-type GJB2, and a sequence having at least 90% sequence identity (e.g., 90%, 91%, 92%, 93%, 94%, 95%, 96%, 97%, 98%, 99%, or more sequence identity) to SEQ ID NO: 28.
E62. The polynucleotide of E61, wherein the human GJB2 coding sequence has at least 90% sequence identity (e.g., 90%, 91%, 92%, 93%, 94%, 95%, 96%, 97%, 98%, 99%, or more sequence identity) to the sequence of SEQ ID NO: 3 and comprises at least 50% fewer CG dinucleotides (e.g., 50%, 60%, 70%, 75%, 80%, 85%, 90%, 95%, or fewer CG dinucleotides) than wild-type GJB2.
E63. The polynucleotide of E62, wherein the human GJB2 coding sequence has at least 90% sequence identity (e.g., 90%, 91%, 92%, 93%, 94%, 95%, 96%, 97%, 98%, 99%, or more sequence identity) to the sequence of SEQ ID NO: 3 and comprises no CG dinucleotides.
E64. The polynucleotide of E62 or E63, wherein the human GJB2 coding sequence has the sequence of SEQ ID NO: 3.
E65. The polynucleotide of E61, wherein the human GJB2 coding sequence has at least 90% sequence identity (e.g., 90%, 91%, 92%, 93%, 94%, 95%, 96%, 97%, 98%, 99%, or more sequence identity) to the sequence of SEQ ID NO: 4.
E66. The polynucleotide of E65, wherein the human GJB2 coding sequence has the sequence of SEQ ID NO: 4.
E67. The polynucleotide of E61, wherein the human GJB2 coding sequence has at least 90% sequence identity (e.g., 90%, 91%, 92%, 93%, 94%, 95%, 96%, 97%, 98%, 99%, or more sequence identity) to the sequence of SEQ ID NO: 28.
E68. The polynucleotide of E67, wherein the human GJB2 coding sequence has the sequence of SEQ ID NO: 28.
E69. The polynucleotide of any one of E61-E68, wherein a stop codon is positioned 3′ of the GJB2 coding sequence (e.g., the stop codon is directly linked to the 3′ end of the GJB2 coding sequence).
E70. The polynucleotide of any one of E61-E69, wherein a GJB2 promoter is operably linked to the GJB2 coding sequence.
E71. The polynucleotide of E70, wherein the GJB2 promoter is positioned 5′ of the GJB2 coding sequence.
E72. The polynucleotide of E70 or E71, wherein the GJB2 promoter is contained in a GJB2 regulatory construct.
E73. The polynucleotide of E72, wherein the GJB2 regulatory construct has at least 90% sequence identity (e.g., 90%, 91%, 92%, 93%, 94%, 95%, 96%, 97%, 98%, 99%, or more sequence identity) to the sequence of SEQ ID NO: 1 or SEQ ID NO: 2.
E74. The polynucleotide of 73, wherein the GJB2 regulatory construct has at least 90% sequence identity (e.g., 90%, 91%, 92%, 93%, 94%, 95%, 96%, 97%, 98%, 99%, or more sequence identity) to the sequence of SEQ ID NO: 1.
E75. The polynucleotide of E74, wherein the GJB2 regulatory construct has the sequence of SEQ ID NO: 1.
E76. The polynucleotide of E75, wherein the GJB2 regulatory construct has the sequence of SEQ ID NO: 1 and the human GJB2 coding sequence has the sequence of SEQ ID NO: 3.
E77. The polynucleotide of E75, wherein the GJB2 regulatory construct has the sequence of SEQ ID NO: 1 and the human GJB2 coding sequence has the sequence of SEQ ID NO: 4.
E78. The polynucleotide of E75, wherein the GJB2 regulatory construct has the sequence of SEQ ID NO: 1 and the human GJB2 coding sequence has the sequence of SEQ ID NO: 28.
E79. The polynucleotide of E73, wherein the GJB2 regulatory construct has at least 90% sequence identity (e.g., 90%, 91%, 92%, 93%, 94%, 95%, 96%, 97%, 98%, 99%, or more sequence identity) to the sequence of SEQ ID NO: 2.
E80. The polynucleotide of E79, wherein the GJB2 regulatory construct has the sequence of SEQ ID NO: 2.
E81. The polynucleotide of E80, wherein the GJB2 regulatory construct has the sequence of SEQ ID NO: 2 and the human GJB2 coding sequence has the sequence of SEQ ID NO: 3.
E82. The polynucleotide of E80, wherein the GJB2 regulatory construct has the sequence of SEQ ID NO: 2 and the human GJB2 coding sequence has the sequence of SEQ ID NO: 4.
E83. The polynucleotide of E80, wherein the GJB2 regulatory construct has the sequence of SEQ ID NO: 2 and the human GJB2 coding sequence has the sequence of SEQ ID NO: 28.
E84. The polynucleotide of any one of E61-E83, wherein the GJB2 coding sequence is operably linked to a polyA signal sequence.
E85. The polynucleotide of E84, wherein the polyA signal sequence is positioned 3′ of the GJB2 coding sequence.
E86. The polynucleotide of E84 or E85, wherein the polyA signal sequence has at least 90% sequence identity (e.g., 90%, 91%, 92%, 93%, 94%, 95%, 96%, 97%, 98%, 99%, or more sequence identity) to the sequence of SEQ ID NO: 6 or SEQ ID NO: 7.
E87. The polynucleotide of E86, wherein the polyA signal sequence has at least 90% sequence identity (e.g., 90%, 91%, 92%, 93%, 94%, 95%, 96%, 97%, 98%, 99%, or more sequence identity) to the sequence of SEQ ID NO: 6.
E88. The polynucleotide of E87, wherein the polyA signal sequence has the sequence of SEQ ID NO: 6.
E89. The polynucleotide of E86, wherein the polyA signal sequence has at least 90% sequence identity (e.g., 90%, 91%, 92%, 93%, 94%, 95%, 96%, 97%, 98%, 99%, or more sequence identity) to the sequence of SEQ ID NO: 7.
E90. The polynucleotide of E89, wherein the polyA signal sequence has the sequence of SEQ ID NO: 7.
E91. The polynucleotide of any one of E71-E90, further comprising a first polynucleotide having at least 90% sequence identity (e.g., 90%, 91%, 92%, 93%, 94%, 95%, 96%, 97%, 98%, 99%, or more sequence identity) to SEQ ID NO: 8 that comprises a first member of an ITR pair and is positioned 5′ of the GJB2 promoter or the GJB2 regulatory construct sequence and a second polynucleotide having at least 90% sequence identity (e.g., 90%, 91%, 92%, 93%, 94%, 95%, 96%, 97%, 98%, 99%, or more sequence identity) to SEQ ID NO: 9 that comprises a second member of an ITR pair and is positioned 3′ of the polyA signal sequence.
E92. The polynucleotide of E91, wherein the first polynucleotide has the sequence of SEQ ID NO: 8 and the second polynucleotide has the sequence of SEQ ID NO: 9.
E93. The polynucleotide of any one of E71-E90, further comprising a first polynucleotide having at least 90% sequence identity (e.g., 90%, 91%, 92%, 93%, 94%, 95%, 96%, 97%, 98%, 99%, or more sequence identity) to SEQ ID NO: 10 that comprises a first member of an ITR pair and is positioned 5′ of the GJB2 promoter or the GJB2 regulatory construct sequence and a second polynucleotide having at least 90% sequence identity (e.g., 90%, 91%, 92%, 93%, 94%, 95%, 96%, 97%, 98%, 99%, or more sequence identity) to SEQ ID NO: 11 that comprises a second member of an ITR pair and is positioned 3′ of the polyA signal sequence.
E94. The polynucleotide of E93, wherein the first polynucleotide has the sequence of SEQ ID NO: 8 and the second polynucleotide has the sequence of SEQ ID NO: 9.
E95. The polynucleotide of E61 or E73, wherein the polynucleotide comprises a polynucleotide sequence comprising nucleotides 157 to 2383 of SEQ ID NO: 12.
E96. The polynucleotide of E61, E73, or E95, wherein the polynucleotide comprises a polynucleotide sequence comprising nucleotides 1 to 2531 of SEQ ID NO: 12.
E97. The polynucleotide of E61 or E73, wherein the polynucleotide comprises a polynucleotide sequence comprising nucleotides 157 to 2383 of SEQ ID NO: 13.
E98. The polynucleotide of E61, E73, or E97, wherein the polynucleotide comprises a polynucleotide sequence comprising nucleotides 1 to 2531 of SEQ ID NO: 13.
E99. The polynucleotide of E61 or E73, wherein the polynucleotide comprises a polynucleotide sequence comprising nucleotides 157 to 3459 of SEQ ID NO: 14.
E100. The polynucleotide of E61, E73, or E99, wherein the polynucleotide comprises a polynucleotide sequence comprising nucleotides 1 to 3607 of SEQ ID NO: 14.
E101. The polynucleotide of E61 or E73, wherein the polynucleotide comprises a polynucleotide sequence comprising nucleotides 157 to 3459 of SEQ ID NO: 15.
E102. The polynucleotide of E61, E73, or E101, wherein the polynucleotide comprises a polynucleotide sequence comprising nucleotides 1 to 3607 of SEQ ID NO: 15.
E103. The polynucleotide of E61 or E73, wherein the polynucleotide comprises a polynucleotide sequence comprising nucleotides 157 to 2462 of SEQ ID NO: 16.
E104. The polynucleotide of E61, E73, or E103, wherein the polynucleotide comprises a polynucleotide sequence comprising nucleotides 1 to 2610 of SEQ ID NO: 16.
E105. The polynucleotide of E61 or E73, wherein the polynucleotide comprises a polynucleotide sequence comprising nucleotides 157 to 2462 of SEQ ID NO: 17.
E106. The polynucleotide of E61, E73, or E104, wherein the polynucleotide comprises a polynucleotide sequence comprising nucleotides 1 to 2610 of SEQ ID NO: 17.
E107. The polynucleotide of E61 or E73, wherein the polynucleotide comprises a polynucleotide sequence comprising nucleotides 157 to 3538 of SEQ ID NO: 18.
E108. The polynucleotide of E61, E73, or E107, wherein the polynucleotide comprises a polynucleotide sequence comprising nucleotides 1 to 3686 of SEQ ID NO: 18.
E109. The polynucleotide of E61 or E73, wherein the polynucleotide comprises a polynucleotide sequence comprising nucleotides 157 to 3538 of SEQ ID NO: 19.
E110. The polynucleotide of E61, E73, or E109, wherein the polynucleotide comprises a polynucleotide sequence comprising nucleotides 1 to 3686 of SEQ ID NO: 19.
E111. The polynucleotide of E61 or E73, wherein the polynucleotide comprises a polynucleotide sequence comprising nucleotides 212 to 2438 of SEQ ID NO: 20.
E112. The polynucleotide of E61, E73, or E111, wherein the polynucleotide comprises a polynucleotide sequence comprising nucleotides 1 to 2650 of SEQ ID NO: 20.
E113. The polynucleotide of E61 or E73, wherein the polynucleotide comprises a polynucleotide sequence comprising nucleotides 212 to 2438 of SEQ ID NO: 21.
E114. The polynucleotide of E61, E73, or E113, wherein the polynucleotide comprises a polynucleotide sequence comprising nucleotides 1 to 2650 of SEQ ID NO: 21.
E115. The polynucleotide of E61 or E73, wherein the polynucleotide comprises a polynucleotide sequence comprising nucleotides 212 to 3514 of SEQ ID NO: 22.
E116. The polynucleotide of E61, E73, or E115, wherein the polynucleotide comprises a polynucleotide sequence comprising nucleotides 1 to 3726 of SEQ ID NO: 22.
E117. The polynucleotide of E61 or E73, wherein the polynucleotide comprises a polynucleotide sequence comprising nucleotides 212 to 3514 of SEQ ID NO: 23.
E118. The polynucleotide of E61, E73, or E117, wherein the polynucleotide comprises a polynucleotide sequence comprising nucleotides 1 to 3726 of SEQ ID NO: 23.
E119. The polynucleotide of E61 or E73, wherein the polynucleotide comprises a polynucleotide sequence comprising nucleotides 212 to 2517 of SEQ ID NO: 24.
E120. The polynucleotide of E61, E73, or E119, wherein the polynucleotide comprises a polynucleotide sequence comprising nucleotides 1 to 2729 of SEQ ID NO: 24.
E121. The polynucleotide of E61 or E73, wherein the polynucleotide comprises a polynucleotide sequence comprising nucleotides 212 to 2517 of SEQ ID NO: 25.
E122. The polynucleotide of E61, E73, or E121, wherein the polynucleotide comprises a polynucleotide sequence comprising nucleotides 1 to 2729 of SEQ ID NO: 25.
E123. The polynucleotide of E61 or E73, wherein the polynucleotide comprises a polynucleotide sequence comprising nucleotides 212 to 3593 of SEQ ID NO: 26.
E124. The polynucleotide of E61, E73, or E123, wherein the polynucleotide comprises a polynucleotide sequence comprising nucleotides 1 to 3805 of SEQ ID NO: 26.
E125. The polynucleotide of E61 or E73, wherein the polynucleotide comprises a polynucleotide sequence comprising nucleotides 212 to 3593 of SEQ ID NO: 27.
E126. The polynucleotide of E61, E73, or E125, wherein the polynucleotide comprises a polynucleotide sequence comprising nucleotides 1 to 3805 of SEQ ID NO: 27.
E127. A nucleic acid vector comprising the polynucleotide of any one of E61-E126.
E128. The nucleic acid vector of any one of E1-E60 and E127, wherein the nucleic acid vector is a viral vector, plasmid, cosmid, or artificial chromosome.
E129. The nucleic acid vector of any one of E1-E60, E127, and E122, wherein the nucleic acid vector is a viral vector.
E130. The nucleic acid vector of E129, wherein the viral vector is selected from the group consisting of an adeno-associated virus (AAV), an adenovirus, and a lentivirus.
E131. The nucleic acid vector of E130, wherein the viral vector is an AAV vector.
E132. The nucleic acid vector of E131, wherein the AAV vector has an AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ, DJ/8, DJ/9, 7m8, PHP.B, PHP.eB, or PHP.S capsid.
E133. The nucleic acid vector of E132, wherein the AAV vector has an AAV1 capsid.
E134. The nucleic acid vector of E132, wherein the AAV vector has an AAV-DJ capsid.
E135. The nucleic acid vector of E132, wherein the AAV vector has an AAV9 capsid.
E136. The nucleic acid vector of E132, wherein the AAV vector has a 7m8 capsid.
E137. The nucleic acid vector of E132, wherein the AAV vector has an Anc80 capsid.
E138. The nucleic acid vector of E132, wherein the AAV vector has an AAV2 capsid.
E139. The nucleic acid vector of E132, wherein the AAV vector has an AAV2quad(Y-F) capsid.
E140. The nucleic acid vector of E132, wherein the AAV vector has an AAV8 capsid.
E141. The nucleic acid vector of E132, wherein the AAV vector has a DJ/9 capsid.
E142. The nucleic acid vector of E132, wherein the AAV vector has a PHP.B capsid.
E143. The nucleic acid vector of E132, wherein the AAV vector has an AAV6 capsid.
E144. The nucleic acid vector of E132, wherein the AAV vector has a PHP.S capsid.
E145. A composition comprising the nucleic acid vector of any one of E1-E60 and E127-E144 and a pharmaceutically acceptable carrier, diluent, or excipient.
E146. A cell comprising the polynucleotide of any one of E61-E126 or the nucleic acid vector of any one of E1-E60 and E127-E144.
E147. The cell of E146, wherein the cell is a GJB2-expressing cell.
E148. The cell of E147, wherein the GJB2-expressing cell is a GJB2-expressing inner ear cell.
E149. The cell of any one of E146-E148, wherein the cell is a mammalian cell.
E150. The cell of E149, wherein the mammalian cell is a human cell.
E151. The cell of any one of E146-E150, wherein the cell is a cochlear supporting cell.
E152. A method of expressing human GJB2 in a GJB2-expressing cell, comprising contacting the GJB2-expressing cell with the nucleic acid vector of any one of E1-E60 and E127-E144 or the composition of E145.
E153. The method of E152, wherein the GJB2-expressing cell is a GJB2-expressing inner ear cell.
E154. The method of E153, wherein the GJB2-expressing inner ear cell is a cochlear supporting cell.
E155. The method of any one of E152-E154, wherein the contacting is in a subject (e.g., in vivo).
E156. A method of treating a subject having or at risk of developing GJB2-related hearing loss, comprising administering to an inner ear of the subject a therapeutically effective amount of the nucleic acid vector of any one of E1-E60 and E127-E144 or the composition of E145.
E157. The method of E156, wherein the GJB2-related hearing loss is DFNB1, DFNA3, or hearing loss associated with Bart-Pumphrey syndrome, hystrix-like ichthyosis with deafness, keratitis-ichthyosis-deafness syndrome, palmoplantar keratoderma with deafness, or Vohwinkel syndrome.
E158. The method of E156, wherein the hearing loss is DFNB1 or DFNA3.
E159. The method of E158, wherein the subject has a mutation in GJB2, a mutation in GJB6, or a mutation in both GJB2 and GJB6.
E160. A method of improving cochlear supporting cell function or cochlear supporting cell survival, comprising contacting the cochlear supporting cell with the nucleic acid vector of any one of E1-E60 and E127-E144 or the composition of E145.
E161. The method of E160, wherein the contacting is in a subject (e.g., in vivo).
E162. A method of improving cochlear supporting cell function or cochlear supporting cell survival in a subject in need thereof, comprising administering to an inner ear of the subject a therapeutically effective amount of the nucleic acid vector of any one of E1-E60 and E127-E144 or the composition of E145.
E163. The method of E161 or E162, wherein the subject has or is at risk of developing GJB2-related hearing loss.
E164. The method of E163, wherein the GJB2-related hearing loss is DFNB1, DFNA3, or hearing loss associated with Bart-Pumphrey syndrome, hystrix-like ichthyosis with deafness, keratitis-ichthyosis-deafness syndrome, palmoplantar keratoderma with deafness, or Vohwinkel syndrome.
E165. The method of E163, wherein the hearing loss is DFNB1 or DFNA3.
E166. The method of any one of E160-E165, wherein the cochlear supporting cell is a mammalian cochlear supporting cell.
E167. The method of E166, wherein the mammalian cochlear supporting cell is a human cochlear supporting cell.
E168. The method of any one of E155-E159 and E161-E167, wherein the method further comprises evaluating the hearing of the subject prior to administering the nucleic acid vector or composition.
E169. The method of any one of E155-E159 and E161-E168, wherein the method further comprises evaluating the hearing of the subject after administering the nucleic acid vector or composition.
E170. The method of any one of E155-E169, wherein the nucleic acid vector or composition is locally administered.
E171. The method of E170, wherein the nucleic acid vector or composition is administered to the inner ear.
E172. The method of E170, wherein the nucleic acid vector or composition is administered to the middle ear.
E173. The method of E170, wherein the nucleic acid vector or composition is administered transtympanically or intratympanically.
E174. The method of E170, wherein the nucleic acid vector or composition is administered into the perilymph.
E175. The method of E170, wherein the nucleic acid vector or composition is administered into the endolymph.
E176. The method of E170, wherein the nucleic acid vector or composition is administered to or through the oval window.
E177. The method of E170, wherein the nucleic acid vector or composition is administered to or through the round window.
E178. The method of any one of E155-E177, wherein the nucleic acid vector or composition is administered in an amount sufficient to prevent or reduce hearing loss, delay the development of hearing loss, slow the progression of hearing loss, improve hearing, increase or induce expression of human GJB2 in GJB2-expressing cells, promote or increase cochlear supporting cell survival, or improve cochlear supporting cell function.
E179. The method of any one of E155-E159 and E161-E178, wherein the subject is a human subject.
E180. A kit comprising the polynucleotide of any one of E61-E126, the nucleic acid vector of any one of E1-E50 and E127-E144, or the composition of E145.

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention. Other embodiments are in the claims.

1. A nucleic acid vector comprising, in 5′-to-3′ order:
(a) a GJB2 regulatory construct having a sequence selected from the group consisting of SEQ ID NO: 1, SEQ ID NO: 2, and a sequence having at least 90% sequence identity to SEQ ID NO: 1 or SEQ ID NO: 2, operably linked to:
(b) a human GJB2 coding sequence selected from the group consisting of SEQ ID NO: 3, SEQ ID NO: 4, SEQ ID NO: 28, a sequence having at least 90% sequence identity to SEQ ID NO: 3 and comprising at least 50% fewer CG dinucleotides than wild-type GJB2, and a sequence having at least 90% sequence identity to SEQ ID NO: 4 or SEQ ID NO: 28, operably linked to:
(c) a polyadenylation (polyA) signal sequence.

2. The nucleic acid vector of claim 1, wherein the GJB2 regulatory construct has the sequence of SEQ ID NO: 1 and the human GJB2 coding sequence has the sequence of SEQ ID NO: 3.

3. The nucleic acid vector of claim 1, wherein the GJB2 regulatory construct has the sequence of SEQ ID NO: 1 and the human GJB2 coding sequence has the sequence of SEQ ID NO: 4.

4. The nucleic acid vector of claim 1, wherein the GJB2 regulatory construct has the sequence of SEQ ID NO: 1 and the human GJB2 coding sequence has the sequence of SEQ ID NO: 28.

5. The nucleic acid vector of claim 1, wherein the GJB2 regulatory construct has the sequence of SEQ ID NO: 2; and the human GJB2 coding sequence has the sequence of SEQ ID NO: 3.

6. The nucleic acid vector of claim 1, wherein the GJB2 regulatory construct has the sequence of SEQ ID NO: 2; and the human GJB2 coding sequence has the sequence of SEQ ID NO: 4.

7. The nucleic acid vector of claim 1, wherein the GJB2 regulatory construct has the sequence of SEQ ID NO: 2; and the human GJB2 coding sequence has the sequence of SEQ ID NO: 28.

8. The nucleic acid vector of any one of claims 1-7, wherein the polyA signal sequence has at least 90% sequence identity to SEQ ID NO: 6 or SEQ ID NO: 7.

9. The nucleic acid vector of claim 8, wherein the polyA signal sequence has the sequence of SEQ ID NO: 6.

10. The nucleic acid vector of claim 8, wherein the polyA signal sequence has the sequence of SEQ ID NO: 7.

11. The nucleic acid vector of any one of claims 1-10, wherein the vector comprises a stop codon positioned at the 3′ end of the GJB2 coding sequence.

12. The nucleic acid vector of any one of claims 1-11, wherein the vector further comprises a first polynucleotide having at least 90% sequence identity to SEQ ID NO: 8 that comprises a first member of an ITR pair and is positioned 5′ of the GJB2 regulatory construct sequence and a second polynucleotide having at least 90% sequence identity to SEQ ID NO: 9 that comprises a second member of an ITR pair and is positioned 3′ of the polyA signal sequence.

13. The nucleic acid vector of any one of claims 1-11, wherein the vector further comprises a first polynucleotide having at least 90% sequence identity to SEQ ID NO: 10 that comprises a first member of an ITR pair and is positioned 5′ of the GJB2 regulatory construct sequence and a second polynucleotide having at least 90% sequence identity to SEQ ID NO: 11 that comprises a second member of an ITR pair and is positioned 3′ of the polyA signal sequence.

14. The nucleic acid vector of any one of claims 1-13, wherein the nucleic acid vector is a viral vector.

15. The nucleic acid vector of claim 14, wherein the viral vector is an adeno-associated virus (AAV) vector.

16. A composition comprising the nucleic acid vector of any one of claims 1-15; and a pharmaceutically acceptable carrier, diluent, or excipient.

17. A method of treating a subject having or at risk of developing GJB2-related hearing loss, comprising administering to an inner ear of the subject a therapeutically effective amount of the nucleic acid vector of any one of claims 1-15 or the composition of claim 16.

18. The method of claim 17, wherein the GJB2-related hearing loss is DFNB1 or DFNA3.

19. The method of claim 18, wherein the subject has a mutation in GJB2, a mutation in GJB6, or a mutation in both GJB2 and GJB6.


SEQUENCE LISTING (29 sequences)

SEQ ID NO: 1 - DNA, 1385 bp, synthetic construct
  source 1..1385: mol_type = other DNA
aaaaactcttacataattgtaacagatttgagtttcctctggttcaggttttcttgcctctttgataatcaaatgatctgaagaaaggcatagaatttcaagggagaatctgcatgacaggattacaataaggctattcatggagactcttttattagcttacacaggatctgcatcattcttcgtctctggctttgctacaaggctccatttaaacttaacccaacttgcaggcttaactgatccaggaatgactcaatacaatgagccagctactgtagctctttttccccttttgataaggggagttaacacaatgggctttacagttcttaaatgaacgcgtacagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctgtttatgatgtgtttaaagattgggtgaattactcaggtgaacaagctactttttatcagagaacacctaaaaacacgttcaagagggtttgggaactatacatttaatcctatgacaaactaagttggttctgtcttcacctgttttggtgaggttgtgtaagagttggtgtttgctcaggaagagatttaagcatgcttgcttacccagactcagagaagtctccctgttctgtcctagctagtgattcctgtgttgtgtgcattcgtcttttccagagcaaaccgcccagagtagaag

SEQ ID NO: 2 - DNA, 2461 bp, synthetic construct
  source 1..2461: mol_type = other DNA
aaaaactcttacataattgtaacagatttgagtttcctctggttcaggttttcttgcctctttgataatcaaatgatctgaagaaaggcatagaatttcaagggagaatctgcatgacaggattacaataaggctattcatggagactcttttattagcttacacaggatctgcatcattcttcgtctctggctttgctacaaggctccatttaaacttaacccaacttgcaggcttaactgatccaggaatgactcaatacaatgagccagctactgtagctctttttccccttttgataaggggagttaacacaatgggctttacagttcttaaatgaccgttcctgcaggtattggaaaacctgcatcgtatgcctgctgctttaggagacaacaaactcagcagcccctcccccgaagctgaagtgccaaacttaaggctccaggtaaactgcccttccgatgagagctttacatttaggcagtagccacaggcctaaacaaagtcaaatggatgaaacttgtgatctctccttggaaatatggccaagagtgaaacaactacttagttattattttaaacgcaggctgctaaatggcttgtgttggtatgtaatggcatcctagataatgggggctgccaagaaaatattcacactggcaagtcaattaaaataccgttctcttcctaagaggcctggctggattccagagcttcctttagggtaacgctcaaacctgtcatagtttattttgtttctgttttcaggcaacagatttggttattgcttttattgtatgagtgttgaaatggtgtcctaacagccatctgtactgccatcccctggtctctctgcctttccagaaaggagatgaagttgctggagcaggcaggttctctgaaaggctccctgagtgtggaagagcagctgtcactcatcagtggttgtcccaatatccaagaagcagtagagggtgccatgcacattcaggtgagtgaaggacactggcgccgcatctgtggggcctgagacattgtctcccagcctctccggaggaacagggcaggatacagagcagtggcaggtgtgggcattttgcatcttctgtaatctccaaacaaagtctgcgctaggccctttcaggctataaaggacagggtactctgatttctgctcttgatgagggttgattgtgataccaggcgagggagtaagagatacaggacaccatctctgcggctgcatctttgtcgcagaaacagacattgttcaggggacacctgagcctctgatagtcagccagtggctctggcagctcacagcagttatagggtggctgtgtccttttctaaagtcctgactttttatggcctgtggtcatggtgactccactacgggctgtgtttctgttgacgcgtacagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctgtttatgatgtgtttaaagattgggtgaattactcaggtgaacaagctactttttatcagagaacacctaaaaacacgttcaagagggtttgggaactatacatttaatcctatgacaaactaagttggttctgtcttcacctgttttggtgaggttgtgtaagagttggtgtttgctcaggaagagatttaagcatgcttgcttacccagactcagagaagtctccctgttctgtcctagctagtgattcctgtgttgtgtgcattcgtcttttccagagcaaaccgcccagagtagaag

SEQ ID NO: 3 - DNA, 678 bp, synthetic construct
  source 1..678: mol_type = other DNA
atggattggggcacactgcagactatcctggggggtgtgaacaaacactccaccagcattggaaagatctggctcacagtcctcttcatttttaggattatgatcctggttgtggctgcaaaggaggtgtggggagatgagcaggctgactttgtctgcaacaccctgcagccaggctgcaagaatgtgtgctatgatcactacttccccatctcccacatcaggctatgggccctgcagctgatctttgtgtccactccagcactcctagtggccatgcatgtggcctacaggagacatgagaagaagaggaagttcatcaagggggagataaagagtgaatttaaggacattgaggagatcaaaacccagaaggtcagaattgaaggctccctgtggtggacctacacaagcagcatcttcttcagggtcatctttgaagcagccttcatgtatgtcttctatgtcatgtatgatggcttctccatgcagaggctggtgaagtgcaatgcctggccttgtcccaacactgtggactgctttgtgtccaggcccactgagaagactgtcttcacagtgttcatgattgcagtgtctggaatttgcatcctgctgaatgtcactgaattgtgttatttgctaattagatattgttctgggaagtcaaaaaagccagtt

SEQ ID NO: 4 - DNA, 678 bp, synthetic construct
  source 1..678: mol_type = other DNA
atggactggggaacactgcaaacaatcctgggaggtgtgaacaagcacagcacaagcattggcaagatctggctgacagtgctgttcatctttagaatcatgatcctggttgtggctgctaaggaagtgtggggagatgagcaggctgactttgtttgtaataccctgcaacctggctgcaagaatgtgtgttatgaccactacttccccatcagccacatcagactgtgggccctgcaactgatctttgtgtccacccctgccctgctggtggccatgcatgtggcctacagaaggcatgagaagaagaggaaattcatcaagggagagatcaagagtgagttcaaggacattgaggaaatcaaaacccagaaggtgaggattgaaggctctctgtggtggacctacaccagcagcatcttcttcagagtgatctttgaggcagcctttatgtatgtgttctatgtgatgtatgatggcttctccatgcagagactggtgaagtgcaatgcctggccatgtcctaacacagtggactgctttgtgtctagacctacagaaaagacagtgtttacagtctttatgattgcagtgtctggcatctgcattctgctgaatgtgactgagctgtgctacctgctcattaggtactgtagtggaaaaagcaagaaacctgtg

SEQ ID NO: 5 - DNA, 678 bp, Homo sapiens
  source 1..678: mol_type = other DNA
atggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtt

SEQ ID NO: 6 - DNA, 208 bp, Bos taurus
  source 1..208: mol_type = genomic DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 7 - DNA, 135 bp, Simian virus 40
  source 1..135: mol_type = other DNA
gatccagacatgataagatacattgatgagtttggacaaaccaaaactagaatgcagtgaaaaaaatgccttatttgtgaaatttgtgatgctattgccttatttgtaaccattataagctgcaataaacaagtt

SEQ ID NO: 8 - DNA, 211 bp, synthetic construct
  source 1..211: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattctggtaccttgctagc

SEQ ID NO: 9 - DNA, 212 bp, synthetic construct
  source 1..212: mol_type = other DNA
tctagaactgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 10 - DNA, 156 bp, synthetic construct
  source 1..156: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgagcagaattctggtaccttgctagc

SEQ ID NO: 11 - DNA, 148 bp, synthetic construct
  source 1..148: mol_type = other DNA
tctagaactgaattcacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 12 - DNA, 5301 bp, synthetic construct
  source 1..5301: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgagcagaattctggtaccttgctagcaaaaactcttacataattgtaacagatttgagtttcctctggttcaggttttcttgcctctttgataatcaaatgatctgaagaaaggcatagaatttcaagggagaatctgcatgacaggattacaataaggctattcatggagactcttttattagcttacacaggatctgcatcattcttcgtctctggctttgctacaaggctccatttaaacttaacccaacttgcaggcttaactgatccaggaatgactcaatacaatgagccagctactgtagctctttttccccttttgataaggggagttaacacaatgggctttacagttcttaaatgaacgcgtacagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctgtttatgatgtgtttaaagattgggtgaattactcaggtgaacaagctactttttatcagagaacacctaaaaacacgttcaagagggtttgggaactatacatttaatcctatgacaaactaagttggttctgtcttcacctgttttggtgaggttgtgtaagagttggtgtttgctcaggaagagatttaagcatgcttgcttacccagactcagagaagtctccctgttctgtcctagctagtgattcctgtgttgtgtgcattcgtcttttccagagcaaaccgcccagagtagaagcctgcaggccaccatggattggggcacactgcagactatcctggggggtgtgaacaaacactccaccagcattggaaagatctggctcacagtcctcttcatttttaggattatgatcctggttgtggctgcaaaggaggtgtggggagatgagcaggctgactttgtctgcaacaccctgcagccaggctgcaagaatgtgtgctatgatcactacttccccatctcccacatcaggctatgggccctgcagctgatctttgtgtccactccagcactcctagtggccatgcatgtggcctacaggagacatgagaagaagaggaagttcatcaagggggagataaagagtgaatttaaggacattgaggagatcaaaacccagaaggtcagaattgaaggctccctgtggtggacctacacaagcagcatcttcttcagggtcatctttgaagcagccttcatgtatgtcttctatgtcatgtatgatggcttctccatgcagaggctggtgaagtgcaatgcctggccttgtcccaacactgtggactgctttgtgtccaggcccactgagaagactgtcttcacagtgttcatgattgcagtgtctggaatttgcatcctgctgaatgtcactgaattgtgttatttgctaattagatattgttctgggaagtcaaaaaagccagtttgataagcttagatctgatccagacatgataagatacattgatgagtttggacaaaccaaaactagaatgcagtgaaaaaaatgccttatttgtgaaatttgtgatgctattgccttatttgtaaccattataagctgcaataaacaagtttctagaactgaattcacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 13 - DNA, 5301 bp, synthetic construct
  source 1..5301: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgagcagaattctggtaccttgctagcaaaaactcttacataattgtaacagatttgagtttcctctggttcaggttttcttgcctctttgataatcaaatgatctgaagaaaggcatagaatttcaagggagaatctgcatgacaggattacaataaggctattcatggagactcttttattagcttacacaggatctgcatcattcttcgtctctggctttgctacaaggctccatttaaacttaacccaacttgcaggcttaactgatccaggaatgactcaatacaatgagccagctactgtagctctttttccccttttgataaggggagttaacacaatgggctttacagttcttaaatgaacgcgtacagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctgtttatgatgtgtttaaagattgggtgaattactcaggtgaacaagctactttttatcagagaacacctaaaaacacgttcaagagggtttgggaactatacatttaatcctatgacaaactaagttggttctgtcttcacctgttttggtgaggttgtgtaagagttggtgtttgctcaggaagagatttaagcatgcttgcttacccagactcagagaagtctccctgttctgtcctagctagtgattcctgtgttgtgtgcattcgtcttttccagagcaaaccgcccagagtagaagcctgcaggccaccatggactggggaacactgcaaacaatcctgggaggtgtgaacaagcacagcacaagcattggcaagatctggctgacagtgctgttcatctttagaatcatgatcctggttgtggctgctaaggaagtgtggggagatgagcaggctgactttgtttgtaataccctgcaacctggctgcaagaatgtgtgttatgaccactacttccccatcagccacatcagactgtgggccctgcaactgatctttgtgtccacccctgccctgctggtggccatgcatgtggcctacagaaggcatgagaagaagaggaaattcatcaagggagagatcaagagtgagttcaaggacattgaggaaatcaaaacccagaaggtgaggattgaaggctctctgtggtggacctacaccagcagcatcttcttcagagtgatctttgaggcagcctttatgtatgtgttctatgtgatgtatgatggcttctccatgcagagactggtgaagtgcaatgcctggccatgtcctaacacagtggactgctttgtgtctagacctacagaaaagacagtgtttacagtctttatgattgcagtgtctggcatctgcattctgctgaatgtgactgagctgtgctacctgctcattaggtactgtagtggaaaaagcaagaaacctgtgtgataagcttagatctgatccagacatgataagatacattgatgagtttggacaaaccaaaactagaatgcagtgaaaaaaatgccttatttgtgaaatttgtgatgctattgccttatttgtaaccattataagctgcaataaacaagtttctagaactgaattcacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 14 - DNA, 6377 bp, synthetic construct
  source 1..6377: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgagcagaattctggtaccttgctagcaaaaactcttacataattgtaacagatttgagtttcctctggttcaggttttcttgcctctttgataatcaaatgatctgaagaaaggcatagaatttcaagggagaatctgcatgacaggattacaataaggctattcatggagactcttttattagcttacacaggatctgcatcattcttcgtctctggctttgctacaaggctccatttaaacttaacccaacttgcaggcttaactgatccaggaatgactcaatacaatgagccagctactgtagctctttttccccttttgataaggggagttaacacaatgggctttacagttcttaaatgaccgttcctgcaggtattggaaaacctgcatcgtatgcctgctgctttaggagacaacaaactcagcagcccctcccccgaagctgaagtgccaaacttaaggctccaggtaaactgcccttccgatgagagctttacatttaggcagtagccacaggcctaaacaaagtcaaatggatgaaacttgtgatctctccttggaaatatggccaagagtgaaacaactacttagttattattttaaacgcaggctgctaaatggcttgtgttggtatgtaatggcatcctagataatgggggctgccaagaaaatattcacactggcaagtcaattaaaataccgttctcttcctaagaggcctggctggattccagagcttcctttagggtaacgctcaaacctgtcatagtttattttgtttctgttttcaggcaacagatttggttattgcttttattgtatgagtgttgaaatggtgtcctaacagccatctgtactgccatcccctggtctctctgcctttccagaaaggagatgaagttgctggagcaggcaggttctctgaaaggctccctgagtgtggaagagcagctgtcactcatcagtggttgtcccaatatccaagaagcagtagagggtgccatgcacattcaggtgagtgaaggacactggcgccgcatctgtggggcctgagacattgtctcccagcctctccggaggaacagggcaggatacagagcagtggcaggtgtgggcattttgcatcttctgtaatctccaaacaaagtctgcgctaggccctttcaggctataaaggacagggtactctgatttctgctcttgatgagggttgattgtgataccaggcgagggagtaagagatacaggacaccatctctgcggctgcatctttgtcgcagaaacagacattgttcaggggacacctgagcctctgatagtcagccagtggctctggcagctcacagcagttatagggtggctgtgtccttttctaaagtcctgactttttatggcctgtggtcatggtgactccactacgggctgtgtttctgttgacgcgtacagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctgtttatgatgtgtttaaagattgggtgaattactcaggtgaacaagctactttttatcagagaacacctaaaaacacgttcaagagggtttgggaactatacatttaatcctatgacaaactaagttggttctgtcttcacctgttttggtgaggttgtgtaagagttggtgtttgctcaggaagagatttaagcatgcttgcttacccagactcagagaagtctccctgttctgtcctagctagtgattcctgtgttgtgtgcattcgtcttttccagagcaaaccgcccagagtagaagcctgcaggccaccatggattggggcacactgcagactatcctggggggtgtgaacaaacactccaccagcattggaaagatctggctcacagtcctcttcatttttaggattatgatcctggttgtggctgcaaaggaggtgtggggagatgagcaggctgactttgtctgcaacaccctgcagccaggctgcaagaatgtgtgctatgatcactacttccccatctcccacatcaggctatgggccctgcagctgatctttgtgtccactccagcactcctagtggccatgcatgtggcctacaggagacatgagaagaagaggaagttcatcaagggggagataaagagtgaatttaaggacattgaggagatcaaaacccagaaggtcagaattgaaggctccctgtggtggacctacacaagcagcatcttcttcagggtcatctttgaagcagccttcatgtatgtcttctatgtcatgtatgatggcttctccatgcagaggctggtgaagtgcaatgcctggccttgtcccaacactgtggactgctttgtgtccaggcccactgagaagactgtcttcacagtgttcatgattgcagtgtctggaatttgcatcctgctgaatgtcactgaattgtgttatttgctaattagatattgttctgggaagtcaaaaaagccagtttgataagcttagatctgatccagacatgataagatacattgatgagtttggacaaaccaaaactagaatgcagtgaaaaaaatgccttatttgtgaaatttgtgatgctattgccttatttgtaaccattataagctgcaataaacaagtttctagaactgaattcacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 15 - DNA, 6377 bp, synthetic construct; identical to SEQ ID NO: 14
  source 1..6377: mol_type = other DNA

SEQ ID NO: 16 - DNA, 5380 bp, synthetic construct
  source 1..5380: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgagcagaattctggtaccttgctagcaaaaactcttacataattgtaacagatttgagtttcctctggttcaggttttcttgcctctttgataatcaaatgatctgaagaaaggcatagaatttcaagggagaatctgcatgacaggattacaataaggctattcatggagactcttttattagcttacacaggatctgcatcattcttcgtctctggctttgctacaaggctccatttaaacttaacccaacttgcaggcttaactgatccaggaatgactcaatacaatgagccagctactgtagctctttttccccttttgataaggggagttaacacaatgggctttacagttcttaaatgaacgcgtacagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctgtttatgatgtgtttaaagattgggtgaattactcaggtgaacaagctactttttatcagagaacacctaaaaacacgttcaagagggtttgggaactatacatttaatcctatgacaaactaagttggttctgtcttcacctgttttggtgaggttgtgtaagagttggtgtttgctcaggaagagatttaagcatgcttgcttacccagactcagagaagtctccctgttctgtcctagctagtgattcctgtgttgtgtgcattcgtcttttccagagcaaaccgcccagagtagaagcctgcaggccaccatggattggggcacactgcagactatcctggggggtgtgaacaaacactccaccagcattggaaagatctggctcacagtcctcttcatttttaggattatgatcctggttgtggctgcaaaggaggtgtggggagatgagcaggctgactttgtctgcaacaccctgcagccaggctgcaagaatgtgtgctatgatcactacttccccatctcccacatcaggctatgggccctgcagctgatctttgtgtccactccagcactcctagtggccatgcatgtggcctacaggagacatgagaagaagaggaagttcatcaagggggagataaagagtgaatttaaggacattgaggagatcaaaacccagaaggtcagaattgaaggctccctgtggtggacctacacaagcagcatcttcttcagggtcatctttgaagcagccttcatgtatgtcttctatgtcatgtatgatggcttctccatgcagaggctggtgaagtgcaatgcctggccttgtcccaacactgtggactgctttgtgtccaggcccactgagaagactgtcttcacagtgttcatgattgcagtgtctggaatttgcatcctgctgaatgtcactgaattgtgttatttgctaattagatattgttctgggaagtcaaaaaagccagtttgataagcttagatctgcctgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatctagaactgaattcacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 17 - DNA, 5380 bp, synthetic construct
  source 1..5380: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgagcagaattctggtaccttgctagcaaaaactcttacataattgtaacagatttgagtttcctctggttcaggttttcttgcctctttgataatcaaatgatctgaagaaaggcatagaatttcaagggagaatctgcatgacaggattacaataaggctattcatggagactcttttattagcttacacaggatctgcatcattcttcgtctctggctttgctacaaggctccatttaaacttaacccaacttgcaggcttaactgatccaggaatgactcaatacaatgagccagctactgtagctctttttccccttttgataaggggagttaacacaatgggctttacagttcttaaatgaacgcgtacagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctgtttatgatgtgtttaaagattgggtgaattactcaggtgaacaagctactttttatcagagaacacctaaaaacacgttcaagagggtttgggaactatacatttaatcctatgacaaactaagttggttctgtcttcacctgttttggtgaggttgtgtaagagttggtgtttgctcaggaagagatttaagcatgcttgcttacccagactcagagaagtctccctgttctgtcctagctagtgattcctgtgttgtgtgcattcgtcttttccagagcaaaccgcccagagtagaagcctgcaggccaccatggactggggaacactgcaaacaatcctgggaggtgtgaacaagcacagcacaagcattggcaagatctggctgacagtgctgttcatctttagaatcatgatcctggttgtggctgctaaggaagtgtggggagatgagcaggctgactttgtttgtaataccctgcaacctggctgcaagaatgtgtgttatgaccactacttccccatcagccacatcagactgtgggccctgcaactgatctttgtgtccacccctgccctgctggtggccatgcatgtggcctacagaaggcatgagaagaagaggaaattcatcaagggagagatcaagagtgagttcaaggacattgaggaaatcaaaacccagaaggtgaggattgaaggctctctgtggtggacctacaccagcagcatcttcttcagagtgatctttgaggcagcctttatgtatgtgttctatgtgatgtatgatggcttctccatgcagagactggtgaagtgcaatgcctggccatgtcctaacacagtggactgctttgtgtctagacctacagaaaagacagtgtttacagtctttatgattgcagtgtctggcatctgcattctgctgaatgtgactgagctgtgctacctgctcattaggtactgtagtggaaaaagcaagaaacctgtgtgataagcttagatctgcctgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatctagaactgaattcacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 18 - DNA, 6456 bp, synthetic construct
  source 1..6456: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgagcagaattctggtaccttgctagcaaaaactcttacataattgtaacagatttgagtttcctctggttcaggttttcttgcctctttgataatcaaatgatctgaagaaaggcatagaatttcaagggagaatctgcatgacaggattacaataaggctattcatggagactcttttattagcttacacaggatctgcatcattcttcgtctctggctttgctacaaggctccatttaaacttaacccaacttgcaggcttaactgatccaggaatgactcaatacaatgagccagctactgtagctctttttccccttttgataaggggagttaacacaatgggctttacagttcttaaatgaccgttcctgcaggtattggaaaacctgcatcgtatgcctgctgctttaggagacaacaaactcagcagcccctcccccgaagctgaagtgccaaacttaaggctccaggtaaactgcccttccgatgagagctttacatttaggcagtagccacaggcctaaacaaagtcaaatggatgaaacttgtgatctctccttggaaatatggccaagagtgaaacaactacttagttattattttaaacgcaggctgctaaatggcttgtgttggtatgtaatggcatcctagataatgggggctgccaagaaaatattcacactggcaagtcaattaaaataccgttctcttcctaagaggcctggctggattccagagcttcctttagggtaacgctcaaacctgtcatagtttattttgtttctgttttcaggcaacagatttggttattgcttttattgtatgagtgttgaaatggtgtcctaacagccatctgtactgccatcccctggtctctctgcctttccagaaaggagatgaagttgctggagcaggcaggttctctgaaaggctccctgagtgtggaagagcagctgtcactcatcagtggttgtcccaatatccaagaagcagtagagggtgccatgcacattcaggtgagtgaaggacactggcgccgcatctgtggggcctgagacattgtctcccagcctctccggaggaacagggcaggatacagagcagtggcaggtgtgggcattttgcatcttctgtaatctccaaacaaagtctgcgctaggccctttcaggctataaaggacagggtactctgatttctgctcttgatgagggttgattgtgataccaggcgagggagtaagagatacaggacaccatctctgcggctgcatctttgtcgcagaaacagacattgttcaggggacacctgagcctctgatagtcagccagtggctctggcagctcacagcagttatagggtggctgtgtccttttctaaagtcctgactttttatggcctgtggtcatggtgactccactacgggctgtgtttctgttgacgcgtacagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctgtttatgatgtgtttaaagattgggtgaattactcaggtgaacaagctactttttatcagagaacacctaaaaacacgttcaagagggtttgggaactatacatttaatcctatgacaaactaagttggttctgtcttcacctgttttggtgaggttgtgtaagagttggtgtttgctcaggaagagatttaagcatgcttgcttacccagactcagagaagtctccctgttctgtcctagctagtgattcctgtgttgtgtgcattcgtcttttccagagcaaaccgcccagagtagaagcctgcaggccaccatggattggggcacactgcagactatcctggggggtgtgaacaaacactccaccagcattggaaagatctggctcacagtcctcttcatttttaggattatgatcctggttgtggctgcaaaggaggtgtggggagatgagcaggctgactttgtctgcaacaccctgcagccaggctgcaagaatgtgtgctatgatcactacttccccatctcccacatcaggctatgggccctgcagctgatctttgtgtccactccagcactcctagtggccatgcatgtggcctacaggagacatgagaagaagaggaagttcatcaagggggagataaagagtgaatttaaggacattgaggagatcaaaacccagaaggtcagaattgaaggctccctgtggtggacctacacaagcagcatcttcttcagggtcatctttgaagcagccttcatgtatgtcttctatgtcatgtatgatggcttctccatgcagaggctggtgaagtgcaatgcctggccttgtcccaacactgtggactgctttgtgtccaggcccactgagaagactgtcttcacagtgttcatgattgcagtgtctggaatttgcatcctgctgaatgtcactgaattgtgttatttgctaattagatattgttctgggaagtcaaaaaagccagtttgataagcttagatctgcctgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatctagaactgaattcacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 19 - DNA, 6456 bp, synthetic construct
  source 1..6456: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgagcagaattctggtaccttgctagcaaaaactcttacataattgtaacagatttgagtttcctctggttcaggttttcttgcctctttgataatcaaatgatctgaagaaaggcatagaatttcaagggagaatctgcatgacaggattacaataaggctattcatggagactcttttattagcttacacaggatctgcatcattcttcgtctctggctttgctacaaggctccatttaaacttaacccaacttgcaggcttaactgatccaggaatgactcaatacaatgagccagctactgtagctctttttccccttttgataaggggagttaacacaatgggctttacagttcttaaatgaccgttcctgcaggtattggaaaacctgcatcgtatgcctgctgctttaggagacaacaaactcagcagcccctcccccgaagctgaagtgccaaacttaaggctccaggtaaactgcccttccgatgagagctttacatttaggcagtagccacaggcctaaacaaagtcaaatggatgaaacttgtgatctctccttggaaatatggccaagagtgaaacaactacttagttattattttaaacgcaggctgctaaatggcttgtgttggtatgtaatggcatcctagataatgggggctgccaagaaaatattcacactggcaagtcaattaaaataccgttctcttcctaagaggcctggctggattccagagcttcctttagggtaacgctcaaacctgtcatagtttattttgtttctgttttcaggcaacagatttggttattgcttttattgtatgagtgttgaaatggtgtcctaacagccatctgtactgccatcccctggtctctctgcctttccagaaaggagatgaagttgctggagcaggcaggttctctgaaaggctccctgagtgtggaagagcagctgtcactcatcagtggttgtcccaatatccaagaagcagtagagggtgccatgcacattcaggtgagtgaaggacactggcgccgcatctgtggggcctgagacattgtctcccagcctctccggaggaacagggcaggatacagagcagtggcaggtgtgggcattttgcatcttctgtaatctccaaacaaagtctgcgctaggccctttcaggctataaaggacagggtactctgatttctgctcttgatgagggttgattgtgataccaggcgagggagtaagagatacaggacaccatctctgcggctgcatctttgtcgcagaaacagacattgttcaggggacacctgagcctctgatagtcagccagtggctctggcagctcacagcagttatagggtggctgtgtccttttctaaagtcctgactttttatggcctgtggtcatggtgactccactacgggctgtgtttctgttgacgcgtacagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctgtttatgatgtgtttaaagattgggtgaattactcaggtgaacaagctactttttatcagagaacacctaaaaacacgttcaagagggtttgggaactatacatttaatcctatgacaaactaagttggttctgtcttcacctgttttggtgaggttgtgtaagagttggtgtttgctcaggaagagatttaagcatgcttgcttacccagactcagagaagtctccctgttctgtcctagctagtgattcctgtgttgtgtgcattcgtcttttccagagcaaaccgcccagagtagaagcctgcaggccaccatggactggggaacactgcaaacaatcctgggaggtgtgaacaagcacagcacaagcattggcaagatctggctgacagtgctgttcatctttagaatcatgatcctggttgtggctgctaaggaagtgtggggagatgagcaggctgactttgtttgtaataccctgcaacctggctgcaagaatgtgtgttatgaccactacttccccatcagccacatcagactgtgggccctgcaactgatctttgtgtccacccctgccctgctggtggccatgcatgtggcctacagaaggcatgagaagaagaggaaattcatcaagggagagatcaagagtgagttcaaggacattgaggaaatcaaaacccagaaggtgaggattgaaggctctctgtggtggacctacaccagcagcatcttcttcagagtgatctttgaggcagcctttatgtatgtgttctatgtgatgtatgatggcttctccatgcagagactggtgaagtgcaatgcctggccatgtcctaacacagtggactgctttgtgtctagacctacagaaaagacagtgtttacagtctttatgattgcagtgtctggcatctgcattctgctgaatgtgactgagctgtgctacctgctcattaggtactgtagtggaaaaagcaagaaacctgtgtgataagcttagatctgcctgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatctagaactgaattcacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 20 - DNA, 5420 bp, synthetic construct
  source 1..5420: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattctggtaccttgctagcaaaaactcttacataattgtaacagatttgagtttcctctggttcaggttttcttgcctctttgataatcaaatgatctgaagaaaggcatagaatttcaagggagaatctgcatgacaggattacaataaggctattcatggagactcttttattagcttacacaggatctgcatcattcttcgtctctggctttgctacaaggctccatttaaacttaacccaacttgcaggcttaactgatccaggaatgactcaatacaatgagccagctactgtagctctttttccccttttgataaggggagttaacacaatgggctttacagttcttaaatgaacgcgtacagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctgtttatgatgtgtttaaagattgggtgaattactcaggtgaacaagctactttttatcagagaacacctaaaaacacgttcaagagggtttgggaactatacatttaatcctatgacaaactaagttggttctgtcttcacctgttttggtgaggttgtgtaagagttggtgtttgctcaggaagagatttaagcatgcttgcttacccagactcagagaagtctccctgttctgtcctagctagtgattcctgtgttgtgtgcattcgtcttttccagagcaaaccgcccagagtagaagcctgcaggccaccatggattggggcacactgcagactatcctggggggtgtgaacaaacactccaccagcattggaaagatctggctcacagtcctcttcatttttaggattatgatcctggttgtggctgcaaaggaggtgtggggagatgagcaggctgactttgtctgcaacaccctgcagccaggctgcaagaatgtgtgctatgatcactacttccccatctcccacatcaggctatgggccctgcagctgatctttgtgtccactccagcactcctagtggccatgcatgtggcctacaggagacatgagaagaagaggaagttcatcaagggggagataaagagtgaatttaaggacattgaggagatcaaaacccagaaggtcagaattgaaggctccctgtggtggacctacacaagcagcatcttcttcagggtcatctttgaagcagccttcatgtatgtcttctatgtcatgtatgatggcttctccatgcagaggctggtgaagtgcaatgcctggccttgtcccaacactgtggactgctttgtgtccaggcccactgagaagactgtcttcacagtgttcatgattgcagtgtctggaatttgcatcctgctgaatgtcactgaattgtgttatttgctaattagatattgttctgggaagtcaaaaaagccagtttgataagcttagatctgatccagacatgataagatacattgatgagtttggacaaaccaaaactagaatgcagtgaaaaaaatgccttatttgtgaaatttgtgatgctattgccttatttgtaaccattataagctgcaataaacaagtttctagaactgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 21 - DNA, 5420 bp, synthetic construct; identical to SEQ ID NO: 20
  source 1..5420: mol_type = other DNA

SEQ ID NO: 22 - DNA, 6496 bp, synthetic construct
  source 1..6496: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattctggtaccttgctagcaaaaactcttacataattgtaacagatttgagtttcctctggttcaggttttcttgcctctttgataatcaaatgatctgaagaaaggcatagaatttcaagggagaatctgcatgacaggattacaataaggctattcatggagactcttttattagcttacacaggatctgcatcattcttcgtctctggctttgctacaaggctccatttaaacttaacccaacttgcaggcttaactgatccaggaatgactcaatacaatgagccagctactgtagctctttttccccttttgataaggggagttaacacaatgggctttacagttcttaaatgaccgttcctgcaggtattggaaaacctgcatcgtatgcctgctgctttaggagacaacaaactcagcagcccctcccccgaagctgaagtgccaaacttaaggctccaggtaaactgcccttccgatgagagctttacatttaggcagtagccacaggcctaaacaaagtcaaatggatgaaacttgtgatctctccttggaaatatggccaagagtgaaacaactacttagttattattttaaacgcaggctgctaaatggcttgtgttggtatgtaatggcatcctagataatgggggctgccaagaaaatattcacactggcaagtcaattaaaataccgttctcttcctaagaggcctggctggattccagagcttcctttagggtaacgctcaaacctgtcatagtttattttgtttctgttttcaggcaacagatttggttattgcttttattgtatgagtgttgaaatggtgtcctaacagccatctgtactgccatcccctggtctctctgcctttccagaaaggagatgaagttgctggagcaggcaggttctctgaaaggctccctgagtgtggaagagcagctgtcactcatcagtggttgtcccaatatccaagaagcagtagagggtgccatgcacattcaggtgagtgaaggacactggcgccgcatctgtggggcctgagacattgtctcccagcctctccggaggaacagggcaggatacagagcagtggcaggtgtgggcattttgcatcttctgtaatctccaaacaaagtctgcgctaggccctttcaggctataaaggacagggtactctgatttctgctcttgatgagggttgattgtgataccaggcgagggagtaagagatacaggacaccatctctgcggctgcatctttgtcgcagaaacagacattgttcaggggacacctgagcctctgatagtcagccagtggctctggcagctcacagcagttatagggtggctgtgtccttttctaaagtcctgactttttatggcctgtggtcatggtgactccactacgggctgtgtttctgttgacgcgtacagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctgtttatgatgtgtttaaagattgggtgaattactcaggtgaacaagctactttttatcagagaacacctaaaaacacgttcaagagggtttgggaactatacatttaatcctatgacaaactaagttggttctgtcttcacctgttttggtgaggttgtgtaagagttggtgtttgctcaggaagagatttaagcatgcttgcttacccagactcagagaagtctccctgttctgtcctagctagtgattcctgtgttgtgtgcattcgtcttttccagagcaaaccgcccagagtagaagcctgcaggccaccatggattggggcacactgcagactatcctggggggtgtgaacaaacactccaccagcattggaaagatctggctcacagtcctcttcatttttaggattatgatcctggttgtggctgcaaaggaggtgtggggagatgagcaggctgactttgtctgcaacaccctgcagccaggctgcaagaatgtgtgctatgatcactacttccccatctcccacatcaggctatgggccctgcagctgatctttgtgtccactccagcactcctagtggccatgcatgtggcctacaggagacatgagaagaagaggaagttcatcaagggggagataaagagtgaatttaaggacattgaggagatcaaaacccagaaggtcagaattgaaggctccctgtggtggacctacacaagcagcatcttcttcagggtcatctttgaagcagccttcatgtatgtcttctatgtcatgtatgatggcttctccatgcagaggctggtgaagtgcaatgcctggccttgtcccaacactgtggactgctttgtgtccaggcccactgagaagactgtcttcacagtgttcatgattgcagtgtctggaatttgcatcctgctgaatgtcactgaattgtgttatttgctaattagatattgttctgggaagtcaaaaaagccagtttgataagcttagatctgatccagacatgataagatacattgatgagtttggacaaaccaaaactagaatgcagtgaaaaaaatgccttatttgtgaaatttgtgatgctattgccttatttgtaaccattataagctgcaataaacaagtttctagaactgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 23 - DNA, 6496 bp, synthetic construct
  source 1..6496: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattctggtaccttgctagcaaaaactcttacataattgtaacagatttgagtttcctctggttcaggttttcttgcctctttgataatcaaatgatctgaagaaaggcatagaatttcaagggagaatctgcatgacaggattacaataaggctattcatggagactcttttattagcttacacaggatctgcatcattcttcgtctctggctttgctacaaggctccatttaaacttaacccaacttgcaggcttaactgatccaggaatgactcaatacaatgagccagctactgtagctctttttccccttttgataaggggagttaacacaatgggctttacagttcttaaatgaccgttcctgcaggtattggaaaacctgcatcgtatgcctgctgctttaggagacaacaaactcagcagcccctcccccgaagctgaagtgccaaacttaaggctccaggtaaactgcccttccgatgagagctttacatttaggcagtagccacaggcctaaacaaagtcaaatggatgaaacttgtgatctctccttggaaatatggccaagagtgaaacaactacttagttattattttaaacgcaggctgctaaatggcttgtgttggtatgtaatggcatcctagataatgggggctgccaagaaaatattcacactggcaagtcaattaaaataccgttctcttcctaagaggcctggctggattccagagcttcctttagggtaacgctcaaacctgtcatagtttattttgtttctgttttcaggcaacagatttggttattgcttttattgtatgagtgttgaaatggtgtcctaacagccatctgtactgccatcccctggtctctctgcctttccagaaaggagatgaagttgctggagcaggcaggttctctgaaaggctccctgagtgtggaagagcagctgtcactcatcagtggttgtcccaatatccaagaagcagtagagggtgccatgcacattcaggtgagtgaaggacactggcgccgcatctgtggggcctgagacattgtctcccagcctctccggaggaacagggcaggatacagagcagtggcaggtgtgggcattttgcatcttctgtaatctccaaacaaagtctgcgctaggccctttcaggctataaaggacagggtactctgatttctgctcttgatgagggttgattgtgataccaggcgagggagtaagagatacaggacaccatctctgcggctgcatctttgtcgcagaaacagacattgttcaggggacacctgagcctctgatagtcagccagtggctctggcagctcacagcagttatagggtggctgtgtccttttctaaagtcctgactttttatggcctgtggtcatggtgactccactacgggctgtgtttctgttgacgcgtacagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctgtttatgatgtgtttaaagattgggtgaattactcaggtgaacaagctactttttatcagagaacacctaaaaacacgttcaagagggtttgggaactatacatttaatcctatgacaaactaagttggttctgtcttcacctgttttggtgaggttgtgtaagagttggtgtttgctcaggaagagatttaagcatgcttgcttacccagactcagagaagtctccctgttctgtcctagctagtgattcctgtgttgtgtgcattcgtcttttccagagcaaaccgcccagagtagaagcctgcaggccaccatggactggggaacactgcaaacaatcctgggaggtgtgaacaagcacagcacaagcattggcaagatctggctgacagtgctgttcatctttagaatcatgatcctggttgtggctgctaaggaagtgtggggagatgagcaggctgactttgtttgtaataccctgcaacctggctgcaagaatgtgtgttatgaccactacttccccatcagccacatcagactgtgggccctgcaactgatctttgtgtccacccctgccctgctggtggccatgcatgtggcctacagaaggcatgagaagaagaggaaattcatcaagggagagatcaagagtgagttcaaggacattgaggaaatcaaaacccagaaggtgaggattgaaggctctctgtggtggacctacaccagcagcatcttcttcagagtgatctttgaggcagcctttatgtatgtgttctatgtgatgtatgatggcttctccatgcagagactggtgaagtgcaatgcctggccatgtcctaacacagtggactgctttgtgtctagacctacagaaaagacagtgtttacagtctttatgattgcagtgtctggcatctgcattctgctgaatgtgactgagctgtgctacctgctcattaggtactgtagtggaaaaagcaagaaacctgtgtgataagcttagatctgatccagacatgataagatacattgatgagtttggacaaaccaaaactagaatgcagtgaaaaaaatgccttatttgtgaaatttgtgatgctattgccttatttgtaaccattataagctgcaataaacaagtttctagaactgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 24 - DNA, 5499 bp, synthetic construct
  source 1..5499: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattctggtaccttgctagcaaaaactcttacataattgtaacagatttgagtttcctctggttcaggttttcttgcctctttgataatcaaatgatctgaagaaaggcatagaatttcaagggagaatctgcatgacaggattacaataaggctattcatggagactcttttattagcttacacaggatctgcatcattcttcgtctctggctttgctacaaggctccatttaaacttaacccaacttgcaggcttaactgatccaggaatgactcaatacaatgagccagctactgtagctctttttccccttttgataaggggagttaacacaatgggctttacagttcttaaatgaacgcgtacagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctgtttatgatgtgtttaaagattgggtgaattactcaggtgaacaagctactttttatcagagaacacctaaaaacacgttcaagagggtttgggaactatacatttaatcctatgacaaactaagttggttctgtcttcacctgttttggtgaggttgtgtaagagttggtgtttgctcaggaagagatttaagcatgcttgcttacccagactcagagaagtctccctgttctgtcctagctagtgattcctgtgttgtgtgcattcgtcttttccagagcaaaccgcccagagtagaagcctgcaggccaccatggattggggcacactgcagactatcctggggggtgtgaacaaacactccaccagcattggaaagatctggctcacagtcctcttcatttttaggattatgatcctggttgtggctgcaaaggaggtgtggggagatgagcaggctgactttgtctgcaacaccctgcagccaggctgcaagaatgtgtgctatgatcactacttccccatctcccacatcaggctatgggccctgcagctgatctttgtgtccactccagcactcctagtggccatgcatgtggcctacaggagacatgagaagaagaggaagttcatcaagggggagataaagagtgaatttaaggacattgaggagatcaaaacccagaaggtcagaattgaaggctccctgtggtggacctacacaagcagcatcttcttcagggtcatctttgaagcagccttcatgtatgtcttctatgtcatgtatgatggcttctccatgcagaggctggtgaagtgcaatgcctggccttgtcccaacactgtggactgctttgtgtccaggcccactgagaagactgtcttcacagtgttcatgattgcagtgtctggaatttgcatcctgctgaatgtcactgaattgtgttatttgctaattagatattgttctgggaagtcaaaaaagccagtttgataagcttagatctgcctgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatctagaactgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 25 - DNA, 5499 bp, synthetic construct
  source 1..5499: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattctggtaccttgctagcaaaaactcttacataattgtaacagatttgagtttcctctggttcaggttttcttgcctctttgataatcaaatgatctgaagaaaggcatagaatttcaagggagaatctgcatgacaggattacaataaggctattcatggagactcttttattagcttacacaggatctgcatcattcttcgtctctggctttgctacaaggctccatttaaacttaacccaacttgcaggcttaactgatccaggaatgactcaatacaatgagccagctactgtagctctttttccccttttgataaggggagttaacacaatgggctttacagttcttaaatgaacgcgtacagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctgtttatgatgtgtttaaagattgggtgaattactcaggtgaacaagctactttttatcagagaacacctaaaaacacgttcaagagggtttgggaactatacatttaatcctatgacaaactaagttggttctgtcttcacctgttttggtgaggttgtgtaagagttggtgtttgctcaggaagagatttaagcatgcttgcttacccagactcagagaagtctccctgttctgtcctagctagtgattcctgtgttgtgtgcattcgtcttttccagagcaaaccgcccagagtagaagcctgcaggccaccatggactggggaacactgcaaacaatcctgggaggtgtgaacaagcacagcacaagcattggcaagatctggctgacagtgctgttcatctttagaatcatgatcctggttgtggctgctaaggaagtgtggggagatgagcaggctgactttgtttgtaataccctgcaacctggctgcaagaatgtgtgttatgaccactacttccccatcagccacatcagactgtgggccctgcaactgatctttgtgtccacccctgccctgctggtggccatgcatgtggcctacagaaggcatgagaagaagaggaaattcatcaagggagagatcaagagtgagttcaaggacattgaggaaatcaaaacccagaaggtgaggattgaaggctctctgtggtggacctacaccagcagcatcttcttcagagtgatctttgaggcagcctttatgtatgtgttctatgtgatgtatgatggcttctccatgcagagactggtgaagtgcaatgcctggccatgtcctaacacagtggactgctttgtgtctagacctacagaaaagacagtgtttacagtctttatgattgcagtgtctggcatctgcattctgctgaatgtgactgagctgtgctacctgctcattaggtactgtagtggaaaaagcaagaaacctgtgtgataagcttagatctgcctgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatctagaactgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 26 - DNA, 6575 bp, synthetic construct
  source 1..6575: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattctggtaccttgctagcaaaaactcttacataattgtaacagatttgagtttcctctggttcaggttttcttgcctctttgataatcaaatgatctgaagaaaggcatagaatttcaagggagaatctgcatgacaggattacaataaggctattcatggagactcttttattagcttacacaggatctgcatcattcttcgtctctggctttgctacaaggctccatttaaacttaacccaacttgcaggcttaactgatccaggaatgactcaatacaatgagccagctactgtagctctttttccccttttgataaggggagttaacacaatgggctttacagttcttaaatgaccgttcctgcaggtattggaaaacctgcatcgtatgcctgctgctttaggagacaacaaactcagcagcccctcccccgaagctgaagtgccaaacttaaggctccaggtaaactgcccttccgatgagagctttacatttaggcagtagccacaggcctaaacaaagtcaaatggatgaaacttgtgatctctccttggaaatatggccaagagtgaaacaactacttagttattattttaaacgcaggctgctaaatggcttgtgttggtatgtaatggcatcctagataatgggggctgccaagaaaatattcacactggcaagtcaattaaaataccgttctcttcctaagaggcctggctggattccagagcttcctttagggtaacgctcaaacctgtcatagtttattttgtttctgttttcaggcaacagatttggttattgcttttattgtatgagtgttgaaatggtgtcctaacagccatctgtactgccatcccctggtctctctgcctttccagaaaggagatgaagttgctggagcaggcaggttctctgaaaggctccctgagtgtggaagagcagctgtcactcatcagtggttgtcccaatatccaagaagcagtagagggtgccatgcacattcaggtgagtgaaggacactggcgccgcatctgtggggcctgagacattgtctcccagcctctccggaggaacagggcaggatacagagcagtggcaggtgtgggcattttgcatcttctgtaatctccaaacaaagtctgcgctaggccctttcaggctataaaggacagggtactctgatttctgctcttgatgagggttgattgtgataccaggcgagggagtaagagatacaggacaccatctctgcggctgcatctttgtcgcagaaacagacattgttcaggggacacctgagcctctgatagtcagccagtggctctggcagctcacagcagttatagggtggctgtgtccttttctaaagtcctgactttttatggcctgtggtcatggtgactccactacgggctgtgtttctgttgacgcgtacagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctgtttatgatgtgtttaaagattgggtgaattactcaggtgaacaagctactttttatcagagaacacctaaaaacacgttcaagagggtttgggaactatacatttaatcctatgacaaactaagttggttctgtcttcacctgttttggtgaggttgtgtaagagttggtgtttgctcaggaagagatttaagcatgcttgcttacccagactcagagaagtctccctgttctgtcctagctagtgattcctgtgttgtgtgcattcgtcttttccagagcaaaccgcccagagtagaagcctgcaggccaccatggattggggcacactgcagactatcctggggggtgtgaacaaacactccaccagcattggaaagatctggctcacagtcctcttcatttttaggattatgatcctggttgtggctgcaaaggaggtgtggggagatgagcaggctgactttgtctgcaacaccctgcagccaggctgcaagaatgtgtgctatgatcactacttccccatctcccacatcaggctatgggccctgcagctgatctttgtgtccactccagcactcctagtggccatgcatgtggcctacaggagacatgagaagaagaggaagttcatcaagggggagataaagagtgaatttaaggacattgaggagatcaaaacccagaaggtcagaattgaaggctccctgtggtggacctacacaagcagcatcttcttcagggtcatctttgaagcagccttcatgtatgtcttctatgtcatgtatgatggcttctccatgcagaggctggtgaagtgcaatgcctggccttgtcccaacactgtggactgctttgtgtccaggcccactgagaagactgtcttcacagtgttcatgattgcagtgtctggaatttgcatcctgctgaatgtcactgaattgtgttatttgctaattagatattgttctgggaagtcaaaaaagccagtttgataagcttagatctgcctgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatctagaactgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 27 - DNA, 6575 bp, synthetic construct
  source 1..6575: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattctggtaccttgctagcaaaaactcttacataattgtaacagatttgagtttcctctggttcaggttttcttgcctctttgataatcaaatgatctgaagaaaggcatagaatttcaagggagaatctgcatgacaggattacaataaggctattcatggagactcttttattagcttacacaggatctgcatcattcttcgtctctggctttgctacaaggctccatttaaacttaacccaacttgcaggcttaactgatccaggaatgactcaatacaatgagccagctactgtagctctttttccccttttgataaggggagttaacacaatgggctttacagttcttaaatgaccgttcctgcaggtattggaaaacctgcatcgtatgcctgctgctttaggagacaacaaactcagcagcccctcccccgaagctgaagtgccaaacttaaggctccaggtaaactgcccttccgatgagagctttacatttaggcagtagccacaggcctaaacaaagtcaaatggatgaaacttgtgatctctccttggaaatatggccaagagtgaaacaactacttagttattattttaaacgcaggctgctaaatggcttgtgttggtatgtaatggcatcctagataatgggggctgccaagaaaatattcacactggcaagtcaattaaaataccgttctcttcctaagaggcctggctggattccagagcttcctttagggtaacgctcaaacctgtcatagtttattttgtttctgttttcaggcaacagatttggttattgcttttattgtatgagtgttgaaatggtgtcctaacagccatctgtactgccatcccctggtctctctgcctttccagaaaggagatgaagttgctggagcaggcaggttctctgaaaggctccctgagtgtggaagagcagctgtcactcatcagtggttgtcccaatatccaagaagcagtagagggtgccatgcacattcaggtgagtgaaggacactggcgccgcatctgtggggcctgagacattgtctcccagcctctccggaggaacagggcaggatacagagcagtggcaggtgtgggcattttgcatcttctgtaatctccaaacaaagtctgcgctaggccctttcaggctataaaggacagggtactctgatttctgctcttgatgagggttgattgtgataccaggcgagggagtaagagatacaggacaccatctctgcggctgcatctttgtcgcagaaacagacattgttcaggggacacctgagcctctgatagtcagccagtggctctggcagctcacagcagttatagggtggctgtgtccttttctaaagtcctgactttttatggcctgtggtcatggtgactccactacgggctgtgtttctgttgacgcgtacagaggacaacgaccacagccatccctgaaccccgcccacggcacagcgccggagccggggtctggggcgccgcttcctggggggtcccgactctcagccgcccccgcttcacccgggccgccaaggggctgggggaggcggcgctcggggtaaccgggggagactcagggcgctgggggcacttggggaactcatgggggctcaaaggaactaggagatcgggacctcgaaggggacttggggggttcggggctttcgggggcggtcgggggttcgcggacccgggaagctctgaggacccagaggccgggcgcgctccgcccgcggcgccgccccctccgtaactttcccagtctccgagggaagaggcggggtgtggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcaggtgagcccgccggccccggactgcccggccaggaacctggcgcggggagggaccgcgagacccagagcggttgcccggccgcgtgggtctcggggaaccggggggctggaccaacacacgtccttgggccggggggcgggggccgccttctggagcgggcgtttctgtttatgatgtgtttaaagattgggtgaattactcaggtgaacaagctactttttatcagagaacacctaaaaacacgttcaagagggtttgggaactatacatttaatcctatgacaaactaagttggttctgtcttcacctgttttggtgaggttgtgtaagagttggtgtttgctcaggaagagatttaagcatgcttgcttacccagactcagagaagtctccctgttctgtcctagctagtgattcctgtgttgtgtgcattcgtcttttccagagcaaaccgcccagagtagaagcctgcaggccaccatggactggggaacactgcaaacaatcctgggaggtgtgaacaagcacagcacaagcattggcaagatctggctgacagtgctgttcatctttagaatcatgatcctggttgtggctgctaaggaagtgtggggagatgagcaggctgactttgtttgtaataccctgcaacctggctgcaagaatgtgtgttatgaccactacttccccatcagccacatcagactgtgggccctgcaactgatctttgtgtccacccctgccctgctggtggccatgcatgtggcctacagaaggcatgagaagaagaggaaattcatcaagggagagatcaagagtgagttcaaggacattgaggaaatcaaaacccagaaggtgaggattgaaggctctctgtggtggacctacaccagcagcatcttcttcagagtgatctttgaggcagcctttatgtatgtgttctatgtgatgtatgatggcttctccatgcagagactggtgaagtgcaatgcctggccatgtcctaacacagtggactgctttgtgtctagacctacagaaaagacagtgtttacagtctttatgattgcagtgtctggcatctgcattctgctgaatgtgactgagctgtgctacctgctcattaggtactgtagtggaaaaagcaagaaacctgtgtgataagcttagatctgcctgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatctagaactgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 28 - DNA, 678 bp, synthetic construct
  source 1..678: mol_type = other DNA
atggactggggaacactgcaaacaatcctgggcggcgtgaacaagcacagcacaagcatcggcaagatctggctgaccgtgctgttcatctttagaatcatgatcctggttgtggctgctaaggaagtgtggggcgatgagcaggccgacttcgtttgtaataccctgcaacctggctgcaagaacgtgtgttacgaccactacttccccatcagccacatcagactgtgggccctgcaactgatcttcgtgtccacccctgccctgctggtggccatgcatgtggcctacagaaggcacgagaagaagcggaaattcatcaagggcgagatcaagagcgagttcaaggacatcgaggaaatcaaaacccagaaggtgcggatcgaaggctctctgtggtggacctacaccagcagcatcttcttcagagtgatcttcgaggccgcctttatgtacgtgttctacgtgatgtatgatggcttctccatgcagagactggtgaagtgcaacgcctggccatgtcctaacaccgtcgactgcttcgtgtctagacctacagaaaagacagtgtttaccgtctttatgatcgccgtgtctggcatctgcattctgctgaacgtgaccgagctgtgctacctgctcattcggtactgtagcggaaaaagcaagaaacctgtg

SEQ ID NO: 29 - protein, 226 aa, Homo sapiens
MDWGTLQTILGGVNKHSTSIGKIWLTVLFIFRIMILVVAAKEVWGDEQADFVCNTLQPGCKNVCYDHYFPISHIRLWALQLIFVSTPALLVAMHVAYRRHEKKRKFIKGEIKSEFKDIEEIKTQKVRIEGSLWWTYTSSIFFRVIFEAAFMYVFYVMYDGFSMQRLVKCNAWPCPNTVDCFVSRPTEKTVFTVFMIAVSGICILLNVTELCYLLIRYCSGKSKKPV